SEQUENCE LISTING (18 sequences)

SEQ ID NO: 1 - DNA, 37 bp, synthetic construct
  source 1..37: mol_type = other DNA
cggccgctaatacgactcactataagggtcagatcgc

SEQ ID NO: 2 - DNA, 138 bp, synthetic construct
  source 1..138: mol_type = other DNA
ttttttttttttttttttttttttttttttttttttttttttttttttttttttttttttttttttttttttttttttttttttttttttttttttttttttttttttttttttttttttgctagctccagggtgtgg

SEQ ID NO: 3 - DNA, 170 bp, synthetic construct
  source 1..170: mol_type = other DNA
gtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtagtttaaaccccttc

SEQ ID NO: 4 - DNA, 74 bp, synthetic construct
  source 1..74: mol_type = other DNA
gtcagatcgcctggagacgccatccacgctgttttgacctccatagaagaccgagctcggatccagtacccttc

SEQ ID NO: 5 - DNA, 1552 bp, synthetic construct
  source 1..1552: mol_type = other DNA
aggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttcgtggctgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagagacaattaacttcgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 6 - DNA, 289 bp, synthetic construct
  source 1..289: mol_type = other DNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 7 - DNA, 720 bp, synthetic construct
  source 1..720: mol_type = other DNA
atggtgtccaagggcgaggaactgttcaccggcgtggtgcccatcctggtggaactggacggcgacgtgaacggccacaagttcagcgtgagaggcgagggcgaaggcgacgccacaaacggaaagctgaccctgaagttcatctgcaccaccggcaagctgcccgtgccttggcctaccctcgtgaccacactgacctacggcgtgcagtgcttcagcagataccccgaccacatcaagagacacgatttcttcaagagcgccctgcccgagggctacgtgcaggaacggaccatcagcttcaaggacgacggcacctacaagacaagagccgaagtgaagttcgagggcgacaccctcgtgaaccggatcgagctgaagggcatcgacttcaaagaggacggcaacatcctgggccacaagctggagtacaacttcaacagccacaacgtgtacatcaccgccgacaagcagaaaaacggcatcaaagccaacttcaagatccggcacaacgtggaggacggcagcgtgcagctggccgaccactaccagcagaacacccccatcggagacggccccgtgctgctgcccgacaaccactacctgagcacacaaagcaagctgagcaaggaccccaacgagaagcgggaccacgccgtgctgctggaatttgtgaccgccgctggcatcacccacggcaaggacgagctgtacaagtga

SEQ ID NO: 8 - DNA, 720 bp, synthetic construct
  source 1..720: mol_type = other DNA
atggttagtaaaggagaagaactcttcaccggcgtagttccgattctcgttgagctggacggagacgttaacgggcacaaattctctgtaaggggtgaaggggaaggcgacgctaccaacggaaaactgaccttaaaatttatttgtacaacaggtaagctcccagttccctggcccacccttgttaccactctgactcacggcgttcagtgtttctctcgttatccagatcatataaagcggcacgacttctttaagtccgctctccccgaaggctacgtccaagaaagaaccattagttttaaggacgacggaacttacaaaaccagagcagaggtcaagttcgagggagatactctcgtcaatcggatcgaactgaaaggaatagactttaaagaggacggcaatatcctgggtcacaagctggagtataatttcaatagccataacgtgtatatcacggccgataagcagaagaacggtattaaggcaaattttaagattcgccataacgtagaagacggttccgtccaattggcggaccattatcaacaaaacactccaattggagacggcccggtattactgcctgataatcactacctgtccacccaatcaaaactctccaaagatcccaacgagaaacgggaccacgcggttctgttagaatttgtaacggccgctggtattacgcacggtaaggacgaactgtataagtga

SEQ ID NO: 9 - DNA, 720 bp, synthetic construct
  source 1..720: mol_type = other DNA
atggtgtccaagggcgaggaactgttcaccggcgtggtgcccatcctggtggaactggatggcgacgtgaacggccacaagttctctgtgcggggagagggcgaaggcgacgccacaaatggcaagctgaccctgaagttcatctgcaccaccggcaagctgcccgtgccttggcctaccctcgtgaccacactgtctcacggcgtgcagtgcttcgccagataccccgaccacatgaagcagcacgatttcttcaagagcgccatgcccgagggctacgtgcaggaacggaccatcttcttcaaggacgacggcacctacaagaccagagccgaagtgaagttcgagggcgacaccctcgtgaaccggatcgagctgaagggcgtggacttcaaagaggacggcaacatcctgggccacaagctggagtacaacttcaacagccacaacatctacatcatggccgtgaagcagaaaaacggcatcaaagtgaacttcaagatccggcacaacgtggaagatggcagcgtgcagctggccgaccactaccagcagaacacccccatcggagatggccccgtgctgctgcctgatagccactacctgagcacccagagcaagctgagcaaggaccccaacgagaagcgggaccacatggtgctgctggaatttcggaccgccgctggcatcaccctgggcatggatgagctgtacaagtga

SEQ ID NO: 10 - DNA, 936 bp, synthetic construct
  source 1..936: mol_type = other DNA
atggtggcggaaggctccgtcgccaggcagcctgacctcttgacctgcgaacatgaagagatccacctcgccggctcgatccagccgcatggcgcgcttctggtcgtcagcgaacatgatcatcgcgtcatccaggccagcgccaacgccgcggaatttctgaatctcggaagcgtactcggcgttccgctcgccgagatcgacggcgatctgttgatcaagatcctgccgcatctcgatcccaccgccgaaggcatgccggtcgcggtgcgctgccggatcggcaatccctctacggagtactgcggtctgatgcatcggcctccggaaggcgggctgatcatcgaactcgaacgtgccggcccgtcgatcgatctgtcaggcacgctggcgccggcgctggagcggatccgcacggcgggttcactgcgcgcgctgtgcgatgacaccgtgctgctgtttcagcagtgcaccggctacgaccgggtgatggtgtatcgtttcgatgagcaaggccacggcctggtattctccgagtgccatgtgcctgggctcgaatcctatttcggcaaccgctatccgtcgtcgactgtcccgcagatggcgcggcagctgtacgtgcggcagcgcgtccgcgtgctggtcgacgtcacctatcagccggtgccgctggagccgcggctgtcgccgctgaccgggcgcgatctcgacatgtcgggctgcttcctgcgctcgatgtcgccgtgccatctgcagttcctgaaggacatgggcgtgcgcgccaccctggcggtgtcgctggtggtcggcggcaagctgtggggcctggttgtctgtcaccattatctgccgcgcttcatccgtttcgagctgcgggcgatctgcaaacggctcgccgaaaggatcgcgacgcggatcaccgcgcttgagagctga

SEQ ID NO: 11 - DNA, 572 bp, synthetic construct
  source 1..572: mol_type = other DNA
ccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 12 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 13 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 14 - DNA, 19 bp, synthetic construct
  source 1..19: mol_type = other DNA
taatacgactcactatagg

SEQ ID NO: 15 - DNA, 8103 bp, synthetic construct
  source 1..8103: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttattctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcactcattaggcaccccaggctttacactttatgcttccggctcgtataatgtgtggaattgtgagcggataacaatttcacacaggaaacagcatcgtgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtagtttaaaccccttctttatggtgtccaagggcgaggaactgttcaccggcgtggtgcccatcctggtggaactggacggcgacgtgaacggccacaagttcagcgtgagaggcgagggcgaaggcgacgccacaaacggaaagctgaccctgaagttcatctgcaccaccggcaagctgcccgtgccttggcctaccctcgtgaccacactgacctacggcgtgcagtgcttcagcagataccccgaccacatcaagagacacgatttcttcaagagcgccctgcccgagggctacgtgcaggaacggaccatcagcttcaaggacgacggcacctacaagacaagagccgaagtgaagttcgagggcgacaccctcgtgaaccggatcgagctgaagggcatcgacttcaaagaggacggcaacatcctgggccacaagctggagtacaacttcaacagccacaacgtgtacatcaccgccgacaagcagaaaaacggcatcaaagccaacttcaagatccggcacaacgtggaggacggcagcgtgcagctggccgaccactaccagcagaacacccccatcggagacggccccgtgctgctgcccgacaaccactacctgagcacacaaagcaagctgagcaaggaccccaacgagaagcgggaccacgccgtgctgctggaatttgtgaccgccgctggcatcacccacggcaaggacgagctgtacaagtgacccgggttaccatggtgtccaagggcgaggaactgttcaccggcgtggtgcccatcctggtggaactggatggcgacgtgaacggccacaagttctctgtgcggggagagggcgaaggcgacgccacaaatggcaagctgaccctgaagttcatctgcaccaccggcaagctgcccgtgccttggcctaccctcgtgaccacactgtctcacggcgtgcagtgcttcgccagataccccgaccacatgaagcagcacgatttcttcaagagcgccatgcccgagggctacgtgcaggaacggaccatcttcttcaaggacgacggcacctacaagaccagagccgaagtgaagttcgagggcgacaccctcgtgaaccggatcgagctgaagggcgtggacttcaaagaggacggcaacatcctgggccacaagctggagtacaacttcaacagccacaacatctacatcatggccgtgaagcagaaaaacggcatcaaagtgaacttcaagatccggcacaacgtggaagatggcagcgtgcagctggccgaccactaccagcagaacacccccatcggagatggccccgtgctgctgcctgatagccactacctgagcacccagagcaagctgagcaaggaccccaacgagaagcgggaccacatggtgctgctggaatttcggaccgccgctggcatcaccctgggcatggatgagctgtacaagtgataatactaaatcgcactgtcggcgtccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggatgagctgtacaagtgaaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttcgtggctgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagagacaattaacttcgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttctcagatcctgcattaatgaatcggccaacgcgcggggagaggcggtttgcgtattggctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttcttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagtactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttc

SEQ ID NO: 16 - DNA, 9058 bp, synthetic construct
  source 1..9058: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttattctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcactcattaggcaccccaggctttacactttatgcttccggctcgtataatgtgtggaattgtgagcggataacaatttcacacaggaaacagcatcgtgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtagtttaaaccccttcaccatggttagtaaaggagaagaactcttcaccggcgtagttccgattctcgttgagctggacggagacgttaacgggcacaaattctctgtaaggggtgaaggggaaggcgacgctaccaacggaaaactgaccttaaaatttatttgtacaacaggtaagctcccagttccctggcccacccttgttaccactctgactcacggcgttcagtgtttctctcgttatccagatcatataaagcggcacgacttctttaagtccgctctccccgaaggctacgtccaagaaagaaccattagttttaaggacgacggaacttacaaaaccagagcagaggtcaagttcgagggagatactctcgtcaatcggatcgaactgaaaggaatagactttaaagaggacggcaatatcctgggtcacaagctggagtataatttcaatagccataacgtgtatatcacggccgataagcagaagaacggtattaaggcaaattttaagattcgccataacgtagaagacggttccgtccaattggcggaccattatcaacaaaacactccaattggagacggcccggtattactgcctgataatcactacctgtccacccaatcaaaactctccaaagatcccaacgagaaacgggaccacgcggttctgttagaatttgtaacggccgctggtattacgcacggtaaggacgaactgtataagtgaagcgcttttttccattgtccaagggcgaggaactgttcaccggcgtggtgcccatcctggtggaactggacggcgacgtgaacggccacaagttcagcgtgagaggcgagggcgaaggcgacgccacaaacggaaagctgaccctgaagttcatctgcaccaccggcaagctgcccgtgccttggcctaccctcgtgaccacactgacctacggcgtgcagtgcttcagcagataccccgaccacatcaagagacacgatttcttcaagagcgccctgcccgagggctacgtgcaggaacggaccatcagcttcaaggacgacggcacctacaagacaagagccgaagtgaagttcgagggcgacaccctcgtgaaccggatcgagctgaagggcatcgacttcaaagaggacggcaacatcctgggccacaagctggagtacaacttcaacagccacaacgtgtacatcaccgccgacaagcagaaaaacggcatcaaagccaacttcaagatccggcacaacgtggaggacggcagcgtgcagctggccgaccactaccagcagaacacccccatcggagacggccccgtgctgctgcccgacaaccactacctgagcacacaaagcaagctgagcaaggaccccaacgagaagcgggaccacgccgtgctgctggaatttgtgaccgccgctggcatcacccacggcaaggacgagctgtacaagtgacccgggtttttccattggcggaaggctccgtcgccaggcagcctgacctcttgacctgcgaacatgaagagatccacctcgccggctcgatccagccgcatggcgcgcttctggtcgtcagcgaacatgatcatcgcgtcatccaggccagcgccaacgccgcggaatttctgaatctcggaagcgtactcggcgttccgctcgccgagatcgacggcgatctgttgatcaagatcctgccgcatctcgatcccaccgccgaaggcatgccggtcgcggtgcgctgccggatcggcaatccctctacggagtactgcggtctgatgcatcggcctccggaaggcgggctgatcatcgaactcgaacgtgccggcccgtcgatcgatctgtcaggcacgctggcgccggcgctggagcggatccgcacggcgggttcactgcgcgcgctgtgcgatgacaccgtgctgctgtttcagcagtgcaccggctacgaccgggtgatggtgtatcgtttcgatgagcaaggccacggcctggtattctccgagtgccatgtgcctgggctcgaatcctatttcggcaaccgctatccgtcgtcgactgtcccgcagatggcgcggcagctgtacgtgcggcagcgcgtccgcgtgctggtcgacgtcacctatcagccggtgccgctggagccgcggctgtcgccgctgaccgggcgcgatctcgacatgtcgggctgcttcctgcgctcgatgtcgccgtgccatctgcagttcctgaaggacatgggcgtgcgcgccaccctggcggtgtcgctggtggtcggcggcaagctgtggggcctggttgtctgtcaccattatctgccgcgcttcatccgtttcgagctgcgggcgatctgcaaacggctcgccgaaaggatcgcgacgcggatcaccgcgcttgagagctgataatctagatacactaaatcgcactgtcggcgtccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggatgagctgtacaagtgaaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttcgtggctgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagagacaattaacttcgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttctcagatcctgcattaatgaatcggccaacgcgcggggagaggcggtttgcgtattggctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttcttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagtactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttc

SEQ ID NO: 17 - DNA, 11935 bp, synthetic construct
  source 1..11935: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttattctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcactcattaggcaccccaggctttacactttatgcttccggctcgtataatgtgtggaattgtgagcggataacaatttcacacaggaaacagcatcgtgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagaccgagctcggatccagtacccttcaccatggccgtggaaaagaccaacagcagcagctccctggccgaagtgatcgacagaatcctggacaagggcatcgtgatcgacgcctgggtgcgcgtgtccctcgtgggaattgagctgctggccatcgaggcccggatcgtgattgccagcgtggaaacctacctgaagtacgccgaggccgtgggcctgacacagagtgctgctgtgcctgcttgattaccatggtgaccaccaccaaagtgaaccacaagcgggccgtgctgagactgaggcctggccagtttgtcgtgacccccgccattgagcgggtggccattagagccctgagatacctgaagtccggcttccccgtgcacctgagaggacctgccggaaccggcaagaccacactggccatgcacctggccaactgcctggacagacccgtgatgctgctgttcggcgacgaccagttcaagagcagcgacctgatcggcagcgagagcggctacacccacaagaaggtgctggacaactacatccacagcgtcgtgaagctggaagatgagttcaagcagaactgggtggacagcagactgaccctggcctgccgggaaggcttcaccctggtgtacgacgagttcaaccggtccagacccgaagtgaacaacgtgctgctgagcgccctggaagagaagatcctgagcctgccccccagcagcaaccagccagagtacctgagcgtgaacccccagttcagagtgatcttcaccagcaaccccgaggaatatgccggcgtgcacagcacccaggacgccctgatggaccggctcgtgaccatctccatgcccgagcccgatgagatcacccagaccgagatcctgatccagaaaacaaacatcgaccgcgagagcgccaacttcatcgtgcggctcgtgaagtccttcagactggccacaggcgccgagaaaaccagcggcctgagaagctgcctgatgatcgccaaagtgtgcgccgacaacaacatccccgtgaccaccgagagcctggacttccccgatatcgccatcgacatcctgttcaaccgcagccacctgagcatgagcgagtccaccaacatctttctggaactgctggataagttcagcgccgaggaactggaaatcctgaacaacagagtgaccggcgacaacgacttcctgatcgacaacagccagttcgtgtcccagcagctggccggacagcccaacggagcgccaggttccggggctactaacttcagcctccttaaacaggctggcgatgtggaagaaaatcccggaccagtgctgcccaccagaccccagaccaacagcagccggaccatcaacaccagcacccagggcagcaccctggccgacatcctggaaagagtgctggacaagggcatcgtgatcgccggggacatcagcatctctatcgccagcaccgagctggtgcacatccggatcagactgctgatctccagcgtggacaaggccaaagagatgggcatcaactggtgggagagcgacccctacctgagcaccaaggcccagagactggtggaagagaaccagcagctccagcacagactggaaagcctggaagccaagctgaacagcctgaccagcagcagcgtgaaagaggaaatccccctggccgccgacgtgaaggacgacctgtatcagaccagcgccaagatccccagccccgtggatacccctatcgaggtgctggacttccaggcccagtctagcggcggcacacccccttacgtgaacacctctatggaaatactggattttcaggctcagacctccgccgagagcagcagccctgtgggctctaccgtggaaatactggacttccaggcacagacaagcgaggaatccagctcccccgtggtgtccacagtggaaatactggatttccaggcccagacttctgaagagtccagctctccagtgggaagcactgtggaaatactggactttcaggcccagaccagtgaagagatccccagctctgtggaccccgccatcgatgtgggtgccccgggatctggcgcaacaaattttagtctcctcaagcaggcaggagatgtcgaggaaaaccctggacccgtggtgtgcacccctgccgagaacttcaacaacagcctgacaatcgccagcaagcccaagaacgaggccggactggctcctctgctgctgaccgtgctggaactcgtgcggcagctgatggaagcccaagtgatccggcggatggaagaggacctgctgagcgagcccgacctggaaagagccgccgatagcctccagaagctggaagaacagatcctgcacctgtgcgagatgttcgaggtggaccccgccgacctgaacatcaacctgggcgagatcggcaccctgctgcctagcagcggcagctactatcctggccagccaagcagcagaccctctgtgctggaactgctggaccggctgctgaacaccggcatcgtggtggacggcgagattgacctgggaatcgcccagatcgacctgatccacgccaagctgagactggtgctgacctccaagcccatcggggcacctggctcaggagcgaccaacttctcattgctcaaacaagccggagatgttgaggagaatccaggccctgtgagcatccccctgtacctgtacggcatcttccccaacaccatccccgagacactggaactggaaggcctggacaagcagcccgtgcacagccaggtggtggacgagttctgcttcctgtacagcgaggcccggcaggaaaagtacctggccagcagacggaacctgctgacccacgagaaggtgctggaacagaccatgcacgccggcttcagagtgctgctgcccctgagatttggcctggtcgtgaaggactgggagacaatcatgagccagctgatcaacccccacaaggaccagctgaaccagctgttccagaagctggccggcaagcgggaagtgtccatcaagattttctgggacgccaaggccgagcttcagaccatgatggaaagccaccaggacctgaagcagcagcgggacaacatggaaggcaagaaactgagcatggaagaagtgatccagatcgggcagctgatcgagatcaacctgctggcccggaagcaggccgtgatcgaggtgttcagccaggaactgaaccccttcgcccaggaaatcgtggtgtccgaccccatgaccgaggaaatgatctacaacgccgccttcctgatcccctgggagagcgagagcgagttcagcgagcgggtggaagtgatcgaccagaagttcggcgaccggctgcggatcagatacaacaacttcaccgccccctacacctttgcccagctggactctggcgctcccgggtcaggtgccactaatttttcattgttgaagcaagctggggatgttgaagagaacccagggcctgtgctgaccaaactcctgctgctgcccatcatgggacccctgaatggcgtcgtgtggatcgccgagcagatccaggaacggaccaacaccgagttcgacgcccaggaaaacctgcacaaacagctgctgagcctccagctgagcttcgacatcggcgagatcggagaggaagagttcgagattcaggaagaggaaatcctgctgaagatccaggccctggaagaagaggcccggctggaactggaagccgagcaggaagaagccagactggaactggaagctgaacaggaagatttcgagtacccaccacagttcaccgccgaagtgaacaaggatcagcatctggtgctgctgcccggtgccccgggatctggcgctactaacttcagcctccttaaacaggctggcgatgtggaagaaaatcccggaccagtggaactggaaaacctgtacacctacgcctttctggaaatccccagcagccccctgatcctgcctcagggcgctgctaatcaggtggtgctgatcaacggcaccgagctggccgccatcgtggaacctggcatcttcctggaatccttccagaacaacgacgagaagatcatccagatggccctgagccacgacagagtgatctgcgagctgttccagcagatcaccgtgctgcccctgagattcggcacctacttcaccagcaccaacaacctgctgaaccacctgaagtcccacgagaaagagtaccagaacaagctggaaaagatcaacgggaagaacgagttcaccctgaagctgatcccccggatgatcgaggaaatcgtgccctctgagggcggaggcaaggactacttcctggccaagaagcagcgctaccagaatcagaacaacttctctatcgcccaggccgccgagaagcagaacctgatcgacctgatcaccaaagtgaaccagctgcccgtggtggtgcaggaacaggaagaacagatccaaatctacctgctggtgtcctgccaggataagaccctgctgctggaacagttcctgacctggcagaaagcctgccccaagtgggatctgctgctgggcgactgcctgcccccctaccactttatcggagcgccaggttccggggcaacaaattttagtctcctcaagcaggcaggagatgtcgaggaaaaccctggacccgtggtgtccaagggcgaggaactgttcaccggcgtggtgcccatcctggtggaactggatggcgacgtgaacggccacaagttcagcgtgtccggcgagggcgaaggcgacgccacatacggaaagctgaccctgaagttcatctgcaccaccggcaagctgcccgtgccttggcctaccctcgtgaccacactgacctacggcgtgcagtgcttcgccagataccccgaccacatgaagcagcacgatttcttcaagagcgccatgcccgagggctacgtgcaggaacggaccatcttcttcaaggacgacggcaactacaagacaagagccgaagtgaagttcgagggcgacaccctcgtgaaccggatcgagctgaagggcatcgacttcaaagaggatggcaacatcctgggccacaagctggagtacaactacaacagccacaaggtgtacatcaccgccgacaagcagaaaaacggcatcaaagtgaacttcaagacccggcacaacatcgaggacggcagcgtgcagctggccgaccactaccagcagaacacccccatcggagatggccccgtgctgctgcccgacaaccactacctgagcacacaaagcgccctgagcaaggaccccaacgagaagcgggaccacatggtgctgctggaatttgtgaccgccgctggcatcaccctgggcatggacgagctgtacaagtgataatactaaatcgcactgtcggcgtccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgccctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggatgagctgtacaagtgaaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattataattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttcgtggctgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagagacaattaacttcgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttctcagatcctgcattaatgaatcggccaacgcgcggggagaggcggtttgcgtattggctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttcttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagtactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttc

SEQ ID NO: 18 - DNA, 5345 bp, synthetic construct
  source 1..5345: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtagtttaaaccccttcaccatggtgtccaagggcgaggaactgttcaccggcgtggtgcccatcctggtggaactggacggcgacgtgaacggccacaagttcagcgtgagaggcgagggcgaaggcgacgccacaaacggaaagctgaccctgaagttcatctgcaccaccggcaagctgcccgtgccttggcctaccctcgtgaccacactgacctacggcgtgcagtgcttcagcagataccccgaccacatcaagagacacgatttcttcaagagcgccctgcccgagggctacgtgcaggaacggaccatcagcttcaaggacgacggcacctacaagacaagagccgaagtgaagttcgagggcgacaccctcgtgaaccggatcgagctgaagggcatcgacttcaaagaggacggcaacatcctgggccacaagctggagtacaacttcaacagccacaacgtgtacatcaccgccgacaagcagaaaaacggcatcaaagccaacttcaagatccggcacaacgtggaggacggcagcgtgcagctggccgaccactaccagcagaacacccccatcggagacggccccgtgctgctgcccgacaaccactacctgagcacacaaagcaagctgagcaaggaccccaacgagaagcgggaccacgccgtgctgctggaatttgtgaccgccgctggcatcacccacggcaaggacgagctgtacaagtgacccgggttaccatggtgtccaagggcgaggaactgttcaccggcgtggtgcccatcctggtggaactggatggcgacgtgaacggccacaagttctctgtgcggggagagggcgaaggcgacgccacaaatggcaagctgaccctgaagttcatctgcaccaccggcaagctgcccgtgccttggcctaccctcgtgaccacactgtctcacggcgtgcagtgcttcgccagataccccgaccacatgaagcagcacgatttcttcaagagcgccatgcccgagggctacgtgcaggaacggaccatcttcttcaaggacgacggcacctacaagaccagagccgaagtgaagttcgagggcgacaccctcgtgaaccggatcgagctgaagggcgtggacttcaaagaggacggcaacatcctgggccacaagctggagtacaacttcaacagccacaacatctacatcatggccgtgaagcagaaaaacggcatcaaagtgaacttcaagatccggcacaacgtggaagatggcagcgtgcagctggccgaccactaccagcagaacacccccatcggagatggccccgtgctgctgcctgatagccactacctgagcacccagagcaagctgagcaaggaccccaacgagaagcgggaccacatggtgctgctggaatttcggaccgccgctggcatcaccctgggcatggatgagctgtacaagtgataatacctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaagagaccaataaaagatctttattttcattagatctgtgtgttggttttttgtgtgggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgtacgggccagatatacgcgtt

(19) United States
(12) Patent Application Publication
(10) Pub. No.: US 2024/0366793 A1
(43) Pub. Date: Nov. 7, 2024

(54) MAMMALIAN POLYCISTRONIC EXPRESSION SYSTEM FOR DIRECT TRANSLATION FROM RNA AND SECRETION OF CARGO

(71) Applicant: California Institute of Technology, Pasadena, CA (US)

(72) Inventors: Ishaan J. Dev, Pasadena, CA (US); Mengtong Duan, Pasadena, CA (US); Mikhail G. Shapiro, Pasadena, CA (US)

(21) Appl. No.: 18/653,868

(22) Filed: May 2, 2024

Related U.S. Application Data
(60) Provisional application No. 63/463,791, filed on May 3, 2023.

(51) Int. Cl. A61K 48/00; C12N 15/11; C12N 15/85

(52) U.S. Cl. CPC A61K 48/005; C12N 15/11; C12N 15/85; C12N 2310/3519; C12N 2840/10

(57) ABSTRACT

Disclosed herein include methods, compositions, and kits enabling expression of multiple payload proteins from a single mRNA configured to achieve a predetermined stoichiometry of first unit payload protein(s) and second unit payload protein(s) in a cell or cell-like environment. There are provided, in some embodiments, nucleic acid compositions comprising a polynucleotide comprising a first nucleic acid unit (encoding one or more first unit payload protein(s)) and a second nucleic acid unit (encoding one or more second unit payload protein(s)). The first nucleic acid unit and the second nucleic acid unit can each comprise an engineered translation initiation site (eTIS) comprising a three-nucleotide tunable element immediately upstream of a start codon. In some embodiments, the nucleic acid composition comprises at least one modified nucleotide and/or at least one nucleotide analogue or nucleotide derivative. In some embodiments, the polynucleotide comprises a disclosed eTIS combination.

### RELATED APPLICATIONS

This application claims the benefit under 35 U.S.C. § 119 (e) of U.S. Provisional Patent Application Ser. No. 63/463,791, filed May 3, 2023, the content of this related application is incorporated herein by reference in its entirety for all purposes.

### STATEMENT REGARDING FEDERALLY SPONSORED R&D

This invention was made with government support under Grant No. EB018975 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365869-US, created May 2, 2024, which is 57,297 bytes in size. The information in the electronic format of the Sequence Listing is incorporated herein by reference in its entirety.

### BACKGROUND

### Field

The present disclosure relates to the field of polynucleotide expression.

### Description of the Related Art

In the nascent era of RNA vaccines and therapeutics, the ability to express multiple proteins from a single RNA transcript and the ability to control translation product stoichiometry from a single polycistronic mammalian RNA is becoming increasingly important. For example, the current Pfizer/BioNTech and Moderna COVID-19 vaccines employ a messenger RNA (mRNA) that encodes for a single polypeptide (the spike protein) which acts as the antigen for the immune response. As mRNA vaccines and therapeutics become increasingly common and complex, there will be an increasing need for methods and compositions enabling expression of multiple polypeptides from a single RNA. Another example is the recent development of protein interaction circuits such as CHOMP. These circuits can be introduced into a cell via a mRNA, bypassing most of the central dogma processes. Proper circuit constituent stoichiometry is important for a reliable and reproducible circuit function.

Expression of multiple proteins can be done by co-introduction of multiple plasmids, RNAs, viral vectors or similar genetic constructs into a pool of cells. Such methods are problematic because the ratios of individual components in each cell do not perfectly correlate with the ratios of the parent mixture due to inherent stochasticity in the co-introduction methods. Controlling gene product stoichiometry is often done by expressing multiple RNAs from promoters of varying strengths or by titrating promoter-specific chemical inducers. These approaches, however, are not applicable for polycistronic mRNAs since transcription is bypassed.

Current technologies that allow for polycistronic expression from mammalian mRNA have limited capabilities for tuning translation stoichiometry from genes on the same mRNA. For example, a viral Internal Ribosome Entry Site (IRES) can provide an additional ribosome flux for expression of a secondary product, but the expression is severely attenuated. Additionally, IRES sequences are long, which limits other sequence content that can be included with the capacity of a vector. Another option is to link the polypeptides with a self-cleaving viral 2A sequence (such as P2A). This approach generally achieves higher expression compared to IRES but the translation rate is locked at equivalence. Additionally, 2A sequences introduce non-native amino acids to each polypeptide, which in many cases negatively affect the proper function of the protein.

There is a need for compositions and methods enabling expression of multiple proteins from a single mRNA which provide a user-friendly framework to arbitrarily and predictably tune translated product stoichiometry.

### SUMMARY

There are provided, in some embodiments, nucleic acid compositions. In some embodiments, the nucleic acid composition comprises: a polynucleotide comprising a first nucleic acid unit and a second nucleic acid unit. In some embodiments, the first nucleic acid unit encodes one or more first unit payload protein(s). In some embodiments, the second nucleic acid unit encodes one or more second unit payload protein(s). In some embodiments, the first nucleic acid unit and the second nucleic acid unit each comprise an engineered translation initiation site (eTIS) comprising a three-nucleotide tunable element immediately upstream of a start codon. In some embodiments, the polynucleotide is capable of being translated to generate the one or more first unit payload protein(s) and the one or more second unit payload protein(s), optionally the polynucleotide is a polycistronic transcript. In some embodiments, the eTIS of each of the first nucleic acid unit and the second nucleic acid unit is configured to achieve a predetermined stoichiometry of the one or more first unit payload protein(s) and one or more second unit payload protein(s) in a cell or cell-like environment. In some embodiments, (i) the nucleic acid composition comprises at least one modified nucleotide and/or at least one nucleotide analogue or nucleotide derivative; and/or (ii) the polynucleotide comprises an eTIS combination of a first eTIS of the first nucleic acid unit and a second eTIS of the second nucleic acid unit, and wherein the tunable element of the first eTIS and the second eTIS are independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

There are provided, in some embodiments, nucleic acid compositions. In some embodiments, the nucleic acid composition comprises: a promoter operably linked to a polynucleotide comprising a first nucleic acid unit and a second nucleic acid unit. In some embodiments, the first nucleic acid unit encodes one or more first unit payload protein(s). In some embodiments, the second nucleic acid unit encodes one or more second unit payload protein(s). In some embodiments, the first nucleic acid unit and the second nucleic acid unit each comprise an engineered translation initiation site (eTIS) comprising a three-nucleotide tunable element immediately upstream of a start codon. In some embodiments, the promoter is capable of inducing transcription of the first nucleic acid unit and the second nucleic acid unit to generate a polycistronic transcript. In some embodiments, the polycistronic transcript is capable of being translated to generate the one or more first unit payload protein(s) and the one or more second unit payload protein(s). In some embodiments, the eTIS of each of the first nucleic acid unit and the second nucleic acid unit is configured to achieve a predetermined stoichiometry of the one or more first unit payload protein(s) and one or more second unit payload protein(s) in a cell or cell-like environment. In some embodiments, (i) the nucleic acid composition comprises at least one modified nucleotide and/or at least one nucleotide analogue or nucleotide derivative; and/or (ii) the polynucleotide comprises an eTIS combination of a first eTIS of the first nucleic acid unit and a second eTIS of the second nucleic acid unit, and wherein the tunable element of the first eTIS and the second eTIS are independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

In some embodiments, the eTIS comprises at least one modified nucleotide and/or at least one nucleotide analogue or nucleotide derivative. In some embodiments, the at least one modified nucleotide and/or at least one nucleotide analogues is selected from a backbone modified nucleotide, a sugar modified nucleotide and/or a base modified nucleotide, or any combination thereof. In some embodiments, the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, N-1-methylpseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl) uridine, 5-(isopentenylaminomethyl)-2-thiouridine, 2-aminoadenosine or 5-(isopentenylaminomethyl)-2′-O-methyluridine or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or 0(6)-methylguanine.

In some embodiments, the eTIS combination comprises: a first eTIS comprising a tunable element consisting of TTT or UUU; and a second eTIS comprising a tunable element consisting of ACC. In some embodiments, the eTIS combination comprises: a first eTIS comprising a tunable element consisting of CCC; and a second eTIS comprising a tunable element consisting of ACC. In some embodiments, the eTIS combination comprises: a first eTIS comprising a tunable element consisting of ACC; and a second eTIS comprising a tunable element consisting of ACC. In some embodiments, the eTIS combination comprises: a first eTIS comprising a tunable element consisting of TTC or UUC; and a second eTIS comprising a tunable element consisting of ACC. In some embodiments, the eTIS combination comprises: a first eTIS comprising a tunable element consisting of GGG; and a second eTIS comprising a tunable element consisting of ACC.

In some embodiments, the polynucleotide further comprises n supplemental nucleic acid unit(s), wherein n is an integer greater than zero. In some embodiments, each supplemental nucleic acid unit encodes one or more supplemental unit payload protein(s). In some embodiments, each supplemental nucleic acid unit comprises an engineered translation initiation site (eTIS) comprising a three-nucleotide tunable element immediately upstream of a start codon. In some embodiments, the promoter is capable of inducing transcription of the first nucleic acid unit, the second nucleic acid unit, and each supplemental nucleic acid unit to generate the polycistronic transcript, In some embodiments, the polycistronic transcript is capable of being translated to generate the one or more first unit payload protein(s), the one or more second unit payload protein(s), and the one or more supplemental unit payload protein(s) encoded by each of the n supplemental nucleic acid unit(s). In some embodiments, the eTIS of each of the first nucleic acid unit, the second nucleic acid unit, and the n supplemental nucleic acid unit(s) is configured to achieve a predetermined stoichiometry of the one or more first unit payload protein(s), the one or more second unit payload protein(s), and the one or more supplemental unit payload protein(s) encoded by each of the n supplemental nucleic acid unit(s) in a cell or cell-like environment.

In some embodiments, the first nucleic acid unit is upstream of the second nucleic acid unit, optionally the second nucleic acid unit is upstream of the n supplemental nucleic acid unit(s). In some embodiments, the first nucleic acid unit, the second nucleic acid unit, and the n supplemental nucleic acid unit(s) each comprise an open reading frame (ORF). In some embodiments, the tunable element modulates the strength of an eTIS of a nucleic acid unit, and wherein the strength of an eTIS of a nucleic acid unit is related to the fraction of the 43S preinitiation complex (PIC) scanning the polycistronic transcript that initiate and translate the open reading frame of said nucleic acid unit by engaging with the 60S ribosomal subunit upon reaching said eTIS. In some embodiments, the tunable element of each (eTIS) of each supplemental nucleic acid unit is independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

In some embodiments, the n supplemental nucleic acid unit(s) comprise one or more of a third nucleic acid unit, a fourth nucleic acid unit, a fifth nucleic acid unit, a sixth nucleic acid unit, a seventh nucleic acid unit, an eight nucleic acid unit, and a ninth nucleic acid unit. In some embodiments, the eTIS combination further comprises a third eTIS of the third nucleic acid unit. In some embodiments, the eTIS combination comprises: a first eTIS comprising a tunable element consisting of ACC; a second eTIS comprising a tunable element consisting of ACC; and a third eTIS comprising a tunable element consisting of ACC. In some embodiments, the eTIS combination comprises: a first eTIS comprising a tunable element consisting of ACC; a second eTIS comprising a tunable element consisting of TTT or UUU; and a third eTIS comprising a tunable element consisting of ACC. In some embodiments, the eTIS combination comprises: a first eTIS comprising a tunable element consisting of CCC; a second eTIS comprising a tunable element consisting of TTT or UUU; and a third eTIS comprising a tunable element consisting of ACC. In some embodiments, the eTIS combination comprises: a first eTIS comprising a tunable element consisting of TTT or UUU; a second eTIS comprising a tunable element consisting of ACC; and a third eTIS comprising a tunable element consisting of ACC. In some embodiments, the eTIS combination comprises: a first eTIS comprising a tunable element consisting of TTT or UUU; a second eTIS comprising a tunable element consisting of CCC; and a third eTIS comprising a tunable element consisting of ACC. In some embodiments, the eTIS combination comprises: a first eTIS comprising a tunable element consisting of TTT or UUU; a second eTIS comprising a tunable element consisting of TTT or UUU; and a third eTIS comprising a tunable element consisting of ACC. In some embodiments, the first eTIS, the second eTIS, the third eTIS, the fourth eTIS, and/or the fifth eTIS comprise a Kozak sequence or derivative thereof. In some embodiments, the first eTIS, the second eTIS, the third eTIS, the fourth eTIS, and/or the fifth eTIS comprise a start codon and about 3-10 neighboring nucleotides.

In some embodiments, one or more of the first unit payload protein(s), the second unit payload protein(s), and the supplemental unit payload protein(s) is a payload, is a component of a payload, or chaperones the assembly of a payload. In some embodiments, two or more of the first unit payload protein(s), the second unit payload protein(s), and the supplemental unit payload protein(s) are components of a multimeric protein complex. In some embodiments, two or more of the first unit payload protein(s), the second unit payload protein(s), and/or the supplemental unit payload protein(s) are capable of forming an antibody or antigen-binding fragment thereof, optionally one or more of the first unit payload protein(s), the second unit payload protein(s), and the supplemental unit payload protein(s) is a light chain, and one or more of the first unit payload protein(s), the second unit payload protein(s), and the supplemental unit payload protein(s) is heavy chain. In some embodiments, one or more of the first unit payload protein(s), the second unit payload protein(s), and the supplemental unit payload protein(s) is a toxin, and wherein one or more of the first unit payload protein(s), the second unit payload protein(s), and the supplemental unit payload protein(s) is an antitoxin. In some embodiments, one or more of the first unit payload protein(s), the second unit payload protein(s), and the supplemental unit payload protein(s) is a structural protein, and wherein one or more of the first unit payload protein(s), the second unit payload protein(s), and the supplemental unit payload protein(s) is a chaperone. In some embodiments, two or more of the first unit payload protein(s), the second unit payload protein(s), and/or the supplemental unit payload protein(s) are capable of one or more of: (i) forming mosaic virus-like particles; (ii) forming all or a portion of a metabolic pathway; (iii) complex and/or polyclonal antigen(s); (iv) a cytokine cocktail; (v) an immunomodulatory cocktail; (vi) all or a portion of an enzymatic pathway, optionally for the production of a small molecule, further optionally a small molecule therapeutic; and (vii) ex vivo and/or in vivo cell fate determination and/or reprogramming, further optionally via expression of two or more transcription factors, optionally expression of two or more transcription factors radiometrically. In some embodiments, two or more of the first unit payload protein(s), the second unit payload protein(s), and/or the supplemental unit payload protein(s) are configured for secretion, optionally via an N-terminal signal peptide, further optionally a signal peptide derived from CD8, IgG1 heavy chain, IgK light chain and/or GM-CSF. In some embodiments, the nucleic acid composition is capable of causing less cellular burden, toxicity, and/or apoptosis as compared to a nucleic acid composition wherein the expression of the first unit payload protein(s), the second unit payload protein(s), and the supplemental unit payload protein(s) is achieved via two or more separate polynucleotides, optionally the separate polynucleotides are separate vectors.

The first nucleic acid unit can be upstream of the second nucleic acid unit. In some embodiments, the second nucleic acid unit is upstream of the n supplemental nucleic acid unit(s). In some embodiments, the first nucleic acid unit, the second nucleic acid unit, and the n supplemental nucleic acid unit(s) each comprise an open reading frame (ORF). In some embodiments, the tunable element modulates the strength of an eTIS of a nucleic acid unit, and wherein the strength of an eTIS of a nucleic acid unit is related to the fraction of the ribosomes scanning the polycistronic transcript that initiate and translate the open reading frame of said nucleic acid unit upon reaching said eTIS. In some embodiments, the expression level of a unit payload protein is inversely related to the number and strength of eTIS situated upstream of the nucleic acid unit from which it derives on the polycistronic transcript. In some embodiments, the strength of the eTIS of the first nucleic acid unit is inversely proportional to the expression level of the second unit payload protein(s). In some embodiments, the expression level of the second unit payload protein(s) is inversely related to the fraction of the ribosomes initiating and translating the open reading frame of the first nucleic acid unit. In some embodiments, the strength of the eTIS of the second nucleic acid unit is greater than the strength of the eTIS of the first nucleic acid unit, and thereby the eTIS of the second nucleic acid unit efficiently captures the ribosomal translational activity that fails to initiate at the eTIS of the first nucleic acid unit.

The tunable element can be AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, or any combination thereof. In some embodiments, the tunable element is ACC, GGG, CCC, TTC, TTT, or any combination thereof. In some embodiments, the tunable element is selected AAA, AAU, AAC, AAG, AUA, AUU, AUC, AUG, ACA, ACU, ACC, ACG, AGA, AGU, AGC, AGG, UAA, UAU, UAC, UAG, UUA, UUU, UUC, UUG, UCA, UCU, UCC, UCG, UGA, UGU, UGC, UGG, CAA, CAU, CAC, CAG, CUA, CUU, CUC, CUG, CCA, CCU, CCC, CCG, CGA, CGU, CGC, CGG, GAA, GAU, GAC, GAG, GUA, GUU, GUC, GUG, GCA, GCU, GCC, GCG, GGA, GGU, GGC, GGG, and any combination thereof.

In some embodiments, the tunable element is ACC, GGG, CCC, UUC, UUU, or any combination thereof. In some embodiments, the first nucleic acid unit, the second nucleic acid unit, and/or n supplemental nucleic acid unit(s) each comprise one or more of a first eTIS, a second eTIS, a third eTIS, a fourth eTIS, and/or a fifth eTIS. In some embodiments, the first eTIS comprises a tunable element consisting of ACC; the second eTIS comprises a tunable element consisting of GGG; the third eTIS comprises a tunable element consisting of CCC; the fourth eTIS comprises a tunable element consisting of TTC or UUC; and the fifth eTIS comprises a tunable element consisting of TTT or UUU. In some embodiments, the first eTIS has greater strength than the second eTIS, wherein the second eTIS has greater strength than the third eTIS, wherein the third eTIS has greater strength than the fourth eTIS, and wherein the fourth eTIS has greater strength than the fifth eTIS. In some embodiments, the eTIS comprises a G nucleotide immediately downstream of the start codon.

In some embodiments, the steady-state levels of one or more of the first unit payload protein(s), the second unit payload protein(s), and/or the supplemental unit payload protein(s) is at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold greater than the steady-state levels of one or more of the first unit payload protein(s), the second unit payload protein(s), and/or the supplemental unit payload protein(s). In some embodiments, difference between the steady-state levels of one or more of the first unit payload protein(s), the second unit payload protein(s), and/or the supplemental unit payload protein(s) and the steady-state levels of one or more of the first unit payload protein(s), the second unit payload protein(s), and/or the supplemental unit payload protein(s) is less than about one order of magnitude. In some embodiments, difference between the steady-state levels of one or more of the first unit payload protein(s), the second unit payload protein(s), and/or the supplemental unit payload protein(s) and the steady-state levels of one or more of the first unit payload protein(s), the second unit payload protein(s), and/or the supplemental unit payload protein(s) is greater than about one order of magnitude. In some embodiments, the expression level of the first unit payload protein(s), the second unit payload protein(s), and the supplemental unit payload protein(s) is related to the strength of the eTIS of the corresponding nucleic acid unit from which it derives. In some embodiments, the predetermined stoichiometry is configured to achieve a therapeutic level of the first unit payload protein(s), the second unit payload protein(s), and the supplemental unit payload protein(s). In some embodiments, the predetermined stoichiometry is configured to achieve efficacious steady-state protein levels of each of the first unit payload protein(s), the second unit payload protein(s), and the supplemental unit payload protein(s). In some embodiments, the predetermined stoichiometry is robust to tissue tropism and stochastic expression.

In some embodiments, one or more of the first nucleic acid unit, the second nucleic acid unit, and the n supplemental nucleic acid unit(s) do not comprise an internal start codon. In some embodiments, one or more of the first nucleic acid unit, the second nucleic acid unit, and the n supplemental nucleic acid unit(s) have been configured to not comprise an internal start codon. In some embodiments, one or more of the first nucleic acid unit, the second nucleic acid unit, and the n supplemental nucleic acid unit(s) is codon-optimized. In some embodiments, the polycistronic transcript does not comprise an upstream ORF (uORF). In some embodiments, the first unit payload protein(s) is not less than about 30, about 25, about 20, about 15, about 10, or about 5, amino acids in length. In some embodiments, one or more of the first unit payload protein(s), the second unit payload protein(s), and/or the supplemental unit payload protein(s) does not comprise an internal methionine residue. In some embodiments, one or more of the first unit payload protein(s), the second unit payload protein(s), and/or the supplemental unit payload protein(s) does not comprise non-native amino acid residues. In some embodiments, one or more of the first nucleic acid unit, the second nucleic acid unit, and the n supplemental nucleic acid unit(s) do not comprise a tandem gene expression element.

The tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, one or more of the first nucleic acid unit, the second nucleic acid unit, and the n supplemental nucleic acid unit(s) encode more than one payload protein. In some embodiments, one or more of the first nucleic acid unit, the second nucleic acid unit, and the n supplemental nucleic acid unit(s) comprise a tandem gene expression element selected from an IRES, F2A, E2A, P2A or T2A, or any combination thereof. The polynucleotide can comprise a 5′UTR and/or a 3′UTR. In some embodiments, the cell-like environment comprises an in vitro environment configured for protein expression. In some embodiments, the promoter comprises a heterologous promoter element and/or an endogenous promoter element. In some embodiments, the heterologous promoter element is capable of being bound by a component of a synthetic protein circuit. In some embodiments, an endogenous promoter element is capable of being bound by an endogenous protein of a cell. In some embodiments, the promoter comprises a minimal promoter (e.g., TATA, miniCMV, and/or miniPromo). In some embodiments, the promoter comprises a ubiquitous promoter, an inducible promoter, a tissue-specific promoter and/or a lineage-specific promoter. In some embodiments, the ubiquitous promoter is a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof. In some embodiments, the polynucleotide is between about 100 and 100000 nucleotides in length. In some embodiments, the first nucleic acid unit, the second nucleic acid unit, and/or the n supplemental nucleic acid unit(s) is between about 100 and 10000 nucleotides in length. In some embodiments, the first unit payload protein(s), the second unit payload protein(s), and/or the supplemental unit payload protein(s) is between about 30 amino acids and 30000 amino acids in length.

In some embodiments, one or more of the first nucleic acid unit, the second nucleic acid unit, and the n supplemental nucleic acid unit(s) does not comprise an out-of-frame AUG, optionally one or more of the first nucleic acid unit, the second nucleic acid unit, and the n supplemental nucleic acid unit(s) has been configured to not comprise an out-of-frame AUG using synonymous mutation(s). In some embodiments, the nucleic acid composition further comprises a second polynucleotide comprising m secondary nucleic acid units. In some embodiments, m is an integer greater than one. In some embodiments, each secondary nucleic acid unit encodes one or more secondary unit payload protein(s). In some embodiments, each secondary nucleic acid unit comprises an engineered translation initiation site (eTIS) comprising a three-nucleotide tunable element immediately upstream of a start codon. In some embodiments, the second polynucleotide is capable of being translated to generate the one or more secondary unit payload protein(s) encoded by each of the m secondary nucleic acid units, optionally the second polynucleotide is a second polycistronic transcript. In some embodiments, the eTIS of each of the m secondary nucleic acid units is configured to achieve a predetermined stoichiometry of the one or more secondary unit payload protein(s) encoded by each of the m secondary nucleic acid unit in a cell or cell-like environment. In some embodiments, the nucleic acid composition further comprises a second polynucleotide comprising m secondary nucleic acid units. In some embodiments, m is an integer greater than one. In some embodiments, a second promoter operably linked to the second polynucleotide. In some embodiments, each secondary nucleic acid unit encodes one or more secondary unit payload protein(s). In some embodiments, each secondary nucleic acid unit comprises an engineered translation initiation site (eTIS) comprising a three-nucleotide tunable element immediately upstream of a start codon. In some embodiments, the second promoter is capable of inducing transcription of each secondary nucleic acid unit to generate to generate a second polycistronic transcript. In some embodiments, the second polycistronic transcript is capable of being translated to generate the one or more secondary unit payload protein(s) encoded by each of the m secondary nucleic acid units. In some embodiments, the eTIS of each of the m secondary nucleic acid units is configured to achieve a predetermined stoichiometry of the one or more secondary unit payload protein(s) encoded by each of the m secondary nucleic acid unit in a cell or cell-like environment.

In some embodiments, the polynucleotide and/or second polynucleotide encode gas vesicle assembly (GVA) genes and/or gas vesicle structural (GVS) genes capable of forming one or more gas vesicle(s) upon expression in the cell or cell-like environment, such as a plurality of gas vesicles, or a plurality of gas vesicles and a plurality of secondary gas vesicles. In some embodiments, the plurality of secondary gas vesicles comprise distinctive mechanical, acoustic, surface and/or magnetic properties as compared to the plurality of gas vesicles. In some embodiments, two or more of the first unit payload protein(s), the second unit payload protein(s), and/or the supplemental unit payload protein(s) are capable of forming gas vesicle(s), such as, for example, gas vesicle(s) derived from a species of Anabaena bacteria, Halobacterium salinarum, and/or Bacillus megaterium. In some embodiments, one or more of the first unit payload protein(s), the second unit payload protein(s), and/or the supplemental unit payload protein(s) are encoded by GVA genes and/or GVS genes, such as, for example, GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Halorubrum vacuolatum, Microcystis aeruginosa, Methanosarcina barkeri, Streptomyces coelicolor, and/or Psychromonas ingrahamii.

The polynucleotide and/or second polynucleotide can comprise: two or more GVA and/or GVS genes derived from different prokaryotic species; GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii; gvpB, gvpN gvpF, gvpG, gvpL gvpS, gvpK, gvpJ, and/or gvpU from B. megaterium; gvpA, gvpC, gvpN, gvpJ, gvpK, gvpF, gvpG, gvpV, and/or gvpW from Anabaena flos-aquae; gvpR, gvpN, gvpF, gvpG, gvpL, gvpS, gvpK, gvpJ, gvpT and/or gvpU from B. megaterium and gvpA from Anabaena flos-aquae; gvpA, and/or gvpC from Anabaena flos-aquae, and gvpN, gvpF, gvpG, gvpL, gvpS, gvpK, gvpJ, and/or gvpU from B. megaterium; and/or gvpA, gvpC and/or gvpN from Anabaena flos-aquae, and gvpF, gvpG, gvpL, gvpS, gvpK, gvpJ, and/or gvpU from B. megaterium.

The GVA genes and GVS genes can have sequences codon optimized for expression in a eukaryotic cell. The gas vesicle(s) can comprise a GVS variant engineered to present a tag enabling clustering in the cell. The gas vesicle(s) can comprise a GvpC variant comprising at least one protease recognition site inserted within the central portion and/or attached to at least one of the N-terminus and the C-terminus of the Gvp. In some embodiments, one or more of the mechanical, acoustic, surface and/or magnetic properties of the gas vesicle(s) are capable of being configured by adjusting the eTIS of one or more of the first nucleic acid unit, the second nucleic acid unit, the n supplemental nucleic acid unit(s), and/or the secondary nucleic acid units. In some embodiments, the gas vesicle(s) are hybrid gas vesicle(s) derived from two or more prokaryotic species. In some embodiments, the plurality of gas vesicles comprises a first collapse pressure profile. In some embodiments, the first collapse pressure profile comprises a collapse function from which a gas vesicle collapse amount can be determined for a given pressure value. In some embodiments, the first collapse pressure profile comprises a first initial collapse pressure where 5% or lower of the plurality of gas vesicles are collapsed, a first midpoint collapse pressure where 50% of the plurality of gas vesicles are collapsed, a first complete collapse pressure where at least 95% of the plurality of gas vesicles are collapsed, any pressure between the first initial collapse pressure and the first midpoint collapse pressure, and any pressure between the first midpoint collapse pressure and the first complete collapse pressure. In some embodiments, a first selectable collapse pressure is: any collapse pressure within the first collapse pressure profile; selected from the first collapse pressure profile at a value between 0.05% collapse of the plurality of gas vesicles and 95% collapse of the plurality of gas vesicles; equal to or greater than the first initial collapse pressure; equal to or greater than the first midpoint collapse pressure; and/or equal to or greater than the first complete collapse pressure.

The plurality of secondary gas vesicles can comprises a second collapse pressure profile. In some embodiments, the second collapse pressure profile comprises a collapse function from which a secondary gas vesicle collapse amount can be determined for a given pressure value. In some embodiments, the first collapse pressure profile and the second collapse pressure profile are different. In some embodiments, the first collapse pressure profile and/or second collapse pressure profile has been configured by engineering a gas vesicle protein C (GvpC) protein of the gas vesicles and/or the secondary gas vesicles. In some embodiments, a midpoint of the second collapse profile has a higher pressure component than a midpoint of the first collapse profile. In some embodiments, the second collapse pressure profile comprises a second initial collapse pressure where 5% or lower of the plurality of secondary gas vesicles are collapsed, a second midpoint collapse pressure where 50% of the plurality of secondary gas vesicles are collapsed, a second complete collapse pressure where at least 95% of the plurality of secondary gas vesicles are collapsed, any pressure between the second initial collapse pressure and the second midpoint collapse pressure, and any pressure between the second midpoint collapse pressure and the second complete collapse pressure. A second selectable collapse pressure can be: any collapse pressure within the second collapse pressure profile; selected from the second collapse pressure profile at a value between 0.05% collapse of the plurality of secondary gas vesicles and 95% collapse of the plurality of secondary gas vesicles; equal to or greater than the second initial collapse pressure; equal to or greater than the second midpoint collapse pressure; and/or equal to or greater than the second complete collapse pressure.

In some embodiments, the promoter, polynucleotide, second promoter, and/or second polynucleotide is configured to express the gas vesicle(s) in response a biochemical event in the cell. In some embodiments, the expression of the gas vesicle(s) is an output of a synthetic protein circuit. In some embodiments, a payload is capable of diminishing the concentration, stability, and/or activity an endogenous protein. In some embodiments, a payload comprises a component of a synthetic protein circuit. In some embodiments, a payload is capable of diminishing the concentration, stability, and/or activity of one or more of the first unit payload protein(s), the second unit payload protein(s), and/or the supplemental unit payload protein(s). In some embodiments, two or more of the first unit payload protein(s), the second unit payload protein(s), and/or the supplemental unit payload protein(s) are components of a synthetic protein circuit. In some embodiments, all components of said synthetic protein circuit are encoded by the first nucleic acid unit, the second nucleic acid unit, and/or the n supplemental nucleic acid unit(s). In some embodiments, a payload comprises a degron and a cut site a protease is capable of cutting to expose the degron, and wherein the degron of the payload being exposed changes the payload to a payload destabilized state. In some embodiments, the degron comprises an N-degron, a dihydrofolate reductase (DHFR) degron, a FKB protein (FKBP) degron, derivatives thereof, or any combination thereof. In some embodiments, a payload comprises a protease or a split protease. In some embodiments, the activation level of the protease is related to one or more input signals. In some embodiments, the protease comprises tobacco etch virus (TEV) protease, tobacco vein mottling virus (TVMV) protease, hepatitis C virus protease (HCVP), derivatives thereof, or any combination thereof. In some embodiments, the synthetic protein circuit is configured to be responsive to changes in: cell environment, optionally cell environment comprises location relative to a target site of a subject and/or changes in the presence and/or absence of target cell(s), optionally said target cell(s) comprise target-specific antigen(s); one or more signal transduction pathways regulating cell survival, cell growth, cell proliferation, cell adhesion, cell migration, cell metabolism, cell morphology, cell differentiation, apoptosis, or any combination thereof; input(s) of a synthetic cell-cell communication system, optionally Synthetic Notch (SynNotch) receptor, a Modular Extracellular Sensor Architecture (MESA) receptor, a synthekine, engineered GFP, and/or auxin; and/or T cell activity, optionally T cell activity comprises one or more of T cell simulation, T cell activation, cytokine secretion, T cell survival, T cell proliferation, CTL activity, T cell degranulation, and T cell differentiation.

In some embodiments, a payload is an antigenic polypeptide (AP). In some embodiments, two or more of the first unit payload protein(s), the second unit payload protein(s), and/or the supplemental unit payload protein(s) is an AP, and thereby the polycistronic transcript is capable of being translated to generate a plurality of disparate AP. In some embodiments, the AP comprises or is derived from an antigenic protein associated with a disease or disorder (e.g., an immunogenic variant and/or an immunogenic fragment of said antigenic protein). In some embodiments, the disease or disorder is an infectious disease or disorder caused by an infectious agent, wherein the AP comprises or is derived from an antigenic protein of said infectious agent, and wherein the antigenic protein of said infectious agent is a pathogenic antigen. In some embodiments, the disease or disorder is a disease is associated with expression of a tumor-associated antigen, and wherein the antigenic protein is a tumor-associated antigen. In some embodiments, the disease or disorder is an autoimmune disease or disorder, and wherein the antigenic protein is an autoimmune antigen. In some embodiments, the disease or disorder is an allergic disease or disorder, and wherein the antigenic protein is an allergenic antigen. In some embodiments, the infectious agent is a bacterium, a fungus, a virus, or a protist. In some embodiments, the infectious agent is a coronavirus (CoV). In some embodiments, the CoV comprises an alphacoronavirus, a betacoronavirus, a gammacoronavirus, or a deltacoronavirus. In some embodiments, the infectious agent is selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157: H7, O111 and O104: H4, Fasciola hepatica and Fasciola gigantica, FFI prion, Filarioidea superfamily, Filoviruses, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

The plurality of disparate AP can comprise between about 2 and about 500 antigenic polypeptides that differ from each other. In some embodiments, the plurality of disparate AP comprises AP of a same protein type. In some embodiments, the plurality of disparate AP comprises AP of different protein types. In some embodiments, the plurality of disparate AP comprise a plurality of coronavirus (CoV) antigens, wherein the plurality of CoV antigens comprises a first CoV antigen of a first CoV and a second CoV antigen of a second CoV that is different from the first CoV. In some embodiments, the plurality of CoV antigens comprise a CoV spike protein (S protein) or a portion thereof, a CoV envelope protein (E protein) or a portion thereof, a CoV nucleocapsid protein (N protein) or a portion thereof, a CoV hemagglutinin-esterase protein (HE protein) or a portion thereof, a CoV papain-like protease or a portion thereof, a CoV 3CL protease or a portion thereof, a CoV membrane protein (M protein) or a portion thereof, or a combination thereof. In some embodiments, the plurality of disparate AP comprise one or more of a 1st pathogenic antigen (PA) of a 1st infectious agent (IA), a 2nd PA of a 2nd IA, a 3rd PA of a 3rd IA, a 4th PA of a 4th IA, a 5th PA of a 5th IA, a 6th PA of a 6th IA, a 7th PA of a 7th IA, a 8th PA of a 8th IA, a 9th PA of a 9th IA, a 10th PA of a 10th IA, a 11th PA of a 11th IA, a 12th PA of a 12th IA, a 13th PA of a 13th IA, a 14th PA of a 14th IA, a 15th PA of a 15th IA, a 16th PA of a 16th IA, a 17th PA of a 17th IA, a 18th PA of a 18th IA, a 19th PA of a 19th IA, a 20th PA of a 20th IA, a 21st PA of a 21st IA, a 22nd PA of a 22nd IA, a 23rd PA of a 23rd IA, a 24th PA of a 24th IA, a 25th PA of a 25th IA, a 26th PA of a 26th IA, a 27th PA of a 27th IA, a 28th PA of a 28th IA, a 29th PA of a 29th IA, a 30th PA of a 30th IA, a 31st PA of a 31st IA, a 32nd PA of a 32nd IA, a 33rd PA of a 33rd IA, a 34th PA of a 34th IA, a 35th PA of a 35th IA, a 36th PA of a 36th IA, a 37th PA of a 37th IA, a 38th PA of a 38th IA, a 39th PA of a 39th IA, a 40th PA of a 40th IA, a 41st PA of a 41st IA, a 42nd PA of a 42nd IA, a 43rd PA of a 43rd IA, a 44th PA of a 44th IA, a 45th PA of a 45th IA, a 46th PA of a 46th IA, a 47th PA of a 47th IA, a 48th PA of a 48th IA, a 49th PA of a 49th IA, and a 50th PA of a 50th IA. In some embodiments, the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th pathogenic antigens are different from one another. In some embodiments, the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th infectious agents are different from one another.

The payload can be a therapeutic protein or a variant thereof (e.g., a therapeutic protein configured to prevent or treat a disease or disorder of a subject). In some embodiments, the subject suffers from a deficiency of said therapeutic protein. In some embodiments, a payload comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, a payload comprises nuclease activity, methyltransferase activity, disulfide isomerase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, a payload comprises a CRE recombinase, GCaMP, a cell therapy component, a knock-down gene therapy component, a cell-surface exposed epitope, or any combination thereof. In some embodiments, a payload comprises a diagnostic agent. In some embodiments, the diagnostic agent comprises a bioluminescent diagnostic agent, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3, rsCherry, rsCherryRev, derivatives thereof, or any combination thereof.

The payload can comprise a bispecific T cell engager (BiTE). In some embodiments, a payload comprises a cytokine, for example interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. In some embodiments, a payload comprises a member of the TGF-β/BMP family selected from TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. In some embodiments, a payload comprises a member of the TNF family of cytokines selected from TNF-alpha, TNF-beta, LT-beta, CD40 ligand, Fas ligand, CD 27 ligand, CD 30 ligand, and 4-1 BBL. In some embodiments, a payload comprises a member of the immunoglobulin superfamily of cytokines selected from B7.1 (CD80) and B7.2 (B70). In some embodiments, a payload comprises an interferon. In some embodiments, the interferon is selected from interferon alpha, interferon beta, or interferon gamma. In some embodiments, a payload comprises a chemokine. In some embodiments, the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. In some embodiments, a payload comprises an interleukin. In some embodiments, the interleukin is selected from IL-10 IL-12, IL-1, IL-6, IL-7, IL-15, IL-2, IL-18 or IL-21. In some embodiments, a payload comprises a tumor necrosis factor (TNF). In some embodiments, the TNF is selected from TNF-alpha, TNF-beta, TNF-gamma, CD252, CD154, CD178, CD70, CD153, or 4-1BBL. In some embodiments, a payload comprises a factor locally down-regulating the activity of endogenous immune cells. In some embodiments, a payload is capable of remodeling a tumor microenvironment and/or reducing immunosuppression at a target site of a subject.

The payload can comprise a chimeric antigen receptor (CAR) or T-cell receptor (TCR). In some embodiments, the CAR and/or TCR comprises one or more of an antigen binding domain, a transmembrane domain, and an intracellular signaling domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof. In some embodiments, the costimulatory domain comprises a functional domain of one or more proteins selected from CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof. In some embodiments, the antigen binding domain binds a tumor antigen. In some embodiments, the tumor antigen is a solid tumor antigen. In some embodiments, the tumor antigen is: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary glad differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-IR, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, B2-Microglobulin, Fc Receptor-like 5 (FcRL5), or molecules expressed by HIV, HCV, HBV, or other pathogens.

In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

The antigen binding domain can be connected to the transmembrane domain by a hinge region. In some embodiments, the transmembrane domain comprises a transmembrane domain of a protein selected from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the CAR or TCR further comprises a leader peptide. In some embodiments, the TCR further comprises a constant region and/or CDR4.

The payload can be an activity regulator. In some embodiments, the activity regulator is capable of reducing T cell activity. In some embodiments, the activity regulator comprises a ubiquitin ligase involved in TCR/CAR signal transduction selected from c-CBL, CBL-B, ITCH, R F125, R F128, WWP2, and any combination thereof. In some embodiments, the activity regulator comprises a negative regulatory enzyme selected from SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. In some embodiments, the activity regulator is a negative regulatory scaffold/adapter protein selected from PAG, LIME, NTAL, LAX31, SIT, GAB2, GRAP, ALX, SLAP, SLAP2, DOK1, DOK2, and any combination thereof. In some embodiments, the activity regulator is a dominant negative version of an activating TCR signaling component selected from ZAP70, LCK, FYN, NCK, VAV1, SLP76, ITK, ADAP, GADS, PLCgammal, LAT, p85, SOS, GRB2, NFAT, p50, p65, API, RAPl, CRKII, C3G, WAVE2, ARP2/3, ABL, ADAP, RIAM, SKAP55, or any combination thereof. In some embodiments, the activity regulator comprises the cytoplasmic tail of a negative co-regulatory receptor selected from CD5, PD1, CTLA4, BTLA, LAG3, B7-H1, B7-1, CD160, TFM3, 2B4, TIGIT, and any combination thereof. In some embodiments, the activity regulator is targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, and any combination thereof. In some embodiments, a payload is capable of modulating the expression, concentration, localization, stability, and/or activity of the one or more endogenous targets of a cell.

In some embodiments, a payload comprises a programmable nuclease. In some embodiments, the programmable nuclease is: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas 100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the nucleic acid composition further comprises a polynucleotide encoding (i) a targeting molecule and/or (ii) a donor nucleic acid. In some embodiments, a payload comprises (i) a targeting molecule and/or (ii) a donor nucleic acid. In some embodiments, the targeting molecule is capable of associating with the programmable nuclease. In some embodiments, the targeting molecule comprises single strand DNA or single strand RNA. In some embodiments, wherein the targeting molecule comprises a single guide RNA (sgRNA).

In some embodiments, the payload comprises a pro-death protein capable of halting cell growth and/or inducing cell death. In some embodiments, the pro-death protein comprises cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments, the pro-death protein comprises Caspase-9 and the pro-death agent comprises AP1903. In some embodiments, the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT). In some embodiments, the pro-death protein comprises Cytosine Deaminase (CD) and the pro-death agent comprises 5-fluorocytosine (5-FC). In some embodiments, the pro-death protein comprises Purine nucleoside phosphorylase (PNP) and the pro-death agent comprises 6-methylpurine deoxyriboside (MEP) and/or fludarabine (FAMP). In some embodiments, the pro-death protein comprises a Cytochrome p450 enzyme (CYP) and the pro-death agent comprises Cyclophosphamide (CPA), Ifosfamide (IFO), and/or 4-ipomeanol (4-IM). In some embodiments, the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy- and amino-aniline mustards, Anthracycline glutamates, and/or Methotrexate α-peptides (MTX-Phe). In some embodiments, the pro-death protein comprises Carboxylesterase (CE) and the pro-death agent comprises Irinotecan (IRT), and/or Anthracycline acetals. In some embodiments, the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones. In some embodiments, the pro-death protein comprises Horse radish peroxidase (HRP) and the pro-death agent comprises Indole-3-acetic acid (IAA) and/or 5-Fluoroindole-3-acetic acid (FIAA). In some embodiments, the pro-death protein comprises Guanine Ribosyltransferase (XGRTP) and the pro-death agent comprises 6-Thioxanthine (6-TX). In some embodiments, the pro-death protein comprises a glycosidase enzyme and the pro-death agent comprises HM1826 and/or Anthracycline acetals. In some embodiments, the pro-death protein comprises Methionine-α,γ-lyase (MET) and the pro-death agent comprises Selenomethionine (SeMET). In some embodiments, the pro-death protein comprises thymidine phosphorylase (TP) and the pro-death agent comprises 5′-Deoxy-5-fluorouridine (5′-DFU).

The payload can comprise one or more receptors and/or a targeting moiety configured to bind a component of a target site of a subject. In some embodiments, the one or more receptors and/or the one or more targeting moieties are selected from mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. In some embodiments, the one or more targeting moieties and/or one or more receptors comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. In some embodiments, the antibody or antigen-binding fragment thereof comprises a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, and any combination thereof.

In some embodiments, the one or more targeting moieties and/or one or more receptors are configured to bind one or more of the following: CD3, CD4, CD5, CD6, CD7, CD8, CD9, CD10, CDl la, CDI 1b, CDl lc, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C-X-C chemokine receptor type 4, cyclic ADP ribose hydrolase, Cyclin B 1, CYP1B 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRVIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αuβ3, αvβ3, α4β7, α5β1, α6β4, α7β7, a11β3, a5B5, αvβ5), Interferon gamma-induced protein, ITGA2, ITGB2, KIR2D, LCK, Le, Legumain, Lewis-Y antigen, LFA-1 (Lymphocyte function-associated antigen 1, CDl la), LHRH, LINGO-1, Lipoteichoic acid, LIVIA, LMP2, LTA, MAD-CT-1, MAD-CT-2, MAGE-1, MAGE-2, MAGE-3, MAGE AI, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In some embodiments, a payload is associated with an agricultural trait of interest selected from increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. In some embodiments, a payload is associated with a biological manufacturing process selected from fermentation, distillation, biofuel production, production of a compound, production of a polypeptide, or any combination thereof.

In some embodiments, a payload is a cellular reprogramming factor capable of converting an at least partially differentiated cell to a less differentiated cell (e.g., Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof), optionally for industrial use and/or for in organismo differentiation, further optionally for therapeutic purposes. In some embodiments, a payload is a cellular reprogramming factor capable of differentiating a given cell into a desired differentiated state (e.g., nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof), optionally for industrial use and/or for in organismo differentiation, further optionally for therapeutic purposes. In some embodiments, a payload comprises an agonistic or antagonistic antibody or antigen-binding fragment thereof specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3). In some embodiments, a payload comprises a constitutive signal peptide for protein degradation (e.g., PEST). In some embodiments, a payload comprises a nuclear localization signal (NLS) or a nuclear export signal (NES). In some embodiments, a payload comprises a dosage indicator protein. In some embodiments, the dosage indicator protein is detectable. The dosage indicator protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

In some embodiments, nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the one or more vectors is a DNA vaccine. In some embodiments, the DNA vaccine is a plasmid-based DNA vaccine, a minicircle-based DNA vaccine, a bacmid-based DNA vaccine, a minigene-based DNA vaccine, a ministring DNA (linear covalently closed DNA vector) vaccine, a closed-ended linear duplex DNA (CELiD or ceDNA) vaccine, a Doggybone™ DNA vaccine, a dumbbell shaped DNA vaccine, or a minimalistic immunological-defined gene expression (MIDGE)-vector DNA vaccine.

In some embodiments, the nucleic acid composition is or comprises mRNA, optionally the mRNA is formulated in a lipid nanoparticle (LNP). In some embodiments, the mRNA comprises a 5′ untranslated region (UTR), a 3′ UTR, and/or a cap. In some embodiments, the mRNA comprises one or more modified nucleotides selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. In some embodiments, the mRNA comprises a modified nucleotide in place of one or more uridines. In some embodiments, the modified nucleoside is selected from pseudouridine (ψ), N 1-methyl-pseudouridine (m 14′), and 5-methyl-uridine (m5U). In some embodiments, the LNP comprises one or more of an ionizable cationic lipid, a non-cationic lipid, a sterol, and a PEG-modified lipid. In some embodiments, the non-cationic lipid is a neutral lipid. In some embodiments, the LNP comprises 0.5-15 mol % PEG-modified lipid, 5-25 mol % non-cationic lipid, 25-55 mol % sterol, and 20-60 mol % ionizable cationic lipid. In some embodiments, the LNP comprises: 40-55 mol % ionizable cationic lipid, 5-15 mol % neutral lipid, 35-45 mol % sterol, and 1-5 mol % PEG-modified lipid. In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid. In some embodiments, the ionizable cationic lipid is heptadecan-9-yl 8 ((2 hydroxyethyl) (6 OXO 6-(undecyloxy)hexyl)amino) octanoate. In some embodiments, the neutral lipid is 1,2 distearoyl sn glycero-3 phosphocholine (DSPC). In some embodiments, the sterol is cholesterol. In some embodiments, the PEG-modified lipid is 1-monomethoxypolyethyleneglycol-2,3-dimyristylglycerol with polyethylene glycol of average molecular weight 2000 (PEG2000 DMG). In some embodiments, the wt/wt ratio of lipid to mRNA is from about 1:100 to about 100:1.

Disclosed herein include engineered cells. In some embodiments, the engineered cells comprise: a nucleic acid composition disclosed herein. In some embodiments, the cell is: a cell of a subject; an in vivo cell, an ex vivo cell, or an in situ cell; and/or an adherent cell or a suspension cell. In some embodiments, the cell comprises a eukaryotic cell (e.g., a mammalian cell). The mammalian cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof. In some embodiments, the cell is the cell of a subject (e.g, a subject suffering from a disease or disorder). In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

Disclosed herein include pharmaceutical compositions. In some embodiments, the pharmaceutical composition comprises: a nucleic acid composition disclosed herein. In some embodiments, the pharmaceutical composition further comprises one or more pharmaceutically acceptable carriers, diluents and/or excipients.

Disclosed herein include methods of imaging a target site of a subject. In some embodiments, the method comprises: administering to the subject an effective amount of a nucleic acid composition disclosed herein, a pharmaceutical composition disclosed herein, or engineered cells disclosed herein. In some embodiments, the method comprises: applying a magnetic field and/or ultrasound (US) to a target site of a subject to obtain an MRI and/or US image of the target site.

The period of time between the administering and applying can be about 50 weeks, 45 weeks, 40 weeks, 35 weeks, 30 weeks, 25 weeks, 20 weeks, 15 weeks, 10 weeks, 8 weeks, 6 weeks, 4 weeks, 3 weeks, about 14 days, about 7 days, about 3 days, about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the nucleic acid composition is capable of expressing gas vesicle(s) having an acoustic collapse pressure threshold, and wherein applying ultrasound comprises: applying ultrasound to the target site at a peak positive pressure less than the acoustic collapse pressure threshold; increasing peak positive pressure (PPP) to above the selective acoustic collapse pressure value as a step function; and imaging the target site in successive frames during the increasing; and extracting a time-series vector for each of at least one pixel of the successive frames.

In some embodiments, the method comprises: performing a signal separation algorithm on the time-series vectors using at least one template vector. In some embodiments, the signal separation algorithm includes template projection and/or template unmixing. In some embodiments, the at least one template vector includes linear scatterers, noise, gas vesicles, or a combination thereof. In some embodiments, the successive frames comprise a frame prior to GVs collapse, a frame during GVs collapse, and a frame after GVs collapse. In some embodiments, the increasing includes increasing the PPP to a hiBURST regime, optionally the PPP in hiBURST regime is 4.3 MPa or higher. In some embodiments, the increasing includes increasing the PPP to a loBURST regime, optionally the PPP in loBURST regime is no higher than 3.7 Mpa In some embodiments, applying US to a target site comprises applying one or more US pulses to the target site over a duration of time. In some embodiments, the duration of time is about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulses each have a pulse duration of about 1 hour, about 30 minutes, about 15 minutes, about 10 minutes, about 5 minutes, about 1 minute, about 1 second, or about 1 millisecond. In some embodiments, applying an US pulse comprises applying a focused US pulse. In some embodiments, applying an US pulse comprises applying US at a frequency of 100 kHz to 100 MHz. In some embodiments, applying an US pulse comprises applying ultrasound at a frequency of 0.2 to 1.5 mHz. In some embodiments, applying an US pulse comprises applying ultrasound having a mechanical index in a range between 0.2 and 0.6. In some embodiments, the US pulse comprises a peak pressure of about 40 kPa to about 800 kPa. In some embodiments, the US pulse comprises a peak pressure of about 70 kPa to about 150 kPa, and/or about 440 kPa to about 605 kPa. In some embodiments, the method comprises the spatial and temporal delivery of payload molecules to a target site of a subject, the method comprising: applying a first ultrasonic (US) pulse to a target site of the subject; detecting the presence of the engineered cells; and applying a second US pulse to the target site of the subject, wherein the second US pulse induces the release of payload molecules from the engineered cells, thereby delivering payload molecules to the target site.

In some embodiments, the payload molecules comprise the first unit payload protein(s), the second unit payload protein(s), and/or the supplemental unit payload protein(s). In some embodiments, detecting the presence of the engineered cells at the target site comprises detecting scattering of the first US pulse by the gas vesicles. In some embodiments, the method comprises: confirming the delivery of payload molecules at the target site. In some embodiments, confirming the delivery of payload molecules comprises detecting reduced scattering of the second US pulse by the gas vesicles. In some embodiments, the gas vesicles are capable of acting as a contrast agent at the first US pulse but not at the second US pulse.

In some embodiments, the first US pulse comprises a pressure value less than the first selectable collapse pressure value. In some embodiments, the second US pulse comprises a pressure value equal to or higher than the first selectable collapse pressure value. In some embodiments, the second US pulse induces gas vesicle collapse. In some embodiments, the gas vesicles are capable of acting as a contrast agent at the first US pulse but not at the second US pulse. In some embodiments, the gas vesicle collapse results in the release of a nanoscale air bubble. In some embodiments, the released nanoscale air bubble undergoes cavitation and is converted into a micron-scale air bubble. In some embodiments, the second US pulse is capable of inducing cavitation. In some embodiments, the cavitation comprises cavitation of the gas vesicles and/or bubbles created by gas vesicle collapse. In some embodiments, the gas vesicles are capable as acting as the nuclei for the formation and/or cavitation of bubbles. In some embodiments, the cavitation comprises stable cavitation. In some embodiments, the cavitation comprises inertial cavitation. In some embodiments, the cavitation triggers the degradation of the engineered cells. In some embodiments, the cavitation induces the release of payload molecules from the engineered cells. In some embodiments, the cavitation exerts mechanical forces and/or thermal forces on the engineered cells, thereby inducing the release of payload molecules. In some embodiments, the target site comprises target cells. In some embodiments, the cavitation exerts mechanical forces and/or thermal forces on target cells proximate to the engineered cells, thereby enhancing uptake of payload molecules by said target cells. In some embodiments, said mechanical forces and/or thermal forces reduce the membrane permeability of target cells proximate to the engineered cells. In some embodiments, the peak positive pressure of the second US pulse is equal to or higher than an initial collapse pressure of the gas vesicles, thereby collapsing the gas vesicles. In some embodiments, the peak negative pressure of the second US pulse is below the critical cavitation pressure of the gas vesicles.

In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released at the target site. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released at a location other than the target site. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released from the engineered cells within about 1 ns, about 10 ns, about 100 ns, about 1 ms, about 10 ms, about 100 ms, or about 1 s after the second US pulse. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released from the engineered cells within about 1 nm, about 10 nm, about 100 nm, about 1 μm, about 10 μm, about 100 μm, about 1 mm, about 10 mm, or about 100 mm of the location of the engineered cells at the time of the second US pulse. In some embodiments, the ratio of the concentration of payload molecules at the subject's target site to the concentration of payload molecules in subject's blood, serum, or plasma is about 2:1 to about 3000:1, about 2:1 to about 2000:1, about 2:1 to about 1000:1, or about 2:1 to about 600:1.

Disclosed herein include methods of stimulating an immune response in a subject in need thereof. In some embodiments, the method comprises: administering to the subject an effective amount of a nucleic acid composition disclosed herein, a pharmaceutical composition disclosed herein, or engineered cells disclosed herein, thereby stimulating an immune response in the subject.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or engineered cells disclosed herein, thereby treating or preventing the disease or disorder in the subject. In some embodiments, the disease or disorder is a disease or disorder caused by an infectious agent.

In some embodiments, administering comprises: (i) isolating one or more cells from the subject; (ii) contacting said one or more cells with a nucleic acid composition provided herein, thereby generating engineered cells (optionally the contacting comprises transduction and/or transfection, further optionally the nucleic acid composition comprises a transient or integrating expression vector); and (iii) administering the one or more engineered cells into a subject after the contacting step. In some embodiments, the method comprises administering to the subject at least two doses of the nucleic acid composition, the pharmaceutical composition, and/or the engineered cells. In some embodiments, the second dose is administered to the subject at least 14 days after a first dose is administered to the subject. In some embodiments, administration of the nucleic acid composition, the pharmaceutical composition, and/or the engineered cells elicits protective and long-lasting immunity against the infectious agent(s) and variants thereof. In some embodiments, the nucleic acid composition, the pharmaceutical composition, and/or the engineered cells is administered in an effective amount to: induce a robust antibody response against the AP in the subject, optionally a robust antibody response comprises a neutralizing antibody response, further optionally a robust antibody response comprises Fc domain effector functions that recruit immune cells to infected cells, optionally said immune cells are macrophages, neutrophils, and/or natural killer cells, further optionally said recruitment induces antibody-dependent cellular cytotoxicity (ADCC) and/or antibody-dependent cellular phagocytosis (ADCP); elicit a robust CD4 and/or CD8 T cell response against the AP in the subject; and/or elicit a balanced Th1/Th2 response against the AP in the subject. In some embodiments, the nucleic acid composition, the pharmaceutical composition, and/or the engineered cells is co-administered with an adjuvant.

In some embodiments, the target site comprises a section or subsection of the GI tract. In some embodiments, the section or subsection of the GI tract is selected from the stomach, proximal duodenum, distal duodenum, proximal jejunum, distal jejunum, proximal ileum, distal ileum, proximal cecum, distal cecum, proximal ascending colon, distal ascending colon, proximal transverse colon, distal transverse colon, proximal descending colon and distal descending colon, and any combination thereof. In some embodiments, the target site comprises a site of disease or disorder or is proximate to a site of a disease or disorder. In some embodiments, the location of the one or more sites of a disease or disorder is predetermined. In some embodiments, the location of the one or more sites of a disease or disorder is determined during the method. In some embodiments, the target site comprises a tissue. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue is inflamed tissue. In some embodiments, the tissue comprises (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

In some embodiments, the disease or disorder is a blood disease, a gut microbiome disease or disorder, an inflammatory disease or disorder of the gut, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof, optionally a solid tumor. In some embodiments, the disease or disorder is an infectious disease selected from an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-COV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-COV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

In some embodiments, the disease is associated with expression of a tumor-associated antigen. In some embodiments, the disease associated with expression of a tumor antigen-associated is a proliferative disease, a precancerous condition, a cancer, and a non-cancer related indication associated with expression of the tumor antigen. In some embodiments, the cancer is colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

There are provided, in some embodiments, engineered cells generated via expression of the one or more first unit payload protein(s), the one or more second unit payload protein(s), and/or the one or more supplemental unit payload protein(s). In some embodiments, the nucleic acid composition is (i) an in vitro transcribed (IVT) mRNA construct and/or (ii) is the product of production in a cell or cell-like environment. In some embodiments, the nucleic acid composition comprises a nucleic acid library, optionally configured for screening, optionally for screening related to the expression and tuning of enzymatic pathways, circuits, and/or assembly of multimeric protein structures. In some embodiments, the nucleic acid composition is or comprises: (i) an mRNA vaccine; (ii) a circular RNA molecule; and/or (iii) mRNA, optionally the mRNA is formulated in a lipid nanoparticle (LNP). In some embodiments, a payload protein comprises an organelle localization sequence, optionally an ER localization sequence, peroxisomal targeting sequence, a lysosomal targeting sequence, a chloroplast targeting sequence, a mitochondrial targeting sequence, a Golgi localization sequence, and/or a ER retention signal.

Disclosed herein include methods comprising: introducing a nucleic acid library into a cell population, optionally a nucleic acid library comprising a set of predetermined eTIS combinations, further optionally a nucleic acid library configured for screening, optionally derived from a nucleic acid composition disclosed herein, further optionally the introducing step comprises one or more of transfection, transduction, plasmid-based expression, mRNA-based expression, or integrated DNA-based expression, optionally the nucleic acid composition comprises a plasmid, an mRNA, a transient expression vector, or an integrating expression vector; and screening for nucleic acid library member(s) yielding user-desired outcome(s) and/or expression ratio(s), optionally an optimal stoichiometry for a particular tissue and/or cell type, optionally screening related to the expression and tuning of enzymatic pathways, circuits, and/or assembly of multimeric protein structures.

Disclosed herein include methods comprising: introducing an effective amount of a nucleic acid composition disclosed herein into cell(s) or a cell-like environment, optionally the cell-like environment is configured for in vitro transcription, further optionally the introducing step comprises one or more of transfection, transduction, plasmid-based expression, mRNA-based expression, or integrated DNA-based expression, optionally the nucleic acid composition comprises a plasmid, an mRNA, a transient expression vector, or an integrating expression vector; and extracting polycistronic transcript(s) expressed in said cell(s) or cell-like environment.

### BRIEF DESCRIPTION OF THE DRAWINGS

FIGS. 1A-1E depict non-limiting exemplary schematics and data related to 2-ORF SEMPER constructs demonstrating tunable, bicistronic expression. (FIG. 1A) Architecture of an mRNA transcript produced by transfected 2-ORF SEMPER plasmid DNA. Cap-dependent ribosomes translate ORF 1, msfGFP [r5M], or ORF 2, mEBFP2, with frequencies dependent on the trinucleotide (NNN) upstream of each ORF. The relative strengths of the trinucleotides and TISs they represent are depicted. *** (a.k.a. TTTCCAT) does not contain a start codon, preventing translation of the ORF. IRES-mCherry is included to normalize fluorescence measurements. (FIG. 1B) mEBFP2 distribution plots for constructs containing different versions of monomeric Superfolder GFP encoded in ORF 1. Removal of internal methionines from the msfGFP leads to much stronger expression of the downstream mEBFP2. Both ORFs used the strong ACC TIS (N=1, representative of four replicates). (FIG. 1C) Flow cytometry plots of mCherry positive HEK293T cells transfected with 2-ORF SEMPER constructs. The legend contains the ORF 1 TIS and the ORF 2 TIS separated by a slash. (Left) Increasing the strength of the first TIS increases msfGFP [r5M] fluorescence relative to that of mEBFP2 (N=1, representative of four replicates). (Right) Binning cells into three mCherry fluorescence ranges produces unique clusters of cells (N=1, representative of four replicates). (FIG. 1D) Min-max normalized fluorescence values for each GOI relative to ACC/*** and ***/ACC. Fluorescence measurements were first normalized by mCherry before min-max normalization. Error bars depict standard error of the mean (SEM) (N=4). Welch's ANOVA tests were conducted for blue and green bars independently (p<0.0001 for all colors across all bins) followed by Dunnett's T3 multiple comparisons tests. All pairwise comparisons are provided in Table 2. (FIG. 1E) Violin plots of log 10 (msfGFP [r5M]/mEBFP2) values for all mCherry positive cells for four combined replicates of TTT/ACC, CCC/ACC, and ACC/ACC plasmids transfected into HEK293T and CHO-K1 cell lines. The median and quartiles of the distribution are represented by the solid and dotted lines respectively. Statistical analysis was conducted using one-way Welch's ANOVA tests (p<0.0001 (Left) and p<0.0001 (Right)) followed by Games-Howell's multiple comparisons test to yield pairwise comparisons.

FIGS. 2A-2B depict non-limiting exemplary schematics and data related to scaling tunable polycistronic expression beyond two ORFs. (FIG. 2A) Architecture of an mRNA transcript produced by transfected 3-ORF SEMPER plasmid DNA. (FIG. 2B) (Left) Min-max normalized relative translation levels for each fluorescent protein. Cells were first clustered into three mCherry fluorescence bins (low, medium, high). Error bars depict SEM (N=4). Welch's ANOVA tests were conducted for blue, green, and red bars independently (p<0.0001 for all colors across all bins). Post-hoc Dunnett's T3 multiple comparisons tests are provided in Table 4. (Right) Comparisons of msfBFP [r5M] relative translation levels between the max control and those TIS combinations with multiple ACC driven ORFs. Error bars depict SEM (N=4). Post-hoc Dunnet's T3 comparisons tests demonstrate a significant upregulation in relative translation of msfBFP [r5M] for depicted constructs compared to that observed for ACC/***/***.

FIGS. 3A-3E depict non-limiting exemplary schematics and data related to utilizing 2-ORF SEMPER constructs to express gas vesicles in mammalian cells. (FIG. 3A) Architecture of an mRNA transcript transcribed from transfected SEMPER mARG plasmid DNA. The first ORF, gvpA, encodes the main structural protein, while the second ORF, gvpNJKFGW-EmGFP, encodes all necessary accessory proteins strung together with P2A self-cleaving peptides. (FIG. 3B) Flow cytometry distributions of Emerald GFP normalized by mCherry values for each TIS combination tested in addition to an IRES-mCherry control. The thick horizontal lines depict the means of the distributions. Error bars depict standard error of the mean (SEM) (N=1, representative of four replicates). p-value <0.0001 (Kruskal-Wallis test), p-values of multiple comparisons are found in Table 6. (FIG. 3C) BURST images of acoustic contrast due to gas vesicle expression within HEK293T cells. Depicted are HEK293T cells loaded into agarose phantoms three days after transfection of SEMPER mARG plasmids or the leading two-plasmid system. The color bar represents the magnitude of BURST signal measured in linear arbitrary units. The floor and ceiling of the images are set to 0 and 5000 respectively (N=1, representative of four replicates). (FIG. 3D) BURST signal quantification of gas vesicle acoustic contrast for HEK293T samples transfected with SEMPER mARG plasmids or the leading two plasmid system. Error bars depict SEM (N=4). Statistical analysis was conducted using a one-way ANOVA (p<0.0001) followed by Fisher's LSD post-hoc test with a single pooled variance to compare each treatment group with every other. Pairwise p-values are reported in Table 7. (FIG. 3E) Annexin V staining assays to quantify the number of apoptotic cells following expression of mARG vectors. HEK293T cells transfected with pgvpA-IRES-mCherry with the start codon removed in front of gvpA were used to establish a baseline (Not Treated). A subset of these cells was treated with Raptinal to induce apoptosis (Raptinal Treated). Other cell populations were transfected solely with a fully functional pgvpA-IRES-mCherry (gvpA-IRES-mCherry), the two-vector system (Two-vector), or the ACC/ACC SEMPER mARG plasmid (SEMPER mARG). Error bars depict SEM (N=4). Statistical analysis was conducted using a one-way ANOVA (p<0.0001) followed by Fisher's LSD post-hoc test with a single pooled variance to compare each treatment group to the not treated control. Pairwise p-values are indicated above the bars and indicate statistical significance.

FIGS. 4A-4B depict non-limiting exemplary schematics and data related to recombinant expression of monoclonal antibodies using SEMPER. (FIG. 4A) Architecture of an mRNA transcript transcribed from transfected SEMPER-b12 plasmid DNA. The first ORF, LC, encodes the b12 IgG light chain, while the second ORF, HC, encodes the b12 IgG heavy chain. Both polypeptides contain a signal peptide for secretion. The TIS of the first ORF, NNN, was varied while the TIS of the second ORF, ACC, remained constant in different constructs. (FIG. 4B) Total human IgG ELISA results following expression and secretion of SEMPER-b12 vectors. The total amount of transfected DNA was held constant across all conditions. HEK293T cells transfected with either the light chain (ACC/***) or heavy chain (***/ACC) produced no detectable IgG, similar to the no plasmid transfection. SEMPER-b12 constructs with varying expression stoichiometry produced intermediate amounts of IgG with CCC/ACC producing the highest yield. Error bars depict SEM (N=6). Statistical analysis was conducted using a one-way ANOVA (p<0.0001) followed by Fisher's LSD post-hoc test with a single pooled variance to compare each treatment group with every other. Pairwise p-values are reported in Table 8.

FIGS. 5A-5C depict non-limiting exemplary schematics and data related to 2-ORF SEMPER using in vitro transcribed mRNA. (FIG. 5A) Schematic of 2-ORF SEMPER mRNA produced from in vitro transcription. The first and second ORFs encode msfGFP [r5M] and mEBFP2 respectively. (FIG. 5B) Flow cytometry data from transient transfection of IVT mRNA encoding various TIS combinations into HEK293T cells (N=1, representative of four replicates). IVT mRNA constructs were made with (Left) standard uridine triphosphate or (Right) N1-methylpseudouridine-5′-triphosphate. (FIG. 5C) Violin plots of log 10 (msfGFP [r5M]/mEBFP2) values for msfGFP [r5M] and mEBFP2 double-positive cells transfected with TTT/ACC, CCC/ACC, or ACC/ACC IVT mRNA produced with different uridine nucleotides. Each distribution represents four combined replicates. The median and quartiles of the distribution are represented by the solid and dotted lines respectively. Statistical analysis was conducted using a one-way Welch's ANOVA (p<0.0001 (Left) and p<0.0001 (Right)) followed by Games-Howell's multiple comparisons test to compare TIS combinations.

FIGS. 6A-6C depict non-limiting exemplary schematics and data related to simulating SEMPER-based gene expression. (FIG. 6A) Depiction of an individual Monte Carlo simulation unit referred to as a “Monte Carlo mRNA”. In each mRNA simulation, scanning ribosomes are sequentially loaded onto the mRNA. Each ribosome traverses the mRNA until it is consumed by one of three Boolean stopping conditions. i) The ribosome may successfully initiate translation of ORF 1 at the first TIS to produce a single unit of Protein 1. If successful, the simulation loads the next ribosome. If unsuccessful, the ribosome continues to the TIS in front of ORF 2 and ii) the ribosome may successfully initiate translation of ORF 2 at the second TIS to produce a single unit of Protein 2. If successful, the simulation loads the next ribosome. If unsuccessful, the ribosome is discarded iii) without producing any translation product. The probability the ribosome will translate a given ORF is modeled by a Bernoulli random variable with success probability p_TIS. p_TIS values are based on the identity of the TIS in front of the ORF. This process is repeated for multiple scanning ribosomes. (FIG. 6B) Simulated flow cytometry plots for TIS combinations depicted in FIG. 1C. For each TIS combination, hundreds of thousands of Monte Carlo mRNAs were simulated. The results of these simulations were aggregated into approximately 10,000 smaller groups or “cells” and then plotted. (FIG. 6C) Modeling expression changes in ORF 2 due to the presence of an internal methionine (iMet) in ORF 1. (Left) Simulated results of the expression distributions for cells transfected with plasmids utilizing TTT and ACC for ORF 1 and ORF 2 respectively. The left-shifted, light-blue distribution is caused by the consumption of ribosomes that initiate translation at the iMet, reducing ribosomal flux to ORF 2. (Right) TTT/ACC constructs were tested experimentally, one with msfGFP [r5M] encoded in ORF 1 and one where an iMet was added back into a canonical position of msfGFP [r5M]. The presence of the iMet led to a reduction in ORF 2 translation, represented by mEBFP2 expression (N=1, representative of four replicates).

FIGS. 7A-7D depict data related to mCherry distribution plots for 2-ORF and 3-ORF fluorescent protein SEMPER libraries. Acting at the level of each mRNA, this IRES mCherry normalization strategy aimed to account for variability in transfection and transcription that could occur between tested constructs. (FIG. 7A) mCherry distributions for 2-ORF SEMPER constructs (for expression of msfGFP [r5M] and mEBFP2) after transfection in HEK293T cells. (FIG. 7B) mCherry distributions for 2-ORF SEMPER constructs (for expression of msfGFP [r5M] and mEBFP2) after transfection in CHO-K1 cells. (FIG. 7C) mCherry distributions for 3-ORF SEMPER constructs (for expression of msfBFP [r5M], msfGFP [r5M], and emiRFP670) after transient transfection in HEK293T cells. (FIG. 7D) mCherry distributions for 3-ORF SEMPER constructs (for expression of msfBFP [r5M], msfGFP [r5M], and emiRFP670) after transient transfection in CHO-K1 cells. mCherry distributions for CHO-KI cells vary less than those for HEK293T, suggesting differences in transfection efficiency and/or burden. However, despite this variation, the rank orders of ORF expression ratios and other trends of mCherry-normalized expression titration were consistent between the two cell types in FIG. 1, FIG. 2, FIG. 11, and FIG. 15 for various TIS combinations tested. Each violin plot contains data from four combined replicates of the depicted TIS combination. The median and quartiles of the distribution are represented by the solid and dotted lines respectively.

FIGS. 8A-8B depict data related to HEK293T 2-ORF compensation controls. (FIG. 8A) Flow cytometry plots for single-color control plasmids after transient transfection and compensation. Compensation was conducted using FlowJo. Depicted are cells that have been gated for by size and then doublet-discriminated. Comp-BFP-A, Comp-GFP-A, and Comp-Cherry-A, represent the channels used for mEBFP2, msfGFP [r5M], and mCherry respectively. mCherry and msfGFP [r5M] single-color controls showed some, yet not substantial, fluorescence crossover into the Comp-BFP-A channel. Measurements were taken using a MACSQuant VYB cytometer. (FIG. 8B) Flow cytometry plots for pUC19 plasmid transfections containing no fluorescent marker. Minimal fluorescence was observed in all channels.

FIGS. 9A-9B depict data related to CHO-K1 2-ORF compensation controls. (FIG. 9A) Flow cytometry plots for single-color control plasmids after transient transfection and compensation. Compensation was conducted using FlowJo. Depicted are cells that have been gated for by size and then doublet-discriminated. Comp-BFP-A, Comp-GFP-A, and Comp-Cherry-A, represent the channels used for EBFP2, msfGFP [r5M], and mCherry respectively. Minimal to no fluorescence spillover was observed in the Comp-BFP-A channel for cells transfected with msfGFP [r5M] or mCherry single-color controls. Measurements were taken using a MACSQuant VYB cytometer. (FIG. 9B) Flow cytometry plots for pUC19 plasmid transfections containing no fluorescent marker.

FIGS. 10A-10B depict non-limiting exemplary schematics and data related to additional TIS combinations for 2-ORF SEMPER. (FIG. 10A) In HEK293T cells, three additional TIS sequences (TTC, GGG, and Kozak) were tested in front of ORF 1 while maintaining a strong ACC TIS on ORF 2. ORF 1 encoded msfGFP [r5M] and ORF 2 encoded msfBFP [r5M]. IRES-mCherry was included on the plasmid as done in other designs herein. (FIG. 10B) Violin plots of log 10 (msfGFP [r5M]/msfBFP [r5M]) values for all mCherry positive cells for four combined replicates of the depicted TIS combinations. The median and quartiles of the distribution are represented by the solid and dotted lines respectively. TTC and GGG provide additional tunability of the system, accessing new expression stoichiometries. ACC TIS used for ORF 1 performs similarly to the Kozak sequence (GCCGCCACCATGG) when implemented for ORF 1. The data shown here were collected using the transfection techniques described in Methods for plasmid transfection of 2-ORF SEMPER fluorescent protein constructs, except these experiments were conducted in 96-well format and therefore transfection reagents and DNA quantities were scaled down by a factor of 4 to accommodate. The Games-Howell's multiple comparisons tests depicted above were conducted as a post-hoc analysis of Welch's ANOVA Test (p<0.0001).

FIGS. 11A-11B depict data related to CHO-K1 plasmid-based 2-ORF SEMPER results. (FIG. 11A) Flow cytometry data for CHO-KI cells transfected with 2-ORF SEMPER constructs. mCherry positive cells are depicted. Transfection conditions were optimized for HEK293T cells and therefore lower transfection efficiency was observed for CHO-KI cells. Replicates for both cell lines demonstrated reproducibility, as shown here for CHO-K1. (FIG. 11B) Min-max normalized fluorescence values for each GOI relative to ACC/*** and ***/ACC. Fluorescence measurements were first normalized by mCherry before min-max normalization. Error bars depict SEM (N=4). Welch's ANOVA tests were conducted for blue and green bars independently (p<0.0001 for all colors across all bins) followed by Dunnett's T3 multiple comparisons tests. All pairwise comparisons are provided in Table 3.

FIGS. 12A-12B depict data related to min-max controls for 3-ORF SEMPER library. (FIG. 12A) HEK293T min-max controls after mCherry normalization and min-max normalization. Error bars depict standard error of the mean (SEM) (N=4). (FIG. 12B) CHO-K1 min-max controls after mCherry normalization and min-max normalization. Error bars depict standard error of the mean (SEM) (N=4). For both cell types, min-max normalization for msfBFP [r5M] was conducted using the fluorescence values in the BFP fluorescence channel for ACC/***/*** (max) and ***/**/ACC (min). Min-max normalization for msfGFP [r5M] was conducted using the fluorescence values in the GFP fluorescence channel for ***/ACC/*** (max) and ***/***/ACC (min). Min-max normalization for emiRFP670 was conducted using the fluorescence values in the iRFP fluorescence channel for *** (min). emiRFP670 demonstrated significant fluorescence even when no start codon was present in front of it. It was observed that constructs with no start codons in front of FPs can still produce fluorescence above what was measured for no-color plasmid controls and mCherry-only controls. This suggests unexpected fluorescent spillover is not the cause. Therefore, it was hypothesized these results are due to the translation of fluorescent protein products from in-frame, non-canonical start codons or from in-frame, internal TISs. In addition, the expression of emiRFP670 from non-canonical or alternate translation initiation sites appeared to be improved when an actively translating ORF was placed further upstream (i.e. ACC/***/***) compared to the case where the actively translating ORF was placed closer to emiRFP670 (i.e. ***/ACC/***).

FIGS. 13A-13B depict data related to HEK293T 3-ORF compensation controls. (FIG. 13A) Flow cytometry plots for single-color control plasmids after transient transfection and compensation. Compensation was conducted using FlowJo. Depicted are cells that have been gated for by size and then doublet-discriminated. Comp-BFP-A, Comp-GFP-A, Comp-iRFP-A, and Comp-Cherry-A represent the channels used for msfBFP [r5M], msfGFP [r5M], emiRFP670 and mCherry respectively. No substantial fluorescence spillover was observed. Measurements were taken using a Cytoflex S cytometer. (FIG. 13B) Flow cytometry plots for pUC19 plasmid transfections containing no fluorescent marker.

FIGS. 14A-14B depict data related to CHO-K1 3-ORF Compensation Controls. (FIG. 14A) Flow cytometry plots for single-color control plasmids after transient transfection and compensation. Compensation was conducted using FlowJo. Depicted are cells that have been gated for by size and then doublet-discriminated. Comp-BFP-A, Comp-GFP-A, Comp-iRFP-A, and Comp-Cherry-A represent the channels used for msfBFP [r5M], msfGFP [r5M], emiRFP670 and mCherry respectively. No substantial fluorescence spillover was observed. Measurements were taken using a Cytoflex S cytometer. (FIG. 14B) Flow cytometry plots for pUC19 plasmid transfections containing no fluorescent marker.

FIG. 15 depicts data related to 3-ORF SEMPER constructs in CHO-K1 cells. (Left) Min-max normalized relative translation levels for each fluorescent protein. Cells were first clustered into three mCherry fluorescence bins (low, medium, high). Error bars depict SEM (N=4). Welch's ANOVA tests were conducted for blue, green, and red bars independently. The ANOVA tests for all colors across all bins yielded p<0.0001. Post-hoc Dunnett's T3 multiple comparisons tests are provided in Table 5. (Right) Comparisons of msfBFP [r5M] relative translation levels between the max control and those TIS combinations with multiple ACC driven ORFs. Error bars depict SEM (N=4). Post-hoc Dunnet's T3 comparisons tests demonstrate a significant upregulation in relative translation of msfBFP [r5M] for depicted constructs compared to that observed for ACC/***/*** for some bins. This suggests that the presence of downstream ORFs with strong TISs can lead to boosted expression of the first ORF.

FIGS. 16A-16C depict data related to SEMPER performance when first two ORFs are interchanged. As described for FIG. 3, 3-ORF SEMPER constructs were created with msfBFP [r5M] (a methionineless BFP variant), msfGFP [r5M], and emiRFP670. The first and second ORFs for some of these 3-ORF constructs were interchanged to understand how robust SEMPER is to position effects and ORF identity. The left column of figures depicts constructs that encoded msfBFP [r5M] first and msfGFP [r5M] second. The right column of figures depicts constructs that encoded msfGFP [r5M] first and msfBFP [r5M] second. emiRFP670 was maintained in the third ORF position under a strong ACC TIS for all constructs. Data was collected on a Cytoflex S flow cytometer and compensated using single-color controls in FlowJo. Cells were then gated by size and then doublet-discriminated. All cells with msfBFP [r5M] fluorescence values less than the negative control were excluded. (FIG. 16A) Min-max normalized mean values for msfBFP [r5M]/mCherry and msfGFP [r5M]/mCherry within the “mCherry Bin”. In the left column, the maximum construct for msfBFP [r5M] was chosen to be ACC/ACC/ACC and the maximum construct for msfGFP [r5M] was chosen to be ***/ACC/ACC (not depicted). In the right column, the maximum construct for msfGFP [r5M] was chosen to be ACC/ACC/ACC and the maximum construct for msfBFP [r5M] was chosen to be ***/ACC/ACC (not depicted). A mCherry single-color control was used as the minimum for all min-max normalization tasks depicted. Error bars depict standard error of the mean (SEM) (N=4). P-values depicted are unpaired two-tailed t-tests. The TTT/ACC/ACC constructs exhibited lower expression of ORF 1 regardless of ORF 1 identity as compared to the expression levels observed for ACC/ACC/ACC. However, the absolute relative translation levels for ORF 1 and ORF 2 did change based on ORF identity, suggesting that users conduct some fine-tuning of their gene circuits that utilize the SEMPER approach in some embodiments. Changing or interchanging the ORF sequences can change the secondary structure of the mRNA which may also contribute to deviations in expected expression levels. (FIG. 16B) mCherry gating strategy. Cells depicted are mCherry positive (mCherry+). As depicted, an “mCherry Bin” was created for these distributions between 4E5 and 5E5 fluorescence units (N=1, representative of four replicates). (FIG. 16C) Flow cytometry plots for cells in the “mCherry+ Bin”. The msfBFP [r5M] variant that was created exhibited dimmer fluorescence compared to msfGFP [r5M], so less separation was observed among conditions in the left column compared to conditions in the right column. Scatter plots in the left column exhibit a “curling” phenotype further from the origin, which can be attributed to high transfection efficiency and transgene burden. These curling phenotypes were mitigated in other experiments by reducing transfection efficiency. To produce the bar plots, mCherry normalization was conducted on cells in these flow cytometry plots, followed by min-max normalization of the resultant distribution means. A PUC19 no-color transfection control is depicted in grey (N=1, representative of four replicates).

FIG. 17 depicts data related to SEMPER mARGs are not inhibited by the stochasticity of co-transfection of two plasmids. Comparison of mCherry and EmGFP fluorescence in HEK293T cells transfected with the two-vector mARG expression system or the SEMPER mARG expression system. The gvpA-IRES-mCherry plasmid was transfected in a 4-fold molar excess relative to pgvpNJKFGW-EmGFP, leading to a substantial portion of cells with solely mCherry positivity compared to those transfected with the ACC/ACC SEMPER mARG construct-which contains both fluorescent proteins on a single vector.

FIGS. 18A-18B depict non-limiting exemplary schematics and data related to comparison of SEMPER-produced antibody yield with 2-vector control. (FIG. 18A) Architecture of the mRNA transcripts transcribed from transfected SEMPER-b12 plasmid DNA used for positive (ACC/***+***/ACC) and negative (ACC/*** or ***/ACC) controls. (FIG. 18B) Total human IgG ELISA results following expression and secretion of b12 vectors. HEK293T cells transfected with the 1:1 mixture of the two plasmids while preserving the total amount of transfected DNA (ACC/***+***/ACC) produced similar yield of IgG compared to SEMPER-b12 construct with CCC/ACC TIS combination. Error bars depict SEM (N=6). Statistical analysis was conducted using a one-way ANOVA (p<0.0001) followed by Fisher's LSD post-hoc test with a single pooled variance to compare each treatment group with every other. Pairwise p-values are reported in Table 8.

FIGS. 19A-19B depict data related to 2-ORF IVT mRNA tests with and without IRES-mCherry as measured by flow cytometry. (FIG. 19A) TTT/ACC and (FIG. 19B) ACC/ACC IVT mRNA constructs were synthesized with and without IRES-mCherry. The IVT mRNA tested was produced with standard UTPs. Negative control cells were transfected solely with Lipofectamine MessengerMAX reagent without mRNA. Cells were first gated by size and then doublet-discriminated. Gates drawn above are based on background fluorescence intensities measured for the negative control cells. For both TIS combinations, the IRES-mCherry mRNA constructs yielded lower mEBFP2 and msfGFP[r5m] expression following transfection into HEK293T cells (N=2). IRES-mCherry was therefore not included in IVT mRNA constructs tested and depicted herein. In some embodiments of the methods and compositions provided herein, to boost expression levels from IVT mRNA, a user may vary or include one or more of i) 5′ UTR sequences and 5′ capping reagents that enhance ribosomal loading at the 5′ end of transcripts, ii) a 3′UTR sequence and poly A tail track that ensures stability of IVT mRNA, and iii) optimizing transfection conditions to use larger amounts of IVT mRNA.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the terms “antigen” or “immunogen” are used interchangeably to refer to a substance, typically a protein, which is capable of inducing an immune response in a subject (e.g. a mammal, such as a human). The term also refers to proteins that are immunologically active in the sense that once administered to a subject, either directly or in the form of a nucleotide sequence or vector that encodes the protein, is able to evoke an immune response of the humoral and/or cellular type directed against that protein or a variant thereof.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; Pearson et al., Meth. Mol. Bio. 24:307-31, 1994; and Altschul et al., J. Mol. Biol. 215:403-10, 1990 (the content of each of these references is incorporated herein in its entirety).

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “contrast enhanced imaging” or “imaging”, as used herein indicates a visualization of a target site performed with the aid of a contrast agent administered to the target site to improve the visibility of structures or fluids by devices process and techniques suitable to provide a visual representation of a target site. Accordingly a contrast agent is a substance that enhances the contrast of structures or fluids within the target site, producing a higher contrast image for evaluation.

The term “ultrasound imaging” or ultrasound scanning” or “sonography” as used herein indicate imaging performed with techniques based on the application of ultrasound. Ultrasound can refer to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein can refer to in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or non-linear propagation depending on the signal level. Harmonic and harmonic transient ultrasound response imaging can be used for increased axial resolution, as harmonic waves are generated from non-linear distortions of the acoustic signal as the ultrasound waves insonate tissues in the body. Other ultrasound techniques and devices suitable to image a target site using ultrasound would be understood by a skilled person.

Mammalian Polycistronic Expression System for Direct Translation from RNA and Secretion of Cargo

The field of mammalian synthetic biology and medicine often necessitates the expression of multiple proteins in user-defined stoichiometries from singular, compactly encoded transcripts. Disclosed herein are innovative methods for expressing several open reading frames (ORFs) from individual transcripts by, in some embodiments, and without being bound by any particular theory, leveraging the leaky scanning model of translation initiation. This approach can enable the translation of adjacent ORFs from a single messenger RNA (mRNA) in adjustable ratios, which can be determined by their position in the sequence and the potency of their translation initiation sites. The method, in some embodiments, is termed Stoichiometric Expression of Messenger Polycistrons by Eukaryotic Ribosomes (SEMPER).

To demonstrate the principles of SEMPER, up to three distinct fluorescent proteins were successfully expressed from a single plasmid in two separate cell lines (Example 1). Also show herein is that the SEMPER paradigm can be used to produce secreted proteins. Following this, the method was applied to create a stoichiometrically fine-tuned polycistronic construct that encodes gas vesicle acoustic reporter genes. The results provided herein show that maintaining the ideal ratio in every cell minimizes cell toxicity. Lastly, the expression of two different fluorescent proteins from individual mRNA transcripts generated through in vitro transcription was shown (Example 1). Also shown is that new stoichiometries are achievable with IVT mRNA by changing the chemistry of nucleotides used in IVT reactions. This approach enables a user to increase the complexity of mRNA vaccines and therapeutics and the protein products they encode to combat disease.

Provided herein are methods and compositions which overcomes all the aforementioned challenges. The disclosed methods and compositions enable expression of multiple proteins from a single mRNA, and provide a user-friendly framework to arbitrarily and predictably tune translated product stoichiometry. There are provided, in some embodiments, compositions. Compositions disclosed herein include nucleic acid compositions, pharmaceutical compositions, engineered cells, mRNA vaccines, and vectors. There are provided, in some embodiments, nucleic acid compositions. In some embodiments, the nucleic acid composition comprises: a polynucleotide comprising a first nucleic acid unit and a second nucleic acid unit. In some embodiments, the first nucleic acid unit encodes one or more first unit payload protein(s). In some embodiments, the second nucleic acid unit encodes one or more second unit payload protein(s). In some embodiments, the first nucleic acid unit and the second nucleic acid unit each comprise an engineered translation initiation site (eTIS) comprising a three-nucleotide tunable element immediately upstream of a start codon. In some embodiments, the polynucleotide is capable of being translated to generate the one or more first unit payload protein(s) and the one or more second unit payload protein(s), optionally the polynucleotide is a polycistronic transcript. In some embodiments, the eTIS of each of the first nucleic acid unit and the second nucleic acid unit is configured to achieve a predetermined stoichiometry of the one or more first unit payload protein(s) and one or more second unit payload protein(s) in a cell or cell-like environment. In some embodiments, (i) the nucleic acid composition comprises at least one modified nucleotide and/or at least one nucleotide analogue or nucleotide derivative; and/or (ii) the polynucleotide comprises an eTIS combination of a first eTIS of the first nucleic acid unit and a second eTIS of the second nucleic acid unit, and wherein the tunable element of the first eTIS and the second eTIS are independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof. There are provided, in some embodiments, nucleic acid compositions. In some embodiments, the nucleic acid composition comprises: a promoter operably linked to a polynucleotide comprising a first nucleic acid unit and a second nucleic acid unit. In some embodiments, the first nucleic acid unit encodes one or more first unit payload protein(s). In some embodiments, the second nucleic acid unit encodes one or more second unit payload protein(s). In some embodiments, the first nucleic acid unit and the second nucleic acid unit each comprise an engineered translation initiation site (eTIS) comprising a three-nucleotide tunable element immediately upstream of a start codon. In some embodiments, the promoter is capable of inducing transcription of the first nucleic acid unit and the second nucleic acid unit to generate a polycistronic transcript. In some embodiments, the polycistronic transcript is capable of being translated to generate the one or more first unit payload protein(s) and the one or more second unit payload protein(s). In some embodiments, the eTIS of each of the first nucleic acid unit and the second nucleic acid unit is configured to achieve a predetermined stoichiometry of the one or more first unit payload protein(s) and one or more second unit payload protein(s) in a cell or cell-like environment. In some embodiments, (i) the nucleic acid composition comprises at least one modified nucleotide and/or at least one nucleotide analogue or nucleotide derivative; and/or (ii) the polynucleotide comprises an eTIS combination of a first eTIS of the first nucleic acid unit and a second eTIS of the second nucleic acid unit, and wherein the tunable element of the first eTIS and the second eTIS are independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

In some embodiments, the eTIS comprises at least one modified nucleotide and/or at least one nucleotide analogue or nucleotide derivative. In some embodiments, the at least one modified nucleotide and/or at least one nucleotide analogues is selected from a backbone modified nucleotide, a sugar modified nucleotide and/or a base modified nucleotide, or any combination thereof. In some embodiments, the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, N-1-methylpseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl) uridine, 5-(isopentenylaminomethyl)-2-thiouridine, 2-aminoadenosine or 5-(isopentenylaminomethyl)-2′-O-methyluridine or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or 0(6)-methylguanine.

In some embodiments, the eTIS combination comprises: a first eTIS comprising a tunable element consisting of TTT or UUU; and a second eTIS comprising a tunable element consisting of ACC. In some embodiments, the eTIS combination comprises: a first eTIS comprising a tunable element consisting of CCC; and a second eTIS comprising a tunable element consisting of ACC. In some embodiments, the eTIS combination comprises: a first eTIS comprising a tunable element consisting of ACC; and a second eTIS comprising a tunable element consisting of ACC. In some embodiments, the eTIS combination comprises: a first eTIS comprising a tunable element consisting of TTC or UUC; and a second eTIS comprising a tunable element consisting of ACC. In some embodiments, the eTIS combination comprises: a first eTIS comprising a tunable element consisting of GGG; and a second eTIS comprising a tunable element consisting of ACC.

In some embodiments, the polynucleotide further comprises n supplemental nucleic acid unit(s), wherein n is an integer greater than zero (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values) . . . . In some embodiments, each supplemental nucleic acid unit encodes one or more supplemental unit payload protein(s). In some embodiments, each supplemental nucleic acid unit comprises an engineered translation initiation site (eTIS) comprising a three-nucleotide tunable element immediately upstream of a start codon. In some embodiments, the promoter is capable of inducing transcription of the first nucleic acid unit, the second nucleic acid unit, and each supplemental nucleic acid unit to generate the polycistronic transcript, In some embodiments, the polycistronic transcript is capable of being translated to generate the one or more first unit payload protein(s), the one or more second unit payload protein(s), and the one or more supplemental unit payload protein(s) encoded by each of the n supplemental nucleic acid unit(s). In some embodiments, the eTIS of each of the first nucleic acid unit, the second nucleic acid unit, and the n supplemental nucleic acid unit(s) is configured to achieve a predetermined stoichiometry of the one or more first unit payload protein(s), the one or more second unit payload protein(s), and the one or more supplemental unit payload protein(s) encoded by each of the n supplemental nucleic acid unit(s) in a cell or cell-like environment.

In some embodiments, the first nucleic acid unit is upstream of the second nucleic acid unit, optionally the second nucleic acid unit is upstream of the n supplemental nucleic acid unit(s). In some embodiments, the first nucleic acid unit, the second nucleic acid unit, and the n supplemental nucleic acid unit(s) each comprise an open reading frame (ORF). In some embodiments, the tunable element modulates the strength of an eTIS of a nucleic acid unit, and wherein the strength of an eTIS of a nucleic acid unit is related to the fraction of the 43S preinitiation complex (PIC) scanning the polycistronic transcript that initiate and translate the open reading frame of said nucleic acid unit by engaging with the 60S ribosomal subunit upon reaching said eTIS. In some embodiments, the tunable element of each (eTIS) of each supplemental nucleic acid unit is independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

There are provided, in some embodiments, engineered cells generated via expression of the one or more first unit payload protein(s), the one or more second unit payload protein(s), and/or the one or more supplemental unit payload protein(s). In some embodiments, the nucleic acid composition is (i) an in vitro transcribed (IVT) mRNA construct and/or (ii) is the product of production in a cell or cell-like environment. In some embodiments, the nucleic acid composition comprises a nucleic acid library, optionally configured for screening, optionally for screening related to the expression and tuning of enzymatic pathways, circuits, and/or assembly of multimeric protein structures. In some embodiments, the nucleic acid composition is or comprises: (i) an mRNA vaccine; (ii) a circular RNA molecule; and/or (iii) mRNA, optionally the mRNA is formulated in a lipid nanoparticle (LNP). In some embodiments, a payload protein comprises an organelle localization sequence, optionally an ER localization sequence, peroxisomal targeting sequence, a lysosomal targeting sequence, a chloroplast targeting sequence, a mitochondrial targeting sequence, a Golgi localization sequence, and/or a ER retention signal.

The first nucleic acid unit can be upstream of the second nucleic acid unit. The second nucleic acid unit can be upstream of the n supplemental nucleic acid unit(s). The first nucleic acid unit, the second nucleic acid unit, and the n supplemental nucleic acid unit(s) each can comprise an open reading frame (ORF). The expression levels of unit payload proteins derived from the same nucleic acid unit can be the same or substantially the same. For example, the first nucleic acid unit can encode three first unit payload proteins, and the expression levels of each of the three first unit payload proteins can be the same or substantially the same. In some embodiments, the tunable element modulates the strength of an eTIS of a nucleic acid unit. In some embodiments, the strength of an eTIS of a nucleic acid unit is related to the fraction of the ribosomes scanning the polycistronic transcript that initiate and translate the open reading frame of said nucleic acid unit upon reaching said eTIS. The expression level of a unit payload protein can be inversely related to the number and strength of eTIS situated upstream of the nucleic acid unit from which it derives on the polycistronic transcript. The strength of the eTIS of the first nucleic acid unit can be inversely proportional to the expression level of the second unit payload protein(s). The expression level of the second unit payload protein(s) can be inversely related to the fraction of the ribosomes initiating and translating the open reading frame of the first nucleic acid unit. The strength of the eTIS of the second nucleic acid unit can be greater than the strength of the eTIS of the first nucleic acid unit, and thereby the eTIS of the second nucleic acid unit efficiently captures the ribosomal translational activity that fails to initiate at the eTIS of the first nucleic acid unit.

The tunable element can be selected from AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, and any combination thereof. The tunable element can be selected from ACC, GGG, CCC, TTC, TTT, and any combination thereof. The tunable element can be AAA, AAU, AAC, AAG, AUA, AUU, AUC, AUG, ACA, ACU, ACC, ACG, AGA, AGU, AGC, AGG, UAA, UAU, UAC, UAG, UUA, UUU, UUC, UUG, UCA, UCU, UCC, UCG, UGA, UGU, UGC, UGG, CAA, CAU, CAC, CAG, CUA, CUU, CUC, CUG, CCA, CCU, CCC, CCG, CGA, CGU, CGC, CGG, GAA, GAU, GAC, GAG, GUA, GUU, GUC, GUG, GCA, GCU, GCC, GCG, GGA, GGU, GGC, GGG, or any combination thereof. The tunable element can be ACC, GGG, CCC, UUC, UUU, or any combination thereof. The eTIS of each nucleic acid unit can be tuned via the tunable element such that the unit payload proteins reach a desired stoichiometry. For example, the first unit payload protein(s) and the second unit payload protein(s) can be in a ratio from 1:100 to 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values).

The first nucleic acid unit, the second nucleic acid unit, and/or n supplemental nucleic acid unit(s) each can comprise one or more of a first eTIS, a second eTIS, a third eTIS, a fourth eTIS, and/or a fifth eTIS. In some embodiments, the first eTIS comprises a tunable element consisting of ACC; the second eTIS comprises a tunable element consisting of GGG; the third eTIS comprises a tunable element consisting of CCC; the fourth eTIS comprises a tunable element consisting of TTC or UUC; and the fifth eTIS comprises a tunable element consisting of TTT or UUU. In some embodiments, the first eTIS has greater strength than the second eTIS, wherein the second eTIS has greater strength than the third eTIS, wherein the third eTIS has greater strength than the fourth eTIS, and wherein the fourth eTIS has greater strength than the fifth eTIS. The eTIS can comprise a G nucleotide immediately downstream of the start codon. The steady-state levels of one or more of the first unit payload protein(s), the second unit payload protein(s), and/or the supplemental unit payload protein(s) can be at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, or a number or a range between any two of these values, greater than the steady-state levels of one or more of the first unit payload protein(s), the second unit payload protein(s), and/or the supplemental unit payload protein(s). The difference between the steady-state levels of one or more of the first unit payload protein(s), the second unit payload protein(s), and/or the supplemental unit payload protein(s) and the steady-state levels of one or more of the first unit payload protein(s), the second unit payload protein(s), and/or the supplemental unit payload protein(s) can be less than about, or can be greater than about, one order of magnitude. The expression level of the first unit payload protein(s), the second unit payload protein(s), and the supplemental unit payload protein(s) can be related to the strength of the eTIS of the corresponding nucleic acid unit from which it derives. The predetermined stoichiometry can be configured to achieve a therapeutic level of the first unit payload protein(s), the second unit payload protein(s), and the supplemental unit payload protein(s). The predetermined stoichiometry can be configured to achieve efficacious steady-state protein levels of each of the first unit payload protein(s), the second unit payload protein(s), and the supplemental unit payload protein(s). The predetermined stoichiometry can be robust to tissue tropism and stochastic expression.

In some embodiments, the n supplemental nucleic acid unit(s) comprise one or more of a third nucleic acid unit, a fourth nucleic acid unit, a fifth nucleic acid unit, a sixth nucleic acid unit, a seventh nucleic acid unit, an eight nucleic acid unit, and a ninth nucleic acid unit. In some embodiments, the eTIS combination further comprises a third eTIS of the third nucleic acid unit. In some embodiments, the eTIS combination comprises: a first eTIS comprising a tunable element consisting of ACC; a second eTIS comprising a tunable element consisting of ACC; and a third eTIS comprising a tunable element consisting of ACC. In some embodiments, the eTIS combination comprises: a first eTIS comprising a tunable element consisting of ACC; a second eTIS comprising a tunable element consisting of TTT or UUU; and a third eTIS comprising a tunable element consisting of ACC. In some embodiments, the eTIS combination comprises: a first eTIS comprising a tunable element consisting of CCC; a second eTIS comprising a tunable element consisting of TTT or UUU; and a third eTIS comprising a tunable element consisting of ACC. In some embodiments, the eTIS combination comprises: a first eTIS comprising a tunable element consisting of TTT or UUU; a second eTIS comprising a tunable element consisting of ACC; and a third eTIS comprising a tunable element consisting of ACC. In some embodiments, the eTIS combination comprises: a first eTIS comprising a tunable element consisting of TTT or UUU; a second eTIS comprising a tunable element consisting of CCC; and a third eTIS comprising a tunable element consisting of ACC. In some embodiments, the eTIS combination comprises: a first eTIS comprising a tunable element consisting of TTT or UUU; a second eTIS comprising a tunable element consisting of TTT or UUU; and a third eTIS comprising a tunable element consisting of ACC. In some embodiments, the first eTIS, the second eTIS, the third eTIS, the fourth eTIS, and/or the fifth eTIS comprise a Kozak sequence or derivative thereof. In some embodiments, the first eTIS, the second eTIS, the third eTIS, the fourth eTIS, and/or the fifth eTIS comprise a start codon and about 3-10 neighboring nucleotides.

In some embodiments, one or more of the first unit payload protein(s), the second unit payload protein(s), and the supplemental unit payload protein(s) is a payload, is a component of a payload, or chaperones the assembly of a payload. In some embodiments, two or more of the first unit payload protein(s), the second unit payload protein(s), and the supplemental unit payload protein(s) are components of a multimeric protein complex. In some embodiments, two or more of the first unit payload protein(s), the second unit payload protein(s), and/or the supplemental unit payload protein(s) are capable of forming an antibody or antigen-binding fragment thereof, optionally one or more of the first unit payload protein(s), the second unit payload protein(s), and the supplemental unit payload protein(s) is a light chain, and one or more of the first unit payload protein(s), the second unit payload protein(s), and the supplemental unit payload protein(s) is heavy chain. In some embodiments, one or more of the first unit payload protein(s), the second unit payload protein(s), and the supplemental unit payload protein(s) is a toxin, and wherein one or more of the first unit payload protein(s), the second unit payload protein(s), and the supplemental unit payload protein(s) is an antitoxin. In some embodiments, one or more of the first unit payload protein(s), the second unit payload protein(s), and the supplemental unit payload protein(s) is a structural protein, and wherein one or more of the first unit payload protein(s), the second unit payload protein(s), and the supplemental unit payload protein(s) is a chaperone. In some embodiments, two or more of the first unit payload protein(s), the second unit payload protein(s), and/or the supplemental unit payload protein(s) are capable of one or more of: (i) forming mosaic virus-like particles; (ii) forming all or a portion of a metabolic pathway; (iii) complex and/or polyclonal antigen(s); (iv) a cytokine cocktail; (v) an immunomodulatory cocktail; (vi) all or a portion of an enzymatic pathway, optionally for the production of a small molecule, further optionally a small molecule therapeutic; and (vii) ex vivo and/or in vivo cell fate determination and/or reprogramming, further optionally via expression of two or more transcription factors, optionally expression of two or more transcription factors radiometrically. In some embodiments, two or more of the first unit payload protein(s), the second unit payload protein(s), and/or the supplemental unit payload protein(s) are configured for secretion, optionally via an N-terminal signal peptide, further optionally a signal peptide derived from CD8, IgG1 heavy chain, IgK light chain and/or GM-CSF. In some embodiments, the nucleic acid composition is capable of causing less cellular burden, toxicity, and/or apoptosis as compared to a nucleic acid composition wherein the expression of the first unit payload protein(s), the second unit payload protein(s), and the supplemental unit payload protein(s) is achieved via two or more separate polynucleotides, optionally the separate polynucleotides are separate vectors.

In some embodiments, one or more of the first nucleic acid unit, the second nucleic acid unit, and the n supplemental nucleic acid unit(s) do not comprise an internal start codon. In some embodiments, one or more of the first nucleic acid unit, the second nucleic acid unit, and the n supplemental nucleic acid unit(s) have been configured to not comprise an internal start codon. One or more of the first nucleic acid unit, the second nucleic acid unit, and the n supplemental nucleic acid unit(s) can be codon-optimized. In some embodiments, the polycistronic transcript does not comprise an upstream ORF (uORF). In some embodiments, the first unit payload protein(s) is not less than about 30, about 25, about 20, about 15, about 10, or about 5, amino acids in length. In some embodiments, one or more of the first unit payload protein(s), the second unit payload protein(s), and/or the supplemental unit payload protein(s) does not comprise an internal methionine residue. In some embodiments, one or more of the first unit payload protein(s), the second unit payload protein(s), and/or the supplemental unit payload protein(s) does not comprise non-native amino acid residues. In some embodiments, one or more of the first nucleic acid unit, the second nucleic acid unit, and the n supplemental nucleic acid unit(s) do not comprise a tandem gene expression element. A tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, one or more of the first nucleic acid unit, the second nucleic acid unit, and the n supplemental nucleic acid unit(s) encode more than one payload protein. One or more of the first nucleic acid unit, the second nucleic acid unit, and the n supplemental nucleic acid unit(s) can comprise a tandem gene expression element selected from an IRES, F2A, E2A, P2A or T2A, or any combination thereof.

The polynucleotide can comprise a 5′UTR and/or a 3′UTR. The promoter can comprise a heterologous promoter element and/or an endogenous promoter element. The heterologous promoter element can be capable of being bound by a component of a synthetic protein circuit. An endogenous promoter element can be capable of being bound by an endogenous protein of a cell. The promoter can comprise a minimal promoter (e.g., TATA, miniCMV, and/or miniPromo). The promoter can comprise a ubiquitous promoter, an inducible promoter, a tissue-specific promoter and/or a lineage-specific promoter. The ubiquitous promoter can be a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

In some embodiments, the polynucleotide, the second polynucleotide, the first nucleic acid unit, the second nucleic acid unit, and/or the n supplemental nucleic acid unit(s) is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, nucleotides in length In some embodiments, first unit payload protein(s), second unit payload protein(s), supplemental unit payload protein(s), and/or secondary unit payload protein(s) is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, or a number or a range between any two of these values, amino acids in length. In some embodiments, the nucleic acid composition further comprises a second polynucleotide comprising m secondary nucleic acid units. The polynucleotide and the second polynucleotide can be situated on the same nucleic acid or different nucleic acids. In some embodiments, m is an integer greater than one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values). In some embodiments, a second promoter operably linked to the second polynucleotide. In some embodiments, each secondary nucleic acid unit encodes one or more secondary unit payload protein(s). Each secondary nucleic acid unit can comprise an eTIS comprising a three-nucleotide tunable element immediately upstream of a start codon. The second promoter can be capable of inducing transcription of each secondary nucleic acid unit to generate to generate a second polycistronic transcript. The second polycistronic transcript can be capable of being translated to generate the one or more secondary unit payload protein(s) encoded by each of the m secondary nucleic acid units. The eTIS of each of the m secondary nucleic acid units can be configured to achieve a predetermined stoichiometry of the one or more secondary unit payload protein(s) encoded by each of the m secondary nucleic acid unit in a cell or cell-like environment.

The systems, methods, compositions, and kits provided herein can, in some embodiments, be employed in concert with the systems, methods, compositions, and kits for described in PCT Application Publication No. WO2023288090A1 and in Duan et al. (“Stoichiometric expression of messenger polycistrons by eukaryotic ribosomes (SEMPER) for compact, ratio-tunable multi-gene expression from single mRNAs.” bioRxiv (2023): 2023-05), the contents of which are incorporated herein by reference in their entirety.

Disclosed herein include methods comprising: introducing a nucleic acid library into a cell population, optionally a nucleic acid library comprising a set of predetermined eTIS combinations, further optionally a nucleic acid library configured for screening, optionally derived from a nucleic acid composition disclosed herein, further optionally the introducing step comprises one or more of transfection, transduction, plasmid-based expression, mRNA-based expression, or integrated DNA-based expression, optionally the nucleic acid composition comprises a plasmid, an mRNA, a transient expression vector, or an integrating expression vector; and screening for nucleic acid library member(s) yielding user-desired outcome(s) and/or expression ratio(s), optionally an optimal stoichiometry for a particular tissue and/or cell type, optionally screening related to the expression and tuning of enzymatic pathways, circuits, and/or assembly of multimeric protein structures.

Disclosed herein include methods comprising: introducing an effective amount of a nucleic acid composition disclosed herein into cell(s) or a cell-like environment, optionally the cell-like environment is configured for in vitro transcription, further optionally the introducing step comprises one or more of transfection, transduction, plasmid-based expression, mRNA-based expression, or integrated DNA-based expression, optionally the nucleic acid composition comprises a plasmid, an mRNA, a transient expression vector, or an integrating expression vector; and extracting polycistronic transcript(s) expressed in said cell(s) or cell-like environment.

### Gas Vesicles

Disclosed herein include nucleic acid compositions encoding one or more types of gas vesicles (GVs). In some embodiments, first unit payload protein(s), second unit payload protein(s), supplemental unit payload protein(s), and/or secondary unit payload protein(s) are gas vesicle assembly (GVA) proteins and/or gas vesicle structural (GVS) proteins. The promoter, polynucleotide, second promoter, and/or second polynucleotide can be configured to express the gas vesicle(s) in response a biochemical event in the cell. The expression of the gas vesicle(s) can be an output of a synthetic protein circuit. In some embodiments, the polynucleotide and/or second polynucleotide encode GVA genes and/or GVS genes capable of forming one or more gas vesicle(s) upon expression in the cell or cell-like environment, such as a plurality of gas vesicles, or a plurality of gas vesicles and a plurality of secondary gas vesicles. The plurality of secondary gas vesicles can comprise distinctive mechanical, acoustic, surface and/or magnetic properties as compared to the plurality of gas vesicles. Two or more of the first unit payload protein(s), the second unit payload protein(s), and/or the supplemental unit payload protein(s) can be capable of forming gas vesicle(s), such as, for example, gas vesicle(s) derived from a species of Anabaena bacteria, Halobacterium salinarum, and/or Bacillus megaterium. Provided herein are nucleic acid compositions encoding gas-filled protein structures (GVPS), also referred to as “gas vesicles” (GVs). The phrases “gas vesicles protein structure” or “GV”, “GVP”, “GVPS” or “Gas Vesicles” as used herein shall be given their ordinary meaning, and shall also refer to a gas-filled protein structure intracellularly expressed by certain bacteria or archea as a mechanism to regulate cellular buoyancy in aqueous environments. GVs are described in Walsby, A. E. ((1994). Gas vesicles. Microbiology and Molecular Biology Reviews, 58 (1), 94-144) hereby incorporated by reference in its entirety. The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as gvpA/B) and optionally also a GVS identified as gvpC. The compositions, methods and systems described herein can be used with compositions, methods and systems (e.g., gas vesicle compositions and ultrasonic methods) previously described in U.S. Patent Application Publication Nos. 2014/0288411, 2014/0288421, 2018/0030501, 2018/0038922, 2019/0175763, 2019/0314001, 2020/0164095, and 2020/0237346, and International Patent Application Publication WO2020/146379; the content of each of these applications is incorporated herein by reference in its entirety.

In particular, a GV in the sense of the disclosure is a structure intracellularly expressed by bacteria or archaea forming a hollow structure wherein a gas is enclosed by a protein shell, which is a shell substantially made of protein (up at least 95% protein). In gas vesicles in the sense of the disclosure, the protein shell is formed by a plurality of proteins herein also indicated as Gvp proteins or Gvps, which are expressed by the bacteria or archaea and form in the bacteria or archaea cytoplasm a gas permeable and liquid impermeable protein shell configuration encircling gas. Accordingly, a protein shell of a GV is permeable to gas but not to surrounding liquid such as water. For example, GVs' protein shells exclude liquid water but permit gas to freely diffuse in and out from the surrounding media making them physically stable despite their usual nanometer size.

GV structures are typically nanostructures with widths and lengths of nanometer dimensions (in particular with widths of 45-250 nm and lengths of 100-800 nm) but can have lengths as large as the dimensions of a cell in which they are expressed, as will be understood by a skilled person. GVs and methods are described in Farhadi et al, Science, 2019, hereby incorporated by reference. In certain embodiments, the gas vesicles protein structure have average dimensions of 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

GVs in the sense of the disclosure have different shapes depending on their genetic origins. For example, GVs in the sense of the disclosure can be substantially spherical, ellipsoid, cylindrical, or have other shapes such as football shape or cylindrical with cone shaped end portions depending on the type of bacteria providing the gas vesicles.

The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as Gvp A/B) and optionally also a GVS identified as gvpC. GvpA is a structural protein that assembles through repeated unites to make up the bulk of GVs. GvpC is a scaffold protein with 5 repeat units that assemble on the outer shell of GVs. GvpC can be engineered to tune the mechanical and acoustic properties of GVs as well as act as a handle for appending moieties on to. A gvpC protein is a hydrophilic protein of a GV shell, which includes repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another.

The optional gvpC gene encodes for a gvpC protein which is a hydrophilic protein of a GV shell, including repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another. In performing alignment steps sequence are identified as repeat when the sequence shows at least 3 or more of the characteristics described in US 2018/0030501 (incorporated herein by reference in its entirety) which also include additional features of gvpC proteins and the related identification.

The phrase “GV type” as used herein shall be given its ordinary meaning, and shall also refer to a gas vesicle having dimensions and shape resulting in distinctive mechanical, acoustic, surface and/or magnetic properties as will be understood by a skilled person upon reading of the present disclosure. In particular, a skilled person will understand that different shapes and dimensions will result in different properties in view of the indications in provided in U.S. application Ser. No. 15/613,104 and U.S. Ser. No. 15/663,600 and additional indications identifiable by a skilled person. In some embodiments, the nucleic acid compositions provided herein encode a combination of different GV types and/or variants thereof, with each expressed GV exhibiting a different acoustic collapse profile with progressively decreased midpoint collapse pressure values. In some embodiments, the percentage difference between the midpoint collapse pressure values of any given two expressed GVs types is at least twenty percent.

The GVs can be capable of withstanding pressures of several kPa. But collapse irreversibly at a pressure at which the GV protein shell is deformed to the point where it flattens or breaks, allowing the gas inside the GV to dissolve irreversibly in surrounding media, herein also referred to as a critical collapse pressure, or selectable critical collapse pressure, as there are various points along a collapse pressure profile (e.g., peak acoustic pressure).

A collapse pressure profile (e.g., peak acoustic pressure) as used herein indicates a range of pressures over which collapse of a population of GVs of a certain type occurs. In particular, a collapse pressure profile in the sense of the disclosure comprise increasing acoustic collapse pressure values, starting from an initial collapse pressure value at which the GV signal/optical scattering by GVs starts to be erased to a complete collapse pressure value at which the GV signal/optical scattering by GVs is completely erased. The collapse pressure profile of a set type of GV is thus characterized by a mid-point pressure where 50% of the GVs of the set type have been collapsed (also known as the “midpoint collapse pressure”), an initial collapse pressure where 5% or lower of the GVs of the type have been collapsed, and a complete collapse pressure where at least 95% of the GVs of the type have been collapsed. In some embodiments herein described a selectable critical collapse pressure can be any of these collapse pressures within a collapse pressure profile, as well as any point between them. The critical collapse pressure profile of a GV is functional to the mechanical properties of the protein shell and the diameter of the shell structure. U.S. Patent Application Publication No. 2020/0164095 describes gas vesicles, protein variants and related compositions methods and systems for singleplexed and/or multiplexed ultrasonic methods (e.g., imaging of a target site in which a gas vesicle provides contrast for the imaging) which is modifiable by application of a selectable acoustic collapse pressure value of the gas vesicle, the content of which is hereby expressly incorporated by reference in its entirety.

The acoustic collapse pressure profile (e.g., peak acoustic pressure) of a given GV type can be determined by imaging GVs with imaging ultrasound energy after collapsing portions of the given GV type population with a collapsing ultrasound energy (e.g. ultrasound pulses) with increasing peak positive pressure amplitudes to obtain acoustic pressure data point of acoustic pressure values, the data points forming an acoustic collapse curve. The acoustic collapse pressure function f(p) can be derived from the acoustic collapse curve by fitting the data with a sigmoid function such as a Boltzmann sigmoid function. An acoustic collapse pressure profile in the sense of the disclosure can include a set of initial collapse pressure values, a midpoint collapse pressure value and a set of complete collapse pressure values. The initial collapse pressures are the acoustic collapse pressures at which 5% or less of the GV signal is erased. A midpoint collapse pressure is the acoustic collapse pressure at which 50% of the GV signal is erased. Complete collapse pressures are the acoustic collapse pressures at which 95% or more of the GV signal is erased. The pressure can be peak pressure. In some embodiments, the peak pressure is peak positive pressure. In some embodiments, the peak pressure is peak negative pressure.

One or more of the first unit payload protein(s), the second unit payload protein(s), and/or the supplemental unit payload protein(s) can be encoded by GVA genes and/or GVS genes, such as, GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Halorubrum vacuolatum, Microcystis aeruginosa, Methanosarcina barkeri, Streptomyces coelicolor, and/or Psychromonas ingrahamii.

The polynucleotide and/or second polynucleotide comprises: two or more GVA and/or GVS genes derived from different prokaryotic species; GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii; gvpB, gvpN gvpF, gvpG, gvpL gvpS, gvpK, gvpJ, and/or gvpU from B. megaterium; gvpA, gvpC, gvpN, gvpJ, gvpK, gvpF, gvpG, gvpV, and/or gvpW from Anabaena flos-aquae; gvpR, gvpN, gvpF, gvpG, gvpL, gvpS, gvpK, gvpJ, gvpT and/or gvpU from B. megaterium and gvpA from Anabaena flos-aquae; gvpA, and/or gvpC from Anabaena flos-aquae, and gvpN, gvpF, gvpG, gvpL, gvpS, gvpK, gvpJ, and/or gvpU from B. megaterium; and/or gvpA, gvpC and/or gvpN from Anabaena flos-aquae, and gvpF, gvpG, gvpL, gvpS, gvpK, gvpJ, and/or gvpU from B. megaterium.

U.S. Patent Application Publication No. 2018/0030501 describes hybrid gas vesicle gene cluster (GVGC) configured for expression in a prokaryotic host comprising gas vesicle assembly (GVA) genes native to a GVA prokaryotic species and capable of being expressed in a functional form in the prokaryotic host, as well as one or more gas vesicle structural (GVS) genes native to one or more GVS prokaryotic species, at least one of the one or more GVS prokaryotic species different from the GVA prokaryotic species, and related gas vesicle reporting (GVR) genetic circuits, genetic, vectors, engineered cells, and related compositions methods and systems to produce GVs, hybrid GVGC and/or image a target site, the content of which is hereby expressly incorporated by reference in its entirety. The term “Gas Vesicle Genes Cluster” or “GVGC” as described herein indicates a gene cluster encoding a set of GV proteins capable of providing a GV upon expression within a cell. In some embodiments, the nucleic acid compositions provided herein encode some or all elements of a GVGC. The term “gene cluster” as used herein means a group of two or more genes found within an organism's DNA that encode two or more polypeptides or proteins, which collectively share a generalized function or are genetically regulated together to produce a cellular structure and are often located within a few thousand base pairs of each other. The size of gene clusters can vary significantly, from a few genes to several hundred genes. Portions of the DNA sequence of each gene within a gene cluster are sometimes found to be similar or identical; however, the resulting protein of each gene is distinctive from the resulting protein of another gene within the cluster. Genes found in a gene cluster can be observed near one another on the same chromosome or native plasmid DNA, or on different, but homologous chromosomes. An example of a gene cluster is the Hox gene, which is made up of eight genes and is part of the Homeobox gene family. In the sense of the disclosure, gene clusters as described herein also comprise gas vesicle gene clusters, wherein the expressed proteins thereof together are able to form gas vesicles.

Engineered GVs and methods of tuning the acoustic properties thereof are provided in U.S. Patent Application Publication No. 2020/0164095, the content of which is incorporated herein by reference in its entirety. In some embodiments, the GVs can be engineered to modulate the GV mechanical, acoustic, surface and targeting properties in order to achieve enhanced harmonic responses and multiplexed imaging to be better distinguished from background tissues. In some embodiments herein described Gas vesicles protein structures can be provided by Gvp genes endogenously expressed in bacteria or archaea. Endogenous expression can refer to expression of Gvp proteins forming the protein shell of the GV in bacteria or archaea that naturally produce gas vesicles encoded (e.g. in their genome or native plasmid DNA). Gvp proteins expressed by bacteria or archaea typically include two primary structural proteins, here also indicated as GvpA and GvpC, and several putative minor components and chaperones as would be understood by a person skilled in the art. In some embodiments, heterologously expressed Gvp proteins to provide a GV type have independently at least 50% sequence identity, preferably at least 80%, more preferably at least 90%, most preferably at least 95% sequence identity compared to a reference sequence of corresponding Gvp protein using one of the alignment programs described using standard parameters.

The GVA genes and GVS genes can have sequences codon optimized for expression in a eukaryotic cell. The gas vesicle(s) can comprise a GVS variant engineered to present a tag enabling clustering in the cell. The gas vesicle(s) can comprise a GvpC variant comprising at least one protease recognition site inserted within the central portion and/or attached to at least one of the N-terminus and the C-terminus of the Gvp. One or more of the mechanical, acoustic, surface and/or magnetic properties of the gas vesicle(s) can be capable of being configured by adjusting the eTIS of one or more of the first nucleic acid unit, the second nucleic acid unit, the n supplemental nucleic acid unit(s), and/or the secondary nucleic acid units. The gas vesicle(s) can be hybrid gas vesicle(s) derived from two or more prokaryotic species.

The plurality of gas vesicles can comprise a first collapse pressure profile. The first collapse pressure profile can comprise a collapse function from which a gas vesicle collapse amount can be determined for a given pressure value. In some embodiments, the first collapse pressure profile comprises a first initial collapse pressure where 5% or lower of the plurality of gas vesicles are collapsed, a first midpoint collapse pressure where 50% of the plurality of gas vesicles are collapsed, a first complete collapse pressure where at least 95% of the plurality of gas vesicles are collapsed, any pressure between the first initial collapse pressure and the first midpoint collapse pressure, and any pressure between the first midpoint collapse pressure and the first complete collapse pressure. In some embodiments, a first selectable collapse pressure is: any collapse pressure within the first collapse pressure profile; selected from the first collapse pressure profile at a value between 0.05% collapse of the plurality of gas vesicles and 95% collapse of the plurality of gas vesicles; equal to or greater than the first initial collapse pressure; equal to or greater than the first midpoint collapse pressure; and/or equal to or greater than the first complete collapse pressure.

The plurality of secondary gas vesicles can comprise a second collapse pressure profile. The second collapse pressure profile can comprise a collapse function from which a secondary gas vesicle collapse amount can be determined for a given pressure value. The first collapse pressure profile and the second collapse pressure profile can be different. In some embodiments, the first collapse pressure profile and/or second collapse pressure profile has been configured by engineering a gas vesicle protein C (GvpC) protein of the gas vesicles and/or the secondary gas vesicles. In some embodiments, a midpoint of the second collapse profile has a higher pressure component than a midpoint of the first collapse profile. In some embodiments, the second collapse pressure profile comprises a second initial collapse pressure where 5% or lower of the plurality of secondary gas vesicles are collapsed, a second midpoint collapse pressure where 50% of the plurality of secondary gas vesicles are collapsed, a second complete collapse pressure where at least 95% of the plurality of secondary gas vesicles are collapsed, any pressure between the second initial collapse pressure and the second midpoint collapse pressure, and any pressure between the second midpoint collapse pressure and the second complete collapse pressure. In some embodiments, a second selectable collapse pressure is: any collapse pressure within the second collapse pressure profile; selected from the second collapse pressure profile at a value between 0.05% collapse of the plurality of secondary gas vesicles and 95% collapse of the plurality of secondary gas vesicles; equal to or greater than the second initial collapse pressure; equal to or greater than the second midpoint collapse pressure; and/or equal to or greater than the second complete collapse pressure.

### Methods of Imaging and Treatment

There are provided, in some embodiments, methods of imaging a target site of a subject. In some embodiments, the method comprises: administering to the subject an effective amount of a nucleic acid composition disclosed herein, a pharmaceutical composition disclosed herein, or engineered cells disclosed herein. In some embodiments, the method comprises: applying a magnetic field and/or ultrasound (US) to a target site of a subject to obtain an MRI and/or US image of the target site. The period of time between the administering and applying can be about 50 weeks, 45 weeks, 40 weeks, 35 weeks, 30 weeks, 25 weeks, 20 weeks, 15 weeks, 10 weeks, 8 weeks, 6 weeks, 4 weeks, 3 weeks, about 14 days, about 7 days, about 3 days, about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes.

The basic physics of sound waves enables ultrasound to visualize biological tissues with high spatial and temporal resolution. This capability has been enhanced by the development of acoustic biomolecules-proteins with physical properties enabling them to scatter sound. The first acoustic biomolecules developed as contrast agents in ultrasound imaging, analogous to GFPs used in optical imaging, were based on a unique class of air-filled protein nanostructures called gas vesicles (GVs). The advancement of GVs has made it possible to use ultrasound to visualize the functions of cells deep inside tissues.

The term “ultrasound” can refer to sound with frequencies higher than the audible limits of human beings, typically over 20 kHz. Ultrasound devices typically can range up to the gigahertz range of frequencies, with most medical ultrasound devices operating in the 0.2 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a person skilled in focused ultrasound. U.S. Patent Application Publication No. 2020/0237346 describes methods comprising the application of a step function increase in acoustic pressure during ultrasound imaging using gas vesicle contrast, along with capturing successive frames of ultrasound imaging and extracting time-series vectors for pixels of the frames, the content of which is hereby expressly incorporated by reference in its entirety. In some embodiments, the first, second, third, fourth, fifth, and/or sixth US pulse(s) each comprise a set of pulses.

Focused ultrasound (“FUS”) can refer to the technology that uses ultrasound energy to target specific areas of a subject, such as a specific area of a brain or body. FUS focuses acoustic waves by employing concave transducers that usually have a single geometric focus, or an array of ultrasound transducer elements which are actuated in a spatiotemporal pattern such as to produce one or more focal zones. At this focus or foci most of the power is delivered during sonication in order to induce mechanical effects, thermal effects, or both. The frequencies used for focused ultrasound are in the range of 200 KHz to 8000 KHz.

As used herein, the term “harmonic signal” or “harmonic frequency” can refer to a frequency in a periodic waveform that is an integer multiple of the frequency of the fundamental signal. In addition, this term encompasses sub-harmonic signals, which are signals with a frequency equal to an integral submultiple of the frequency of the fundamental signal. In ultrasound imaging, the transmitted pulse is typically centered around a fundamental frequency, and received signals may be processed to isolate signals centered around the fundamental frequency or one or more harmonic frequencies.

The term “fundamental signal” or “fundamental wave” can refer to the primary frequency of the transmitted ultrasound pulse. All GVs can backscatter ultrasound at the fundamental frequency, allowing their detection by ultrasound.

The term “non-linear signal” can refer to a signal that does not obey superposition and scaling properties, with regards to the input. The term “linear signal” can refer to a signal that does obey those properties. One example of non-linearity is the production of harmonic signals in response to ultrasound excitation at a certain fundamental frequency. Another example is a non-linear response to acoustic pressure. One embodiment of such a non-linearity is the acoustic collapse profile of GVs, in which there is a non-linear relationship between the applied pressure and the disappearance of subsequent ultrasound contrast from the GVs as they collapse. Another example of a non-linear signal that does not involve the destruction of GVs, is the increase in both fundamental and harmonic signals with increasing pressure of the transmitted imaging pulse, wherein certain GVs exhibit a super-linear relationship between these signals and the pulse pressure.

The term “applying ultrasound” shall be given its ordinary meaning, and shall also refer to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging/delivery task and, in some methods described herein, collapsing GVs (thereby inducing engineered cells herein to release payload molecules at a target site). To create images, particularly 2D and 3D imaging, scanning techniques can be used where the ultrasound energy is applied in lines or slices which are composited into an image.

The nucleic acid composition can be capable of expressing gas vesicle(s) having an acoustic collapse pressure threshold, and wherein applying ultrasound comprises: applying ultrasound to the target site at a peak positive pressure less than the acoustic collapse pressure threshold; increasing peak positive pressure (PPP) to above the selective acoustic collapse pressure value as a step function; and imaging the target site in successive frames during the increasing; and extracting a time-series vector for each of at least one pixel of the successive frames. In some embodiments, the method comprises: performing a signal separation algorithm on the time-series vectors using at least one template vector. In some embodiments, the signal separation algorithm includes template projection and/or template unmixing. In some embodiments, the at least one template vector includes linear scatterers, noise, gas vesicles, or a combination thereof. The successive frames can comprise a frame prior to GVs collapse, a frame during GVs collapse, and a frame after GVs collapse. In some embodiments, the increasing includes increasing the PPP to a hiBURST regime, optionally the PPP in hiBURST regime is 4.3 MPa or higher. In some embodiments, the increasing includes increasing the PPP to a loBURST regime, optionally the PPP in loBURST regime is no higher than 3.7 MPa

In some embodiments, applying US to a target site can comprise applying one or more US pulses to the target site over a duration of time. The duration of time can be about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulses each have a pulse duration of about 1 hour, about 30 minutes, about 15 minutes, about 10 minutes, about 5 minutes, about 1 minute, about 1 second, or about 1 millisecond. Applying an US pulse can comprise applying a focused US pulse. Applying an US pulse can comprise applying US at a frequency of 100 kHz to 100 MHz. Applying an US pulse can comprise applying ultrasound at a frequency of 0.2 to 1.5 mHz. Applying an US pulse can comprise applying ultrasound having a mechanical index in a range between 0.2 and 0.6. The US pulse can comprise a peak pressure of about 40 kPa to about 800 kPa. The US pulse can comprise a peak pressure of about 70 kPa to about 150 kPa, and/or about 440 kPa to about 605 kPa.

There are provided, in some embodiments, methods of treating or preventing a disease or disorder in a subject. In some embodiments, the method comprises: administering to the subject an effective amount of a nucleic acid composition disclosed herein, a pharmaceutical composition disclosed herein, or engineered cells disclosed herein. In some embodiments, the method comprises the spatial and temporal delivery of payload molecules to a target site of a subject, the method comprising: applying a first ultrasonic (US) pulse to a target site of the subject; detecting the presence of the engineered cells; and applying a second US pulse to the target site of the subject, wherein the second US pulse induces the release of payload molecules from the engineered cells, thereby delivering payload molecules to the target site. The payload molecules can comprise the first unit payload protein(s), the second unit payload protein(s), and/or the supplemental unit payload protein(s).

Multiplexed imaging and payload delivery methods are provided herein. The term “multiplex” can refer to the presence of two or more distinct GVPS types, each of which exhibits an acoustic collapse pressure profile distinct from one another, in the engineered cells. The two or more distinct GVPSs can be derived from different organisms or variants of GVPSs from the same or different organisms (e.g., archaea).

In multiplexing methods herein described, both the collapsing pressure of the collapsing ultrasound and the imaging pressure of the imaging ultrasound are selected based on the acoustic collapse pressure profiles of the GVPS types (e.g., peak acoustic pressure) to selectively collapse one GVPS type over the other GVPS types. The term “selectively collapse” can refer to collapsing at least a portion of one GVPS type in a greater amount that any other GVPS type in a mixture containing a plurality of GVPS types.

The method can include applying a set of imaging pulses from an ultrasound transmitter to the target site, and receiving ultrasound signal at a receiver. In some instances, the ultrasound signal detected by the receiver includes an ultrasound echo signal.

Methods for performing ultrasound imaging are known in the art and can be employed in methods of the current disclosure. In certain aspects, an ultrasound transducer, which comprises piezoelectric elements, transmits an ultrasound imaging signal (or pulse) in the direction of the target site. Variations in the acoustic impedance (or echogenicity) along the path of the ultrasound imaging signal causes backscatter (or echo) of the imaging signal, which is received by the piezoelectric elements. The received echo signal is digitized into ultrasound data and displayed as an ultrasound image. Conventional ultrasound imaging systems comprise an array of ultrasonic transducer elements that are used to transmit an ultrasound beam, or a composite of ultrasonic imaging signals that form a scan line. The ultrasound beam is focused onto a target site by adjusting the relative phase and amplitudes of the imaging signals. The imaging signals are reflected back from the target site and received at the transducer elements. The voltages produced at the receiving transducer elements are summed so that the net signal is indicative of the ultrasound energy reflected from a single focal point in the subject. An ultrasound image is then composed of multiple image scan lines.

In some embodiments, imaging the target site is performed by applying or transmitting an imaging ultrasound signal from an ultrasound transmitter to the target site and receiving a set of ultrasound data at a receiver. The ultrasound data can be obtained using a standard ultrasound device, or can be obtained using an ultrasound device configured to specifically detect the contrast agent used. Obtaining the ultrasound data can include detecting the ultrasound signal with an ultrasound detector. In some embodiments, the imaging step further comprises analyzing the set of ultrasound data to produce an ultrasound image.

In some embodiments, the ultrasound signal has a transmit frequency of at least 1 MHz, 5 MHz, 10 MHZ, 20 MHz, 30 MHz, 40 MHz or 50 MHz. For example, an ultrasound data is obtained by applying to the target site an ultrasound signal at a transmit frequency from 4 to 11 MHZ, or from 14 to 22 MHz. For example, the imaging frequency can be selected so as to maximize the contrast generated by the administered contrast agent.

The collapsing ultrasound and imaging ultrasound can be selected to have a collapsing pressure and an imaging pressure amplitude based on the acoustic collapse pressure profile (e.g., peak acoustic pressure) of the GVPS type used. In some instances, the ultrasound pressure, including the collapsing ultrasound pressure and the imaging ultrasound pressure can be referred to as the “peak positive pressure” of the ultrasound pulses. The term “peak positive pressure” can refer to the maximum pressure amplitude of the positive pulse of a pressure wave, typically in terms of the difference between the peak pressure and the ambient pressure at the location in the person or specimen that is being imaged.

Detecting the presence of the engineered cells at the target site can comprise detecting scattering of the first US pulse by the gas vesicles. In some embodiments, the method comprises: confirming the delivery of payload molecules at the target site. In some embodiments, confirming the delivery of payload molecules can comprise detecting reduced scattering of the second US pulse by the gas vesicles. The gas vesicles can be capable of acting as a contrast agent at the first US pulse but not at the second US pulse. The first US pulse can comprise a pressure value less than the first selectable collapse pressure value. The second US pulse can comprise a pressure value equal to or higher than the first selectable collapse pressure value. In some embodiments, the second US pulse induces gas vesicle collapse. The gas vesicles can be capable of acting as a contrast agent at the first US pulse but not at the second US pulse.

In some embodiments, the gas vesicle collapse results in the release of a nanoscale air bubble. In some embodiments, the released nanoscale air bubble undergoes cavitation and is converted into a micron-scale air bubble. The second US pulse can be capable of inducing cavitation. The cavitation can comprise cavitation of the gas vesicles and/or bubbles created by gas vesicle collapse. The gas vesicles can be capable as acting as the nuclei for the formation and/or cavitation of bubbles. The cavitation can comprise stable cavitation. The cavitation can comprise inertial cavitation. In some embodiments, the cavitation triggers the degradation of the engineered cells. In some embodiments, the cavitation induces the release of payload molecules from the engineered cells. In some embodiments, the cavitation exerts mechanical forces and/or thermal forces on the engineered cells, thereby inducing the release of payload molecules. The target site can comprise target cells. In some embodiments, the cavitation exerts mechanical forces and/or thermal forces on target cells proximate to the engineered cells, thereby enhancing uptake of payload molecules by said target cells. In some embodiments, said mechanical forces and/or thermal forces reduce the membrane permeability of target cells proximate to the engineered cells. The peak positive pressure of the second US pulse can be equal to or higher than an initial collapse pressure of the gas vesicles, thereby collapsing the gas vesicles. The peak negative pressure of the second US pulse can be below the critical cavitation pressure of the gas vesicles.

In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules can be released at the target site. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules can be released at a location other than the target site. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules can be released from the engineered cells within about 1 ns, about 10 ns, about 100 ns, about 1 ms, about 10 ms, about 100 ms, or about 1 s after the second US pulse. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules can be released from the engineered cells within about 1 nm, about 10 nm, about 100 nm, about 1 μm, about 10 μm, about 100 μm, about 1 mm, about 10 mm, or about 100 mm of the location of the engineered cells at the time of the second US pulse. The ratio of the concentration of payload molecules at the subject's target site to the concentration of payload molecules in subject's blood, serum, or plasma can be about 2:1 to about 3000:1, about 2:1 to about 2000:1, about 2:1 to about 1000:1, or about 2:1 to about 600:1.

The target site can comprise a site of disease or disorder or can be proximate to a site of a disease or disorder. The location of the one or more sites of a disease or disorder can be predetermined. The location of the one or more sites of a disease or disorder can be determined during the method (e.g., by an imaging-based method). The target site can comprise a tissue, such as, for example, adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The tissue can be inflamed tissue. The tissue can comprise (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. Exemplary target sites include collections of microorganisms, including, bacteria or archaea in a solution in vitro, as well as cells grown in an in vitro culture, including, primary mammalian, cells, immortalized cell lines, tumor cells, stem cells, and the like. Additional exemplary target sites include tissues and organs in an ex vivo colture and tissue, organs, or organs systems in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person. The term “individual” or “subject” or “patient” as used herein in the context of imaging includes a single plant or animal and in particular higher plants or animals and in particular vertebrates such as mammals and more particularly human beings. Types of ultrasound imaging of biological target sites include abdominal ultrasound, vascular ultrasound, obstetrical ultrasound, hysterosonography, pelvic ultrasound, renal ultrasound, thyroid ultrasound, testicular ultrasound, and pediatric ultrasound as well as additional ultrasound imaging as would be understood by a skilled person.

### Payloads

Provided herein include nucleic acid compositions encoding payloads (e.g., payload proteins, first unit payload protein(s), second unit payload protein(s), supplemental unit payload protein(s), secondary unit payload protein(s), or any combination thereof). The terms “payload”, “payload protein”, and “unit payload protein” shall be given their ordinary meaning and shall also refer to first unit payload protein(s), second unit payload protein(s), supplemental unit payload protein(s), and/or secondary unit payload protein(s) described herein, or a combination thereof (e.g., a multimeric protein complex). In some embodiments one or more of the first unit payload protein(s), the second unit payload protein(s), and the supplemental unit payload protein(s) is a payload, is a component of a payload, or chaperones the assembly of a payload. A payload can comprise two or more of the first unit payload protein(s), the second unit payload protein(s), the supplemental unit payload protein(s).

Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in WO2022/125590 entitled “A synthetic circuit for cellular multistability,” the content of which is incorporated herein by reference in its entirety. A payload (e.g., first unit payload protein(s), second unit payload protein(s), supplemental unit payload protein(s), secondary unit payload protein(s), or a combination thereof) can comprise a synthetic protein circuit component. In some embodiments, one or more components of the disclosed synthetic protein circuits interfaces with (e.g., modulates and/or is modulated by) another synthetic protein circuit component. The first unit payload protein(s), second unit payload protein(s), supplemental unit payload protein(s), and/or secondary unit payload protein(s) described herein can comprise, be under the control of, or modulate (directly or indirectly) a synthetic protein circuit component. A payload protein can be capable of diminishing the concentration, stability, and/or activity an endogenous protein. A payload protein can comprise a component of a synthetic protein circuit. A payload protein can be capable of diminishing the concentration, stability, and/or activity of one or more of the first unit payload protein(s), the second unit payload protein(s), and/or the supplemental unit payload protein(s). Two or more of the first unit payload protein(s), the second unit payload protein(s), and/or the supplemental unit payload protein(s) can be components of a synthetic protein circuit. All components of said synthetic protein circuit can be encoded by the first nucleic acid unit, the second nucleic acid unit, and/or the n supplemental nucleic acid unit(s). A payload can comprise a degron and a cut site a protease can be capable of cutting to expose the degron, and wherein the degron of the payload being exposed changes the payload to a payload destabilized state. The degron can comprise an N-degron, a dihydrofolate reductase (DHFR) degron, a FKB protein (FKBP) degron, derivatives thereof, or any combination thereof. A payload can comprise a protease or a split protease. The activation level of the protease can be related to one or more input signals. The protease can comprise tobacco etch virus (TEV) protease, tobacco vein mottling virus (TVMV) protease, hepatitis C virus protease (HCVP), derivatives thereof, or any combination thereof. The synthetic protein circuit can be configured to be responsive to changes in: cell environment, optionally cell environment comprises location relative to a target site of a subject and/or changes in the presence and/or absence of target cell(s), optionally said target cell(s) comprise target-specific antigen(s); one or more signal transduction pathways regulating cell survival, cell growth, cell proliferation, cell adhesion, cell migration, cell metabolism, cell morphology, cell differentiation, apoptosis, or any combination thereof; input(s) of a synthetic cell-cell communication system, optionally Synthetic Notch (SynNotch) receptor, a Modular Extracellular Sensor Architecture (MESA) receptor, a synthekine, engineered GFP, and/or auxin; and/or T cell activity, optionally T cell activity comprises one or more of T cell simulation, T cell activation, cytokine secretion, T cell survival, T cell proliferation, CTL activity, T cell degranulation, and T cell differentiation. A payload can be capable of modulating the expression, concentration, localization, stability, and/or activity of the one or more endogenous targets of a cell.

In some embodiments, the payload comprises a bispecific T cell engager (BiTE). In some embodiments, the orthogonal signal triggers cellular differentiation. The payload can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload can comprise nuclease activity, methyltransferase activity, disulfide isomerase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload can comprise a CRE recombinase, GCaMP, a cell therapy component, a knock-down gene therapy component, a cell-surface exposed epitope, or any combination thereof. The payload can comprise a diagnostic agent (e.g., green fluorescent protein (GFP), EGFP, YFP, enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

A payload can comprise a constitutive signal peptide for protein degradation (e.g., PEST). A payload can comprise a nuclear localization signal (NLS) or a nuclear export signal (NES). A payload can comprise a dosage indicator protein. The dosage indicator protein can be detectable. The dosage indicator protein can comprise green fluorescent protein (GFP), EGFP, YFP, EYFP, BFP, red fluorescent protein (RFP), TagRFP, Dronpa, Padron, mApple, mCherry, mruby3, rsCherry, rsCherryRev, derivatives thereof, or any combination thereof.

A payload (e.g., first unit payload protein(s), second unit payload protein(s), supplemental unit payload protein(s), secondary unit payload protein(s), or a combination thereof) can be capable of modulating the concentration, localization, stability, and/or activity of the one or more targets. A payload can be capable of repressing the transcription of the one or more targets. A target transcript can be capable of being translated to generate a target protein. A payload can be capable of reducing the concentration, localization, stability, and/or activity of the target protein. The concentration, localization, stability, and/or activity of the target protein can be inversely related to the concentration, localization, stability, and/or activity of a payload. A payload can comprise a protease. The target protein can comprise a degron and a cut site the protease can be capable of cutting to expose the degron. In some embodiments, the degron of the target protein being exposed changes the target protein to a target protein destabilized state. The protease can comprise tobacco etch virus (TEV) protease, tobacco vein mottling virus (TVMV) protease, hepatitis C virus protease (HCVP), derivatives thereof, or any combination thereof. In some embodiments, the target protein comprises a cage polypeptide, wherein the cage polypeptide comprises: (a) a helical bundle, comprising between 2 and 7 alpha-helices, wherein the helical bundle comprises: (i) a structural region; and (ii) a latch region, wherein the latch region comprises a degron located within the latch region, wherein the structural region interacts with the latch region to prevent activity of the degron; and (b) amino acid linkers connecting each alpha helix. A payload can comprise a key polypeptide capable of binding to the cage polypeptide structural region, thereby displacing the latch region and activating the degron.

A payload (e.g., first unit payload protein(s), second unit payload protein(s), supplemental unit payload protein(s), secondary unit payload protein(s), or a combination thereof) can comprise a pro-death protein capable of halting cell growth and/or inducing cell death. The pro-death protein can comprise cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. The pro-death protein can be capable of halting cell growth and/or inducing cell death in the presence of a pro-death agent. In some embodiments, the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy- and amino-aniline mustards, Anthracycline glutamates, and/or Methotrexate α-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-α,γ-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU). Methods disclosed herein can comprise administering a pro-death agent (e.g., administering to a subject).

A payload can be associated with an agricultural trait of interest selected from increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. A payload can be associated with a biological manufacturing process selected from fermentation, distillation, biofuel production, production of a compound, production of a polypeptide, or any combination thereof.

In some embodiments, a payload (e.g., first unit payload protein(s), second unit payload protein(s), supplemental unit payload protein(s), secondary unit payload protein(s), or a combination thereof) can diminish immune cell function. A payload can be an activity regulator. The activity regulator can be capable of reducing T cell activity. The activity regulator can comprise a ubiquitin ligase involved in TCR/CAR signal transduction selected from c-CBL, CBL-B, ITCH, R F125, R F128, WWP2, or any combination thereof. The activity regulator can comprise a negative regulatory enzyme selected from SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. The activity regulator can be a negative regulatory scaffold/adapter protein selected from PAG, LIME, NTAL, LAX31, SIT, GAB2, GRAP, ALX, SLAP, SLAP2, DOK1, DOK2, or any combination thereof. The activity regulator can be a dominant negative version of an activating TCR signaling component selected from ZAP70, LCK, FYN, NCK, VAV1, SLP76, ITK, ADAP, GADS, PLCgammal, LAT, p85, SOS, GRB2, NFAT, p50, p65, API, RAPl, CRKII, C3G, WAVE2, ARP2/3, ABL, ADAP, RIAM, SKAP55, or any combination thereof. The activity regulator can comprise the cytoplasmic tail of a negative co-regulatory receptor selected from CD5, PD1, CTLA4, BTLA, LAG3, B7-H1, B7-1, CD160, TFM3, 2B4, TIGIT, or any combination thereof. The activity regulator can be targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

A payload can comprise a factor locally down-regulating the activity of endogenous immune cells. In some embodiments, a payload protein(s) comprises a prodrug-converting enzyme (e.g., HSV thymidine kinase (TK), Cytosine Deaminase (CD), Purine nucleoside phosphorylase (PNP), Cytochrome p450 enzymes (CYP), Carboxypeptidases (CP), Caspase-9, Carboxylesterase (CE), Nitroreductase (NTR), Horse radish peroxidase (HRP), Guanine Ribosyltransferase (XGRTP), Glycosidase enzymes, Methionine-α,γ-lyase (MET), Thymidine phosphorylase (TP)).

A payload (e.g., first unit payload protein(s), second unit payload protein(s), supplemental unit payload protein(s), secondary unit payload protein(s), or a combination thereof) can comprise a cytokine. The cytokine can be interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof.

A payload (e.g., first unit payload protein(s), second unit payload protein(s), supplemental unit payload protein(s), secondary unit payload protein(s), or a combination thereof) can comprise a member of the TGF-β/BMP family selected from TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. A payload can comprise a member of the TNF family of cytokines selected from TNF-alpha, TNF-beta, LT-beta, CD40 ligand, Fas ligand, CD 27 ligand, CD 30 ligand, and 4-1 BBL. A payload can comprise a member of the immunoglobulin superfamily of cytokines selected from of B7.1 (CD80) and B7.2 (B70). A payload can comprise an interferon. The interferon can be selected from interferon alpha, interferon beta, or interferon gamma. A payload can comprise a chemokine. The chemokine can be selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. A payload can comprise a interleukin. The interleukin can be selected from IL-10 IL-12, IL-1, IL-6, IL-7, IL-15, IL-2, IL-18 or IL-21. A payload can comprise a tumor necrosis factor (TNF). The TNF can be selected from TNF-alpha, TNF-beta, TNF-gamma, CD252, CD154, CD178, CD70, CD153, or 4-1BBL.

A payload (e.g., first unit payload protein(s), second unit payload protein(s), supplemental unit payload protein(s), secondary unit payload protein(s), or a combination thereof) can comprise a CRE recombinase, GCaMP, a cell therapy component, a knock-down gene therapy component, a cell-surface exposed epitope, or any combination thereof. A payload can comprise a chimeric antigen receptor.

A payload (e.g., first unit payload protein(s), second unit payload protein(s), supplemental unit payload protein(s), secondary unit payload protein(s), or a combination thereof) can comprise a programmable nuclease. The programmable nuclease can be: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The nucleic acid composition can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA). A payload can comprise (i) a targeting molecule and/or (ii) a donor nucleic acid.

In some embodiments, a payload (e.g., first unit payload protein(s), second unit payload protein(s), supplemental unit payload protein(s), secondary unit payload protein(s), or a combination thereof) is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as-glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I B, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein(s) include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, a payload (e.g., first unit payload protein(s), second unit payload protein(s), supplemental unit payload protein(s), secondary unit payload protein(s), or a combination thereof) is an active fragment of a protein, such as any of the aforementioned proteins. In some embodiments, unit payload protein(s) is a fusion protein comprising some or all of two or more proteins. In some embodiments a fusion protein can comprise all or a portion of any of the aforementioned proteins.

In some embodiments, a payload (e.g., first unit payload protein(s), second unit payload protein(s), supplemental unit payload protein(s), secondary unit payload protein(s), or a combination thereof) is a multi-subunit protein. For examples, A payload can comprise two or more subunits, or two or more independent polypeptide chains. In some embodiments, A payload can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, a payload is a pro-survival protein (e.g., Bcl-2, Bcl-XL, Mcl-1 and A1). In some embodiments, the payload is a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-X_{L}, Bcl-xs, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, a payload (e.g., first unit payload protein(s), second unit payload protein(s), supplemental unit payload protein(s), secondary unit payload protein(s), or a combination thereof) is a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, a payload is a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, a payload (e.g., first unit payload protein(s), second unit payload protein(s), supplemental unit payload protein(s), secondary unit payload protein(s), or a combination thereof) is a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, ClqR, CIINH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 R1, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding a payload (e.g., first unit payload protein(s), second unit payload protein(s), supplemental unit payload protein(s), secondary unit payload protein(s), or a combination thereof) can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload protein(s) (e.g., first unit payload protein(s), second unit payload protein(s), supplemental unit payload protein(s), secondary unit payload protein(s)) expression in the cell can vary. The amount of unit payload protein(s) expressed in the subject (e.g., the serum of the subject) can vary. For example, in some embodiments the protein can be expressed in the serum of the subject in the amount of, at least about, at most about, 9 μg/ml, 10 μg/ml, 50 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1000 μg/ml, or a number or a range between any two of these values. In some embodiments, unit payload protein(s) is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein(s) is needed for the method to be effective can vary depending on non-limiting factors such as the particular payload protein(s) and the subject receiving the treatment, and an effective amount of the protein can be readily determined by a skilled artisan using conventional methods known in the art without undue experimentation.

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload can comprise a CRE recombinase, GCaMP, a cell therapy component, a knock-down gene therapy component, a cell-surface exposed epitope, or any combination thereof.

### Chimeric Antigen Receptors and Engineered T Cell Receptors

Payload(s) (e.g., first unit payload protein(s), second unit payload protein(s), supplemental unit payload protein(s), secondary unit payload protein(s), or a combination thereof) can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. The intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to, an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof.

The intracellular signaling domain can be designed to comprise two or more, e.g., 2, 3, 4, 5, or more, costimulatory signaling domains. In some embodiments, the two or more, e.g., 2, 3, 4, 5, or more, costimulatory signaling domains, are separated by a linker molecule, e.g., a linker molecule described herein. In some embodiments, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, and a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

The CAR can comprise a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

The CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

The antigen binding domain can be a T cell receptor (“TCR”), or a fragment thereof, for example, a single chain TCR (scTCR). Methods to make such TCRs are known in the art. See, e.g., Willemsen R A et al, Gene Therapy 7:1369-1377 (2000); Zhang T et al, Cancer Gene Ther 11:487-496 (2004); Aggen et al, Gene Ther. 19 (4): 365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

The antigen binding domain can be a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARS comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see e.g., Sastry et al., J Virol. 2011 85 (5): 1935-1942). For example, TCR-like antibody can be identified from screening a library, such as a human scFv phage displayed library.

The tumor antigen can be a solid tumor antigen. The tumor antigen can be selected from: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-IR, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, B2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Targeting

A payload (e.g., first unit payload protein(s), second unit payload protein(s), supplemental unit payload protein(s), secondary unit payload protein(s)) can comprise one or more receptors and/or a targeting moiety configured to bind a component of a target site of a subject The one or more receptors and/or one or more targeting moieties can be mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. The one or more receptors and/or one or more targeting moieties can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, or a receptor ligand, and analogs and derivatives thereof.

The antibody or antigen-binding fragment thereof can comprise a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

A payload (e.g., first unit payload protein(s), second unit payload protein(s), supplemental unit payload protein(s), secondary unit payload protein(s)) can comprise one or more receptors and/or a targeting moiety configured to bind a component of a target site of a subject. The one or more receptors and/or one or more targeting moieties can be configured to bind one or more of the following: CD3, CD4, CD5, CD6, CD7, CD8, CD9, CD10, CDl la, CDl lb, CDl lc, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C-X-C chemokine receptor type 4, cyclic ADP ribose hydrolase, Cyclin B 1, CYP1B 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRVIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, αvβ3, α₄β₇, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CDl la), LHRH, LINGO-1, Lipoteichoic acid, LIV1A, LMP2, LTA, MAD-CT-1, MAD-CT-2, MAGE-1, MAGE-2, MAGE-3, MAGE AI, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-βI, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

### Antigenic Polypeptides

A payload can be an antigenic polypeptide (AP). In some embodiments, two or more of the first unit payload protein(s), the second unit payload protein(s), and/or the supplemental unit payload protein(s) is an antigenic polypeptide (AP), and thereby the polycistronic transcript is capable of being translated to generate a plurality of disparate AP. In some embodiments, the compositions provided herein are mRNA vaccines. The AP can comprise or can be derived from an antigenic protein associated with a disease or disorder, optionally an immunogenic variant and/or an immunogenic fragment of said antigenic protein. The AP can comprise or be derived from at least a portion of an antigenic protein. The AP can comprise or can be derived from a conserved portion of said antigenic protein. The AP can comprise or can be derived from at least about 5 percent (e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the full length of said antigenic protein. In some embodiments, the AP comprises or does not comprise one or more mutations configured to enhance its solubility and/or stability.

Accordingly, the APs can contain amino acid substitutions relative to the antigenic proteins disclosed herein. Any amino acid substitution is permissible so long as the immunogenic activity of the protein is not significantly altered (e.g., at most 10%, 20%, 30%, 40% or 50% decrease relative to the coronavirus protein antigens disclosed herein) and the variants retain the desired activity. Preferred variants typically contains substitutions with one or more amino acids substituted with their functional equivalents.

Disclosed herein include methods of stimulating an immune response in a subject in need thereof. In some embodiments, the method comprises: administering to the subject an effective amount of a nucleic acid composition disclosed herein, a pharmaceutical composition disclosed herein, or engineered cells disclosed herein, thereby stimulating an immune response in the subject. Provided herein include a vaccine composition comprising compositions (e.g., a nucleic acid composition) as herein described. Vaccine compositions can comprise the compositions provided herein (e.g., a nucleic acid composition) in combination with one or more compatible and pharmaceutically acceptable carriers. For example, a vaccine composition can be or can comprise an mRNA vaccine and/or a DNA vaccine. A vaccine composition is a pharmaceutical composition that can elicit a prophylactic (e.g., to prevent or delay the onset of a disease, or to prevent the manifestation of clinical or subclinical symptoms thereof) or therapeutic (e.g., suppression or alleviation of symptoms) immune response in a subject.

The second dose can be administered to the subject at least 14 days after a first dose is administered to the subject. In some embodiments, administration of the nucleic acid composition, the pharmaceutical composition, and/or the engineered cells elicits protective and long-lasting immunity against the infectious agent(s) and variants thereof. The nucleic acid composition, the pharmaceutical composition, and/or the engineered cells can be administered in an effective amount to: induce a robust antibody response against the AP in the subject, optionally a robust antibody response comprises a neutralizing antibody response, further optionally a robust antibody response comprises Fc domain effector functions that recruit immune cells to infected cells, optionally said immune cells are macrophages, neutrophils, and/or natural killer cells, further optionally said recruitment induces antibody-dependent cellular cytotoxicity (ADCC) and/or antibody-dependent cellular phagocytosis (ADCP); elicit a robust CD4 and/or CD8 T cell response against the AP in the subject; and/or elicit a balanced Th1/Th2 response against the AP in the subject.

### Pathogenic Antigens

The AP can comprise or can be derived from the protein of an infectious agent. The disease or disorder can be an infectious disease or disorder caused by an infectious agent, the AP can comprise or can be derived from an antigenic protein of said infectious agent, and the antigenic protein of said infectious agent can be a pathogenic antigen. In some embodiments, the pathogenic antigen is: Outer membrane protein A OmpA, biofilm associated protein Bap, transport protein Muck (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, Gal/GaINAc lectin, protein CRT, 125 kDa immunodominant antigen, protein M17, adhesin ADH112, protein STIRP (Entamoeba histolytica, Amoebiasis); Major surface proteins 1-5 (MSP1a, MSP1b, MSP2, MSP3, MSP4, MSP5), type IV secreotion system proteins (VirB2, VirB7, VirB11, VirD4) (Anaplasma genus, Anaplasmosis); protective Antigen PA, edema factor EF, lethal factor LF, the S-layer homology proteins SLH (Bacillus anthracis, Anthrax); acranolysin, phospholipase D, collagen-binding protein CbpA (Arcanobacterium haemolyticum, Arcanobacterium haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa surface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp f3, major conidial surface protein rodlet A, protease Pep1p, GPI-anchored protein Gel1p, GPI-anchored protein Crap (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, c), 12D3, 1105, 21134, P29, variant erythrocyte surface antigen VESA1, Apical Membrane Antigen 1 AMA-1 (Babesia genus, Babesiosis); hemolysin, enterotoxin C, PX01-51, glycolate oxidase, ABC-transporter, penicillin-binding protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocystis hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BoNT/A3, BONT/B, BONT/C, BONT/D, BONT/E, BONT/F, BoNT/G, recombinant botulinum toxin F Hc domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-binding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B laIB, trigger factor Tig, molecular chaperone DnaK, putative peptidyl-prolyl cis-trans isomerase SurA, lipoprotein Omp19, outer membrane protein MotY Omp16, conserved outer membrane protein D15, malate dehydrogenase Mdh, component of the Type-IV secretion system (TOSS) VirJ, lipoprotein of unknown function BAB1_0187 (Brucella genus, Brucellosis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, genome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspA1, protein FspA2 (Campylobacter genus, Campylobacteriosis); glycolytic enzyme enolase, secreted aspartyl proteinases SAP1-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LaIB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Part, mucin-Associated Surface Proteins MPSP (Trypanosoma cruzi, Chagas Disease (American trypanosomiasis)); envelope glycoproteins (gB, gC, gE, gH, gI, gK, gL) (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis protein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwp13, cysteine protease Cwp19, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: spike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GaIXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein pp15, capsid-proximal tegument protein pp150, protein M45, DNA polymerase UL54, helicase UL105, glycoprotein gM, glycoprotein gN, glycoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphtheria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vCJD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DItA, lipoprotein Hip, major outer membrane protein OMP, outer membrane protein OmpA2 (Haemophilus ducreyi, Chancroid); aspartyl protease 1 Pep1, phospholipase B PLB, alpha-mannosidase 1 AMN1, glucanosyltransferase GEL1, urease URE, peroxisomal matrix protein Pmp1, proline-rich antigen Pra, human T-cell reactive protein TcrP (Coccidioides immitis and Coccidioides posadasii, Coccidioidomycosis); allergen Tri r2, heat shock protein 60 Hsp60, fungal actin Act, antigen Tri r2, antigen Tri r4, antigen Tri t1, protein IV, glycerol-3-phosphate dehydrogenase Gpd1, osmosensor HwSho1A, osmosensor HwSho1B, histidine kinase HwHhk7B, allergen Mala s 1, allergen Mala s 11, thioredoxin Trx Mala s 13, allergen Mala f, allergen Mala s (Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 (MSP2), major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins Sa1A and Sa1B, collagen adhesin Scm, surface proteins Fms1 (EbpA(fm), Fms5 (EbpB(fm), Fms9 (EpbC(fm) and Fms10, protein EbpC(fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCA1, Invasion protein invA, cell division protein fts, secretion proteins sec Ofamily, virulence proteins virB, tlyA, tlyC, parvulin-like protein Plp, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein glycoprotein gp350, glycoprotein gp110, glycoprotein gp42, glycoprotein gHgL, glycoprotein gB (Epstein-Barr Virus (EBV), Epstein-Barr Virus Infectious Mononucleosis); cpasid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein KTM, leucine aminopeptidase LAP, cysteine proteinase Fast, saposin-like protein-2 SAP-2, thioredoxin peroxidases TPx, Prx-1, Prx-2, cathepsin I cysteine proteinase CL3, protease cathepsin L CL1, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin L1g and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component Ib, protein CPE1281 pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component Ib, protein CPE1281, pyruvate ferredoxin oxidoreductase, elongation factor G EF-G, perfringolysin O (PFO), glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Dec1 (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alpha1-guiardin, beta-giardin, cysteine proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emm1, M protein type 6 Emm6, immunoglobulin-binding protein 35 Sib35, Surface protein R28 Spr28, superoxide dismutase SOD, C5a peptidase ScpA, antigen I/II AgI/II, adhesin AspA, G-related alpha2-macroglobulin-binding protein GRAB, surface fibrillar protein M5 (Streptococcus pyogenes, Group A streptococcal infection); C protein β antigen, arginine deiminase proteins, adhesin BibA, 105 kDA protein BPS, surface antigens c, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alpha-like protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein P1, outer membrane protein P2, outer membrane lipoprotein Pcp, Lipoprotein E, outer membrane protein P4, fuculokinase fucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein 5, envelope glycoprotein G1, nucleoprotein NP, protein N, polyprotein M (Sin Nombre virus, Hantavirus, Hantavirus Pulmonary Syndrome (HPS)); heat shock protein HspA, heat shock protein HspB, citrate synthase GItA, protein UreB, heat shock protein Hsp60, neutrophil-activating protein NAP, catalase KatA, vacuolating cytotoxin VacA, urease alpha UreA, urease beta Ureb, protein Cpn10, protein groES, heat shock protein Hsp10, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, O-antigen, toxin-antigens Stx2B, toxin-antigen Stx1B, adhesion-antigen fragment Int28, protein EspA, protein EspB, Intimin, protein Tir, protein IntC300, protein Eae (Escherichia coli 0157: H7, 0111 and 0104: H4, Hemolytic-uremic syndrome (HUS)); RNA polymerase L, protein L, glycoprotein Gn, glycoprotein Gc, nucleocapsid protein 5, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, protein C, phosphoprotein p, non-structural protein V (Henipavirus (Hendra virus Nipah virus), Henipavirus infections); polyprotein, glycoprotein Gp2, hepatitis A surface antigen HBAg, protein 2A, virus protein VP1, virus protein VP2, virus protein VP3, virus protein VP4, protein PIB, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35 envelope glycoprotein E2 NS1 gp68 gp70, capsid protein C core protein Core, polyprotein, virus protein VP1, virus protein VP2, virus protein VP3, virus protein VP4, antigen G, protein NS3, protein NSSA, (Hepatitis C Virus, Hepatitis C); virus protein VP1, virus protein VP2, virus protein VP3, virus protein VP4, large hepatitis delta antigen, small hepatitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein 1E63 (ICP27, UL54), protein UL55, protein UL56, viral replication protein ICP22 (1E68, US1), protein U52, serine/threonine-protein kinase U53, glycoprotein G (U54), glycoprotein J (U55), glycoprotein D (U56), glycoprotein I (U57), glycoprotein E (U58), tegument protein U59, capsid/tegument protein US10, Vmw21 protein (US11), ICP47 protein (IE12, US12), major transcriptional activator ICP4 (1E175, RS1), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein HIC, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, 5-transferase GST, aspartic protease APR-1, acetylcholinesterase AChE (Ancylostoma duodena le and Necator americanus, Hookworm infection); protein NS1, protein NP1, protein VP1, protein VP2, protein VP3 (Human bocavirus (HBOV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSP1a, MSP1b, MSP2, MSP3, MSP4, MSP5), type IV secretion system proteins VirB2, VirB7, VirB11, VirD4 (Anaplasma phagocytophilum, Human granulocytic anaplasmosis (HGA)); protein NS1, small hydrophobic protein N52, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrane protein 19 OMP-19, major antigenic protein MAPI, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1., regulatory protein E2, protein E3, protein E4, protein ES, protein E6, protein E7, protein E8, major capsid protein L1, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), MI protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein B1, ribosomal protein P1-like protein P1, sterol 24-c-methyltransferase SMT, LACK protein, histone H1, SPB1 protein, thiol specific antioxidant TSA, protein antigen STII, signal peptidase SP, histone H2B, surface antigen PSA-2, cysteine proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein htrA2 or ML0176 coding protein htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membrane lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin 0 precursor Hly (LL0), invasion-associated protein Iap (P60), Listeriolysin regulatory protein PrfA, Zinc metalloproteinase Mpl, Phosphatidylinositol-specific phospholipase C PLC (PICA, PlcB), 0-acetyltransferase Oat, ABC-transporter permease Im.G_1771, adhesion protein LAP, LAP receptor Hsp60, adhesin LapB, haemolysin listeriolysin 0 LLO, protein ActA, Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP5, enolase PF10, hepatocyte erythrocyte protein 17 kDa HEP17, erythrocyte membrane protein 1 EMP1, protein Kbetamerozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar of integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly (A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RPO7 (Molluscum contagiosum virus (MCV), Molluscum contagiosum (MC)); matrix protein M, phosphoprotein P/V, small hydrophobic protein SH, nucleoprotein N, protein V, fusion glycoprotein hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin P1, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gp0, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PIPE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein Tbpl, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Prn, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Pm, protein Fim2, protein Fim3;” (Bordetella pertussis, Pertussis (Whooping cough)); “F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YP00612, heat shock protein 90 HtpG, putative sulfatase protein YdeN, outer-membrane lipoprotein carrier protein LoIA, secretion chaperone YerA, putative lipoprotein YP00420, hemolysin activator protein HpmB, pesticin/yersiniabactin outer membrane receptor Psn, secreted effector protein YopE, secreted effector protein YopF, secreted effector protein YopK, outer membrane protein YopN outer membrane protein YopM, Coagulase/fibrinolysin precursor Pla;” (Yersinia pestis, Plague); protein PhpA, surface adhesin PsaA, pneumolysin Ply, ATP-dependent protease CIp, lipoate-protein ligase LpIA, cell wall surface anchored protein psrP, sortase SrtA, glutamyl-tRNA synthetase GItX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfa1, exendin-3, secretory lipase, cathepsin B-like protease, cysteine protease, cathepsin, peroxiredoxin, protein CrylAc (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-ma lonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein PI, heat shock protein B HspB, peptide ABC transporter, GTP-binding protein, protein IcmB, ribonuclease R, phosphatas SixA, protein DsbD, outer membrane protein ToIC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCA1, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCA1, intracytoplasmic protein D (Rickettsia rickettsii, Rocky mountain spotted fever (RMSF)); non-structural protein 6 N56, non-structural protein 2 N52, intermediate capsid protein VP6, inner capsid protein VP2, non-structural protein 3 NS3, RNA-directed RNA polymerase L, protein VP3, non-structural protein 1 NS1, non-structural protein 5 N55, outer capsid glycoprotein VP7, non-structural glycoprotein 4 N54, outer capsid protein VP4; (Rotavirus, Rotavirus infection); polyprotein P200, glycoprotein E1, glycoprotein E2, protein N52, capsid protein C (Rubella virus, Rubella); chaperonin GroEL (MopA), inositol phosphate phosphatase SopB, heat shock protein HsIU, chaperone protein DnaJ, protein TviB, protein IroN, flagellin FliC, invasion protein SipC, glycoprotein gp43, outer membrane protein LamB, outer membrane protein PagC, outer membrane protein ToIC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein N56, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASAI, glutathione 5-transferases GST, cystein protease, serine protease, apolipoprotein (Sarcoptes scabiei, Scabies); glutathione 5-transferases GST, paramyosin, hemoglbinase SM32, major egg antigen, 14 kDa fatty acid-binding protein Sm 14, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator 1E63, transcriptional transactivator 1E62, protease P33, alpha trans-inducing factor 74 kDa protein, deoxyuridine 5′-triphosphate nucleotidohydrolase, transcriptional transactivator 1E4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tripartite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein C1, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ ATPase Sseat-6, tropomysin SsTmy-1, protein LEC-5, 41 kDa antigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF1, repeat protein Tpr, repeat protein F TprF, repeat protein G TprG, repeat protein I Tprl, repeat protein J TprJ, repeat protein KTprK, treponemal membrane protein A TmpA, lipoprotein, 15 kDa Tpp15, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MICE, MIC7, MICE), rhoptry protein Rop2, rhoptry proteins (Rop1, Rop2, Rop3, Rop4, Rop5, Rop6, Rop7, Rop16, Rjop17), protein SR1, surface antigen P22, major antigen p24, major surface antigen p30, dense granule proteins (GRA1, GRA2, GRA3, GRA4, GRA5, GRA6, GRA7, GRAB, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60) immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTBE, Rpf-like proteins, protein MTB32, protein MTB39, crystallin, heat-shock protein HSP65, protein PST-S(usually Mycobacterium tuberculosis, Tuberculosis); outer membrane protein FobA, outer membrane protein FobB, intracellular growth locus IgIC1, intracellular growth locus IgIC2, aminotransferase Wbt1, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein toIC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen;” (Ureaplasma urealyticum, Ureaplasma urealyticum infection); non-structural polyprotein, structural polyprotein, capsid protein CP, protein E1, protein E2, protein E3, protease Pb, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); capsid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FIhA, peptidase M48, potassium efflux system KefA, transcriptional regulatoer RovA, adhesin Ifp, translocator protein LcrV, protein PcrV, invasin Inv, outer membrane protein OmpF-like porin, adhesin YadA, protein kinase C, phospholipase C1, protein PsaA, mannosyltransferase-like protein WbyK, protein YscU, antigen YPMa (Yersinia pseudotuberculosis, Yersinia pseudotuberculosis infection); and effector protein YopB, 60 kDa chaperonin, protein WbcP, tyrosin-protein phosphatase YopH, protein YopQ, enterotoxin, Galactoside permease, reductase NrdE, protein YasN, Invasin Inv, adhesin YadA, outer membrane porin F OmpF, protein UspA1, protein EibA, protein Hia, cell surface protein Ail, chaperone SycD, protein LcrD, protein LerG, protein LcrV, protein SycE, protein YopE, regulator protein TyeA, protein YopM, protein YopN, protein YopO, protein YopT, protein YopD, protease CIpP, protein MyfA, protein FilA, and protein PsaA (Yersinia enterocolitica, Yersiniosis).

The infectious agent can be a bacterium, a fungus, a virus, or a protist. The infectious agent can be a coronavirus (CoV) (e.g., an alphacoronavirus, a betacoronavirus, a gammacoronavirus, or a deltacoronavirus). The infectious agent can be Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, (Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 0157: H7, O111 and 0104: H4, Fasciola hepatica and Fasciola gigantica, FFI prion, Filarioidea superfamily, Filoviruses, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

The AP can comprise or can be derived from the protein of a coronavirus. The disease or disorder can be an infectious disease or disorder caused by a coronavirus, and the AP can comprise or can be derived from an antigenic protein of a coronavirus. The term “coronavirus” as used herein refers to a virus in the family Coronaviridae, which is in turn classified within the order Nidovirales. The coronaviruses are large, enveloped, positive-stranded RNA viruses. The coronaviruses have the largest genomes of the RNA viruses known in the art and replicate by a unique mechanism that results in a high frequency of recombination. The coronaviruses include antigenic groups I, II, and III. Nonlimiting examples of coronaviruses include SARS coronavirus (e.g., SARS-CoV and SARS-COV-2), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras thereof. Additional information related to coronavirus including classification, virion structure, genome structure, genetics and pathology is described, for example, in KV Holmes, Encyclopedia of Virology, 1999:291-298, the content of which is incorporated herein by reference.

In some embodiments, a coronavirus described herein is in the genus of Alpha-coronavirus and the coronavirus antigens can be of or derived from any species or strains in the genus of Alpha-coronavirus. In some embodiments, a coronavirus described herein is in the genus of Beta-coronavirus and the coronavirus antigens can be of or derived from any species or strains in the genus of Beta-coronavirus. Member viruses in the genus of Alpha-coronavirus and Beta-coronavirus are enveloped, positive-strand RNA viruses that can infect mammals.

### Tumor-Associated Antigens, Autoimmune Antigens, and Allergenic Antigens

The disease or disorder can be a disease associated with expression of a tumor-associated antigen, and the antigenic protein can be a tumor-associated antigen. A tumor-associated antigen can be a tumor-specific antigen. In some embodiments, the tumor-associated antigen is: 1A01_HLA-A/m (UniProtKB: P30443); 1A02 (UniProtKB: P01892); 5T4 (UniProtKB: Q13641); ACRBP (UniProtKB: Q8NEB7); AFP (UniProtKB: P02771); AKAP4 (UniProtKB: Q5JQC9); alpha-actinin-_4/m (UniProtKB: B4DSX0); alpha-actinin-_4/m (UniProtKB: B4E337); alpha-actinin-_4/m (UniProtKB: 043707); alpha-methylacyl-coenzyme_A_racemase (UniProtKB: A0A024RE16); alpha-methylacyl-coenzyme_A_racemase (UniProtKB: A8KAC3); ANDR (UniProtKB: P10275); ART-4 (UniProtKB: Q9ULX3); ARTC1/m (UniProtKB: P52961); AURKB (UniProtKB: Q96GD4); B2MG (UniProtKB: P61769); B3GN5 (UniProtKB: Q9BYG0); B4GN1 (UniProtKB: Q00973); B7H4 (UniProtKB: Q7Z7D3); BAGE-1 (UniProtKB: Q13072); BASI (UniProtKB: P35613); BCL-2 (UniProtKB: A9QXG9); bcr/abl (UniProtKB: A9UEZ4); bcr/abl (UniProtKB: A9UEZ7); bcr/abl (UniProtKB: A9UEZ8); bcr/abl (UniProtKB: A9UEZ9); bcr/abl (UniProtKB: A9UF00); bcr/abl (UniProtKB: A9UF01); bcr/abl (UniProtKB: A9UF03); bcr/abl (UniProtKB: A9UF04); bcr/abl (UniProtKB: A9UF05); bcr/abl (UniProtKB: A9UF06); bcr/abl (UniProtKB: A9UF08); beta-catenin/m (UniProtKB: P35222); beta-catenin/m (UniProtKB: Q8WYA6); BING-4 (UniProtKB: 015213); BIRC7 (UniProtKB: Q96CA5); BRCA1/m (UniProtKB: A0A024R1V0); BRCA1/m (UniProtKB: A0A024R1V7); BRCA1/m (UniProtKB: A0A024R1Z8); BRCA1/m (UniProtKB: A0A068BFX7); BRCA1/m (UniProtKB: C6YB45); BRCA1/m (UniProtKB: C6YB47); BRCA1/m (UniProtKB: G3XAC3); BY55 (UniProtKB: 095971); calreticulin (UniProtKB: B4DHR1); calreticulin (UniProtKB: B4E2Y9); calreticulin (UniProtKB: P27797); calreticulin (UniProtKB: Q96L12); CAMEL (UniProtKB: 095987); CASP-8/m (UniProtKB: Q14790); CASPA (UniProtKB: Q92851-4); cathepsin_B (UniProtKB: A0A024R374); cathepsin_B (UniProtKB: P07858); cathepsin_L (UniProtKB: A0A024R276); cathepsin_L (UniProtKB: P07711); cathepsin_L (UniProtKB: Q9HBQ7); CDIA (UniProtKB: P06126); CDIB (UniProtKB: P29016); CDIC (UniProtKB: P29017); CDID (UniProtKB: P15813); CDIE (UniProtKB: P15812); CD20 (UniProtKB: P11836); CD22 (UniProtKB: 060926); CD22 (UniProtKB: P20273); CD22 (UniProtKB: Q0EAF5); CD276 (UniProtKB: Q5ZPR3); CD33 (UniProtKB: B4DF51); CD33 (UniProtKB: P20138); CD33 (UniProtKB: Q546G0); CD3E (UniProtKB: P07766); CD3Z (UniProtKB: P20963); CD44_Isoform_1 (UniProtKB: P16070); CD44_Isoform_6 (UniProtKB: P16070-6); CD4 (UniProtKB: P01730); CD52 (UniProtKB: P31358); CD52 (UniProtKB: Q6IBD0); CD52 (UniProtKB: V9HWN9); CD55 (UniProtKB: B1AP15); CD55 (UniProtKB: D3DT85); CD55 (UniProtKB: D3DT86); CD55 (UniProtKB: P08174); CD56 (UniProtKB: P13591); CD80 (UniProtKB: AONOP2); CD80 (UniProtKB: P33681); CD86 (UniProtKB: P42081); CD8A (UniProtKB: P01732); CDCl27/m (UniProtKB: G5EA36); CDCl27/m (UniProtKB: P30260); CDE30 (UniProtKB: P28908); CDK4/m (UniProtKB: A0A024RBB6); CDK4/m (UniProtKB: P11802); CDK4/m (UniProtKB: Q6LC83); CDK4/m (UniProtKB: Q96BE9); CDKN2A/m (UniProtKB: DILYX3); CDKN2A/m (UniProtKB: G3XAG3); CDKN2A/m (UniProtKB: K7PML8); CDKN2A/m (UniProtKB: L8E941); CDKN2A/m (UniProtKB: Q8N726); CEA (RefSeq: NP_004354); CEAM6 (UniProtKB: P40199); CH3L2 (UniProtKB: Q15782); CLCA2 (UniProtKB: Q9UQC9); CML28 (UniProtKB: Q9NQT4); CML66 (UniProtKB: Q96RS6); COA-1/m (UniProtKB: Q5T124); coactosin-like_protein (UniProtKB: Q14019); collagen_XXIII (UniProtKB: L8EAS4); collagen_XXIII (UniProtKB: Q86Y22); COX-2 (UniProtKB: Q6ZYK7); CPIB1 (UniProtKB: Q16678); CSAG2 (UniProtKB: Q9Y5P2-2); CSAG2 (UniProtKB: Q9Y5P2); CT45A1 (UniProtKB: Q5HYN5); CT55 (UniProtKB: Q8WUE5); CT-_9/BRD6 (UniProtKB: Q58F21); CTAG2_Isoform_LAGE-1A (UniProtKB: 075638-2); CTAG2_Isoform_LAGE-1B (UniProtKB: 075638); CTCFL (UniProtKB: Q8NI51); Cten (UniProtKB: Q8IZW8); cyclin_B1 (UniProtKB: P14635); cyclin_D1 (UniProtKB: P24385); cyp-B (UniProtKB: P23284); DAM-10 (UniProtKB: P43366); DEP1A (UniProtKB: Q5TB30); E7 (UniProtKB: P03129); E7 (UniProtKB: P06788); E7 (UniProtKB: P17387); E7 (UniProtKB: P06429); E7 (UniProtKB: P27230); E7 (UniProtKB: P24837); E7 (UniProtKB: P21736); E7 (UniProtKB: P26558); E7 (UniProtKB: P36831); E7 (UniProtKB: P36833); E7 (UniProtKB: Q9QCZ1); E7 (UniProtKB: Q81965); E7 (UniProtKB: Q80956); EF1A2 (UniProtKB: Q05639); EFTUD2/m (UniProtKB: Q15029); EGFR (UniProtKB: A0A0B4J1Y5); EGFR (UniProtKB: E7BSVO); EGFR (UniProtKB: LOR6G1); EGFR (UniProtKB: P00533-2); EGFR (UniProtKB: P00533); EGFR (UniProtKB: Q147T7); EGFR (UniProtKB: Q504U8); EGFR (UniProtKB: Q8NDU8); EGLN3 (UniProtKB: Q9H6Z9); ELF2/m (UniProtKB: B7Z720); EMMPRIN (UniProtKB: Q54A51); EpCam (UniProtKB: P16422); EphA2 (UniProtKB: P29317); EphA3 (UniProtKB: P29320); EphA3 (UniProtKB: Q6P4R6); ErbB3 (UniProtKB: B3KWG5); ErbB3 (UniProtKB: B4DGQ7); ERBB4 (UniProtKB: Q15303); ERG (UniProtKB: P11308); ETV6 (UniProtKB: P41212); EWS (UniProtKB: Q01844); EZH2 (UniProtKB: F2YMM1); EZH2 (UniProtKB: G3XAL2); EZH2 (UniProtKB: LOR855); EZH2 (UniProtKB: Q15910); EZH2 (UniProtKB: S4S3R8); FABP7 (UniProtKB: 015540); FCGR3A_Version_1 (UniProtKB: P08637); FCGR3A_Version_2 (CCDS: CCDS1232.1); FGFS (UniProtKB: P12034); FGFS (UniProtKB: Q60518); FGFR2 (UniProtKB: P21802); fibronectin (UniProtKB: A0A024R516); fibronectin (UniProtKB: A0A024RB01); fibronectin (UniProtKB: A0A024RDT9); fibronectin (UniProtKB: A0A024RDV5); fibronectin (UniProtKB: A6NH44); fibronectin (UniProtKB: A8K6A5); fibronectin (UniProtKB: B2R627); fibronectin (UniProtKB: B3KXM5); fibronectin (UniProtKB: B4DIC5); fibronectin (UniProtKB: B4DN21); fibronectin (UniProtKB: B4DS98); fibronectin (UniProtKB: B4DTH2); fibronectin (UniProtKB: B4DTK1); fibronectin (UniProtKB: B4DU16); fibronectin (UniProtKB: B7Z3W5); fibronectin (UniProtKB: B7Z939); fibronectin (UniProtKB: G5E9X3); fibronectin (UniProtKB: Q9H382); FOS (UniProtKB: P01100); FOXP3 (UniProtKB: Q9BZS1); FUT1 (UniProtKB: P19526); G250 (UniProtKB: Q16790); GAGE-1 (Genbank: AAA82744); GAGE-2 (UniProtKB: Q6NT46); GAGE-3 (UniProtKB: Q13067); GAGE-4 (UniProtKB: Q13068); GAGE-5 (UniProtKB: Q13069); GAGE-6 (UniProtKB: Q13070); GAGE7b (UniProtKB: 076087); GAGE-8_(GAGE-2D) (UniProtKB: Q9UEU5); GASR (UniProtKB: P32239); GnT-V (UniProtKB: Q09328); GPC3 (UniProtKB: 16QTG3); GPC3 (UniProtKB: P51654); GPC3 (UniProtKB: Q8IYG2); GPNMB/m (UniProtKB: A0A024RA55); GPNMB/m (UniProtKB: Q14956); GPNMB/m (UniProtKB: Q8IXJ5); GPNMB/m (UniProtKB: Q96F58); GRM3 (UniProtKB: Q14832); HAGE (UniProtKB: Q9NXZ2); hepsin (UniProtKB: B2ZDQ2); hepsin (UniProtKB: P05981); Her2/neu (UniProtKB: B4DTR1); Her2/neu (UniProtKB: L8E8G2); Her2/neu (UniProtKB: P04626); Her2/neu (UniProtKB: Q9UK79); HLA-A2/m (UniProtKB: Q95387); HLA-A2/m (UniProtKB: Q9MYF8); homeobox_NKX3.1 (UniProtKB: Q99801); HOM-TES-85 (UniProtKB: B2RBQ6); HOM-TES-85 (UniProtKB: Q9P127); HPG1 (Pubmed: 12543784); HS71A (UniProtKB: PODMV8); HS71B (UniProtKB: PODMV9); HST-2 (UniProtKB: P10767); hTERT (UniProtKB: 094807); iCE (UniProtKB: 000748); IF2B3 (UniProtKB: 000425); IL10 (UniProtKB: P22301); IL-13Ra2 (UniProtKB: Q14627); IL2-RA (UniProtKB: P01589); IL2-RB (UniProtKB: P14784); IL2-RG (UniProtKB: P31785); IL-5 (UniProtKB: P05113); IMP3 (UniProtKB: Q9NV31); ITA5 (UniProtKB: P08648); ITB1 (UniProtKB: P05556); ITB6 (UniProtKB: P18564); kallikrein-2 (UniProtKB: A0A024R4J4); kallikrein-2 (UniProtKB: A0A024R4N3); kallikrein-2 (UniProtKB: BOAZU9); kallikrein-2 (UniProtKB: B4DU77); kallikrein-2 (UniProtKB: P20151); kallikrein-2 (UniProtKB: Q6T774); kallikrein-2 (UniProtKB: Q6T775); kallikrein-4 (UniProtKB: A0A0C4DFQ5); kallikrein-4 (UniProtKB: Q5BQA0); kallikrein-4 (UniProtKB: Q96PTO); kallikrein-4 (UniProtKB: Q96PT1); kallikrein-4 (UniProtKB: Q9Y5K2); KI20A (UniProtKB: 095235); KIAA0205 (UniProtKB: Q92604); KIF2C (UniProtKB: Q99661); KK-LC-1 (UniProtKB: Q5H943); LDLR (UniProtKB: P01130); LGMN (UniProtKB: Q99538); LIRB2 (UniProtKB: Q8N423); LY6K (UniProtKB: Q17RY6); MAGAS (UniProtKB: P43359); MAGA8 (UniProtKB: P43361); MAGAB (UniProtKB: P43364); MAGE-A10 (UniProtKB: A0A024RC14); MAGE-A12 (UniProtKB: P43365); MAGE-A1 (UniProtKB: P43355); MAGE-A2 (UniProtKB: P43356); MAGE-A3 (UniProtKB: P43357); MAGE-A4 (UniProtKB: A0A024RC12); MAGE-A4 (UniProtKB: P43358); MAGE-A4 (UniProtKB: Q1RN33); MAGE-A6 (UniProtKB: A8K072); MAGE-A6 (UniProtKB: P43360); MAGE-A6 (UniProtKB: Q6FHI5); MAGE-A9 (UniProtKB: P43362); MAGE-B10 (UniProtKB: Q96LZ2); MAGE-B16 (UniProtKB: A2A368); MAGE-B17 (UniProtKB: A8MXT2); MAGE-B1 (UniProtKB: Q96TG1); MAGE-B2 (UniProtKB: 015479); MAGE-B3 (UniProtKB: 015480); MAGE-B4 (UniProtKB: 015481); MAGE-B5 (UniProtKB: Q9BZ81); MAGE-B6 (UniProtKB: Q8N7X4); MAGE-CI (UniProtKB: 060732); MAGE-C2 (UniProtKB: Q9UBF1); MAGE-C3 (UniProtKB: Q8TD91); MAGE-DI (UniProtKB: Q9Y5V3); MAGE-D2 (UniProtKB: Q9UNF1); MAGE-D4 (UniProtKB: Q96JG8); MAGE-_E1 (UniProtKB: Q6IAI7); MAGE-E1_(MAGE1) (UniProtKB: Q9HCI5); MAGE-E2 (UniProtKB: Q8TD90); MAGE-F1 (UniProtKB: Q9HAY2); MAGE-HI (UniProtKB: Q9H213); MAGEL2 (UniProtKB: Q9U355); mammaglobin_A (UniProtKB: Q13296); mammaglobin_A (UniProtKB: Q6NX70); MART-1/melan-A (UniProtKB: Q16655); MART-2 (UniProtKB: Q5VTY9); MCI_R (UniProtKB: Q01726); MC1_R (UniProtKB: Q1JUL4); MC1_R (UniProtKB: Q1JUL6); MC1_R (UniProtKB: Q1JUL8); MC1_R (UniProtKB: Q1JUL9); MCI_R (UniProtKB: Q1JUM0); MCI_R (UniProtKB: Q1JUM2); MC1_R (UniProtKB: Q1JUM3); MCI_R (UniProtKB: Q1JUM4); MC1_R (UniProtKB: Q1JUM5); MC1_R (UniProtKB: Q6UR92); MCI_R (UniProtKB: Q6UR94); MC1_R (UniProtKB: Q6UR95); MCI_R (UniProtKB: Q6UR96); MC1_R (UniProtKB: Q6UR97); MC1_R (UniProtKB: Q6UR98); MCI_R (UniProtKB: Q6UR99); MC1_R (UniProtKB: Q6URA0); MCI_R (UniProtKB: Q86YW1); MC1_R (UniProtKB: V9Q5S2); MC1_R (UniProtKB: V9Q671); MCI_R (UniProtKB: V9Q783); MC1_R (UniProtKB: V9Q7F1); MCI_R (UniProtKB: V9Q8N1); MCI_R (UniProtKB: V9Q977); MC1_R (UniProtKB: V9Q9P5); MC1_R (UniProtKB: V9Q9R8); MC1_R (UniProtKB: V9QAE0); MCI_R (UniProtKB: V9QAR2); MC1_R (UniProtKB: V9QAW3); MC1_R (UniProtKB: V9QB02); MC1_R (UniProtKB: V9QB58); MC1_R (UniProtKB: V9QBY6); MC1_R (UniProtKB: V9QC17); MC1_R (UniProtKB: V9QC66); MC1_R (UniProtKB: V9QCQ4); MC1_R (UniProtKB: V9QDF4); MCI_R (UniProtKB: V9QDN7); MCI_R (UniProtKB: V9QDQ6); M-CSF (UniProtKB: P09603); mesothelin (UniProtKB: Q13421); MITF (UniProtKB: 075030-8); MITF (UniProtKB: 075030-9); MITF (UniProtKB: 075030); MMP1_1 (UniProtKB: B3KQS8); MMP7 (UniProtKB: P09237); MUC-1 (Genbank: AAA60019); MUM-1/m (RefSeq: NP_116242); MUM-2/m (UniProtKB: Q9Y5R8); MYCN (UniProtKB: P04198); MY01A (UniProtKB: Q9UBC5); MY01B (UniProtKB: 043795); MY01C (UniProtKB: 000159); MYO1D (UniProtKB: 094832); MY01E (UniProtKB: Q12965); MY0IF (UniProtKB: 000160); MY01G (UniProtKB: B0I1T2); MY01H (RefSeq: NP_001094891); NA17 (UniProtKB: Q3V5L5); NA88-A (Pubmed: 10790436); Neo-PAP (UniProtKB: Q9BWT3); NFYC/m (UniProtKB: Q13952); NGEP (UniProtKB: Q6IWH7); NPM (UniProtKB: P06748); NRCAM (UniProtKB: Q92823); NSE (UniProtKB: P09104); NUF2 (UniProtKB: Q9BZD4); NY-ESO-1 (UniProtKB: P78358); 0A1 (UniProtKB: P51810); OGT (UniProtKB: 015294); OS-9 (UniProtKB: B4DH11); OS-9 (UniProtKB: B4E321); OS-9 (UniProtKB: B7Z8E7); OS-9 (UniProtKB: Q13438); osteocalcin (UniProtKB: P02818); osteopontin (UniProtKB: A0A024RDE2); osteopontin (UniProtKB: A0A024RDE6); osteopontin (UniProtKB: A0A024RDJ0); osteopontin (UniProtKB: B7Z351); osteopontin (UniProtKB: F2YQ21); osteopontin (UniProtKB: P10451); p53 (UniProtKB: P04637); PAGE-4 (UniProtKB: 060829); PAI-1 (UniProtKB: P05121); PAI-2 (UniProtKB: P05120); PAP (UniProtKB: Q06141); PAP (UniProtKB: Q53S56); PATE (UniProtKB: Q8WXA2); PAX3 (UniProtKB: P23760); PAXS (UniProtKB: Q02548); PD1L1 (UniProtKB: Q9NZQ7); PDCD1 (UniProtKB: Q15116); PDEF (UniProtKB: 095238); PECA1 (UniProtKB: P16284); PGCB (UniProtKB: Q96GW7); PGFRB B (UniProtKB: P09619); Pim-1-Kinase (UniProtKB: A0A024RD25); Pin-1 (UniProtKB: 015428); Pin-1 (UniProtKB: Q13526); Pin-1 (UniProtKB: Q49AR7); PLAC1 (UniProtKB: Q9HBJ0); PMEL (UniProtKB: P40967); PML (UniProtKB: P29590); POTEF (UniProtKB: A5A3E0); POTE (UniProtKB: Q86YR6); PRAME (UniProtKB: A0A024R1E6); PRAME (UniProtKB: P78395); PRDX5/m (UniProtKB: P30044); PRM2 (UniProtKB: P04554); prostein (UniProtKB: Q96JT2); proteinase-3 (UniProtKB: D6CHE9); proteinase-3 (UniProtKB: P24158); PSA (UniProtKB: P55786); PSB9 (UniProtKB: P28065); PSCA (UniProtKB: D3DWI6); PSCA (UniProtKB: 043653); PSGR (UniProtKB: Q9H255); PSM (UniProtKB: Q04609); PTPRC (RefSeq: NP_002829); RAB8A (UniProtKB: P61006); RAGE-1 (UniProtKB: Q9UQ07); RARA (UniProtKB: P10276); RASH (UniProtKB: P01112); RASK (UniProtKB: P01116); RASN (UniProtKB: P01111); RGSS (UniProtKB: 015539); RHAMM/CD168 (UniProtKB: 075330); RHOC (UniProtKB: P08134); RSSA (UniProtKB: P08865); RUI (UniProtKB: Q9UHJ3); RU2 (UniProtKB: Q9UHG0); RUNX1 (UniProtKB: Q01196); S-100 (UniProtKB: V9HW39); SAGE (UniProtKB: Q9NXZ1); SART-_1 (UniProtKB: 043290); SART-2 (UniProtKB: Q9UL01); SART-3 (UniProtKB: Q15020); SEPR (UniProtKB: Q12884); SERPINBS (UniProtKB: P36952); SIA7F (UniProtKB: Q969X2); SIA8A (UniProtKB: Q92185); SIAT9 (UniProtKB: Q9UNP4); SIRT2/m (UniProtKB: A0A024ROG8); SIRT2/m (UniProtKB: Q8IXJ6); SOX10 (UniProtKB: P56693); SP17 (UniProtKB: Q15506); SPNXA (UniProtKB: Q9NS26); SPXN3 (UniProtKB: Q5MJ09); SSX-1 (UniProtKB: Q16384); SSX-2 (UniProtKB: Q16385); SSX3 (UniProtKB: Q99909); SSX-4 (UniProtKB: 060224); ST1A1 (UniProtKB: P50225); STAG2 (UniProtKB: Q8N3U4-2); STAMP-1 (UniProtKB: Q8NFT2); STEAP-1 (UniProtKB: A0A024RA63); STEAP-1 (UniProtKB: Q9UHE8); Survivin-2B (UniProtKB: 015392-2); survivin (UniProtKB: 015392); SYCP1 (UniProtKB: A0A024R012); SYCP1 (UniProtKB: B7ZLS9); SYCP1 (UniProtKB: Q15431); SYCP1 (UniProtKB: Q3MHC4); SYT-SSX-1 (UniProtKB: A4PIV7); SYT-SSX-1 (UniProtKB: A4PIV8); SYT-SSX-2 (UniProtKB: A4PIV9); SYT-SSX-2 (UniProtKB: A4PIWO); TARP (UniProtKB: QOVGM3); TCRg (UniProtKB: A2JGV3); TF2AA (UniProtKB: P52655); TGFB1 (UniProtKB: P01137); TGFR2 (UniProtKB: P37173); TGM-4 (UniProtKB: B2R7D1); TIE2 (UniProtKB: Q02763); TKIL1 (UniProtKB: P51854); TPI/m (UniProtKB: P60174); TRGV11 (UniProtKB: Q99601); TRGV9 (UniProtKB: A4D1X2); TRGV9 (UniProtKB: Q99603); TRGV9 (UniProtKB: Q99604); TRPC1 (UniProtKB: P48995); TRP-p8 (UniProtKB: Q7Z2W7); TSG10 (UniProtKB: Q9BZW7); TSPY1 (UniProtKB: Q01534); TVC_(TRGV3) (Genbank: M13231.1); TX101 (UniProtKB: Q9BY14-2); tyrosinase (UniProtKB: A0A024DBG7); tyrosinase (UniProtKB: L8B082); tyrosinase (UniProtKB: L8B086); tyrosinase (UniProtKB: L8B0B9); tyrosinase (UniProtKB: 075767); tyrosinase (UniProtKB: P14679); tyrosinase (UniProtKB: U3M8N0); tyrosinase (UniProtKB: U3M9D5); tyrosinase (UniProtKB: U3M9J2); TYRP1 (UniProtKB: P17643); TYRP2 (UniProtKB: P40126); UPA (UniProtKB: Q96NZ9); VEGFR1 (UniProtKB: B5A924); WT1 (UniProtKB: A0A0H5AUY0); WT1 (UniProtKB: P19544); WT1 (UniProtKB: Q06250); and XAGE1 (UniProtKB: Q9HD64).

The disease or disorder can be an autoimmune disease or disorder, and the antigenic protein can be an autoimmune antigen. The autoimmune antigen can comprise: myelin basic protein (MBP), proteolipid protein (PLP), and myelin oligodendrocyte glycoprotein (MOG), in each case associated with multiple sclerosis (MS); CD44, preproinsulin, proinsulin, insulin, glutamic acid decaroxylase (GAD65), tyrosine phosphatase-like insulinoma antigen 2 (IA2), zinc transporter ((ZnT8), and heat shock protein 60 (HSP60), in each case associated with diabetes Typ I; interphotoreceptor retinoid-binding protein (IRBP) associated with autoimmune uveitis; acetylcholine receptor AchR, and insulin-like growth factor-1 receptor (IGF-IR), in each case associated with Myasthenia gravis; M-protein from beta-hemolytic streptocci (pseudo-autoantigen) associated with Rheumatic Fever, or any combination thereof. Autoimmune antigens (antigens associated with autoimmune disease or autoantigens) are selected from autoantigens associated with autoimmune diseases selected from Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinine disease, cold autoimmune hemolytic anemia (AIHA) (cold agglutinin disease), (CHAD)), autoimmune hemolytic anemia (AIHA) of the warm type (warm AIHA, warm autoimmune haemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), cardiomyopathy, particularly autoimmune cardiomyopathy, idiopathic dilated cardiomyopathy (DCM), celiac sprue dermatitis (gluten mediated enteropathia), chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrom with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, Diabetes, Diabetes mellitus Type 1 (type I diabetes, insuline dependent Diabetes mellitus), Diabetes mellitus Type 2 (type II diabetes), essential mixed cryoglobulinemia, essential mixed cryoglobulinemia, fibromyalgia, fibromyositis, Goodpasture syndrome (anti-GBM mediated glomerulonephritis), graft versus host disease, Guillain-Barre syndrome (GBM, Polyradikuloneuritis), haematologic autoimmune diseases, Hashimoto thyroiditis, hemophilia, acquired hemophilia, hepatitis, autoimmune hepatitis, particularly autoimmune forms of chronic hepatitis, idiopathic pulmonary fibrosis (IPF), idiopathic thrombocytopenia purpura, Immuno-thrombocytopenia purpura (Morbus Werlhof; ITP), IgA nephropathy, infertility, autoimmune infertility, juvenile rheumatoid arthritis (Morbus Still, Still syndrome), Lambert-Eaton syndrome, lichen planus, lichen sclerosus, lupus erythematosus, systemic lupus erythematosus (SLE), lupus erythematosus (discoid form), Lyme arthritis (Lyme disease, borrelia arthritis), Ménierè's disease (Morbus Ménierè); mixed connective tissue disease (MCTD) multiple sclerosis (MS, encephalomyelitis disseminate, Charcot's disease), Myasthenia gravis (myasthenia, MG), myosits, polymyositis, neural autoimmune diseases, neurodermitis, pemphigus vulgaris, bullous pemphigoid, scar forming pemphigoid; polyarteriitis nodosa (periarteiitis nodosa), polychondritis (panchondritis), polyglandular (autoimmune) syndrome (PGA syndrome, Schmidt's syndrome), Polymyalgia rheumatica, primary agammaglobulinemia, primary biliary cirrhosis PBC, primary autoimmune cholangitis), progressive systemic sclerosis (PSS), Psoriasis, Psoriasis vulgaris, Raynaud's phenomena, Reiter's syndrome (Morbus Reiter, urethral conjunctive synovial syndrome)), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, sympathetic ophtalmia; Transient gluten intolerance, transplanted organ rejection, uveitis, autoimmune uveiitis, Vasculitis, Vitiligo, (leucoderma, piebold skin), and Wegner's disease (Morbus Wegner, Wegner's granulomatosis).

The disease or disorder can be an allergic disease or disorder, and the antigenic protein can be an allergenic antigen. In some embodiments, the allergenic antigen is: Acarus spp (Aca s 1, Aca s 10, Aca s 10.0101, Aca s 13, Aca s 13.0101, Aca s 2, Aca s 3, Aca s 7, Aca s 8), Acanthocybium spp (Aca so 1), Acanthocheilonema spp (Aca v 3, Aca v 3.0101), Acetes spp (Ace ja 1), Actinidia spp (Act a 1, Act c 1, Act c 10, Act c 10.0101, Act c 2, Act c 4, Act c 5, Act c 5.0101, Act c 8, Act c 8.0101, Act c Chitinase, Act d 1, Act d 1.0101, Act d 10, Act d 10.0101, Act d 10.0201, Act d 11, Act d 11.0101, Act d 2, Act d 2.0101, Act d 3, Act d 3.0101, Act d 3.02, Act d 4, Act d 4.0101, Act d 5, Act d 5.0101, Act d 6, Act d 6.0101, Act d 7, Act d 7.0101, Act d 8, Act d 8.0101, Act d 9, Act d 9.0101, Act d Chitinase, Act e 1, Act e 5), Acyrthosiphon spp (Acy pi 7, Acy pi 7.0101, Acy pi 7.0102), Adenia spp (Ade v RIP), Aedes spp (Aed a 1, Aed a 1.0101, Aed a 2, Aed a 2.0101, Aed a 3, Aed a 3.0101, Aed a 4, Aed a 7, Aed a 7.0101, Aed a 7.0102, Aed a 7.0103, Aed a 7.0104, Aed a 7.0105, Aed a 7.0106, Aed a 7.0107, Aed a 7.0108, Aed a 7.0109, Aed a 7.0110, Aed a 7.0111, Aed al 1, Aed al 3, Aed al 37 kD, Aed v 37 kD, Aed v 63 kD), Aegilops spp (Aeg ta 28, Aeg ta alpha_Gliadin, Aeg um 28, Aeg un 28), Aethaloperca spp (Aet ro 1), Agropyron spp (Agr c 7), Agrostis spp (Agr ca 1, Agr ca 5, Agr g 1, Agr g 4, Agr s 5), Agrobacterium spp (Agr sp CP4 EPSPS), Ailuropoda spp (Ail me Phosvitin, Ail me TCTP), Aix spp (Aix ga 1, Aix sp 1), Aleuroglyphus spp (Ale o 1, Ale o 10, Ale o 10.0101, Ale o 10.0102, Ale o 13, Ale o 14, Ale o 2, Ale o 20, Ale o 3, Ale o 5, Ale o 7, Ale o 8, Ale o 9), Allium spp (All a 3, All a Alliin lyase, All c 3, All c 30 kD, All c 4, All c Alliin lyase, All p Alliin lyase, All s Alliin lyase), Alnus spp (Aln g 1, Aln g 1.0101, Aln g 1/Bet v 1/Cor a 1 TPC7, Aln g 1/Bet v 1/Cor a 1 TPC9, Aln g 2, Aln g 4, Aln g 4.0101), Alopochen spp (Alo ae 1), Alopecurus spp (Alo p 1, Alo p 5), Alternaria spp (Alt a 1, Alt a 1.0101, Alt a 1.0102, Alt a 10, Alt a 10.0101, Alt a 12, Alt a 12.0101, Alt a 13, Alt a 13.0101, Alt a 2, Alt a 3, Alt a 3.0101, Alt a 4, Alt a 4.0101, Alt a 5, Alt a 5.0101, Alt a 6, Alt a 6.0101, Alt a 7, Alt a 7.0101, Alt a 70 kD, Alt a 8, Alt a 8.0101, Alt a 9, Alt a MnSOD, Alt a NTF2, Alt a TCTP, Alt ar 1, Alt arg 1, Alt b 1, Alt bl 1, Alt br 1, Alt c 1, Alt ca 1, Alt ce 1, Alt ch 1, Alt ci 1, Alt co 1, Alt cr 1, Alt ct 1, Alt cu 1, Alt cy 1, Alt d 1, Alt du 1, Alt e 1, Alt et 1, Alt eu 1, Alt ga 1, Alt gr 1, Alt j 1, Alt 11, Alt lo 1, Alt m 1, Alt me 1, Alt mi 1, Alt mo 1, Alto 1, Alt p 1, Alt ph 1, Alt po 1, Alt ps 1, Alt r 1, Alt s 1, Alt se 1, Alt sm 1, Alt so 1, Alt su 1, Alt t 1, Alt to 1, Alt to 1), Amaranthus spp (Ama r 2, Amar 2.0101, Ama v 2, Ama v 2.0101, Ama v 2.0201), Ambrosia spp (Amb a 1, Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, Amb a 10, Amb a 10.0101, Amb a 3, Amb a 3.0101, Amb a 4, Amb a 4.0101, Amb a 5, Amb a 5.0101, Amb a 6, Amb a 6.0101, Amb a 7, Amb a 7.0101, Amb a 8, Amb a 8.0101, Amb a 8.0102, Amb a 9, Amb a 9.0101, Amb a 9.0102, Amb a CPI, Amb p 1, Amb p 5, Amb p 5.0101, Amb p 5.0201, Amb t 5, Amb t 5.0101, Amb t 8), Ammothea spp (Amm h 7, Amm h 7.0101), Anadara spp (Ana br 1), Ananas spp (Ana c 1, Ana c 1.0101, Ana c 2, Ana c 2.0101, Ana c 2.0101 (MUXF3)), Anas spp (Ana ca 1), Anarhichas spp (Ana I 1), Anacardium spp (Ana o 1, Ana o 1.0101, Ana o 1.0102, Ana o 2, Ana o 2.0101, Ana o 3, Ana o 3.0101), Anas spp (Ana p 1, Ana p 2, Ana p 3), Anguilla spp (Ang a 1, Ang j 1), Anisakis spp (Ani s 1, Ani s 1.0101, Ani s 10, Ani s 10.0101, Ani s 11, Ani s 11.0101, Ani s 12, Ani s 12.0101, Ani s 2, Ani s 2.0101, Ani s 24 kD, Ani s 3, Ani s 3.0101, Ani s 4, Ani s 4.0101, Ani s 5, Ani s 5.0101, Ani s 6, Ani s 6.0101, Ani s 7, Ani s 7.0101, Ani s 8, Ani s 8.0101, Ani s 9, Ani s 9.0101, Ani s CCOS3, Ani s Cytochrome B, Ani s FBPP, Ani s NADHDS4L, Ani s NARaS, Ani s PEPB, Ani s Troponin), Annona spp (Ann c Chitinase), Anopheles spp (Ano da 17, Ano da 17.0101, Ano da 27, Ano da 27.0101, Ano da 7, Ano da 7.0101, Ano g 7, Ano g 7.0101), Anser spp (Ans a 1, Ans a 2, Ans a 3, Ans in 1), Anthoxanthum spp (Ant o 1, Ant o 1.0101, Ant o 12, Ant o 13, Ant o 2, Ant o 4, Ant o 5, Ant o 6, Ant o 7), Apis spp (Api c 1, Api c 1.0101, Api c 10, Api c 2, Api c 4, Api d 1, Api d 1.0101, Api d 4, Api fl 4), Apium spp (Api g 1, Api g 1.0101, Api g 1.0201, Api g 2, Api g 2.0101, Api g 3, Api g 3.0101, Api g 4, Api g 4.0101, Api g 5, Api g 5.0101, Api g 6, Api g 6.0101), Apis spp (Api m 1, Api m 1.0101, Api m 10, Api m 10.0101, Api m 11, Api m 11.0101, Api m 11.0201, Api m 13 kD, Api m 2, Api m 2.0101, Api m 3, Api m 3.0101, Api m 4, Api m 4.0101, Api m 5, Api m 5.0101, Api m 6, Api m 6.0101, Api m 7, Api m 7.0101, Api m 8, Api m 8.0101, Api m 9, Api m 9.0101, Api m A1-A2, Api m A1-A2-A3, Api m Apalbumin 1, Api m Apalbumin 2, Api me 1, Api me 4), Arachis spp (Ara d 2, Ara d 6, Ara f 3, Ara f 4, Ara h 1, Ara h 1.0101, Ara h 10, Ara h 10.0101, Ara h 10.0102, Ara h 11, Ara h 11.0101, Ara h 2, Ara h 2.0101, Ara h 2.0102, Ara h 2.0201, Ara h 2.0202, Ara h 3, Ara h 3.0101, Ara h 4, Ara h 4.0101, Ara h 5, Ara h 5.0101, Ara h 6, Ara h 6.0101, Ara h 7, Ara h 7.0101, Ara h 7.0201, Ara h 7.0202, Ara h 8, Ara h 8.0101, Ara h 8.0201, Ara h 9, Ara h 9.0101, Ara h 9.0201, Ara h Agglutinin, Ara h Oleosin 18 kD, Ara i 2, Ara i 6), Arabidopsis spp (Ara t 3, Ara t 8, Ara t GLP), Archosargus spp (Arc pr 1), Archaeopotamobius spp (Arc s 8, Arc s 8.0101), Aequipecten spp (Arg i 1), Argas spp (Arg r 1, Arg r 1.0101), Ariopsis spp (Ari fe 1), Armoracia spp (Arm r HRP), Arrhenatherum spp (Arr e 1, Arr e 5), Artemisia spp (Art a 1, Art ap 1), Artemia spp (Art fr 1, Art fr 1.0101, Art fr 5, Art fr 5.0101), Arthrobacter spp (Art gl CO), Achorion spp (Art gy 7), Artocarpus spp (Art h 17 kD, Art h 4), Arthrospira spp (Art pl beta_Phycocyanin), Artemisia spp (Art v 1, Art v 1.0101, Art v 1.0102, Art v 1.0103, Art v 1.0104, Art v 1.0105, Art v 1.0106, Art v 1.0107, Art v 2, Art v 2.0101, Art v 3, Art v 3.0101, Art v 3.0201, Art v 3.0202, Art v 3.0301, Art v 4, Art v 4.0101, Art v 4.0201, Art v 47 kD, Art v 5, Art v 5.0101, Art v 6, Art v 6.0101, Art v 60 kD), Arthroderma spp (Art va 4), Ascaris spp (Asc 13, Asc 1 3.0101, Asc 1 3.0102, Asc 1 34 kD, Asc s 1, Asc s 1.0101, Asc s 3, Asc s 3.0101, Asc s GST), Aspergillus spp (Asp aw Glucoamylase, Asp c 22, Asp f 1, Asp f 1.0101, Asp f 10, Asp f 10.0101, Asp f 11, Asp f 11.0101, Asp f 12, Asp f 12.0101, Asp f 13, Asp f 13.0101, Asp f 15, Asp f 15.0101, Asp f 16, Asp f 16.0101, Asp f 17, Asp f 17.0101, Asp f 18, Asp f 18.0101, Asp f 2, Asp f 2.0101, Asp f 22, Asp f 22.0101, Asp f 23, Asp f 23.0101, Asp f 27, Asp f 27.0101, Asp f 28, Asp f 28.0101, Asp f 29, Asp f 29.0101, Asp f 3, Asp f 3.0101, Asp f 34, Asp f 34.0101, Asp f 4, Asp f 4.0101, Asp f 5, Asp f 5.0101, Asp f 56 kD, Asp f 6, Asp f 6.0101, Asp f 7, Asp f 7.0101, Asp f 8, Asp f 8.0101, Asp f 9, Asp f 9.0101, Asp f AfCalAp, Asp f AT_V, Asp f Catalase, Asp f Chitosanase, Asp f CP, Asp f DPPV, Asp f FDH, Asp f gamma_Actin, Asp f Glucosidase, Asp f GPI, Asp f GST, Asp f GT, Asp f IAO, Asp f IPMI, Asp f LPL1, Asp f LPL3, Asp f Mannosidase, Asp f MDH, Asp f PL, Asp f PUP, Asp f RPS3, Asp f SXR, Asp fl 13, Asp fl 13.0101, Asp fl 18, Asp fl 2, Asp fl 21, Asp fl 3, Asp fl 4, Asp fl 7, Asp fl 8, Asp fl 9, Asp me Sea prose, Asp n 14, Asp n 14.0101, Asp n 18, Asp n 18.0101, Asp n 25, Asp n 25.0101, Asp n 30, Asp n Glucoamylase, Asp n Hemicellulase, Asp n Pectinase, Asp o 13, Asp o 13.0101, Asp o 21, Asp o 21.0101, Asp o 3, Asp o 4, Asp o 7, Asp o 8, Asp o Lactase, Asp o Lipase, Asp oc 13, Asp r 1, Asp sa AP, Asp sp Glucoamylase, Asp sp Glucoseoxidase, Asp sp PL, Asp sp PME, Asp sy 13, Asp v 13, Asp v 13.0101, Asp v Catalase A, Asp v Enolase, Asp v GAPDH, Asp v MDH, Asp v SXR), Asparagus spp (Aspa o 1, Aspa o 1.01, Aspa o 1.02, Aspa o 17 kD, Aspa o 4), Aspergillus spp (Aspe ni 2, Aspe ni 3, Aspe ni 4, Aspe ni 7, Aspe ni 8, Aspe ni 9), Avena spp (Ave s 1, Ave s 12, Ave s 13, Ave s 2, Ave s 4, Ave s 5, Ave s 7), Babylonia spp (Bab ja 1), Bacillus spp (Bac al Subtilisin, Bac cl Subtilisin, Bac I Subtilisin, Bac li aA, Bac Ii Subtilisin), Bactrocera spp (Bac ol 27, Bac ol 27.0101), Bacillus spp (Bac sp aA1, Bac sp aA3, Bac sp Decarboxylase, Bac st amyM, Bac su Subtilisin, Bac t CrylAb, Bac t CrylFa, Bac t Cry3Bb1, Bac t Cry9c), Bagre spp (Bag ma 1), Balistes spp (Bal ca 1), Balanus spp (Bal r 1, Bal r 1.0101), Beauveria spp (Bea b AId, Bea b Enol, Bea b f2, Bea b Hex), Bertholletia spp (Ber e 1, Ber e 1.0101, Ber e 2, Ber e 2.0101), Beryx spp (Ber sp 1), Betula spp (Bet ab 1, Bet al 1, Bet ch 1, Bet co 1, Bet da 1, Bet gr 1, Bet hu 1, Bet le 1, Bet me 1, Bet n 1, Bet p 1, Bet pa 1, Bet po 1, Bet pu 1, Bet pu 2, Bet pu 4, Bet pu 6, Bet pu 7, Bet sc 1, Bet ut 1, Bet v 1, Bet v 1 B1-131-131, Bet v 1 fv Mal 4×, Bet v 1.0101, Bet v 1.0102, Bet v 1.0103, Bet v 1.0201, Bet v 1.0301, Bet v 1.0401, Bet v 1.0402, Bet v 1.0501, Bet v 1.0601, Bet v 1.0602, Bet v 1.0701, Bet v 1.0801, Bet v 1.0901, Bet v 1.1001, Bet v 1.1101, Bet v 1.1201, Bet v 1.1301, Bet v 1.1401, Bet v 1.1402, Bet v 1.1501, Bet v 1.1502, Bet v 1.1601, Bet v 1.1701, Bet v 1.1801, Bet v 1.1901, Bet v 1.2001, Bet v 1.2101, Bet v 1.2201, Bet v 1.2301, Bet v 1.2401, Bet v 1.2501, Bet v 1.2601, Bet v 1.2701, Bet v 1.2801, Bet v 1.2901, Bet v 1.3001, Bet v 1.3101, Bet v 2, Bet v 2.0101, Bet v 3, Bet v 3.0101, Bet v 4, Bet v 4.0101, Bet v 6, Bet v 6.0101, Bet v 6.0102, Bet v 7, Bet v 7.0101, Bet v 8, Bet v Glucanase), Beta spp (Beta v 1, Beta v 1.0101, Beta v 2, Beta v 2.0101), Blattella spp (Bla g 1, Bla g 1.0101, Bla g 1.0102, Bla g 1.0103, Bla g 1.0201, Bla g 1.0202, Bla g 2, Bla g 2.0101, Bla g 2.0201, Bla g 36 kD, Bla g 4, Bla g 4.0101, Bla g 4.0201, Bla g 5, Bla g 5.0101, Bla g 5.0201, Bla g 6, Bla g 6.0101, Bla g 6.0201, Bla g 6.0301, Bla g 7, Bla g 7.0101, Bla g 8, Bla g 8.0101, Bla g 9, Bla g Enolase, Bla g GSTD1, Bla g RACK1, Bla g TPI, Bla g Trypsin, Bla g Vitellogenin), Blatta spp (Bla o 1, Bla o 7), Blomia spp (Blo t 1, Blo t 1.0101, Blo t 1.0201, Blo t 10, Blo t 10.0101, Blo t 10.0102, Blot 11, Blot 11.0101, Blot 12, Blo t 12.0101, Blot 12.0102, Blo t 13, Blo t 13.0101, Blot 14, Blot 15, Blot 18, Blot 19, Blot 19.0101, Blo t 2, Blo t 2.0101, Blo t 2.0102, Blo t 2.0103, Blo t 20, Blo t 21, Blo t 21.0101, Blot 3, Blo t 3.0101, Blo t 4, Blo t 4.0101, Blo t 5, Blo t 5.0101, Blot 6, Blo t 6.0101, Blot 7, Blot 8, Blot 9, Blot HSP70), Bombus spp (Bom ar 4, Bom by 4, Bom p 1, Bom p 1.0101, Bom p 2, Bom p 3, Bom p 4, Bom p 4.0101, Bom t 1, Bom t 1.0101, Bom t 4, Bom t 4.0101), Bombyx spp (Bomb m 1, Bomb m 1.0101, Bomb m 7, Bomb m 7.0101, Bomb m 7.0102, Bomb m 7.0103, Bomb m 7.0104, Bomb m 7.0105, Bomb m 7.0106), Boophilus spp (Boo m 1, Boo m 7, Boo m 7.0101), Bos spp (Bos d 2, Bos d 2.0101, Bos d 2.0102, Bos d 2.0103, Bos d 3, Bos d 3.0101, Bos d 4, Bos d 4.0101, Bos d 5, Bos d 5.0101, Bos d 5.0102, Bos d 6, Bos d 6 (MDA), Bos d 6.0101, Bos d 7, Bos d 7.0101, Bos d 8, Bos d 8 alphaS1, Bos d 8 alphaS2, Bos d 8 beta, Bos d 8 kappa, Bos d alpha2I, Bos d alpha21.0101, Bos d Chymosin, Bos d Fibrin, Bos d Gelatin, Bos d HG, Bos d Insulin, Bos d Lactoferrin, Bos d Lactoperoxidase, Bos d Myoglobin, Bos d OBP, Bos d OSCP, Bos d Phosvitin, Bos d PLA2, Bos d PRVB, Bos d Thrombin, Bos d TI, Bos gr ALA, Bos gr Myoglobin), Bothrops spp (Bot as 1, Bot at 1), Bouteloua spp (Bou g 1), Biting spp (Boy ov 1), Brama spp (Bra du 1), Brassica spp (Bra j 1, Bra j 1.0101, Bra n 1, Bra n 1.0101, Bra n 4, Bra n 7, Bra n 8, Bra n PG, Bra ni 8, Bra o 3, Bra o 3.0101, Bra r 1, Bra r 1.0101, Bra r 2, Bra r 2.0101, Bra r 3, Bra r 4, Bra r 7), Bromus spp (Bro a 1, Bro a 4), Brosme spp (Bro br 1), Bromus spp (Bro i 1, Bro i 5, Bro i 7), Brugia spp (Bru m 3, Bru m 3.0101, Bru m Bm33), Bubalus spp (Bub b ALA, Bub b BLG, Bub b Casein, Bub b Casein alphaS1, Bub b Casein alphaS2, Bub b Casein beta, Bub b Casein kappa), Caenorhabditis spp (Cae b 3, Cae b 3.0101, Cae br 3, Cae br 3.0101, Cae e 3, Cae e 3.0101, Cae e 3.0102, Cae re 13, Cae re 13.0101), Cajanus spp (Caj c 1), Caligus spp (Cal cl 1, Cal cl 1.0101, Cal cl 1.0102), Calamus spp (Cal le 1), Callinectes spp (Cal s 2), Camelus spp (Cam d ALA, Cam d Casein, Cam d Casein alphaS1, Cam d Casein alphaS2, Cam d Casein beta, Cam d Casein kappa), Camponotus spp (Cam fl 7, Cam fl 7.0101), Canis spp (Can f 1, Can f 1.0101, Can f2, Can f 2.0101, Can f 3, Can f 3.0101, Can f 4, Can f 4.0101, Can f 5, Can f 5.0101, Can f 6, Can f 6.0101, Can f Feld1-like, Can f Homs2-like, Can f Phosvitin, Can f TCTP), Canthidermis spp (Can ma 1), Cancer spp (Can mg 2, Can p 1), Cannabis spp (Can s 3), Candida spp (Cand a 1, Cand a 1.0101, Cand a 3, Cand a 3.0101, Cand a CAAP, Cand a CyP, Cand a Enolase, Cand a FPA, Cand a MnSOD, Cand a PGK, Cand b 2, Cand b 2.0101, Cand b FDH, Cand r Lipase), Capsicum spp (Cap a 1, Cap a 1.0101, Cap a 17 kD, Cap a 2, Cap a 2.0101, Cap a 30 kD, Cap a Glucanase, Cap ch 17 kD), Caprella spp (Cap e 1), Capra spp (Cap h ALA, Cap h BLG, Cap h Casein, Cap h Casein alphaS1, Cap h Casein alphaS2, Cap h Casein beta, Cap h Casein kappa, Cap h GSA), Capitulum spp (Cap m 1), Carassius spp (Car au 1), Carpinus spp (Car b 1, Car b 1.0101, Car b 1.0102, Car b 1.0103, Carb 1.0104, Car b 1.0105, Car b 1.0106, Car b 1.0107, Car b 1.0108, Car b 1.0109, Car b 1.0110, Carb 1.0111, Carb 1.0112, Car b 1.0113, Carb 1.0201, Car b 1.0301, Car b 1.0302, Car b 2, Car b 4), Caranx spp (Car cr 1), Carya spp (Car i 1, Car i 1.0101, Car i 2, Car i 4, Car i 4.0101), Carcinus spp (Car ma 2), Caryota spp (Car mi 2), Carica spp (Car p 1, Car p Chitinase, Car p Chymopapain, Car p Endoproteinase), Castanea spp (Cas c 24 kD, Cas s 1, Cas s 1.0101, Cas s 1.0102, Cas s 1.0103, Cas s 2, Cas s 5, Cas s 5.0101, Cas s 8, Cas s 8.0101, Cas s 9, Cas s 9.0101), Catharanthus spp (Cat r 1, Cat r 1.0101, Cat r 17 kD, Cat r 2), Caulolatilus spp (Cau ch 1), Cavia spp (Cav p 1, Cav p 1.0101, Cav p 2, Cav p 2.0101, Cav p 3, Cav p 3.0101, Cav p Gelatin, Cav p GSA), Centropristis spp (Cen s 1), Cephalopholis spp (Cep so 1), Charybdis spp (Cha f 1, Cha f 1.0101), Chaetodipterus spp (Cha fa 1), Chamaecyparis spp (Cha o 1, Cha o 1.0101, Cha o 2, Cha o 2.0101), Chenopodium spp (Che a 1, Che a 1.0101, Che a 2, Che a 2.0101, Che a 3, Che a 3.0101), Chironomus spp (Chi k 1, Chi k 10, Chi k 10.0101), Chinchilla spp (Chi I 21 kD_a, Chi I 21 kD_b), Chionoecetes spp (Chi o 1, Chi o 1.0101, Chi o 2, Chi o 4, Chi o 6, Chi o alpha_Actin, Chi o SERCA), Chironomus spp (Chi t 1, Chi t 1.0101, Chi t 1.0201, Chi t 2, Chi t 2.0101, Chi t 2.0102, Chi t 3, Chi t 3.0101, Chi t 4, Chi t 4.0101, Chi t 5, Chi t 5.0101, Chi t 6, Chi t 6.0101, Chi t 6.0201, Chi t 7, Chi t 7.0101, Chi t 8, Chi t 8.0101, Chi t 9, Chi t 9.0101), Chlamys spp (Chl n 1), Chloephaga spp (Chl pi 1), Chortoglyphus spp (Cho a 10), Chrysomela spp (Chr tr 7, Chr tr 7.0101), Cicer spp (Cic a 2S Albumin, Cic a Albumin), Cichorium spp (Cic i 1), Cimex spp (Cim I Nitrophorin), Citrus spp (Cit 11, Cit I 3, Cit I 3.0101), Citrullus spp (Cit la 2, Cit la MDH, Cit la TPI), Citrus spp (Cit r 3, Cit r 3.0101, Cits 1, Cits 1.0101, Cit s 2, Cit s 2.0101, Cit s 3, Cit s 3.0101, Cit s 3.0102, Cit s IFR), Cladosporium spp (Cla c 14, Cla c 14.0101, Cla c 9, Cla c 9.0101, Cla h 1, Cla h 10, Cla h 10.0101, Cla h 12, Cla h 12.0101, Cla h 2, Cla h 2.0101, Cla h 42 kD, Cla h 5, Cla h 5.0101, Cla h 6, Cla h 6.0101, Cla h 7, Cla h 7.0101, Cla h 8, Cla h 8 CSP, Cla h 8.0101, Cla h 9, Cla h 9.0101, Cla h abH, Cla h GST, Cla h HCh1, Cla h HSP70, Cla h NTF2, Cla h TCTP), Clostridium spp (Clo hi Collagenase, Clo t Toxoid), Clupea spp (Clu h 1, Clu h 1.0101, Clu h 1.0201, Clu h 1.0301), Cocos spp (Coc n 2, Coc n 4, Coc n 5), Coccidioides spp (Coc po 8), Coffea spp (Cof a 1, Cof a 1.0101), Columba spp (Col I PSA), Coprinus spp (Cop c 1, Cop c 1.0101, Cop c 2, Cop c 2.0101, Cop c 3, Cop c 3.0101, Cop c 4, Cop c 5, Cop c 5.0101, Cop c 6, Cop c 7, Cop c 7.0101), Corylus spp (Cor a 1, Cor a 1.0101, Cor a 1.0102, Cor a 1.0103, Cor a 1.0104, Cor a 1.0201, Cor a 1.0301, Cor a 1.0401, Cor a 1.0402, Cor a 1.0403, Cor a 1.0404, Cor a 10, Cor a 10.0101, Cor a 11, Cor a 11.0101, Cor a 12, Cor a 12.0101, Cor a 13, Cor a 13.0101, Cor a 14, Cor a 14.0101, Cor a 2, Cor a 2.0101, Cor a 2.0102, Cor a 8, Cor a 8.0101, Cor a 9, Cor a 9.0101), Corynebacterium spp (Cor d Toxoid), Corylus spp (Cor he 1), Coryphaena spp (Cor hi 1), Coriandrum spp (Cor s 1, Cor s 11 kD, Cor s 2), Cotoneaster spp (Cot I 3), Crangon spp (Cra c 1, Cra c 1.0101, Cra c 2, Cra c 2.0101, Cra c 4, Cra c 4.0101, Cra c 5, Cra c 5.0101, Cra c 6, Cra c 6.0101, Cra c 8, Cra c 8.0101), Crassostrea spp (Cra g 1), Cricetus spp (Cri c HSA), Crivellia spp (Cri pa 1), Crocus spp (Cro s 1, Cro s 1.0101, Cro s 2, Cro s 2.0101, Cro s 3, Cro s 3.01, Cro s 3.02), Cryptomeria spp (Cry j 1, Cry j 1.0101, Cry j 1.0102, Cry j 1.0103, Cry j 2, Cry j 2.0101, Cry j 2.0102, Cry j 3, Cry j 3.1, Cry j 3.2, Cry j 3.3, Cry j 3.4, Cry j 3.5, Cry j 3.6, Cry j 3.7, Cry j 3.8, Cry j 4, Cry j AP, Cry j Chitinase, Cry j CPA9, Cry j IFR, Cry j LTP, Cry j P1-P2), Cryphonectria spp (Cry p AP), Ctenocephalides spp (Cte f 1, Cte f 1.0101, Cte f 2, Cte f 2.0101, Cte f 3, Cte f 3.0101), Ctenopharyngodon spp (Cte id 1), Cucumis spp (Cuc m 1, Cuc m 1.0101, Cuc m 2, Cuc m 2.0101, Cuc m 3, Cuc m 3.0101, Cuc m Lec17, Cuc m MDH), Cucurbita spp (Cuc ma 18 kD, Cuc ma 2, Cuc p 2, Cuc p AscO), Cucumis spp (Cuc s 2), Culicoides spp (Cul n 1, Cul n 10, Cul n 11, Cul n 2, Cul n 3, Cul n 4, Cul n 5, Cul n 6, Cul n 7, Cul n 8, Cul n 9, Cul n HSP70), Culex spp (Cul q 28 kD, Cul q 35 kD, Cul q 7, Cul q 7.0101, Cul q 7.0102), Culicoides spp (Cul so 1), Cuminum spp (Cum c 1, Cum c 2), Cupressus spp (Cup a 1, Cup a 1.0101, Cup a 1.02, Cup a 2, Cup a 3, Cup a 4, Cup a 4.0101, Cups 1, Cups 1.0101, Cups 1.0102, Cups 1.0103, Cup s 1.0104, Cup s 1.0105, Cup s 3, Cup s 3.0101, Cup s 3.0102, Cup s 3.0103, Cup s 8), Cochliobolus spp (Cur 11, Cur 11.0101, Cur I 2, Cur I 2.0101, Cur I 3, Cur 1 3.0101, Cur 1 4, Cur 1 4.0101, Cur I ADH, Cur I GST, Cur I MnSOD, Cur I Oryzin, Cur I Trx, Cur I ZPS1), Cyanochen spp (Cya cy 1), Cynoscion spp (Cyn ar 1), Cynosurus spp (Cyn cr 1, Cyn cr 5), Cynodon spp (Cyn d 1, Cyn d 1.0101, Cyn d 1.0102, Cyn d 1.0103, Cyn d 1.0104, Cyn d 1.0105, Cyn d 1.0106, Cyn d 1.0107, Cyn d 1.0201, Cyn d 1.0202, Cyn d 1.0203, Cyn d 1.0204, Cyn d 10, Cyn d 11, Cyn d 12, Cyn d 12.0101, Cyn d 13, Cyn d 15, Cyn d 15.0101, Cyn d 2, Cyn d 22, Cyn d 22.0101, Cyn d 23, Cyn d 23.0101, Cyn d 24, Cyn d 24.0101, Cyn d 4, Cyn d 5, Cyn d 6, Cyn d 7, Cyn d 7.0101), Cynoscion spp (Cyn ne 1), Cynomys spp (Cyn sp Lipocalin), Cyprinus spp (Cyp c 1, Cyp c 1.01, Cyp c 1.02), Daboia spp (Dab ru 1), Dactylis spp (Dac g 1, Dac g 1.01, Dac g 1.0101, Dac g 1.02, Dac g 12, Dac g 13, Dac g 2, Dac g 2.0101, Dac g 3, Dac g 3.0101, Dac g 4, Dac g 4.0101, Dac g 5, Dac g 5.0101, Dac g 7), Dama spp (Dam d CSA), Danio spp (Dan re 1, Dan re 2, Dan re alpha21, Dan re CK), Dasyatis spp (Das ak 1, Das am 1, Das sa 1), Daucus spp (Dau c 1, Dau c 1.0101, Dau c 1.0102, Dau c 1.0103, Dau c 1.0104, Dau c 1.0105, Dau c 1.0201, Dau c 1.0301, Dau c 3, Dau c 4, Dau c 4.0101, Dau c CyP), Decapterus spp (Dec ru 1), Dendronephthya spp (Den n 1, Den n 1.0101), Dermatophagoides spp (Der f 1, Der f 1.0101, Der f 1.0102, Der f 1.0103, Der f 1.0104, Der f 1.0105, Der f 1.0106, Der f 1.0107, Der f 1.0108, Der f 1.0109, Der f 1.0110, Der f 10, Der f 10.0101, Der f 10.0102, Der f 11, Der f 11.0101, Der f 13, Der f 13.0101, Der f 14, Der f 14.0101, Der f 15, Der f 15.0101, Der f 16, Der f 16.0101, Der f 17, Der f 17.0101, Der f 18, Der f 18.0101, Der f 2, Der f 2.0101, Der f 2.0102, Der f 2.0103, Der f 2.0104, Der f 2.0105, Der f 2.0106, Der f 2.0107, Der f 2.0108, Der f 2.0109, Der f 2.0110, Der f 2.0111, Der f 2.0112, Der f 2.0113, Der f 2.0114, Der f 2.0115, Der f 2.0116, Der f 2.0117, Der f 20, Der f 21, Der f 22, Der f 22.0101, Der f 3, Der f 3.0101, Der f 4, Der f 5, Der f 6, Der f 6.0101, Der f 7, Der f 7.0101, Der f 8, Der f 9, Der f HSP70), Dermanyssus spp (Der g 10, Der g 10.0101), Dermatophagoides spp (Der m 1, Der m 1.0101, Der p 1, Der p 1.0101, Der p 1.0102, Der p 1.0103, Der p 1.0104, Der p 1.0105, Der p 1.0106, Der p 1.0107, Der p 1.0108, Der p 1.0109, Der p 1.0110, Der p 1.0111, Der p 1.0112, Der p 1.0113, Der p 1.0114, Der p 1.0115, Der p 1.0116, Der p 1.0117, Der p 1.0118, Der p 1.0119, Der p 1.0120, Der p 1.0121, Der p 1.0122, Der p 1.0123, Der p 1.0124, Der p 10, Der p 10.0101, Der p 10.0102, Der p 10.0103, Der p 11, Der p 11.0101, Der p 13, Der p 14, Der p 14.0101, Der p 15, Der p 18, Der p 2, Der p 2.0101, Der p 2.0102, Der p 2.0103, Der p 2.0104, Der p 2.0105, Der p 2.0106, Der p 2.0107, Der p 2.0108, Der p 2.0109, Der p 2.0110, Der p 2.0111, Der p 2.0112, Der p 2.0113, Der p 2.0114, Der p 2.0115, Der p 20, Der p 20.0101, Der p 21, Der p 21.0101, Der p 23, Der p 23.0101, Der p 3, Der p 3.0101, Der p 4, Der p 4.0101, Der p 5, Der p 5.0101, Der p 5.0102, Der p 6, Der p 6.0101, Der p 7, Der p 7.0101, Der p 8, Der p 8.0101, Der p 9, Der p 9.0101, Der p 9.0102, Der p P1-P2, Der p P2-P1, Der s 1, Der s 2, Der s 3), Dianthus spp (Dia c RIP), Dicranopteris spp (Dic I 2S Albumin), Diospyros spp (Dio k 17 kD, Dio k 4, Dio k IFR), Dioscorea spp (Dio p TSP), Diplodus spp (Dip ho 1), Distichlis spp (Dis s 1, Dis s 7), Ditrema spp (Dit to 1), Dolichovespula spp (Dol a 1, Dol a 2, Dol a 5, Dol a 5.0101), Dolichos spp (Dol b Agglutinin), Dolichovespula spp (Dol m 1, Dol m 1.0101, Dol m 1.02, Dol m 2, Dol m 2.0101, Dol m 5, Dol m 5.0101, Dol m 5.02), Drosophila spp (Dro an 7, Dro an 7.0101, Dro er 7, Dro er 7.0101, Dro er 7.0102, Dro gr 7, Dro gr 7.0101, Dro gr 7.0102, Dro m 7, Dro m 7.0101, Dro m 7.0102, Dro m 7.0103, Dro m 7.0104, Dro m 7.0105, Dro m 7.0106, Dro m 7.0107, Dro m 7.0108, Dro m 7.0109, Dro m 7.0110, Dro m 7.0111, Dro m 7.0112, Dro m 7.0113, Dro m 9, Dro m MnSOD, Dro mo 7, Dro mo 7.0101, Dro pp 7, Dro se 7.0101, Dro se 7, Dro se 7.0101, Dro si 7, Dro si 7.0101, Dro si 7.0102, Dro vi 7, Dro vi 7.0101, Dro wi 7, Dro wi 7.0101, Dro y 7, Dro y 7.0101, Dro y 7.0102, Dro y 7.0103), Echium spp (Ech p Cytochrome C), Elaeis spp (Ela g 2, Ela g Bd31 kD), Elops spp (Elo sa 1), Embellisia spp (Emb a 1, Emb i 1, Emb nz 1, Emb t 1), Engraulis spp (Eng e 1), Enteroctopus spp (Ent d 1), Epinephelus spp (Epi bl 1, Epi co 1, Epi fl 1, Epi mc 1, Epi mo 1), Epicoccum spp (Epi p 1, Epi p 1.0101, Epi p 12 kD, Epi p GST), Epinephelus spp (Epi po 1, Epi un 1), Equisetum spp (Equ a 17 kD), Equus spp (Equ as 4, Equ as DSA, Equ bu 4, Equ c 1, Equ c 1.0101, Equ c 2, Equ c 2.0101, Equ c 2.0102, Equ c 3, Equ c 3.0101, Equ c 4, Equ c 4.0101, Equ c 5, Equ c 5.0101, Equ c ALA, Equ c BLG, Equ c Casein, Equ c Casein beta, Equ c Casein kappa, Equ c PRVB, Equ he 4, Equ z ZSA), Erimacrus spp (En i 1, En i 1.0101, Eri i 1.0102), Eriocheir spp (Eri s 1, Eri s 1.0101, En s 2), Erwinia spp (Erw ch Asparaginase), Escherichia spp (Esc c Asparaginase, Esc c beta GAL), Esox spp (Eso 11), Euphausia spp (Eup p 1, Eup p 1.0101), Euphasia spp (Eup s 1, Eup s 1.0101), Euroglyphus spp (Eur m 1, Eur m 1.0101, Eur m 1.0102, Eur m 1.0103, Eur m 10, Eur m 14, Eur m 14.0101, Eur m 2, Eur m 2.0101, Eur m 2.0102, Eur m 3, Eur m 3.0101, Eur m 4, Eur m 4.0101), Evynnis spp (Evy j 1), Fagopyrum spp (Fag e 1, Fag e 1.0101, Fag e 10 kD, Fag e 19 kD, Fag e 2, Fag e 2.0101, Fag e TI), Fagus spp (Fag s 1, Fag s 1.0101, Fag s 2, Fag s 4), Fagopyrum spp (Fag t 1, Fag t 10 kD, Fag t 2, Fag t 2.0101), Felis spp (Fel d 1, Fel d 1.0101, Fel d 2, Fel d 2.0101, Fel d 3, Fel d 3.0101, Fel d 4, Fel d 4.0101, Fel d 5, Fel d 5.0101, Fel d 6, Fel d 6.0101, Fel d 7, Fel d 7.0101, Fel d 8, Fel d 8.0101, Fel d IgG), Fenneropenaeus spp (Fen c 1, Fen c 2, Fen me 1, Fen me 1.0101), Festuca spp (Fes e 1, Fes e 13, Fes e 4, Fes e 5, Fes e 7, Fes p 1, Fes p 13, Fes p 4, Fes p 4.0101, Fes p 5, Fes r 1, Fes r 5), Ficus spp (Fic c 17 kD, Fic c 4, Fic c Ficin), Foeniculum spp (Foe v 1, Foe v 2), Forsythia spp (For s 1), Forcipomyia spp (Fort 1, Fort 1.0101, Fort 2, Fort 2.0101, Fort 7, Fort FPA, Fort Myosin, Fort TPI), Fragaria spp (Fra a 1, Fra a 1.0101, Fra a 3, Fra a 3.0101, Fra a 3.0102, Fra a 3.0201, Fra a 3.0202, Fra a 3.0203, Fra a 3.0204, Fra a 3.0301, Fra a 4, Fra a 4.0101, Fra c 1), Fraxinus spp (Fra e 1, Fra e 1.0101, Fra e 1.0102, Fra e 1.0201, Fra e 12, Fra e 2, Fra e 3, Fra e 9), Fragaria spp (Fra v 1), Fusarium spp (Fus c 1, Fus c 1.0101, Fus c 2, Fus c 2.0101, Fus c 3, Fus s 1, Fus s 45 kD, Fus sp Lipase), Gadus spp (Gad c 1, Gad c 1.0101, Gad c APDH, Gad m 1, Gad m 1.0101, Gad m 1.0102, Gad m 1.0201, Gad m 1.0202, Gad m 45 kD, Gad m Gelatin, Gad ma 1), Gallus spp (Gal d 1, Gal d 1.0101, Gal d 2, Gal d 2.0101, Gal d 3, Gal d 3.0101, Gal d 4, Gal d 4.0101, Gal d 5, Gal d 5.0101, Gal d 6, Gal d 6.0101, Gal d Apo I, Gal d Apo VI, Gal d GPI, Gal d HG, Gal d IgY, Gal d L-PGDS, Gal d Ovomucin, Gal d Phosvitin, Gal d PRVB, Gal la 4), Galleria spp (Gal m 18 kD, Gal m 24 kD), Gallus spp (Gal so 4), Gammarus spp (Gam s TM), Gelonium spp (Gel m RIP), Geothelphusa spp (Geo de 1), Glossina spp (Glo m 5, Glo m 5.0101, Glo m 7, Glo m 7.0101, Glo m 7.0102, Glo m 7.0103), Glycine spp (Gly a Bd30K, Gly ar Bd30K, Gly ca Bd30K, Gly cl Bd30K, Gly cu Bd30K, Gly cy Bd30K), Glycyphagus spp (Gly d 10, Gly d 10.0101, Gly d 13, Gly d 2, Gly d 2.0101, Gly d 2.0201, Gly d 2.03, Gly d 2/Lep d 2 L1, Gly d 2/Lep d 2 L2, Gly d 2/Lep d 2 L3, Gly d 2/Lep d 2 L4, Gly d 2/Lep d 2 R1, Gly d 2/Lep d 2 R2, Gly d 2/Lep d 2 R3, Gly d 2/Lep d 2 R4, Gly d 2/Lep d 2 R5, Gly d 20, Gly d 3, Gly d 5, Gly d 5.01, Gly d 5.02, Gly d 7, Gly d 8), Glycine spp (Gly f Bd30K, Gly I Bd30K, Gly m 1, Gly m 1.0101, Gly m 1.0102, Gly m 2, Gly m 2.0101, Gly m 2S Albumin, Gly m 3, Gly m 3.0101, Gly m 3.0102, Gly m 39 kD, Gly m 4, Gly m 4.0101, Gly m 5, Gly m 5.0101, Gly m 5.0201, Gly m 5.0301, Gly m 5.0302, Gly m 50 kD, Gly m 6, Gly m 6.0101, Gly m 6.0201, Gly m 6.0301, Gly m 6.0401, Gly m 6.0501, Gly m 68 kD, Gly m Agglutinin, Gly m Bd28K, Gly m Bd30K, Gly m Bd60K, Gly m CPI, Gly m EAP, Gly m TI, Gly mi Bd30K, Gly s Bd30K, Gly t Bd30K, Gly to Bd30K), Gossypium spp (Gos h Vicilin), Haemophilus spp (Hae in P6), Haemaphysalis spp (Hae 17, Hae 1 7.0101, Hae q 7, Hae q 7.0101), Haliotis spp (Hal a 1, Hal d 1, Hal di 1, Hal di PM, Hal m 1, Hal m 1.0101, Hal r 1, Hal r 49 kD, Hal ru 1), Harmonia spp (Har a 1, Har a 1.0101, Har a 2, Har a 2.0101), Harpegnathos spp (Har sa 7, Har sa 7.0101, Har sa 7.0102), Helianthus spp (Hel a 1, Hel a 1.0101, Hel a 2, Hel a 2.0101, Hel a 2S Albumin, Hel a 3, Hel a 3.0101, Hel a 4), Helix spp (Hel ap 1, Hel as 1, Hel as 1.0101), Heligmosomoides spp (Hel p 3, Hel p 3.0101), Helianthus spp (Hel to 1), Hemanthias spp (Hem le 1), Hemifusus spp (Hem t 1), Heterodera spp (Het g 3, Het g 3.0101), Hevea spp (Hey b 1, Hey b 1.0101, Hey b 10, Hey b 10.0101, Hey b 10.0102, Hey b 10.0103, Hey b 11, Hey b 11.0101, Hey b 11.0102, Hey b 12, Hey b 12.0101, Hey b 13, Hey b 13.0101, Hey b 14, Hey b 14.0101, Hey b 2, Hey b 2.0101, Hey b 3, Hey b 3.0101, Hey b 4, Hey b 4.0101, Hey b 5, Hey b 5.0101, Hey b 6, Hey b 6.01, Hey b 6.02, Hey b 6.0202, Hey b 6.03, Hey b 7, Hey b 7.01, Hey b 7.02, Hey b 7.D2, Hey b 7.S2, Hey b 8, Hey b 8.0101, Hey b 8.0102, Hey b 8.0201, Hey b 8.0202, Hey b 8.0203, Hey b 8.0204, Hey b 9, Hey b 9.0101, Hey b Citrate binding Protein, Hey b GAPDH, Hey b HSP80, Hey b IFR, Hey b Proteasome subunit, Hey b Rotamase, Hey b SPI, Hey b Trx, Hey b UDPGP), Hexagrammos spp (Hex of 1), Hippoglossus spp (Hip h 1), Hippoglossoides spp (Hip pl 1), Hippoglossus spp (Hip st 1), Hirudo spp (Hir me Hirudin), Holcus spp (Hol 1 1, Hol 1 1.0101, Hol 1 1.0102, Hol 1 2, Hol 1 4, Hol 15, Hol 1 5.0101, Hol 1 5.0201), Holocnemus spp (Hol pl 9, Hol pl Hemocyanin), Homarus spp (Hom a 1, Hom a 1.0101, Hom a 1.0102, Hom a 1.0103, Hom a 3, Hom a 3.0101, Hom a 4, Hom a 6, Hom a 6.0101, Hom g 1, Hom g 2), Homo spp (Hom s 1, Hom s 1.0101, Hom s 2, Hom s 2.0101, Hom s 3, Hom s 3.0101, Hom s 4, Hom s 4.0101, Hom s 5, Hom s 5.0101, Hom s AAT, Hom s ACTH, Hom s Adalimumab, Hom s ALA, Hom s alpha_Actin, Hom s alpha-Galactosidase, Hom s APDH, Hom s Arylsulfatase B, Hom s Casein, Hom s CyP A, Hom s CyP B, Hom s CyP C, Hom s DSF70, Hom s DSG3, Hom s eIF6, Hom s Etanercept, Hom s Factor IX, Hom s Factor VII, Hom s Factor VIII, Hom s G-CSF, Hom s Glucocerebrosidase, Hom s Glucosidase, Hom s HLA-DR-alpha, Hom s HSA, Hom s Iduronidase, Hom s Idursulfase, Hom s IgA, Hom s Insulin, Hom s Lactoferrin, Hom s Laminin gamma_2, Hom s MnSOD, Hom s Oxytocin, Hom s P2, Hom s Phosvitin, Hom s Profilin, Hom s PSA, Hom s RP1, Hom s TCTP, Hom s TL, Hom s TPA, Hom s TPO, Hom s Transaldolase, Hom s Trx, Hom s Tubulin-alpha, Hom s/Mus m Basiliximab, Hom s/Mus m Cetuximab, Hom s/Mus m Cetuximab (Gal-Gal), Hom s/Mus m Infliximab, Hom s/Mus m Natalizumab, Hom s/Mus m Omalizumab, Hom s/Mus m Palivizumab, Hom s/Mus m Rituximab, Hom s/Mus m Tocilizumab, Hom s/Mus m Trastuzumab), Hoplostethus spp (Hop a 1), Hordeum spp (Hor v 1, Hor v 12, Hor v 12.0101, Hor v 13, Hor v 14, Hor v 15, Hor v 15.0101, Hor v 16, Hor v 16.0101, Hor v 17, Hor v 17.0101, Hor v 18 KD, Hor v 2, Hor v 21, Hor v 21.0101, Hor v 28, Hor v 33, Hor v 4, Hor v 5, Hor v 5.0101, Hor v BDAI, Hor v BTI), Humicola spp (Hum in Cellulase), Humulus spp (Hum j 1, Hum j 1.0101, Hum j 10 kD, Hum j 2), Huso spp (Hus h 1), Hylocereus spp (Hyl un LTP), Hymenocephalus spp (Hym st 1), Hyperoglyphe spp (Hyp by 1), Hypophthalmichthys spp (Hyp mo 1), Hypophthalmichthy spp (Hyp no 1), Ictalurus spp (Ict fu 1, Ict p 1), Imperata spp (Imp c 4, Imp c 5, Imp c VIIIel), Ixodes spp (Ixo r 2, Ixo sc 7, Ixo sc 7.0101), Jasus spp (Jas la 1, Jas la 1.0101, Jas la 1.0102), Juglans spp (Jug ca 1, Jug ca 2, Jug ci 1, Jug ci 2, Jug n 1, Jug n 1.0101, Jug n 2, Jug n 2.0101, Jug r 1, Jug r 1.0101, Jug r 2, Jug r 2.0101, Jug r 3, Jug r 3.0101, Jug r 4, Jug r 4.0101, Jug r 5), Juniperus spp (Jun a 1, Jun a 1.0101 Jun a 1.0102 Jun a 2, Jun a 2.0101 Jun a 3, Jun a 3.0101 Jun c 1, Jun o 1, Jun o 4, Jun o 4.0101 Jun r 3, Jun r 3.1, Jun r 3.2, Jun v 1, Jun v 1.0101 Jun v 1.0102 Jun v 3, Jun v 3.0101 Jun v 3.0102 Jun v 4), Katsuwonus spp (Kat p 1), Kyphosus spp (Kyp se 1), Lachnolaimus spp (Lac ma 1), Lachesis spp (Lac mu 1), Lactuca spp (Lac s 1, Lac s 1.0101), Lagocephalus spp (Lag la 1), Larus spp (Lar a 1, Lar a 2, Lar a 3), Larimichthys spp (Lar po 1), Lates spp (Lat c 1), Lateolabrax spp (Lat ja 1), Lathyrus spp (Lat oc Agglutinin), Leiostomus spp (Lei xa 1), Lens spp (Len c 1, Len c 1.0101, Len c 1.0102, Len c 1.0103, Len c 2, Len c 2.0101, Len c 3, Len c 3.0101, Len c Agglutinin), Leopardus spp (Leo p 1), Lepidoglyphus spp (Lep d 10, Lep d 10.0101, Lep d 12, Lep d 13, Lep d 13.0101, Lep d 2, Lep d 2.0101, Lep d 2.0102, Lep d 2.0201, Lep d 2.0202, Lep d 3, Lep d 39 kD, Lep d 5, Lep d 5.0101, Lep d 5.0102, Lep d 5.0103, Lep d 7, Lep d 7.0101, Lep d 8, Lep d alpha Tubulin), Lepomis spp (Lep gi 1), Leptomelanosoma spp (Lep i 1), Lepomis spp (Lep ma 1), Lepisma spp (Lep s 1, Lep s 1.0101, Lep s 1.0102), Lepeophtheirus spp (Lep sa 1, Lep sa 1.0101, Lep sa 1.0102, Lep sa 1.0103), Leptailurus spp (Lep se 1), Lepidorhombus spp (Lep w 1, Lep w 1.0101), Lethocerus spp (Let in 7, Let in 7.0101, Let in 7.0102), Leuciscus spp (Leu ce 1), Lewia spp (Lew in 1), Ligustrum spp (Lig v 1, Lig v 1.0101, Lig v 1.0102, Lig v 2), Lilium spp (Lil 12, Lil I PG), Limanda spp (Lim fe 1), Limnonectes spp (Lim m 1), Limulus spp (Lim p 1, Lim p 1.0101, Lim p 2, Lim p LPA), Liposcelis spp (Lip b 1, Lip b 1.0101), Litchi spp (Lit c 1, Lit c 1.0101, Lit c IFR, Lit c TPI), Lithobates spp (Lit ca 1), Litopenaeus spp (Lit se 1, Lit v 1, Lit v 1.0101, Lit v 2, Lit v 2.0101, Lit v 3, Lit v 3.0101, Lit v 4, Lit v 4.0101), Filiaria spp (Loa lo 3, Loa lo 3.0101), Lobotes spp (Lob su 1), Locusta spp (Loc m 7, Loc m 7.0101), Loligo spp (Lol b 1, Lol e 1), Lolium spp (Lol m 2, Lol m 5, Lol p 1, Lol p 1.0101, Lol p 1.0102, Lol p 1.0103, Lol p 10, Lol p 11, Lol p 11.0101, Lol p 12, Lol p 13, Lol p 2, Lol p 2.0101, Lol p 3, Lol p 3.0101, Lol p 4, Lol p 4.0101, Lol p 5, Lol p 5.0101, Lol p 5.0102, Lol p 7, Lol p CyP, Lol p FT, Lol p Legumin), Lonomia spp (Lon o 7, Lon o 7.0101), Lophodytes spp (Lop cu 1), Lophonetta spp (Lop sp 1), Lupinus spp (Lup a 1, Lup a alpha_Conglutin, Lup a delta_Conglutin, Lup a gamma_Conglutin, Lup an 1, Lup an 1.0101, Lup an alpha_Conglutin, Lup an delta_Conglutin, Lup an gamma_Conglutin, Lup 117 kD), Lutjanus spp (Lut a 1, Lut c 1, Lut cy 1, Lut gr 1, Lut gu 1, Lut jo 1), Lutraria spp (Lut p 1), Lutjanus spp (Lut pu 1, Lut sy 1), Lycopersicon spp (Lyc e 1, Lyc e 1.0101, Lyc e 11S Globulin, Lyc e 2, Lyc e 2.0101, Lyc e 2.0102, Lyc e 3, Lyc e 3.0101, Lyc e 4, Lyc e 4.0101, Lyc e ARP60S, Lyc e Chitinase, Lyc e Glucanase, Lyc e Peroxidase, Lyc e PG, Lyc e PME, Lyc e PR23, Lyc e Vicilin), Maconellicoccus spp (Mac h 7, Mac h 7.0101), Macruronus spp (Mac ma 1, Mac n 1), Maclura spp (Mac po 17 kD), Macrobrachium spp (Mac ro 1, Mac ro 1.0101, Mac ro Hemocyanin), Macropus spp (Marr s Gelatin), Malus spp (Mal d 1, Mal d 1.0101, Mal d 1.0102, Mal d 1.0103, Mal d 1.0104, Mal d 1.0105, Mal d 1.0106, Mal d 1.0107, Mal d 1.0108, Mal d 1.0109, Mal d 1.0201, Mal d 1.0202, Mal d 1.0203, Mal d 1.0204, Mal d 1.0205, Mal d 1.0206, Mal d 1.0207, Mal d 1.0208, Mal d 1.0301, Mal d 1.0302, Mal d 1.0303, Mal d 1.0304, Mal d 1.0401, Mal d 1.0402, Mal d 1.0403, Mal d 2, Mal d 2.0101, Mal d 3, Mal d 3.0101, Mal d 3.0102, Mal d 3.0201, Mal d 3.0202, Mal d 3.0203, Mal d 4, Mal d 4.0101, Mal d 4.0102, Mal d 4.0201, Mal d 4.0202, Mal d 4.0301, Mal d 4.0302), Malpighia spp (Mal g 4, Mal g Hevein), Malus spp (Mal p 1), Malassezia spp (Mala f 2, Mala f 2.0101, Mala f 3, Mala f 3.0101, Mala f 4, Mala f 4.0101, Mala g 10, Mala s 1, Mala s 1.0101, Mala s 10, Mala s 10.0101, Mala s 11, Mala s 11.0101, Mala s 12, Mala s 12.0101, Mala s 13, Mala s 13.0101, Mala s 5, Mala s 5.0101, Mala s 6, Mala s 6.0101, Mala s 7, Mala s 7.0101, Mala s 8, Mala s 8.0101, Mala s 9, Mala s 9.0101), Manihot spp (Man e 5, Man e 5.0101, Man e FPA, Man e GAPDH), Mangifera spp (Man i 1, Man i 14 kD, Man i 2, Man i 3, Man i 3.01, Man i 3.02, Man i Chitinase), Marsupenaeus spp (Mar j 1, Mar j 1.0101 Mar j 2, Mar j 4), Matricaria spp (Mat c 17 kD), Mecopoda spp (Mec e 7), Megalobrama spp (Meg am 2, Meg am CK), Megathura spp (Meg c Hemocyanin), Megalops spp (Meg sp 1), Melanogrammus spp (Mel a 1), Meleagris spp (Mel g 1, Mel g 2, Mel g 3, Mel g PRVB, Mel g TSA), Melicertus spp (Mel 11), Menticirrhus spp (Men am 1), Mercurialis spp (Mer a 1, Mer a 1.0101), Merluccius spp (Mer ap 1, Mer au 1, Mer bi 1, Mer ca 1, Mer ga 1, Mer hu 1), Merlangius spp (Mer me 1), Merluccius spp (Mer mr 1, Mer pa 1, Mer po 1, Mer pr 1, Mer se 1), Meriones spp (Mer un 23 kD), Metarhizium spp (Met a 30), Metapenaeopsis spp (Met ba 1), Metapenaeus spp (Mete 1, Mete 1.0101, Met e 2), Metasequoia spp (Met gl 2), Metapenaeus spp (Met j 1, Met j 2), Metanephrops spp (Met ja 1), Metapenaeopsis spp (Met la 1), Metanephrops spp (Met t 2), Micromesistius spp (Mic po 1), Micropogonias spp (Mic un 1), Mimachlamys spp (Mim n 1), Momordica spp (Mom c RIP), Morus spp (Mor a 17 kD, Mor a 4), Morone spp (Mor am 1), Morus spp (Mor n 3, Mor n 3.0101), Morone spp (Mor sa 1, Mor sc 1), Mugil spp (Mug c 1), Muraenolepis spp (Mur mi 1), Musa spp (Mus a 1, Mus a 1.0101, Mus a 2, Mus a 2.0101, Mus a 3, Mus a 3.0101, Mus a 4, Mus a 4.0101, Mus a 5, Mus a 5.0101, Mus a 5.0102), Mus spp (Mus m 1, Mus m 1.0101, Mus m 1.0102, Mus m 2, Mus m Gelatin, Mus m IgG, Mus m MSA, Mus m Muromonab, Mus m Phosvitin), Mustela spp (Mus p 17 kD), Musa spp (Mus xp 1, Mus xp 2, Mus xp 5), Mycteroperca spp (Myc bo 1, Myc mi 1, Myc ph 1), Myceliophthora spp (Myc sp Laccase), Myrmecia spp (Myr p 1, Myr p 1.0101, Myr p 2, Myr p 2.0101, Myr p 2.0102, Myr p 3, Myr p 3.0101), Mytilus spp (Myt e 1, Myt g 1, Myt g PM), Myzus spp (Myz p 7, Myz p 7.0101), Nemorhedus spp (Nae go Hya), Necator spp (Nec a Calreticulin), Nemipterus spp (Nem vi 1), Neosartorya spp (Neo fi 1, Neo fi 22), Neochen spp (Neo ju 1), Neoscona spp (Neo n 7, Neo n 7.0101), Nephelium spp (Nep I GAPDH), Nephrops spp (Nep n 1, Nep n DF9), Neptunea spp (Nep po 1, Nep po 1.0101), Nicotiana spp (Nic t 8, Nic t Osmotin, Nic t Villin), Nimbya spp (Nim c 1, Nim s 1), Nippostrongylus spp (Nip b Ag1), Nycticebus spp (Nyc c 1), Octopus spp (Oct f 1, October 11, Oct v 1, Oct v 1.0101 Oct v PM), Ocyurus spp (Ocy ch 1), Olea spp (Ole e 1, Ole e 1.0101, Ole e 1.0102, Ole e 1.0103, Ole e 1.0104, Ole e 1.0105, Ole e 1.0106, Ole e 1.0107, Ole e 10, Ole e 10.0101, Ole e 11, Ole e 11.0101, Ole e 11.0102, Ole e 12, Ole e 13, Ole e 2, Ole e 2.0101, Ole e 3, Ole e 3.0101, Ole e 36 kD, Ole e 4, Ole e 4.0101, Ole e 5, Ole e 5.0101, Ole e 6, Ole e 6.0101, Ole e 7, Ole e 7.0101, Ole e 8, Ole e 8.0101, Ole e 9, Ole e 9.0101), Ommastrephes spp (Omm b 1, Omm b 1.0101), Oncorhynchus spp (Onc ke 1, Onc ke 18 kD, Onc ke alpha2I, Onc ke Vitellogenin, Onc m 1, Onc m 1.0101, Onc m 1.0201, Onc m alpha21, Onc m Protamine, Onc m Vitellogenin, Onc ma 1, Onc ma FPA, Onc ma FSA, Onc ma TPI, Onc n 1), Onchocerca spp (Onc o 3, Onc o 3.0101), Oncorhynchus spp (Onc is 1), Onchocerca spp (Onc v 3, Onc v 3.0101), Oratosquilla spp (Ora o 1, Ora o 1.0101), Oreochromis spp (Ore a 1, Ore mo 1, Ore mo 2, Ore mo FPA, Ore mo SCAF7145, Ore ni 1, Ore ni 18 kD, Ore ni 45 kD), Ornithonyssus spp (Orn sy 10, Orn sy 10.0101, Orn sy 10.0102), Oryctolagus spp (Ory c 1, Ory c 1.0101, Ory c 2, Ory c Casein, Ory c Phosvitin, Ory c RSA), Oryza spp (Ory s 1, Ory s 1.0101, Ory s 11, Ory s 12, Ory s 12.0101, Ory s 13, Ory s 14, Ory s 17 kD, Ory s 19 kD, Ory s 2, Ory s 23, Ory s 3, Ory s 7, Ory s akTI, Ory s GLP52, Ory s GLP63, Ory s Glyoxalase I, Ory s NRA), Ostrya spp (Ost c 1, Ost c 1.0101), Ovis spp (Ovi a ALA, Ovi a BLG, Ovi a Casein, Ovi a Casein alphaS1, Ovi a Casein alphaS2, Ovi a Casein beta, Ovi a Casein kappa, Ovi a Phosvitin, Ovi a SSA), Pachycondyla spp (Pac c 3), Pagrus spp (Pag m 1, Pag pa 1), Pampus spp (Pam ar 1, Pam c 1), Pandalus spp (Pan b 1, Pan b 1.0101), Pangasius spp (Pan bo 1), Pandalus spp (Pan e 1, Pan e 1.0101, Pan e 4), Panulirus spp (Pan h 1, Pan hy 1), Pangasius spp (Pan hy 18 kD, Pan hy 45 kD), Panulirus spp (Pan j 1), Panthera spp (Pan 11, Pan o 1, Pan p 1), Panulirus spp (Pan s 1, Pan s 1.0101), Panthera spp (Pan t 1), Pan spp (Pan tr TCTP), Papaver spp (Pap s 17 kD, Pap s 2, Pap s 34 kD), Papilio spp (Pap xu 7, Pap xu 7.0101, Pap xu 7.0102), Paralichthys spp (Par a 1), Parasilurus spp (Par as 1, Par c 1), Paralithodes spp (Par c 1.0101, Par c 1.0102, Par f 1), Parthenium spp (Par h 1), Parietaria spp (Par j 1, Par j 1.0101, Par j 1.0102, Par j 1.0103, Par j 1.0201, Par j 2, Par j 2.0101, Par j 2.0102, Par j 3, Par j 3.0101, Par j 3.0102, Par j 4, Par j 4.0101, Par j J1-J2), Paralichthys spp (Par le 1), Parietaria spp (Par m 1, Par o 1, Par o 1.0101), Paralichthys spp (Par of 1, Par of alpha21), Parahucho spp (Par pe Vitellogenin), Passiflora spp (Pas e Chitinase, Pas e Hevein), Paspalum spp (Pas n 1, Pas n 1.0101, Pas n 13), Patinopecten spp (Pat y 1), Pediculus spp (Ped h 7, Ped h 7.0101), Penaeus spp (Pen a 1, Pen a 1.0101, Pen a 1.0102, Pen a 1.0102 (103-117), Pen a 1.0102 (109-123), Pen a 1.0102 (1-15), Pen a 1.0102 (115-129), Pen a 1.0102 (121-135), Pen a 1.0102 (127-141), Pen a 1.0102 (13-27), Pen a 1.0102 (133-147), Pen a 1.0102 (139-153), Pen a 1.0102 (145-159)), Farfantepenaeus spp (Pen a 1.0102 (151-165)), Penaeus spp (Pen a 1.0102 (157-171), Pen a 1.0102 (163-177), Pen a 1.0102 (169-183), Pen a 1.0102 (175-189), Pen a 1.0102 (181-195), Pen a 1.0102 (187-201), Pen a 1.0102 (193-207), Pen a 1.0102 (19-33), Pen a 1.0102 (199-213), Pen a 1.0102 (205-219), Pen a 1.0102 (211-225), Pen a 1.0102 (217-231), Pen a 1.0102 (223-237), Pen a 1.0102 (229-243)), Farfantepenaeus spp (Pen a 1.0102 (235-249)), Penaeus spp (Pen a 1.0102 (241-255), Pen a 1.0102 (247-261), Pen a 1.0102 (253-267), Pen a 1.0102 (25-39), Pen a 1.0102 (259-273), Pen a 1.0102 (265-279), Pen a 1.0102 (270-284), Pen a 1.0102 (31-45), Pen a 1.0102 (37-51), Pen a 1.0102 (43-57), Pen a 1.0102 (49-63)), Farfantepenaeus spp (Pen a 1.0102 (55-69)), Penaeus spp (Pen a 1.0102 (61-75), Pen a 1.0102 (67-81), Pen a 1.0102 (7-21), Pen a 1.0102 (73-87), Pen a 1.0102 (79-93), Pen a 1.0102 (85-99), Pen a 1.0102 (91-105), Pen a 1.0102 (97-111), Pen a 1.0103), Penicillium spp (Pen b 13, Pen b 13.0101, Pen b 26, Pen b 26.0101, Pen c 1, Pen c 13, Pen c 13.0101, Pen c 18, Pen c 19, Pen c 19.0101, Pen c 2, Pen c 22, Pen c 22.0101, Pen c 24, Pen c 24.0101, Pen c 3, Pen c 3.0101, Pen c 30, Pen c 30.0101, Pen c 32, Pen c 32.0101, Pen c MnSOD, Pen ch 13, Pen ch 13.0101, Pen ch 18, Pen ch 18.0101, Pen ch 20, Pen ch 20.0101, Pen ch 31, Pen ch 31.0101, Pen ch 33, Pen ch 33.0101, Pen ch 35, Pen ch 35.0101, Pen ch MnSOD), Penaeus spp (Pen i 1, Pen i 1.0101, Pen m 1, Pen m 1.0101, Pen m 1.0102, Pen m 2, Pen m 2.0101, Pen m 3, Pen m 3.0101, Pen m 4, Pen m 4.0101, Pen m 6, Pen m 6.0101), Penicillium spp (Pen o 18, Pen o 18.0101), Penaeus spp (Pena o 1, Pena o 1.0101), Periplaneta spp (Per a 1, Per a 1.0101, Per a 1.0102, Per a 1.0103, Per a 1.0104, Per a 1.0105, Per a 1.0201, Per a 10, Per a 10.0101, Per a 2, Per a 3, Per a 3.0101, Per a 3.0201, Per a 3.0202, Per a 3.0203, Per a 4, Per a 5, Per a 6, Per a 6.0101, Per a 7, Per a 7.0101, Per a 7.0102, Per a 7.0103, Per a 9, Per a 9.0101, Per a Cathepsin, Per a FABP, Per a Trypsin, Per f 1, Per f 7, Per f 7.0101), Perna spp (Per v 1), Persea spp (Pers a 1, Pers a 1.0101, Pers a 4), Petroselinum spp (Pet c 1, Pet c 2, Pet c 3), Phalaris spp (Pha a 1, Pha a 1.0101, Pha a 5, Pha a 5.0101, Pha a 5.02, Pha a 5.03, Pha a 5.04), Phaseolus spp (Pha v 3, Pha v 3.0101, Pha v 3.0201, Pha vaAI, Pha vaAI.0101, Pha v Chitinase, Pha v PHA, Pha v Phaseolin), Phleum spp (Phl p 1, Phl p 1.0101, Phl p 1.0102, Phl p 11, Phl p 11.0101, Phl p 12, Phl p 12.0101, Phl p 12.0102, Phl p 12.0103, Phl p 13, Phl p 13.0101, Phl p 2, Phl p 2.0101, Phl p 3, Phl p 3.0101, Phl p 3.0102, Phl p 4, Phl p 4.0101, Phl p 4.0102, Phl p 4.0201, Phl p 4.0202, Phl p 4.0203, Phl p 4.0204, Phl p 5, Phl p 5.0101, Phl p 5.0102, Phl p 5.0103, Phl p 5.0104, Phl p 5.0105, Phl p 5.0106, Phl p 5.0107, Phl p 5.0108, Phl p 5.0109, Phl p 5.0201, Phl p 5.0202, Phl p 5.0203, Phl p 5.0204, Phl p 5.0205, Phl p 5.0206, Phl p 5.0207, Phl p 6, Phl p 6.0101, Phl p 6.0102, Phl p 7, Phl p 7.0101, Phl p P1-P2-P5-P6, Phl p P2-P6, Phl p P5-P1, Phl p P6-P2), Phoenix spp (Pho d 2, Pho d 2.0101, Pho d 40 kD, Pho d 90 kD), Phodopus spp (Pho s 21 kD), Phoma spp (Pho t 1), Phragmites spp (Phr a 1, Phr a 12, Phr a 13, Phr a 4, Phr a 5), Phytolacca spp (Phy a RIP), Pimpinella spp (Pim a 1, Pim a 2), Pinna spp (Pin a 1), Piper spp (Pip n 14 kD, Pip n 28 kD), Pisum spp (Pis s 1, Pis s 1.0101, Pis s 1.0102, Pis s 2, Pis s 2.0101, Pis s 5, Pis s Agglutinin, Pis s Albumin), Pistacia spp (Pis v 1, Pis v 1.0101, Pis v 2, Pis v 2.0101, Pis v 2.0201, Pis v 3, Pis v 3.0101, Pis v 4, Pis v 4.0101, Pis v 5, Pis v 5.0101), Platanus spp (Pla a 1, Pla a 1.0101, Pla a 2, Pla a 2.0101, Pla a 3, Pla a 3.0101, Pla a 8), Platichthys spp (Pla f 1), Plantago spp (Pla 11, Pla 11.0101, Pla I 1.0102, Pla 11.0103, Pla I Cytochrome C), Platanus spp (Pla oc 1, Pla or 1, Pla or 1.0101, Pla or 2, Pla or 2.0101, Pla or 3, Pla or 3.0101, Pla or 4, Pla or CyP, Pla r 1), Plectropomus spp (Ple ar 1), Pleospora spp (Ple h 1), Plectropomus spp (Ple le 1), Plodia spp (Plo i 1, Plo i 1.0101, Plo i 2, Plo i 2.0101), Poa spp (Poa p 1, Poa p 1.0101, Poa p 10, Poa p 12, Poa p 13, Poa p 2, Poa p 4, Poa p 5, Poa p 5.0101, Poa p 6, Poa p 7), Polistes spp (Pol a 1, Pol a 1.0101, Pol a 2, Pol a 2.0101, Pol a 5, Pol a 5.0101, Pol d 1, Pol d 1.0101, Pol d 1.0102, Pol d 1.0103, Pol d 1.0104, Pol d 4, Pol d 4.0101, Pol d 5, Pol d 5.0101, Pol e 1, Pol e 1.0101, Pol e 2, Pol e 4, Pol e 4.0101, Pol e 5, Pol e 5.0101, Pol f 5, Pol f 5.0101, Pol g 1, Pol g 1.0101, Pol g 2, Pol g 4, Pol g 5, Pol g 5.0101, Pol he MLT, Pol m 5, Pol m 5.0101), Polypedilum spp (Pol n 1), Pollicipes spp (Pol po 1), Pollachius spp (Pol vi 1), Polybia spp (Poly p 1, Poly p 1.0101, Poly p 2, Poly p 5, Poly s 5, Poly s 5.0101), Pomatomus spp (Porn sa 1), Pongo spp (Pon ab HSA), Pontastacus spp (Pon I 4, Pon 1 4.0101, Pon I 7, Pon 1 7.0101), Portunus spp (Por s 1, Por s 1.0101, Por s 1.0102, Por tr 1, Por tr 1.0101), Protortonia spp (Pro ca 38 kD), Procumbarus spp (Pro cl 1, Pro cl 1.0101, Pro cl 21 kD), Prosopis spp (Pro j 20 kD), Prunus spp (Pru ar 1, Pru ar 1.0101, Pru ar 3, Pru ar 3.0101, Pru av 1, Pru av 1.0101, Pru av 1.0201, Pru av 1.0202, Pru av 1.0203, Pru av 2, Pru av 2.0101, Pru av 3, Pru av 3.0101, Pru av 4, Pru av 4.0101, Pru c 1, Pru d 1, Pru d 2, Pru d 3, Pru d 3.0101, Pru d 4, Pru du 1, Pru du 2, Pru du 2S Albumin, Pru du 3, Pru du 3.0101, Pru du 4, Pru du 4.0101, Pru du 4.0102, Pru du 5, Pru du 5.0101, Pru du 6, Pru du 6.0101, Pru du 6.0201, Pru du Conglutin, Pru p 1, Pru p 1.0101, Pru p 2, Pru p 2.0101, Pru p 2.0201, Pru p 2.0301, Pru p 3, Pru p 3.0101, Pru p 3.0102, Pru p 4, Pru p 4.0101, Pru p 4.0201, Pru sa 3), Psilocybe spp (Psi c 1, Psi c 1.0101, Psi c 2, Psi c 2.0101), Psoroptes spp (Pso o 1, Pso o 10, Pso o 10.0101, Pso o 11, Pso o 13, Pso o 14, Pso o 2, Pso o 21, Pso o 3, Pso o 5, Pso o 7), Puma spp (Pum c 1), Punica spp (Pun g 3), Pyrus spp (Pyr c 1, Pyr c 1.0101, Pyr c 3, Pyr c 3.0101, Pyr c 4, Pyr c 4.0101, Pyr c 5, Pyr c 5.0101, Pyr py 2), Quercus spp (Que a 1, Que a 1.0101, Que a 1.0201, Que a 1.0301, Que a 1.0401, Que a 2, Que a 4), Rachycentron spp (Rac ca 1), Rana spp (Ran e 1, Ran e 1.0101, Ran e 2, Ran e 2.0101), Ranina spp (Ran ra 1), Rangifer spp (Ran t BLG), Rattus spp (Rat n 1, Rat n 1.0101, Rat n Casein, Rat n Gelatin, Rat n IgG, Rat n Phosvitin, Rat n RSA, Rat n Transferrin), Rhizomucor spp (Rhi m AP), Rhizopus spp (Rhi nv Lipase, Rhi o Lipase), Rhomboplites spp (Rho au 1), Rhodotorula spp (Rho m 1, Rho m 1.0101, Rho m 2, Rho m 2.0101), Ricinus spp (Ric c 1, Ric c 1.0101, Ric c 2, Ric c 3, Ric c 8, Ric c RIP), Rivulus spp (Riv ma 1), Robinia spp (Rob p 2, Rob p 4, Rob p Glucanase), Rosa spp (Ros r 3), Roystonea spp (Roy e 2), Rubus spp (Rub i 1, Rub i 1.0101, Rub i 3, Rub i 3.0101, Rub i Chitinase, Rub i CyP), Saccharomyces spp (Sac c Carboxypeptidase Y, Sac c CyP, Sac c Enolase, Sac c Glucosidase, Sac c Invertase, Sac c MnSOD, Sac c P2, Sac c Profilin), Salvelinus spp (Sal f 1), Salsola spp (Sal k 1, Sal k 1.0101, Sal k 1.0201, Sal k 1.0301, Sal k 1.0302, Sal k 2, Sal k 2.0101, Sal k 3, Sal k 3.0101, Sal k 4, Sal k 4.0101, Sal k 4.0201, Sal k 5, Sal k 5.0101), Salvelinus spp (Sal le Vitellogenin), Salmo spp (Sal s 1, Sal s 1.0101, Sal s 1.0201, Sal s 2, Sal s 2.0101, Sal s Gelatin), Sambucus spp (Sam n 1), Sander spp (San lu 1), Saponaria spp (Sap o RIP), Sardinops spp (Sar m 1), Sarkidiornis spp (Sar ml 1), Sardina spp (Sar p 1), Sarcoptes spp (Sar s 1, Sar s 14, Sar s 3, Sar s GST, Sar s PM), Sardinops spp (Sar sa 1, Sar sa 1.0101), Schistosoma spp (Sch j GST, Sch j PM, Sch j Sj22, Sch j Sj67, Sch ma Sm20, Sch ma Sm21, Sch ma Sm22, Sch ma Sm31), Sciaenops spp (Sci oc 1), Scomber spp (Sco a 1), Scombermorus spp (Sco ca 1), Scomberomorus spp (Sco g 1), Scomber spp (Sco j 1, Sco ma 1, Scos 1), Scolopendra spp (Sco y 7, Sco y 7.0101), Scylla spp (Scy o 1, Scy o 1.0101, Scy o 2, Scy pa 1, Scy pa 2, Scy s 1, Scy s 1.0101, Scy s 2), Sebastes spp (Seb fa 1, Seb in 1, Seb m 1, Seb m 1.0101, Seb m 1.0201), Secale spp (Sec c 1, Sec c 12, Sec c 13, Sec c 2, Sec c 20, Sec c 20.0101, Sec c 20.0201, Sec c 28, Sec c 3, Sec c 4, Sec c 4.0101, Sec c 4.0201, Sec c 5, Sec c 5.0101, Sec c akTI, Sec c akTI.0101), Senecio spp (Sen j MDH, Sen j PL), Sepia spp (Sep e 1, Sep e 1.0101), Sepioteuthis spp (September 11, Sep I 1.0101), Sepia spp (Sep m 1), Seriola spp (Ser d 1, Ser la 1), Sergestes spp (Ser lu 1), Seriola spp (Ser q 1, Ser ri 1), Sesamum spp (Ses i 1, Ses i 1.0101, Ses i 2, Ses i 2.0101, Ses i 3, Ses i 3.0101, Ses i 4, Ses i 4.0101, Ses i 5, Ses i 5.0101, Ses i 6, Ses i 6.0101, Ses i 7, Ses i 7.0101, Ses i 8), Shigella spp (Shi bo GST, Shi dy GST), Simulia spp (Sim vi 1, Sim vi 2, Sim vi 3, Sim vi 4, Sim vi 70 KD), Sinapis spp (Sin a 1, Sin a 1.0101, Sin a 1.0104, Sin a 1.0105, Sin a 1.0106, Sin a 1.0107, Sin a 1.0108, Sin a 2, Sin a 2.0101, Sin a 3, Sin a 3.0101, Sin a 4, Sin a 4.0101), Sinonovacula spp (Sin c 1, Sin c 1.0101), Solenopsis spp (Sol g 2, Sol g 2.0101, Sol g 3, Sol g 3.0101, Sol g 4, Sol g 4.0101, Sol g 4.0201, Sol i 1, Sol i 1.0101, Sol i 2, Sol i 2.0101, Sol i 3, Sol i 3.0101, Sol i 4, Sol i 4.0101), Solenocera spp (Sol me 1), Solenopsis spp (Sol r 1, Sol r 2, Sol r 2.0101, Sol r 3, Sol r 3.0101, Sol s 2, Sol s 2.0101, Sol s 3, Sol s 3.0101, Sol s 4), Solea spp (Sol so 1, Sol so TPI), Solanum spp (Sola t 1, Sola t 1.0101, Sola t 2, Sola t 2.0101, Sola t 3, Sola t 3.0101, Sola t 3.0102, Sola t 4, Sola t 4.0101, Sola t 8, Sola t Glucanase), Sorghum spp (Sor b 1, Sor h 1, Sor h 1.0101, Sor h 12, Sor h 7), Sparus spp (Spa a 1), Sphyrna spp (Sph ti 1), Spirulina spp (Spi mx beta_Phycocyanin), Spinacia spp (Spi o 2, Spi o RuBisCO), Squilla spp (Squ ac 1, Squ ac 1.0101, Squ o 1, Squ o 1.0101), Staphylococcus spp (Sta a FBP, Sta a SEA, Sta a SEB, Sta a SEC, Sta a SED, Sta a SEE, Sta a TSST), Stachybotrys spp (Sta c 3, Sta c 3.0101, Sta c Cellulase, Sta c Hemolysin, Sta c SchS34, Sta c Stachyrase A), Stemphylium spp (Ste b 1, Ste c 1, Ste v 1), Stolephorus spp (Sto i 1), Struthio spp (Str c 1, Str c 2, Str c 3), Streptococcus spp (Str dy Streptokinase), Streptomyces spp (Str g Pronase), Streptococcus spp (Str pn PspC), Strongylocentrotus spp (Str pu 18 kD, Str pu Vitellogenin), Streptococcus spp (Str py SPEA, Str py SPEC, Str py Streptokinase), Strongyloides spp (Str st 45 kD), Streptomyces spp (Str v PAT), Styela spp (Sty p 1), Suidasia spp (Sui m 1, Sui m 13, Sui m 2, Sui m 3, Sui m 5, Sui m 5.01, Sui m 5.02, Sui m 5.03, Sui m 6, Sui m 7, Sui m 8, Sui m 9), Sus spp (Sus s ACTH, Sus s ALA, Sus s Amylase, Sus s BLG, Sus s Casein, Sus s Casein alphaS1, Sus s Casein alphaS2, Sus s Casein beta, Sus s Casein kappa, Sus s Gelatin, Sus s HG, Sus s Insulin, Sus s Lipase, Sus s Pepsin, Sus s Phosvitin, Sus s PRVB, Sus s PSA, Sus s TCTP), Syntelopodeuma spp (Syn y 7, Syn y 7.0101), Syringa spp (Syr v 1, Syr v 1.0101, Syr v 1.0102, Syr v 1.0103, Syr v 2, Syr v 3, Syr v 3.0101), Tabanus spp (Tab y 1, Tab y 1.0101, Tab y 2, Tab y 2.0101, Tab y 5, Tab y 5.0101), Tadorna spp (Tad ra 1), Talaromyces spp (Tal st 22, Tal st 3, Tal st 8), Taraxacum spp (Tar o 18 kD), Taxodium spp (Tax d 2), Tegenaria spp (Teg d Hemocyanin), Teladorsagia spp (Tel ci 3), Thaumetopoea spp (Tha p 1, Tha p 1.0101, Tha p 2, Tha p 2.0101), Theragra spp (The c 1), Thermomyces spp (The I Lipase, The sp Lipase, The sp Xylanase), Thunnus spp (Thu a 1, Thu a 1.0101, Thu a Collagen, Thu al 1, Thu at 1, Thu o 1, Thu o Collagen), Thuja spp (Thu oc 3, Thu p 1), Thunnus spp (Thu t 1, Thu to 1), Thyrsites spp (Thy at 1), Thyrophygus spp (Thy y 7, Thy y 7.0101), Todarodes spp (Tod p 1, Tod p 1.0101, Tod p 1.0102), Toxoptera spp (Tox c 7, Tox c 7.0101), Toxocara spp (Tox ca TES120, Tox ca TES26, Tox ca TES30), Toxoplasma spp (Tox g HSP70), Trachypenaeus spp (Tra c 1), Trachinotus spp (Tra ca 1), Trachurus spp (Tra j 1, Tra j Gelatin, Tra tr Gelatin), Triticum spp (Tri a 1, Tri a 10 kD, Tri a 12, Tri a 12.0101, Tri a 12.0102, Tri a 12.0103, Tri a 12.0104, Tri a 13, Tri a 14, Tri a 14.0101, Tri a 14.0201, Tri a 15, Tri a 15.0101, Tri a 18, Tri a 18.0101, Tri a 19, Tri a 19.0101, Tri a 2, Tri a 21, Tri a 21.0101, Tri a 23kd, Tri a 25, Tri a 25.0101, Tri a 26, Tri a 26.0101, Tri a 27, Tri a 27.0101, Tri a 28, Tri a 28.0101, Tri a 29, Tri a 29.0101, Tri a 29.0201, Tri a 3, Tri a 30, Tri a 30.0101, Tri a 31, Tri a 31.0101, Tri a 32, Tri a 32.0101, Tri a 33, Tri a 33.0101, Tri a 34, Tri a 34.0101, Tri a 35, Tri a 35.0101, Tri a 36, Tri a 36.0101, Tri a 37, Tri a 37.0101, Tri a 4, Tri a 4.0101, Tri a 4.0201, Tri a 5, Tri a 7, Tri a aA_SI, Tri a alpha_Gliadin, Tri a bA, Tri a Bd36K, Tri a beta_Gliadin, Tri a Chitinase, Tri a CM16, Tri a DH, Tri a Endochitinase, Tri a gamma_Gliadin, Tri a Germin, Tri a Gliadin, Tri a GST, Tri a LMW Glu, Tri a LMW-GS B16, Tri a LMW-GS P42, Tri a LMW-GS P73, Tri a LTP2, Tri a omega2_Gliadin, Tri a Peroxidase, Tri a Peroxidase 1, Tri a SPI, Tri a TLP, Tri a Tritin, Tri a XI), Tritirachium spp (Tri al Proteinase K), Tribolium spp (Tri ca 17, Tri ca 17.0101, Tri ca 7, Tri ca 7.0101), Trichostrongylus spp (Tri co 3, Tri co 3.0101), Trichophyton spp (Tri eq 4), Trigonella spp (Tri fg 1, Tri fg 2, Tri fg 3, Tri fg 4), Trichosanthes spp (Tri k RIP), Trichiurus spp (Tri le 1), Triticum spp (Tri m Peroxidase), Trichophyton spp (Tri me 2, Tri me 4), Trisetum spp (Tri p 1, Tri p 5), Trichinella spp (Tri ps 3, Tri ps 3.0101), Trichophyton spp (Tri r 2, Tri r 2.0101, Tri r 4, Tri r 4.0101), Trichoderma spp (Tri rs Cellulase), Triticum spp (Tri s 14), Trichophyton spp (Tri sc 2, Tri sc 4, Tri so 2), Trichinella spp (Tri sp 3, Tri sp 3.0101, Tri sp 3.0102, Tri sp 3.0103, Tri sp 3.0104, Tri sp 3.0105, Tri sp 3.0106), Trichophyton spp (Tri t 1, Tri t 1.0101, Tri t 4, Tri t 4.0101), Triticum spp (Tri td 14, Tri td akTI), Trichoderma spp (Tri v Cellulase), Trichophyton spp (Tri ye 4), Triatoma spp (Tria p 1, Tria p 1.0101), Triplochiton spp (Trip s 1), Turbo spp (Tur c 1, Tur c PM), Tyrophagus spp (Tyr p 1, Tyrp 10, Tyrp 10.0101, Tyrp 10.0102, Tyr p 13, Tyrp 13.0101, Tyr p 2, Tyr p 2.0101, Tyr p 24, Tyr p 24.0101, Tyr p 3, Tyr p 3.0101, Tyr p 4, Tyr p 5, Tyr p 5.01, Tyr p 5.02, Tyr p 5.03, Tyr p 7, Tyr p alpha Tubulin), Illocladium spp (Ulo a 1, Ulo at 1, Ulo b 1, Ulo c 1, Ulo co 1, Ulo cu 1, Ulo mu 1, Ulo ob 1, Ulo se 1, Ulo su 1, Ulo to 1), Uncia spp (Unc u 1), Urophycis spp (Uro to 1), Vaccinium spp (Vac m 3), Varroa spp (Var j 13 kD), Venerupis spp (Ven ph 1, Ven ph 1.0101), Vespula spp (Ves f 1, Ves f 2, Ves f 5, Ves f 5.0101, Ves g 1, Ves g 2, Ves g 5, Ves g 5.0101, Ves m 1, Ves m 1.0101, Ves m 2, Ves m 2.0101, Ves m 5, Ves m 5.0101, Ves m MLT, Ves p 1, Ves p 2, Ves p 5, Ves p 5.0101, Ves s 1, Ves s 1.0101, Ves s 2, Ves s 5, Ves s 5.0101, Ves v 1, Ves v 1.0101, Ves v 2, Ves v 2.0101, Ves v 2.0201, Ves v 3, Ves v 3.0101, Ves v 5, Ves v 5.0101, Ves v 5-Pol a 5, Ves vi 5, Ves vi 5.0101), Vespa spp (Vesp c 1, Vesp c 1.0101, Vesp c 2, Vesp c 5, Vesp c 5.0101, Vesp c 5.0102, Vesp m 1, Vesp m 1.0101, Vesp m 5, Vesp m 5.0101, Vesp ma 1, Vesp ma 2, Vesp ma 5, Vesp ma MLT, Vesp v MLT), Vigna spp (Vig r 1, Vigr 1.0101, Vig r 17 kD, Vig r 5, Vig r 8S Globulin, Vig r Albumin, Vig r beta-Conglycinin), Vitis spp (Vit v 1, Vit v 1.0101, Vit v 4, Vit v 5, Vit v Glucanase, Vit v TLP), Xiphias spp (Xip g 1, Xip g 1.0101, Xip g 25 kD), Zea spp (Zea m 1, Zea m 1.0101, Zea m 11, Zea m 12, Zea m 12.0101, Zea m 12.0102, Zea m 12.0103, Zea m 12.0104, Zea m 12.0105, Zea m 13, Zea m 14, Zea m 14.0101, Zea m 14.0102, Zea m 2, Zea m 20S, Zea m 22, Zea m 25, Zea m 25.0101, Zea m 27 kD Zein, Zea m 3, Zea m 4, Zea m 5, Zea m 50 kD Zein, Zea m 7, Zea m Chitinase, Zea m G1, Zea m G2, Zea m PAO, Zea m Zm13), Zeus spp (Zeu fa 1), Ziziphus spp (Ziz m 1, Ziz m 1.0101), Zoarces spp (Zoa a ISP III), and Zygophyllum spp (Zyg f 2).

### Heterologous Antigens

The nucleic acid compositions herein described can encode a plurality of heterologous antigens (e.g, a plurality of disparate AP). In some embodiments, “heterologous antigens” are antigens that are of different origins, such as derived from pathogens of different taxonomic groups such as different strains, species, subgenera, genera, subfamilies or families and/or from antigenically divergent pathogens (e.g., variants thereof). Classification of viruses into various taxonomic groups is well understood by those skilled in the art. Each of the disparate AP of the plurality of disparate AP can differ with respect to each other.

In some embodiments, the plurality of disparate AP comprises: between about 2 and about 500 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values) antigenic polypeptides that differ from each other; AP of a same protein type; and/or AP of different protein types.

The plurality of AP can be of a same protein type or corresponding proteins. AP of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins. In some embodiments, coronavirus antigens of a same protein type have at least 50% sequence identity, for example at least 65%, 70%, 80%, 90%, 95%, 98%, 99%, or more sequence identity.

Alternatively, in some embodiments the plurality of AP can comprise coronavirus proteins of different protein types. AP of different protein types typically have different functions. For example, the plurality of AP can comprise coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, and/or a coronavirus M protein or a portion thereof.

The plurality of disparate (e.g., heterologous) AP can comprise at least m pathogenic antigens of an mth infectious agent, wherein m is an integer greater than 2 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values), and wherein each mth pathogenic antigen is different from one another (e.g., heterologous). In some embodiments, m is an integer greater than 50. The plurality of disparate AP can comprise two or more of a 1st pathogenic antigen (PA) of a 1st infectious agent (IA), a 2nd PA of a 2nd IA, a 3rd PA of a 3rd IA, a 4th PA of a 4th IA, a 5th PA of a 5th IA, a 6th PA of a 6th IA, a 7th PA of a 7th IA, a 8th PA of a 8th IA, a 9th PA of a 9th IA, a 10th PA of a 10th IA, a 11th PA of a 11th IA, a 12th PA of a 12th IA, a 13th PA of a 13th IA, a 14th PA of a 14th IA, a 15th PA of a 15th IA, a 16th PA of a 16th IA, a 17th PA of a 17th IA, a 18th PA of a 18th IA, a 19th PA of a 19th IA, a 20th PA of a 20th IA, a 21st PA of a 21st IA, a 22nd PA of a 22nd IA, a 23rd PA of a 23rd IA, a 24th PA of a 24th IA, a 25th PA of a 25th IA, a 26th PA of a 26th IA, a 27th PA of a 27th IA, a 28th PA of a 28th IA, a 29th PA of a 29th IA, a 30th PA of a 30th IA, a 31st PA of a 31st IA, a 32nd PA of a 32nd IA, a 33rd PA of a 33rd IA, a 34th PA of a 34th IA, a 35th PA of a 35th IA, a 36th PA of a 36th IA, a 37th PA of a 37th IA, a 38th PA of a 38th IA, a 39th PA of a 39th IA, a 40th PA of a 40th IA, a 41st PA of a 41st IA, a 42nd PA of a 42nd IA, a 43rd PA of a 43rd IA, a 44th PA of a 44th IA, a 45th PA of a 45th IA, a 46th PA of a 46th IA, a 47th PA of a 47th IA, a 48th PA of a 48th IA, a 49th PA of a 49th IA, and a 50th PA of a 50th IA. The 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th pathogenic antigens can be the same or different from one another. The 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th infectious agents can be the same or different from one another. The plurality of disparate (e.g., heterologous) AP can comprise a plurality of CoV antigens, a plurality of influenza antigens, and/or a plurality of HIV antigens.

The compositions provided herein can induce broadly protective anti-infectious agent responses by eliciting broadly neutralizing antibodies. As an example, broadly neutralizing antibodies are antibodies that can neutralize coronaviruses from a taxonomic group that is not only the same as but also differs from the taxonomic groups of the coronaviruses from which the coronavirus antigens used to elicit the antibodies are derived. Broadly neutralizing response can also be referred to as heterologously neutralizing response. In some embodiments, the compositions herein described can elicit broadly neutralizing antibodies that neutralize one or more infectious agents from a subfamily, genus, subgenus, species, and/or strain that differ from the subfamily, genus, subgenus, species, and/or strain of the infectious agents from which AP are derived.

### Vectors and Carriers

The nucleic acid composition (e.g., a promoter operably linked to a polynucleotide comprising a first nucleic acid unit and a second nucleic acid unit) can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition (e.g., a promoter operably linked to a polynucleotide comprising a first nucleic acid unit and a second nucleic acid unit) is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The polynucleotide(s) described herein can be comprised in the one or more vectors. The polynucleotide(s) described herein can be comprised in the same vector and/or different vectors. The polynucleotide(s) described herein can be situated on the same nucleic acid and/or different nucleic acids.

Vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The one or more vectors can be a DNA vaccine. The DNA vaccine can be a plasmid-based DNA vaccine, a minicircle-based DNA vaccine, a bacmid-based DNA vaccine, a minigene-based DNA vaccine, a ministring DNA (linear covalently closed DNA vector) vaccine, a closed-ended linear duplex DNA (CELiD or ceDNA) vaccine, a Doggybone™ DNA vaccine, a dumbbell shaped DNA vaccine, or a minimalistic immunological-defined gene expression (MIDGE)-vector DNA vaccine.

The nucleic acid composition can be or can comprise mRNA. The composition (e.g., nucleic acid composition) can be an mRNA vaccine. The mRNA can be formulated in a lipid nanoparticle (LNP). The term “lipid nanoparticle”, also referred to as LNP, refers to a particle having at least one dimension on the order of nanometers (e.g., 1-1,000 nm) which includes one or more lipids. In some embodiments, such lipid nanoparticles comprise a cationic lipid and one or more excipient selected from neutral lipids, charged lipids, steroids and polymer conjugated lipids (e.g., a pegylated lipid). In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. In some embodiments, the mRNA or a portion thereof is associated with the lipid nanoparticles. An LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

The LNP can comprise one or more of an ionizable cationic lipid, a non-cationic lipid (e.g., a neutral lipid), a sterol, and a PEG-modified lipid. The LNP can comprise 0.5-15 mol % PEG-modified lipid, 5-25 mol % non-cationic lipid, 25-55 mol % sterol, and 20-60 mol % ionizable cationic lipid. The LNP can comprise 40-55 mol % ionizable cationic lipid, 5-15 mol % neutral lipid, 35-45 mol % sterol, and 1-5 mol % PEG-modified lipid. In some embodiments, the RNA (e.g., mRNA) of the disclosure is formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491 all of which are incorporated by reference herein in their entirety.

In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid.

The ionizable cationic lipid can be heptadecan-9-yl 8 ((2 hydroxyethyl) (6 oxo 6-(undecyloxy)hexyl)amino) octanoate. The neutral lipid can be 1,2 distearoyl sn glycero-3 phosphocholine (DSPC). The sterol can be cholesterol. The PEG-modified lipid can be 1-monomethoxypolyethyleneglycol-2,3-dimyristylglycerol with polyethylene glycol of average molecular weight 2000 (PEG2000 DMG).

The wt/wt ratio of lipid to mRNA can be from about 1:100 to about 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values).

The LNP can comprise a cationic lipid. The cationic lipid is can be cationisable, i.e. it becomes protonated as the pH is lowered below the pKa of the ionizable group of the lipid, but is progressively more neutral at higher pH values. When positively charged, the lipid is then able to associate with negatively charged nucleic acids. In some embodiments, the cationic lipid comprises a zwitterionic lipid that assumes a positive charge on pH decrease. The LNP may comprise any lipid capable of forming a particle to which the one or more nucleic acid molecules are attached, or in which the one or more nucleic acid molecules are encapsulated. In some embodiments, the LNP may comprise any further cationic or cationisable lipid, i.e. any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3dioleoyloxy) propyl)-N,N,N-trimethylammonium chloride (DOTAP); 3-(N-(N′,N′dimethylaminoethane)-carbamoyl) cholesterol (DC-Chol), N-(1-(2,3-dioleoyloxy) propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), and N-(1,2dimyristyloxyprop-3-yl)-N,N-dimethyl-N-hydroxyethyl ammonium bromide (DMRIE).

Additionally, a number of commercial preparations of cationic lipids are available which can be used in embodiments provided herein. These include, for example, LIPOFECTIN® (commercially available cationic liposomes comprising DOTMA and 1,2-dioleoyl-sn-3 phosphoethanolamine (DOPE), from GIBCO/BRL, Grand Island, N.Y.); LIPOFECTAMINE®(commercially available cationic liposomes comprising N-(1-(2,3dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-lcarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In some embodiments, the neutral lipid is 1,2-distearoyl-sn-glycero-3phosphocholine (DSPC).

In some embodiments, the cationic lipid is an amino lipid. Suitable amino lipids useful include those described in WO2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG. In some embodiments, a sterol comprises cholesterol, fecosterol, sitosterol, ergosterol, campesterol, stigmasterol, brassicasterol, tomatidine, ursolic acid, alpha-tocopherol, and mixtures thereof.

In some embodiments, the nucleic acid composition comprising an mRNA sequence is a modified mRNA sequence. In this context, a modification as defined herein can lead to a stabilization of the mRNA sequence provided herein. In some embodiments, there is thus provided a stabilized mRNA sequence comprising at least one coding region as defined herein (e.g. polynucleotide(s) encoding unit payload protein(s)). In some embodiments, the nucleic acid composition comprising an mRNA sequence may thus be provided as a “stabilized mRNA sequence”, that is to say as an mRNA that is essentially resistant to in vivo degradation (e.g. by an exo- or endo-nuclease). Such stabilization can be effected, for example, by a modified phosphate backbone of an mRNA provided herein. A backbone modification can be a modification in which phosphates of the backbone of the nucleotides contained in the mRNA are chemically modified. Nucleotides that can be used in this connection contain e.g. a phosphorothioate-modified phosphate backbone, such as at least one of the phosphate oxygens contained in the phosphate backbone being replaced by a sulfur atom. Stabilized mRNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from methylphosphonates, phosphoramidates and phosphorothioates (e.g. cytidine-5′-O-(1-thiophosphate)). The term “mRNA modification” as used herein may refer to chemical modifications comprising backbone modifications as well as sugar modifications or base modifications. In this context, a modified mRNA (sequence) as defined herein may contain nucleotide analogues/modifications, e.g. backbone modifications, sugar modifications or base modifications. A backbone modification can be a modification, in which phosphates of the backbone of the nucleotides contained in an mRNA compound comprising an mRNA sequence as defined herein are chemically modified. A sugar modification can be a chemical modification of the sugar of the nucleotides of the mRNA compound comprising an mRNA sequence as defined herein. Furthermore, a base modification can be a chemical modification of the base moiety of the nucleotides of the mRNA compound comprising an mRNA sequence. In this context, nucleotide analogues or modifications can be selected from nucleotide analogues, which are applicable for transcription and/or translation.

The mRNA provided herein can comprise a 5′ untranslated region (UTR), a 3′ UTR, and/or a cap (e.g., a CAP analogue). A modified mRNA sequence as defined herein, can be modified by the addition of a so-called “5′-CAP structure”, which can stabilize the mRNA as described herein. A 5′-CAP is an entity, typically a modified nucleotide entity, which generally “caps” the 5′-end of a mature mRNA. A 5′-CAP may typically be formed by a modified nucleotide, particularly by a derivative of a guanine nucleotide. In some embodiments, the 5′-CAP is linked to the 5′-terminus via a 5′-5′-triphosphate linkage. A 5′-CAP may be methylated, e.g. m7GpppN, wherein N is the terminal 5′ nucleotide of the nucleic acid carrying the 5′-CAP, typically the 5′-end of an mRNA. m7GpppN is the 5′-CAP structure, which naturally occurs in mRNA transcribed by polymerase II and is therefore in some embodiments is not considered as modification comprised in a modified mRNA in this context. Accordingly, a modified mRNA sequence may comprise a m7GpppN as 5′-cap, but additionally the modified mRNA sequence typically comprises at least one further modification as defined herein. A CAP analogue refers to a non-polymerizable di-nucleotide that has CAP functionality in that it facilitates translation or localization, and/or prevents degradation of the RNA molecule when incorporated at the 5′-end of the RNA molecule. Non-polymerizable means that the CAP analogue will be incorporated only at the 5′-terminus because it does not have a 5′ triphosphate and therefore cannot be extended in the 3′-direction by a template-dependent RNA polymerase. CAP analogues include, but are not limited to, a chemical structure selected from m7GpppG, m7GpppA, m7GpppC; unmethylated CAP analogues (e.g., GpppG); dimethylated CAP analogue (e.g., m2,7GpppG), trimethylated CAP analogue (e.g., m2,2,7GpppG), dimethylated symmetrical CAP analogues (e.g., m7Gpppm7G), or anti reverse CAP analogues (e.g., ARCA; m7,2′OmeGpppG, m7,2′dGpppG, m7,3′OmeGpppG, m7,3′dGpppG and their tetraphosphate derivatives) (Stepinski et al., 2001. RNA 7 (10): 1486-95). Further CAP analogues have been described previously (U.S. Pat. No. 7,074,596, WO2008/016473, WO2008/157688, WO2009/149253, WO2011/015347, and WO2013/059475).

The polynucleotide can comprise one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. The polynucleotide can comprise a modified nucleotide in place of one or more uridines. The modified nucleoside can be selected from pseudouridine (ψ), N 1-methyl-pseudouridine (m 1Ψ), and 5-methyl-uridine (m5U). In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB 2017/051367 all of which are incorporated by reference herein.

### Engineered Cells

Disclosed herein include engineered cells. In some embodiments, the engineered cells comprise a nucleic acid composition disclosed herein. In some embodiments, the cell is: a cell of a subject; an in vivo cell, an ex vivo cell, or an in situ cell; and/or an adherent cell or a suspension cell. The cell can comprise a eukaryotic cell (e.g., a mammalian cell). The mammalian cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof. The cell can be the cell of a subject (e.g, a subject suffering from a disease or disorder). The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

### Pharmaceutically Acceptable Compositions and Methods of Treatment

Disclosed herein include pharmaceutical compositions. In some embodiments, the pharmaceutical composition comprises: a nucleic acid composition disclosed herein and/or engineered cells disclosed herein. In some embodiments, the pharmaceutical composition further comprises one or more pharmaceutically acceptable carriers, diluents and/or excipients.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or engineered cells disclosed herein, thereby treating or preventing the disease or disorder in the subject. The disease or disorder can be a disease or disorder caused by an infectious agent.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

In some embodiments, administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, administering comprises: (i) isolating one or more cells from the subject; (ii) contacting said one or more cells with a nucleic acid composition provided herein, thereby generating engineered cells (optionally the contacting comprises transduction and/or transfection, further optionally the nucleic acid composition comprises a transient or integrating expression vector); and (iii) administering the one or more engineered cells into a subject after the contacting step. The method can comprise administering to the subject at least two doses of the nucleic acid composition, the pharmaceutical composition, and/or the engineered cells. The nucleic acid composition, the pharmaceutical composition, and/or the engineered cells can be co-administered with an adjuvant.

The disease or disorder can be a blood disease, a gut microbiome disease or disorder, an inflammatory disease or disorder of the gut, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a solid tumor, a disorder cause by aberrant DNA damage repair, or any combination thereof.

The disease or disorder can be an infectious disease selected from an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-COV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-COV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen (e.g., a proliferative disease, a precancerous condition, a cancer, and a non-cancer related indication associated with expression of the tumor antigen). The cancer can be colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein. The compositions can be in the form of kits of parts. In a kit of parts, one or more components of the compositions disclosed herein are provided independent of one another (e.g., the mRNA and LNP lipids are provided as separate compositions) and are then employed (e.g., by a user) to generate the compositions.

### EXAMPLES

Some aspects of the embodiments discussed above are disclosed in further detail in the following examples, which are not in any way intended to limit the scope of the present disclosure.

### Example 1

### Stoichiometric Expression of Messenger Polycistrons by Eukaryotic Ribosomes (SEMPER) for Compact, Ratio-Tunable Multi-Gene Expression from Single mRNAs

Applications of mammalian synthetic biology increasingly require the ability to express multiple proteins at user-determined stoichiometries from single, compactly encoded transcripts. Provided herein are approaches for expressing multiple open reading frames (ORFs) from a single transcript, taking advantage of the leaky scanning model of translation initiation. In this method, adjacent ORFs are translated from a single messenger RNA at tunable ratios determined by their order in the sequence and the strength of their translation initiation sites. This approach is termed “Stoichiometric Expression of Messenger Polycistrons by Eukaryotic Ribosomes” (SEMPER) in some embodiments. The principles of this approach were demonstrated by expressing up to three fluorescent proteins from one plasmid in two different cell lines. Then it was used to encode a stoichiometrically tuned polycistronic construct encoding gas vesicle acoustic reporter genes, showing that enforcing the optimal ratio in every cell enables efficient formation of the multi-protein complex while minimizing cellular toxicity. It was also demonstrated that SEMPER enables polycistronic expression of recombinant monoclonal antibodies from plasmid DNA and of two fluorescent proteins from single mRNAs made through in vitro transcription. Finally, a probabilistic model is provided herein to support the mechanisms underlying SEMPER and aid with future construct design. Overall, SEMPER can enable a broad range of applications requiring tunable expression from compact eukaryotic constructs.

### INTRODUCTION

Mammalian cell engineering promises to enable treatment of age-related degeneration, reversal of genetic diseases, and even transformation of cells into living therapeutics and sensors. Realizing this promise requires the development of genetic circuits capable of finely tuning the relative expression stoichiometries of multiple proteins to produce functional multimeric protein assemblies, multi-component signaling systems, or multi-enzyme biosynthetic pathways. Current approaches to doing so at the DNA level (e.g., varying promoter strength or titrating copy numbers of each gene) yield lengthy DNA constructs that often must be packaged into multiple delivery vectors and lead to undesirable cell-to-cell variability due to both stochastic gene delivery and integration across the population. Additionally, attainable protein expression stoichiometries are limited by the transcriptional strengths of a relatively small set of curated promoters that often demonstrate cell-to-cell variability.

Post-transcriptionally, sequence motifs such as internal ribosome entry sites (IRES) or 2A self-cleaving peptides may be used to encode multiple open reading frames (ORF) into a single transcript and tune protein stoichiometries using relative translation levels. Such post-transcriptional mechanisms are also useful for mRNA-based, multi-gene expression systems, which are of relevance to mRNA vaccine and therapeutic development. Although powerful tools, these genetic parts have significant disadvantages. For instance, IRES sequences have a significant genetic footprint (˜200-600 bps), leading to lengthier genetic constructs which may reduce viral packaging efficiency. Likewise, 2A self-cleaving peptides leave peptide scars and can yield undesired fusion proteins, both of which can be detrimental to protein function. In addition, when employed in bicistronic vectors, these genetic parts can strongly attenuate the expression of the second ORF relative to the expression of the first ORF. Here, an alternative approach is provided that enables compact and robustly tunable polycistronic expression in mammalian cells using plasmid- and mRNA-based expression systems. This approach is called “Stoichiometric Expression of Messenger Polycistrons by Eukaryotic Ribosomes” (SEMPER) in some embodiments provided herein.

SEMPER uses the canonical cap-dependent ribosome recruitment and translation mechanism in mammalian systems, which begins when the 43S preinitiation complex (PIC) of the ribosome is loaded onto the 5′ end of mRNA. This complex then scans the 5′ untranslated region (5′UTR) until it encounters a translation initiation site (TIS), consisting of the start codon (AUG) and ˜3-10 neighboring nucleotides. The TIS sets the translational reading frame and initiates translation by engaging with the 60S ribosomal subunit. The full ribosome then translates the mRNA into protein until it encounters a stop codon, where it terminates translation and disengages the transcript. With some frequency, the 43S PIC may scan through the first TIS and initiate translation from a downstream ORF starting at another TIS, a phenomenon called leaky ribosomal scanning (LRS). As determined by its sequence, a strong TIS (e.g. the Kozak consensus sequence) will reliably initiate translation while weaker ones will more frequently allow the 43S PIC to scan past.

By employing a short ORF (uORF) upstream of a gene of interest (GOI), mammalian cells naturally use LRS and alternate TISs to divert a portion of the ribosome flux away from a GOI, effectively downregulating its translation. Recently, Ferreira and colleagues demonstrated that it is possible to use synthetic uORFs to regulate the expression of a downstream recombinant GOI. They also empirically determined the strength of various translation initiation sequences and showed that it is possible to divert varying amounts of ribosomal flux away from the GOI by varying the uORF TIS strength. uORFs have also been used in synthetic systems to tune an endoribonuclease-based feedforward controller to manage resource limitation. Others have also utilized translation initiation strength variability to tune the expression of a GOI in the context of modular genetic design. The SEMPER approach replaces non-protein-coding uORFs with longer, functional coding sequences. In this study, it is shown that by chaining together multiple ORFs while varying the translation initiation strength of each ORF, this approach achieves polycistronic expression of multiple proteins with tunable translation rates. It is further demonstrated that this framework is functional in in vitro transcribed mRNA, paving the way for advances in mRNA-based protein therapeutics of higher complexity.

### Results

### Tunable, Plasmid-Based SEMPER Framework for Expressing Two OREs

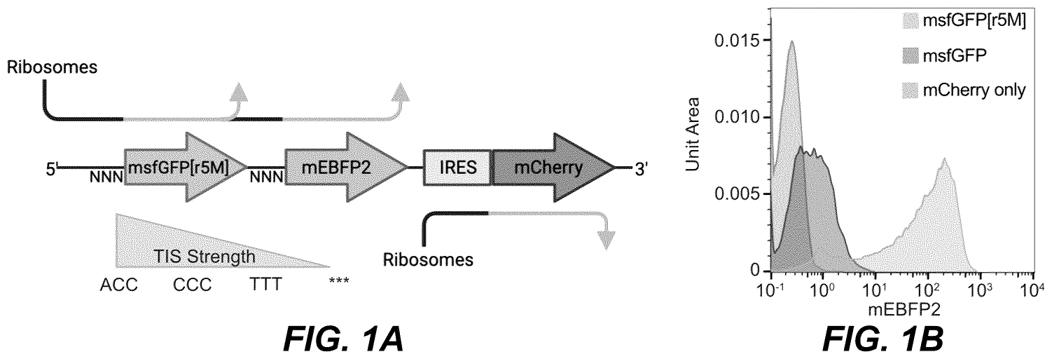

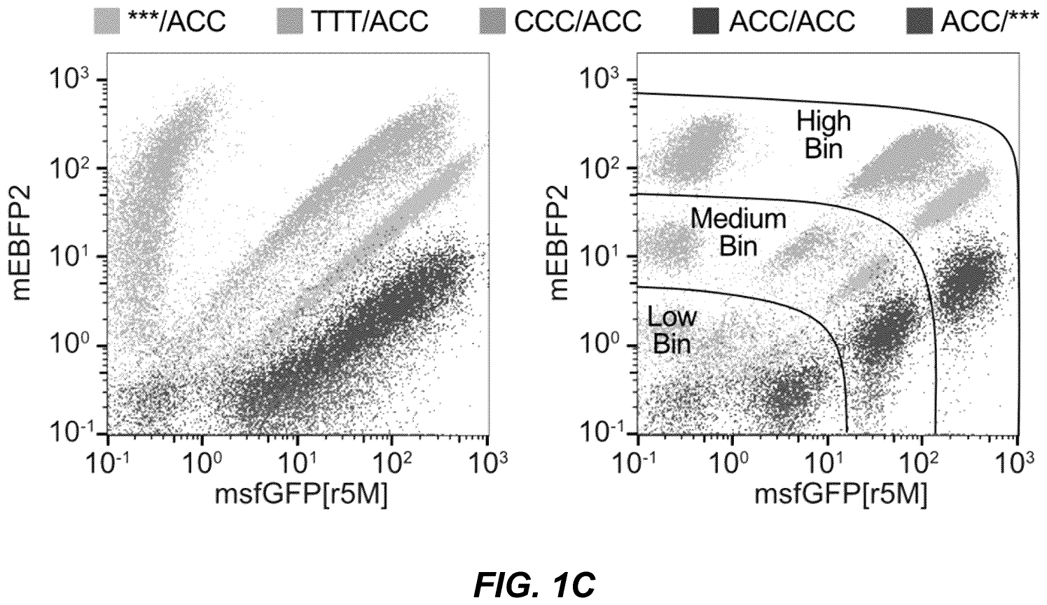

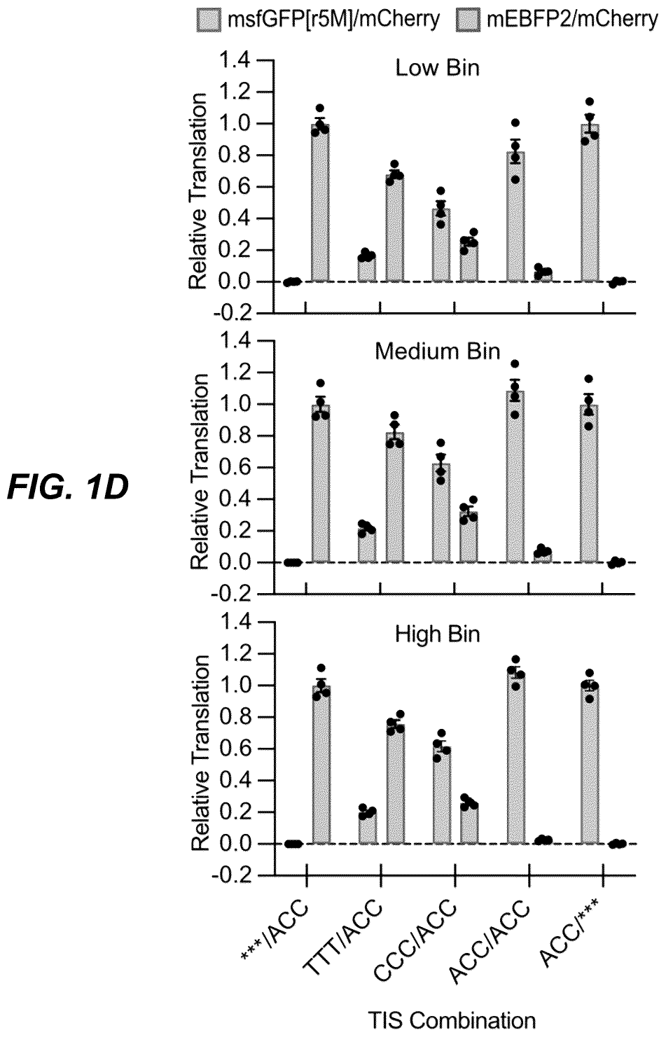

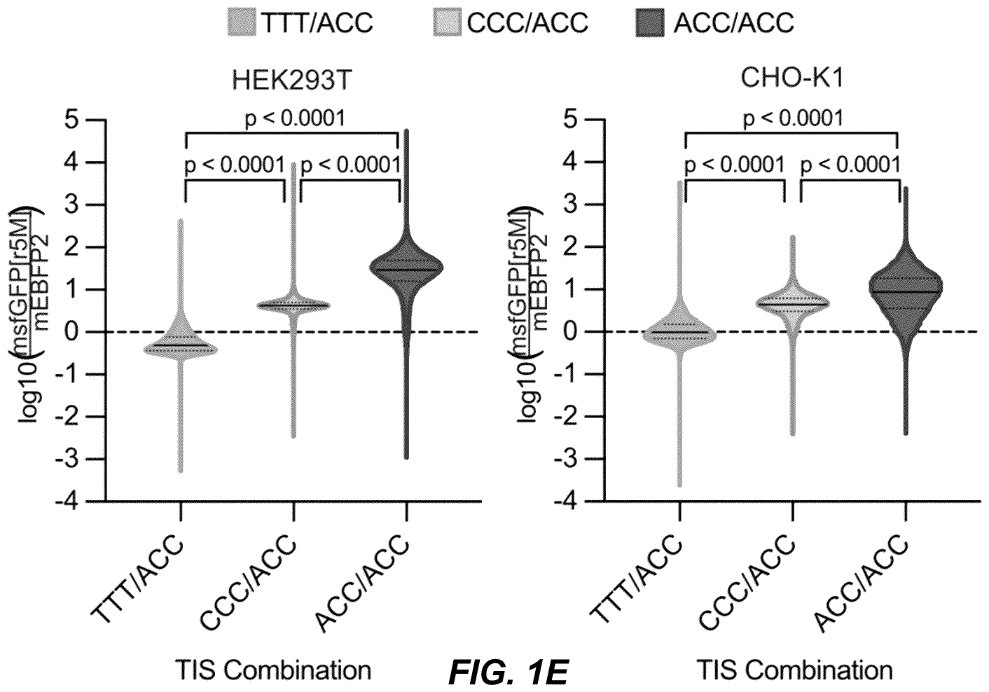

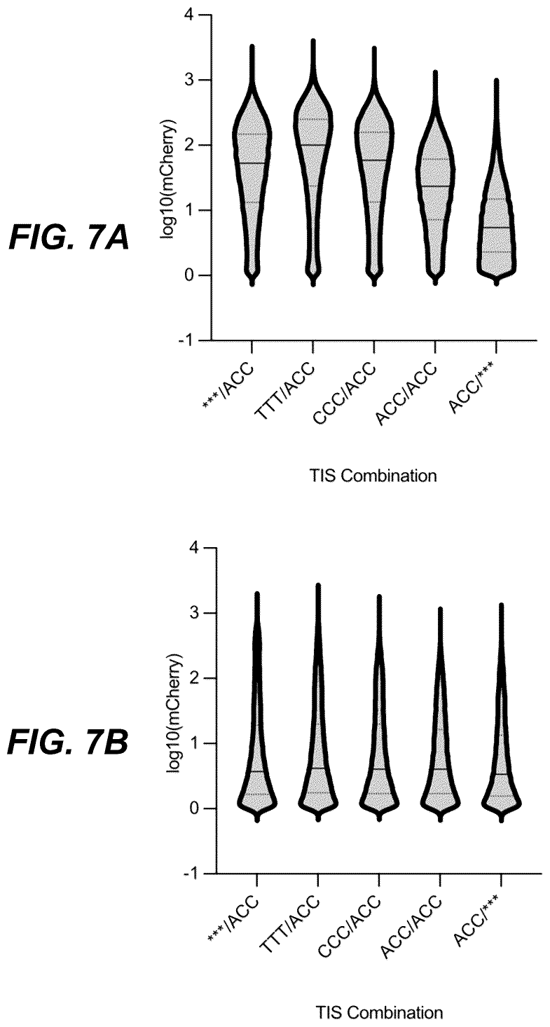

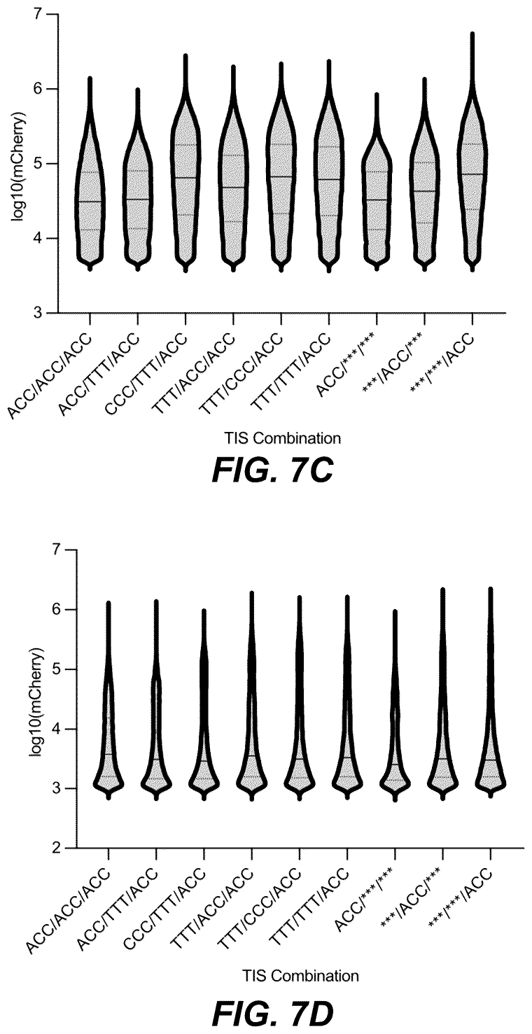

To test tunable translation levels of two recombinant proteins from single transcripts, fluorescent proteins (FPs) were encoded with minimal spectral overlap into the first two ORFs of the SEMPER plasmid vector (FIG. 1A). 11 bps of distance was included between these ORFs to ensure that read-through of the stop codon of the first ORF would not result in a fusion containing both proteins. The TIS sequence NNNAUGG was used to initiate translation of the ORFs, where NNN represents one of the following sequences: ACC, CCC, TTT. These sequences were described as having strong, medium, and weak translation initiation efficiency respectively by Ferreira et al. All the FPs were engineered to contain a valine residue (GTG or GTT) following the N-terminal methionine to ensure that changes in translation initiation were due to the trinucleotide preceding the start codon and not the dinucleotide succeeding it. TIS sequences will be referred to by their variable NNN sequence. One other sequence (TTTCCAT) was used, referred to as ***, to scrub the TIS entirely and prevent the ORF from being translated. For the first ORF, a methionine-less monomeric Superfolder GFP (msfGFP [r5M]) was generated in which all methionines-except the N-terminal one-were mutated to other amino acids. In addition, out-of-frame AUGs were removed from the msfGFP [r5M] coding sequence using synonymous mutations. These mutations effectively removed all internal TISs within ORF 1 that could reduce ribosomal flux to downstream ORFs (FIG. 1B). In the second ORF, mEBFP2 was encoded with all its natural methionines. Finally, downstream of the SEMPER ORFs, an IRES followed by mCherry (IRES-mCherry) was included for normalization. As ribosomal binding to the IRES and subsequent translation of mCherry are conducted independently of ORF 1 and ORF 2 translation, the mCherry allowed us to normalize single-cell fluorescence measurements for msfGFP [r5M] and mEBFP2, a strategy common to LRS-focused studies. Because the analyte ORFs and IRES-mCherry were encoded on the same transcript, this normalization scheme accounted for variations in transfection efficiency, transcription, and mRNA decay. mCherry fluorescence also served as a proxy for transcript abundance in each cell (FIG. 7 and Tables 1A-1D).

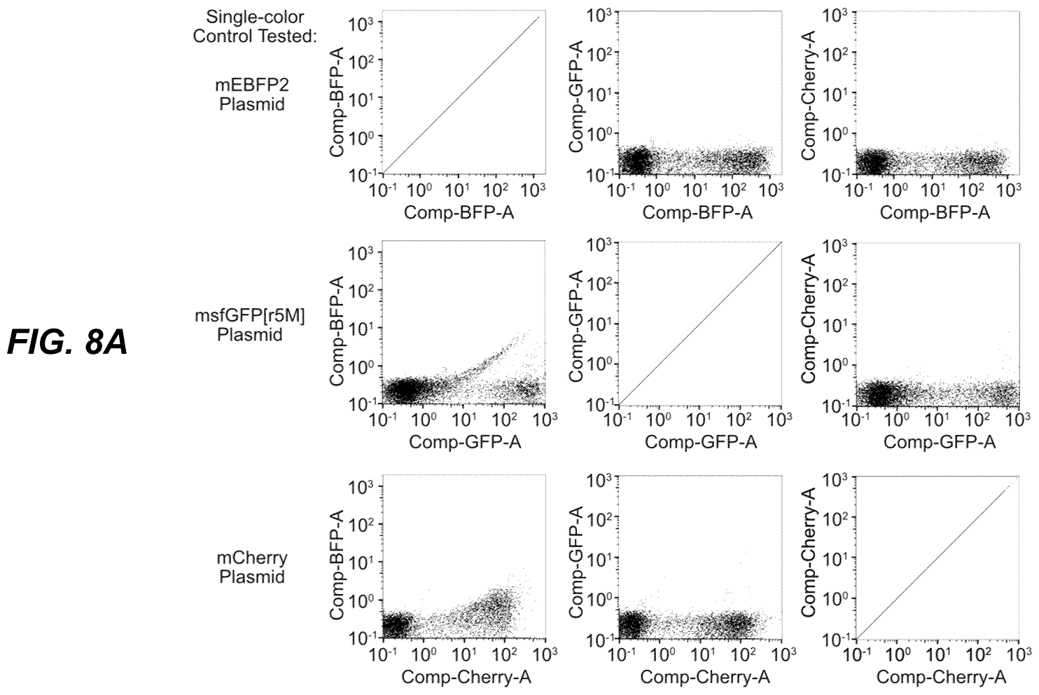

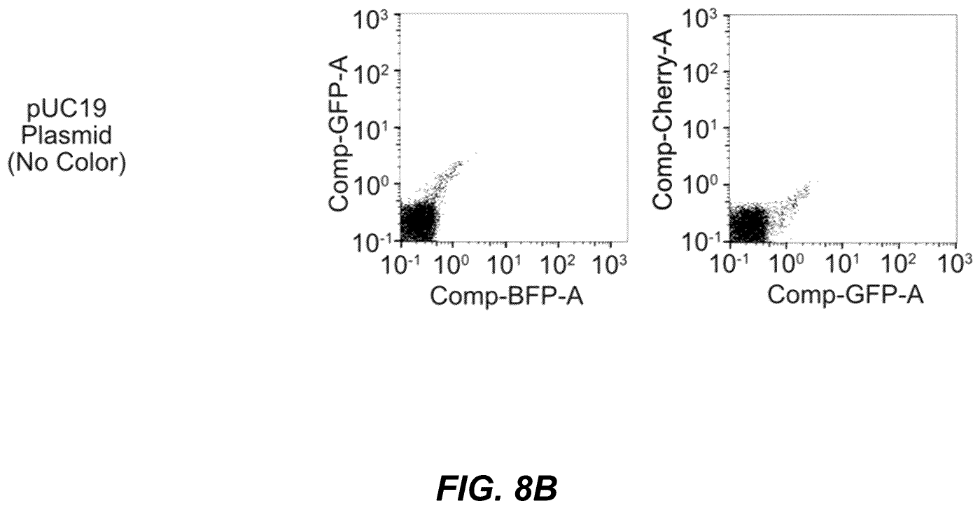

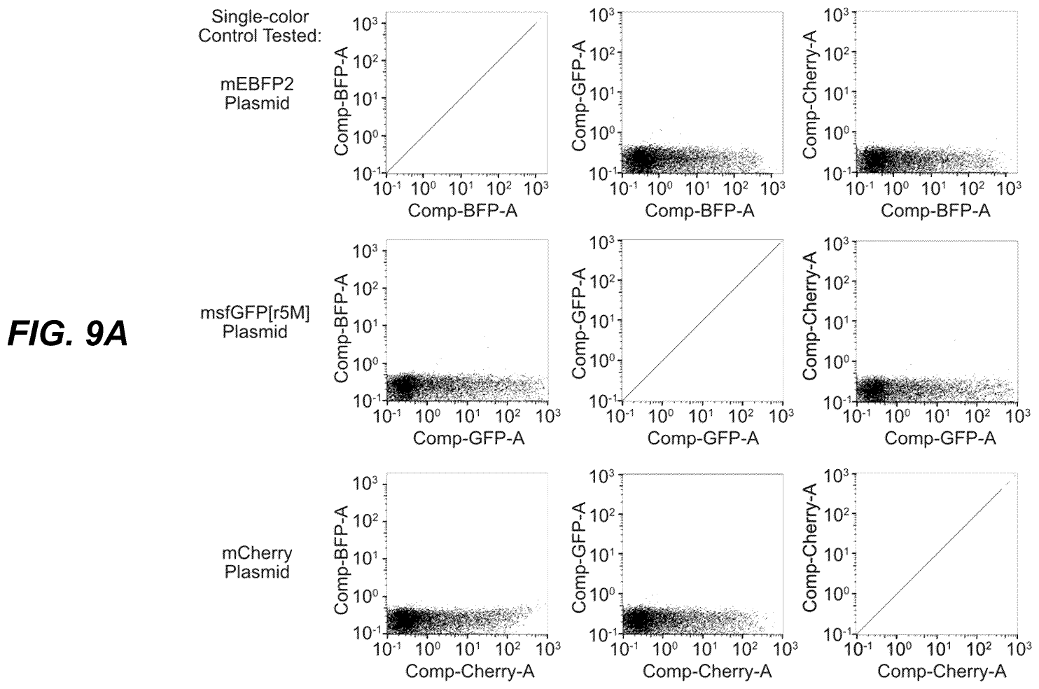

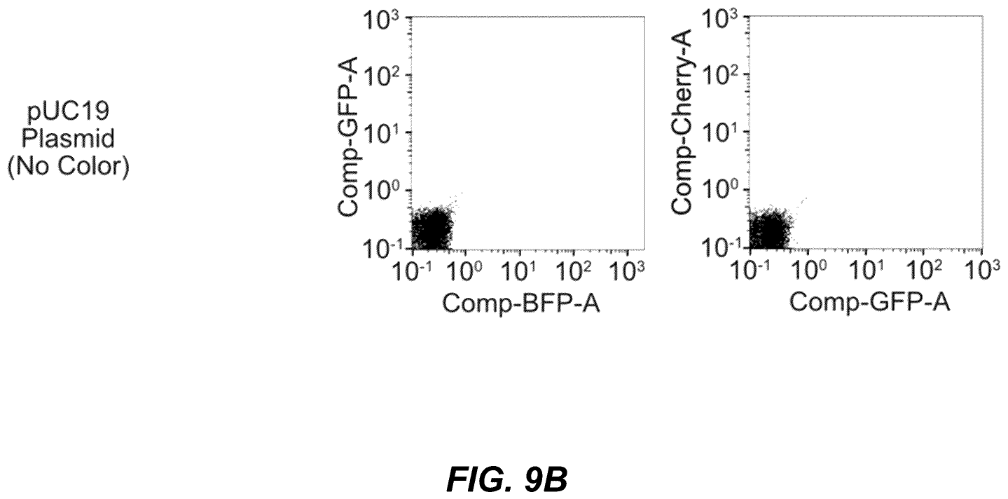

After cloning various combinations of TISs in front of the two ORFs, these “2-ORF SEMPER” constructs were transfected into human HEK293T cells-a widely used research model for mammalian cell biology. Three single-color control plasmids with msfGFP [r5M], mEBFP2, and mCherry were also transfected. The transfected cells were screened using flow cytometry, utilizing the single-color control plasmids for compensation and correction of fluorescence spillover emissions (FIGS. 8-9). As hypothesized, the cell lines produced both msfGFP [r5M] and mEBFP2 from single transcripts (FIG. 1C). Strikingly, the tested TIS combinations (TIS for ORF 1/TIS for ORF 2) yielded unique relationships between the fluorescence of the first ORF and that of the second ORF, with the relative expression of the former vs the latter following the strength of the first TIS. To analyze the 2-ORF SEMPER performance as a function of transcript abundance or “copy number”, cells were binned into three categories (low copy, medium copy, and high copy) based on their mCherry fluorescence. This yielded distinct clusters. The means of msfGFP [r5M]/mCherry, mEBFP2/mCherry, and msfGFP [r5M]/mEBFP2 for the cells in each bin were then calculated for each tested TIS combination. Min-max normalization for each ORF was conducted separately using two constructs from the screen (***/ACC and ACC/***). It was reasoned that ***/ACC would produce the maximum amount of mEBFP2 and the minimum amount of msfGFP [r5M], as all 43S PIC flux should bypass the first ORF and initiate translation at the second. Conversely, it was expected ACC/*** to produce the maximum amount of msfGFP [r5M] and the minimum amount of mEBFP2. Conducted for each mCherry bin independently, this analysis enabled us to compare translation levels of a particular FP across various TIS combinations relative to its hypothesized maximum and minimum (FIG. 1D). Pairwise statistical comparisons for these TIS combinations are provided in Table 2.

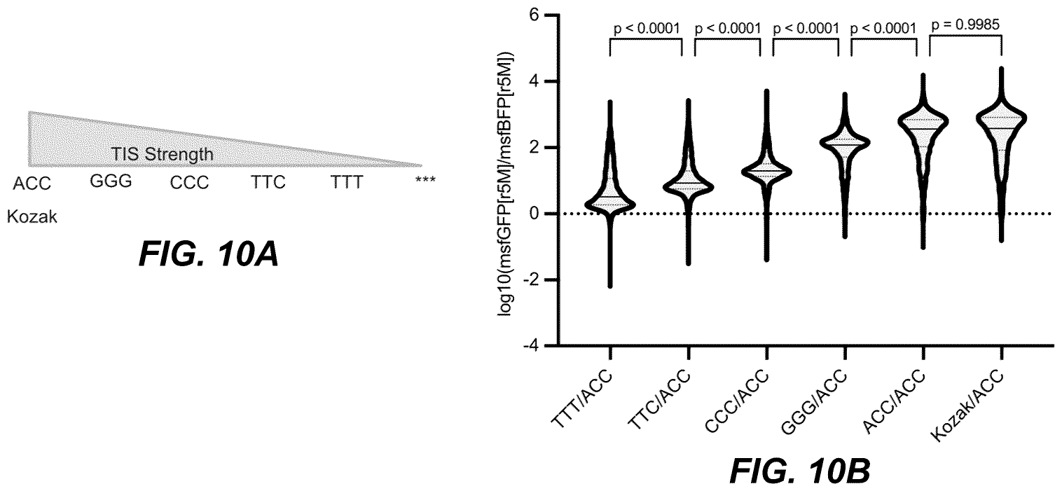

As the TIS strength in front of msfGFP [r5M] was increased, increases in msfGFP [r5M] translation levels and decreases in mEBFP2 translation levels were observed for all mCherry bins. Across mCherry bins, it was found that the rank order of relative translation levels for mEBFP2 for the different TIS combinations was conserved. Additional TIS combinations were also tested in HEK293T cells, showing that they can provide additional degrees of tuning in expression ratios (FIG. 10).

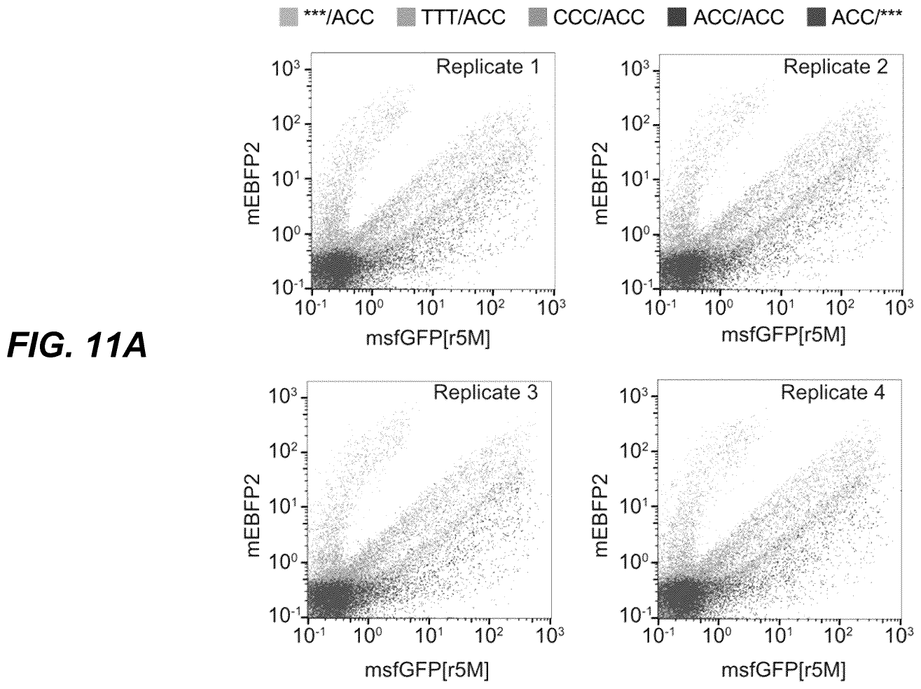

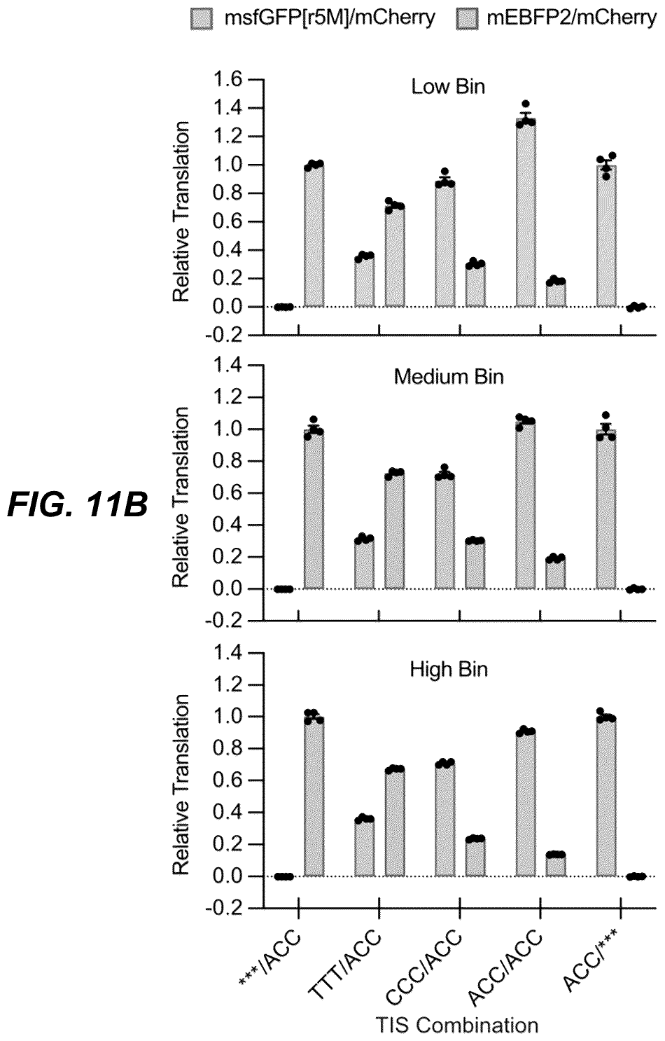

To confirm generalizability across species and cell types, it was also demonstrated that the 2-ORF SEMPER constructs yielded TIS combination-dependent relative translation levels of the ORFs in CHO-KI cells, a widely used Chinese hamster ovary cell line for the production of biologics (FIG. 11, Table 3). Upon comparing the distributions of log 10 (msfGFP [r5M]/mEBFP2) values between cell types (FIG. 1E), it was determined that the three TIS combinations tested maintained the same rank order in both HEK293T and CHO-K1 cell lines.

### Scaling Plasmid-Based SEMPER Constructs to Three ORE's

Enzymatic pathways and macromolecular assemblies are often composed of more than two proteins or peptide subunits. To determine if the SEMPER system could be scaled beyond two ORFs to meet the needs of increasingly complex pathways and assemblies, a variety of “3-ORF SEMPER” constructs were screened. Following a similar strategy to the 2-ORF system, a methionine-less monomeric Superfolder BFP (msfBFP [r5M]) was first cloned and tested.

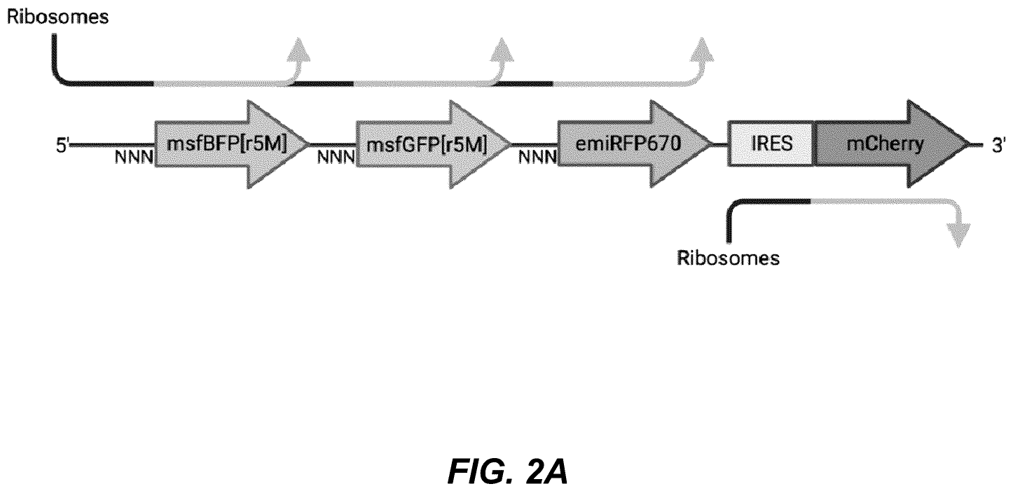

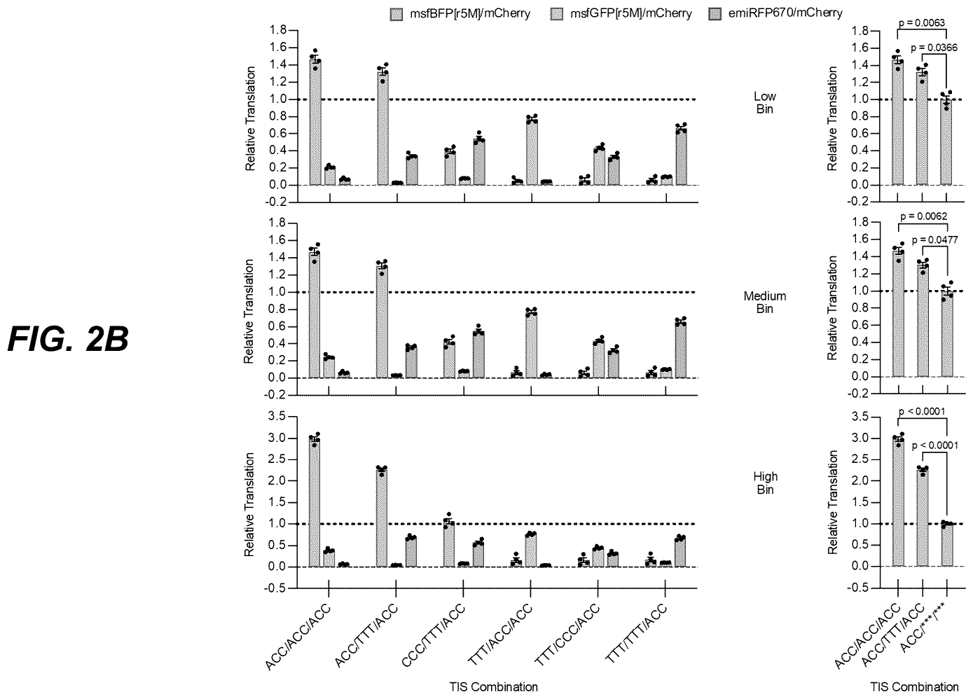

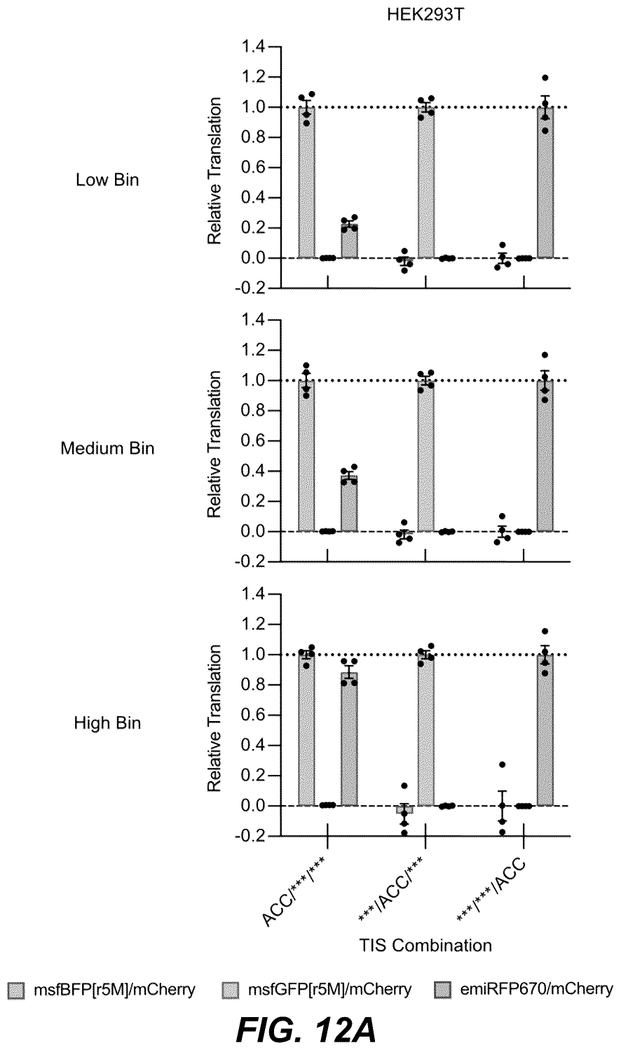

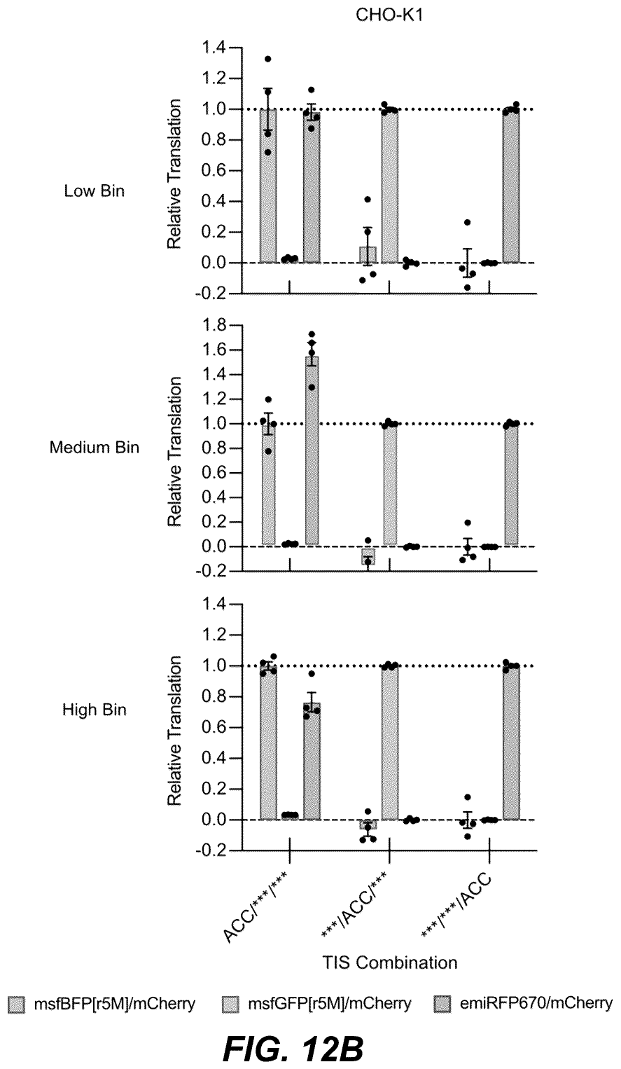

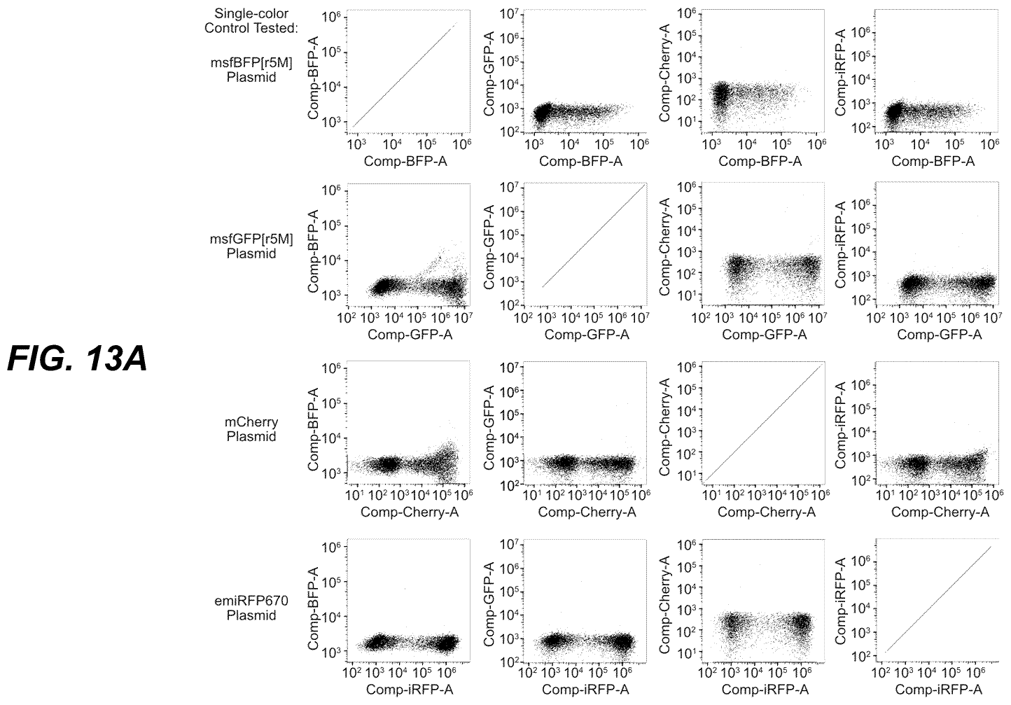

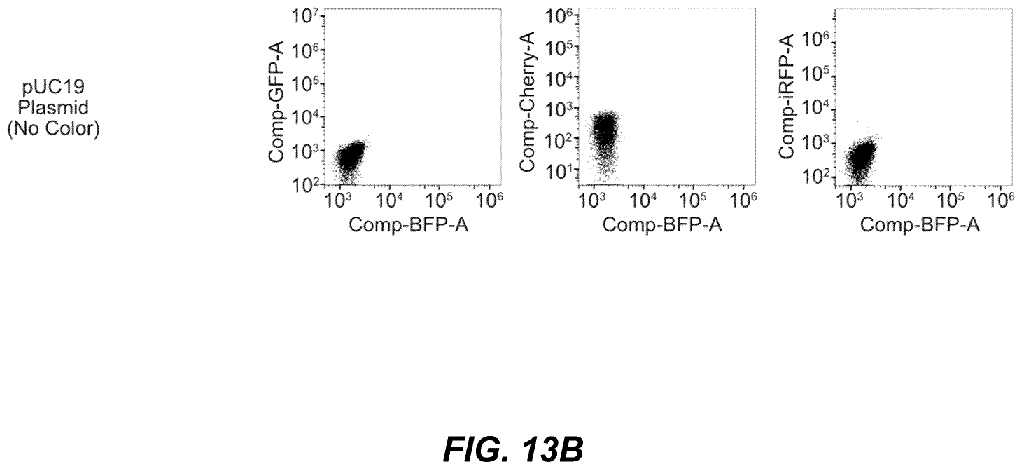

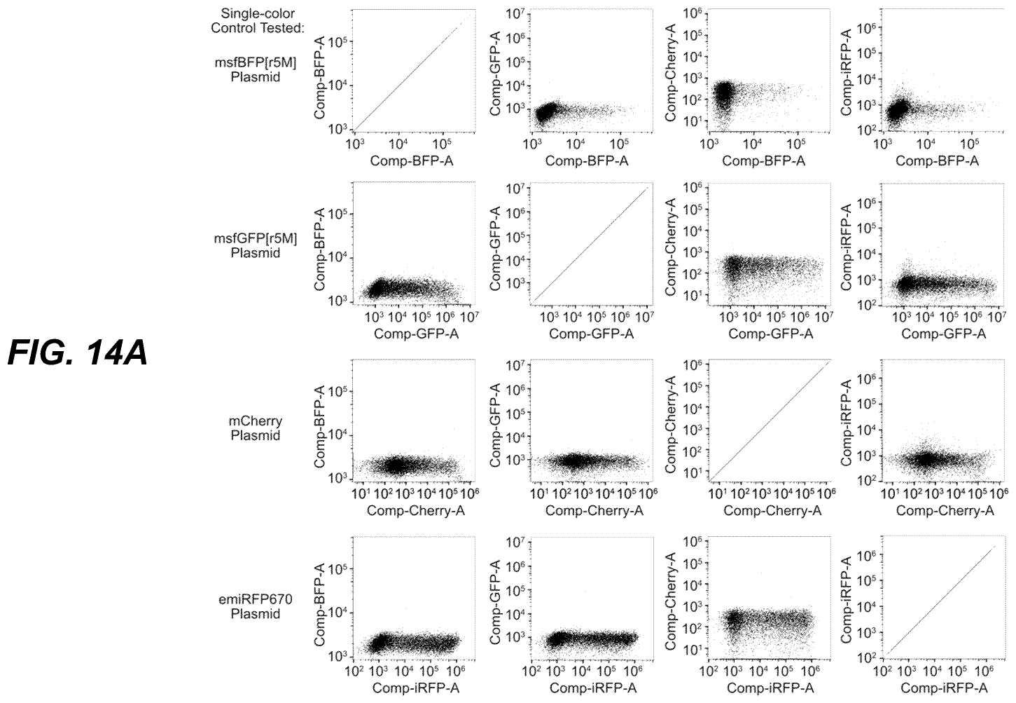

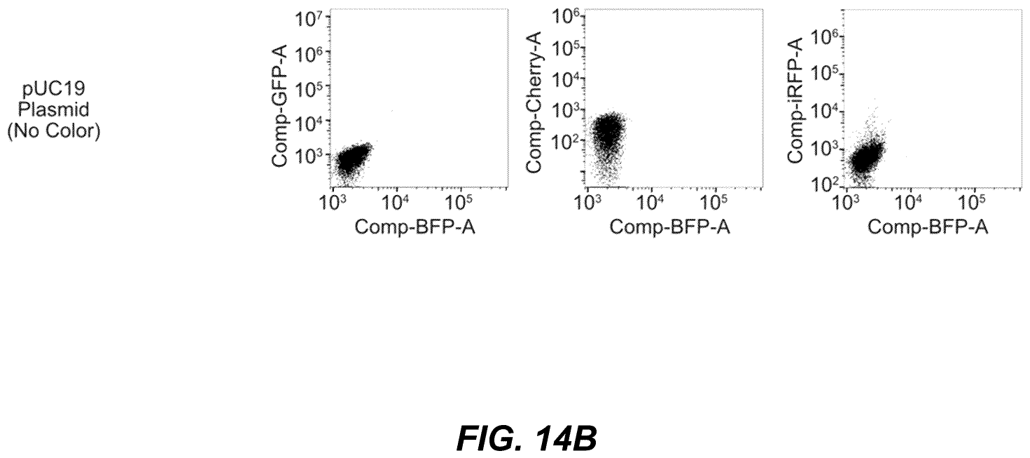

Next msfBFP [r5M], msfGFP [r5M], and emiRFP670-a far-red fluorescent protein-were then encoded into a variety of 3-ORF SEMPER plasmids, maintaining IRES-mCherry for normalization (FIG. 2A). To conduct min-max normalization, three plasmids were cloned where the starting TISs were removed from two of the ORFs using the *** sequence, while maintaining a strong TIS on the third: i) */**/ACC, ii) **/ACC/***, iii) ACC/**/* (FIG. 12). The emiRFP670 coding sequence still encoded alternative translation initiation sites within it, yielding some observable far-red fluorescence in constructs ii) and iii). Additionally, six other 3-ORF SEMPER plasmids with unique TIS combinations were constructed and transiently transfected into HEK293T cells and measured their output using flow cytometry. Four single-color controls (msfBFP [r5M], msfGFP [r5M], emiRFP670, and mCherry) were used for fluorescence compensation (FIGS. 13-14). Following an mCherry binning strategy similar to that employed for 2-ORF SEMPER constructs, the mCherry-normalized translation levels of the three ORFs were analyzed relative to their own theoretical maxima and minima from i), ii), iii). With the tested TIS combinations, a wide range of relative translation levels for each ORF was achieved, demonstrating tunable co-expression of three genes from a single transcriptional unit (FIG. 2B, Left).

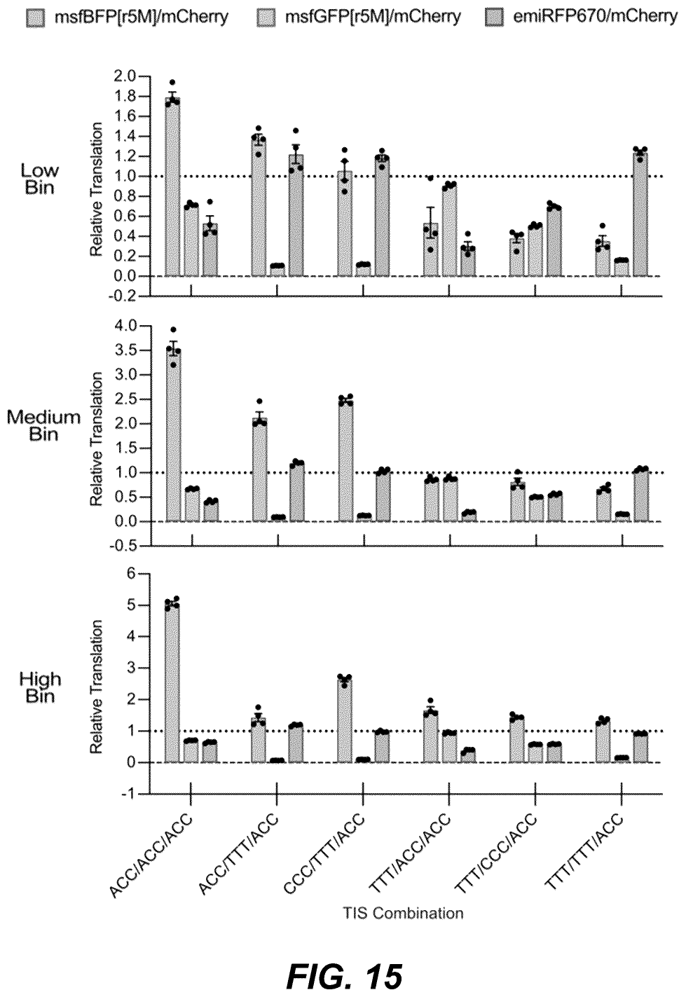

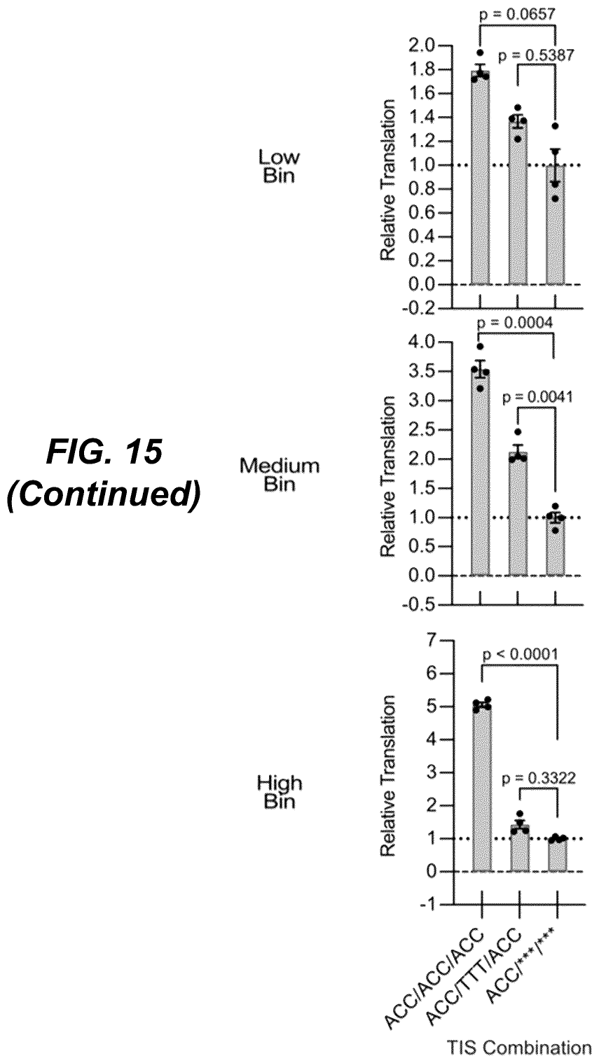

Interestingly, two combinations, ACC/TTT/ACC and ACC/ACC/ACC, yielded relative translation levels for msfBFP [r5M] greater than 1.0 in HEK293T cells across all mCherry bins (FIG. 2B, Right). It was also observed relative translation values for msfBFP [r5M] greater than 1.0 in CHO-KI cells for TIS combinations in both medium and high bins (FIG. 15, Table 5). Consistent with observations herein, Wu et al. previously reported that the presence of downstream ORFs can increase the translation of upstream ones. It was hypothesize that constructs containing multiple ACC TISs have a higher prevalence of translating ribosomes, allowing these ribosomes and subunits—with their documented helicase activity—to maintain the mRNA secondary structure in an open conformation that is more amenable for ribosomal scanning, translation initiation, and subsequent protein expression. However, more work is required to uncover the exact mechanisms that elicit this observed phenotype. For multiple TIS combinations, it was found that the second and third ORFs achieved relative translation levels greater than 50% while still producing substantial levels of upstream ORF products. These results suggest there may be sufficient ribosomal flux to effectively scale the SEMPER paradigm to 4+ ORFs.

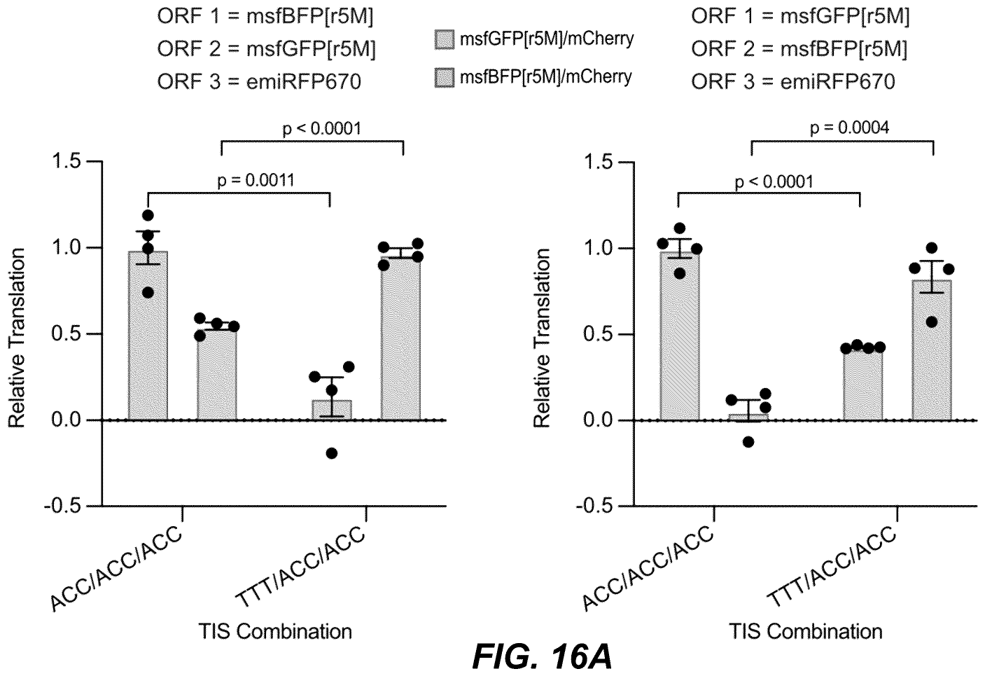

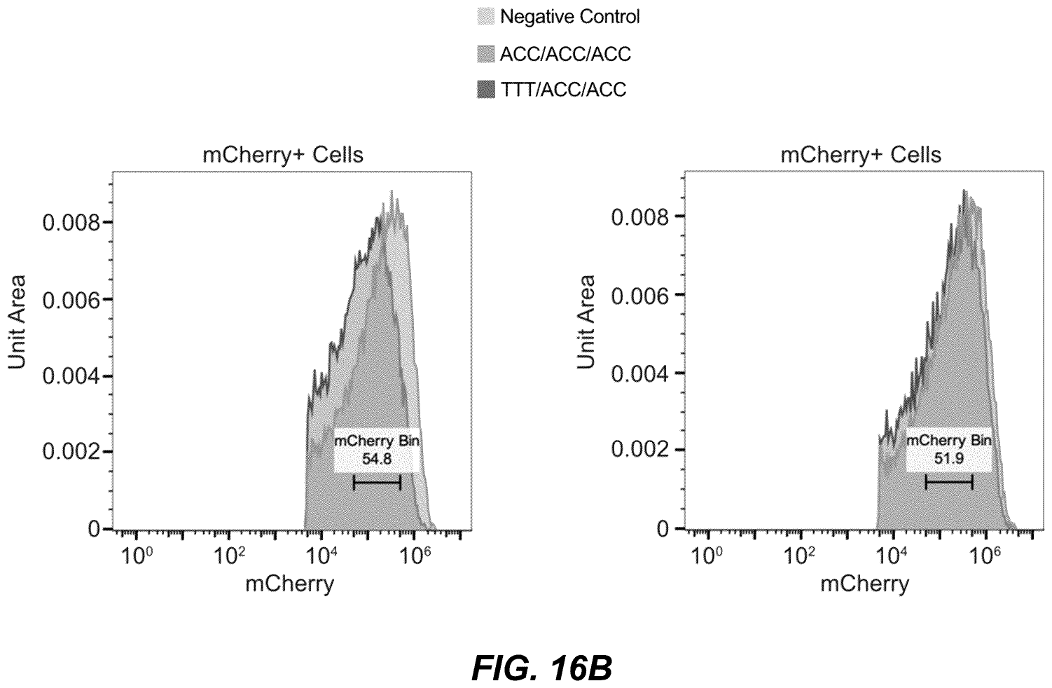

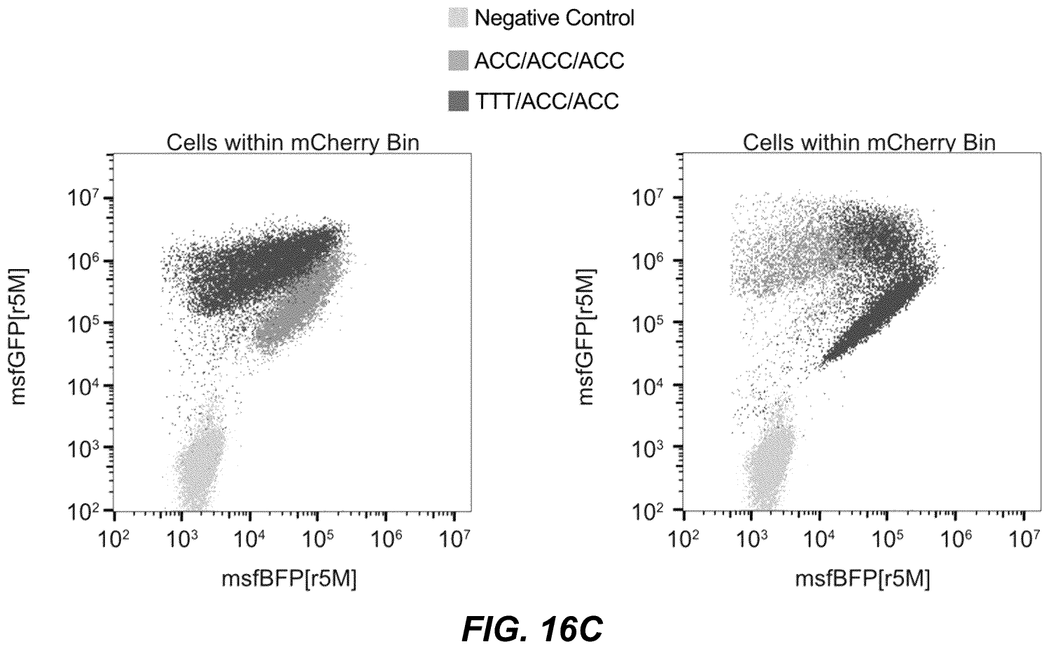

Based on the SEMPER mechanism, the relative expression of ORFs is expected to be tunable regardless of their order in the construct, although the exact expression levels could be affected by any resulting differences in RNA structure and stability. To examine this possibility, the first and second ORFs in the 3-ORF constructs were interchanged and found that the expected rank of relative expression was preserved (FIG. 16).

### Producing Gas Vesicle Ultrasound Reporters Using 2-ORF SEMPER

Next, it was set out to demonstrate that 2-ORF SEMPER constructs could be applied to encoding a multimeric protein complex by expressing gas vesicles (GV) using mammalian acoustic reporter genes (mARGs). Originally evolved in prokaryotes, GVs were recently introduced as genetically-encodable reporters for ultrasound imaging, enabling the noninvasive imaging of dynamic cellular processes in living organisms. A single GV is made up of many GvpA structural units that are assembled together in a helical pattern through the cooperative activity of six heterologous assembly factors and minor constituents, referred to collectively as GvpNJKFGW. Using a two-vector system, this group has previously expressed GVs in mammalian cells by co-transfecting one plasmid encoding the structural unit upstream of IRES-mCherry (pgvpA-IRES-mCherry) along with another plasmid encoding the assembly factors and a terminal Emerald GFP (EmGFP), all linked together by P2A elements (pgvpNJKFGW-EmGFP). Additionally, this group has found that co-transfecting the pgvpA-IRES-mCherry in excess of pgvpNJKFGW-EmGFP improves acoustic contrast. Likewise, Anabaena flos-aquae—the organism from which mARGs used in this study are derived-contains more copies of gvpA relative to the other gvps in its GV gene cluster.

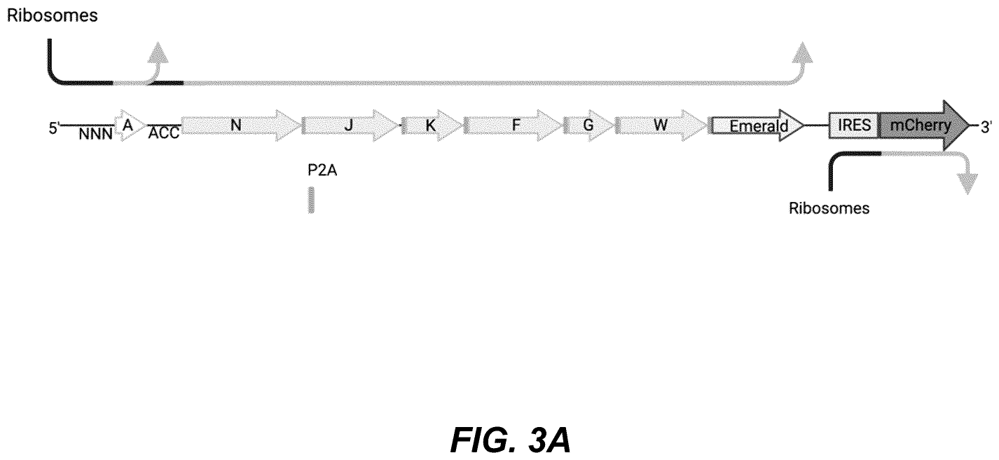

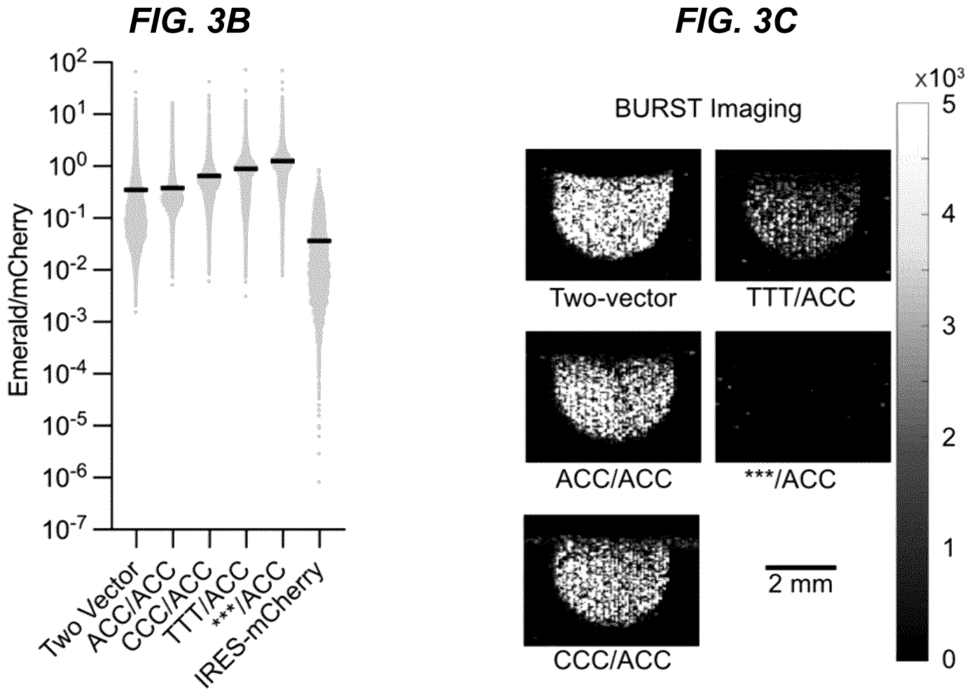

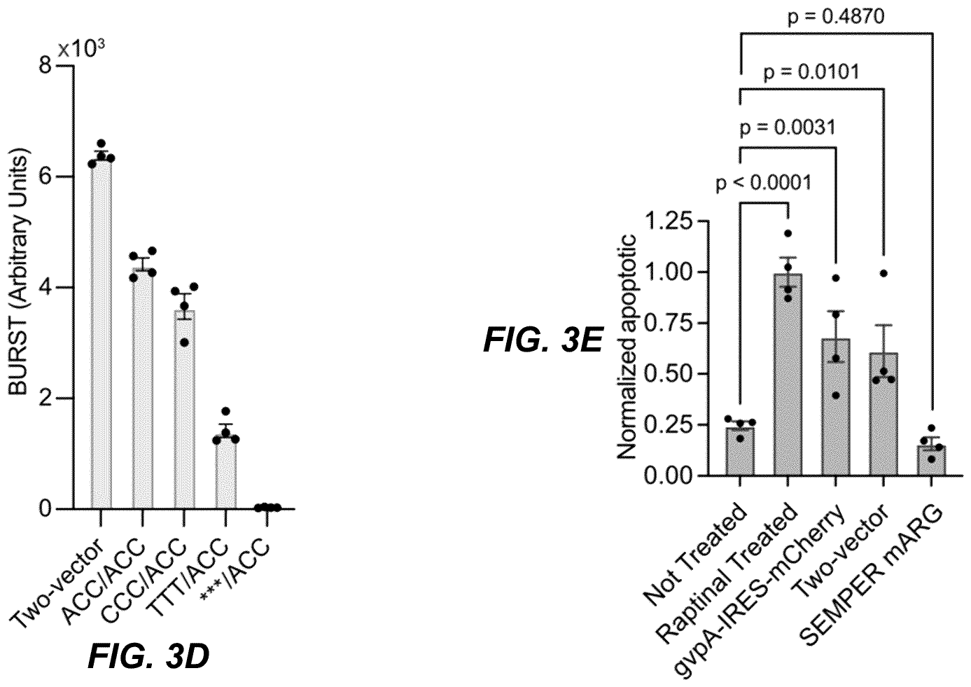

Using the 2-ORF SEMPER strategy, single-vector systems for producing GVs was cloned in mammalian cells. These constructs are referred to herein as SEMPER mARGs. As gvpA does not contain any internal methionines, it was encoded directly into the first ORF. The panel of TIS sequences in front of gvpA was tested while maintaining the strong ACC TIS in front of the gvpNJKFGW-EmGFP ORF (FIG. 3A). These SEMPER constructs were compared to published two-vector expression system, mixing the gvpA-IRES-mCherry plasmid in 4-fold molar excess of the gvpNJKFGW-EmGFP plasmid. These plasmids were transfected into HEK293T cells, maintaining the same total mass of plasmid for each transient transfection. As the strength of the TIS in front of gvpA increased, it was found that the translation level of gvpNJKFGW-EmGFP, as measured by Emerald/mCherry, decreased (FIG. 3B), as expected from the 2-ORF SEMPER FP experiments. As measured by BURST ultrasound imaging, the ACC/ACC combination-predicted to yield the highest ratio of GvpA to GvpNJKFGW-produced the strongest acoustic contrast compared to the other SEMPER mARG plasmids (FIGS. 3C-3D).

### SEMPER Gas Vesicle Expression System Reduces Cell Toxicity

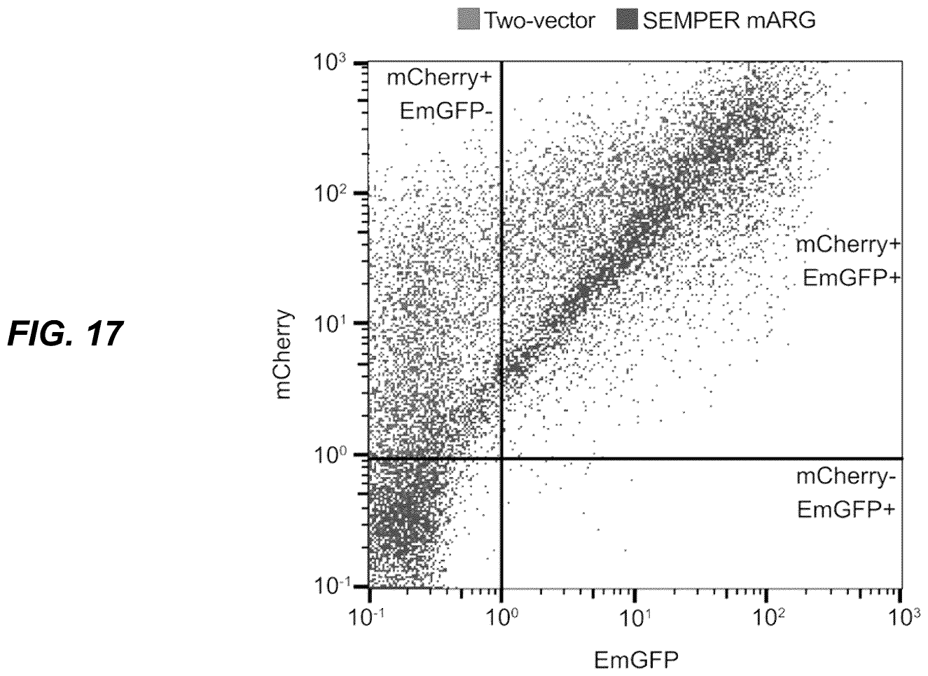

A significant issue with multimeric protein assemblies is the potential for cellular burden or toxicity due to imperfect stoichiometry or the absence of an essential assembly component or chaperone. In the experiments with mARGs, it was observed that samples transfected with the two-vector system contained a larger fraction of cells that were positive for pgvpA-IRES-mCherry but negative for pgvpNJKFGW-EmGFP (39.03%±1.42%) compared to cells transfected with SEMPER mARG plasmids (13.40%±0.85%) (FIG. 17). This is not surprising due to the inherent stochasticity of transient co-transfection. As GvpA subunits have been speculated to nonspecifically aggregate when expressed without GV assembly factors, it was hypothesized that cells receiving a sub-optimal ratio of pgvpA-IRES-mCherry: pgvpNJKFGW-EmGFP or only pgvpA-IRES-mCherry may have higher incidences of apoptosis due to the formation of cytotoxic GvpA aggregates in the cytoplasm.

To test this hypothesis, Annexin V-based apoptosis assays were performed on cells transfected with either pgvpA-IRES-mCherry alone, the two-vector mARG expression system at optimal transfection ratio, or the ACC/ACC SEMPER mARG (FIG. 3E). In each condition, molar amounts of gvpA gene were equalized in each transfection mixture. In addition, pgvpA-IRES-mCherry plasmid without a start codon in front of gvpA was transfected into HEK293T cells to establish a negative control. A subset of these samples was then treated with Raptinal to induce apoptosis, establishing a positive control. Notably, expressing GvpA without its assembly factors led to high levels of apoptosis. While co-transfecting pgvpNJKFGW-EmGFP reduced some of the observed toxicity, the ACC/ACC SEMPER mARG transfected cells were significantly healthier, with apoptosis levels indistinguishable from untreated negative controls. Taken together, these results suggest that the SEMPER mARG plasmid reduces cell toxicity by ensuring that assembly factors are consistently co-expressed with structural proteins in an appropriate ratio.

SEMPER Enables Recombinant Monoclonal Antibody Expression from a Single Construct

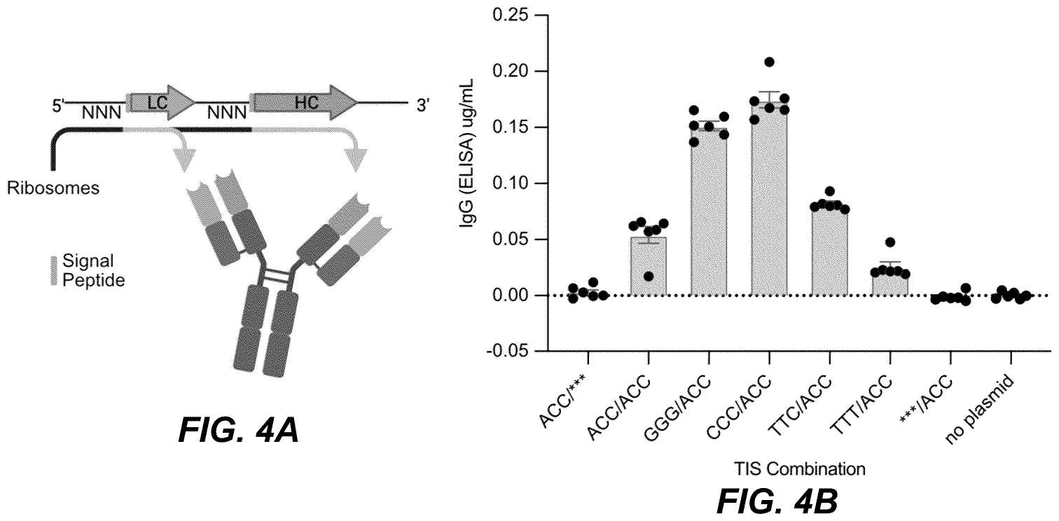

As a further demonstration of the generalizability and biotechnological utility of SEMPER, the system was used to express secreted recombinant monoclonal antibodies in HEK293T cells. As a model construct, the b12 antibody was used, which was discovered as part of early efforts to identify broadly neutralizing antibodies against HIV-1. Contrary to the traditional approaches, the SEMPER-b12 plasmid was designed to express both the heavy and the light chains of the b12 IgG from a single mRNA transcript with tunable heavy chain (HC) to light chain (LC) stoichiometry (FIG. 4A). IgG production was quantified from a sample of media supernatant via an enzyme-linked immunosorbent assay (ELISA).

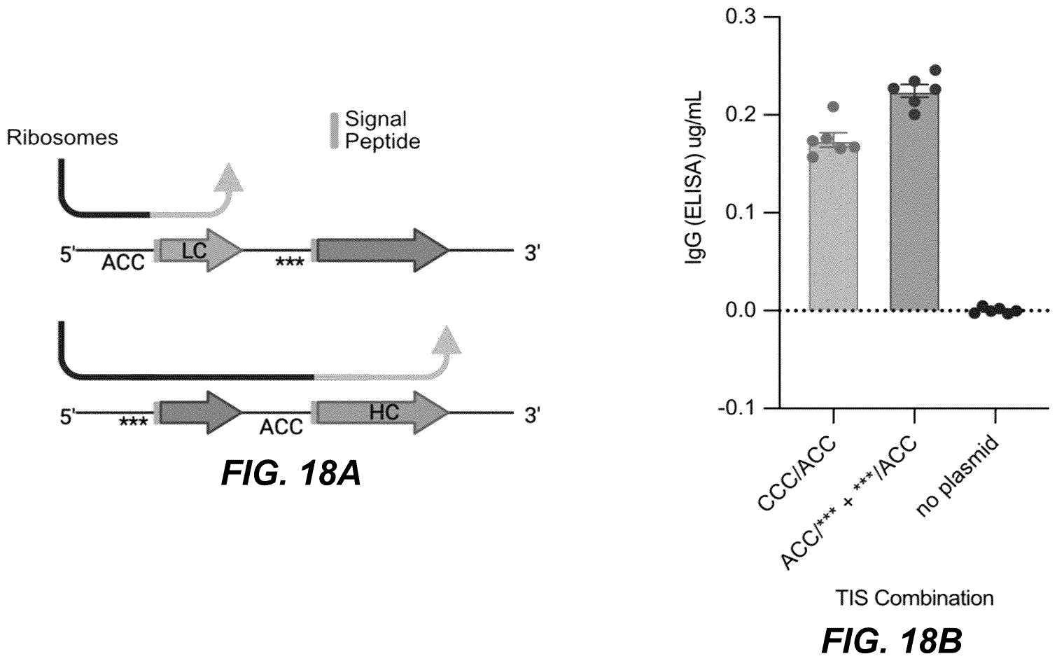

The SEMPER system was able to produce high levels of functional IgG due to the ability to tune the ratio of HC to LC expression, reaching a maximum using the CCC/ACC SEMPER-b12 construct (FIG. 4B). This production level was similar to that achieved by co-transfecting two separate plasmids (FIG. 18). This finding demonstrates the ability of SEMPER to streamline the production of a valuable biologic product by simplifying the genetic engineering required for complex protein expression, while highlighting the importance of being able to tune component protein stoichiometry.

Demonstrating SEMPER 2-ORF Efficacy from Transfected Synthetic mRNA

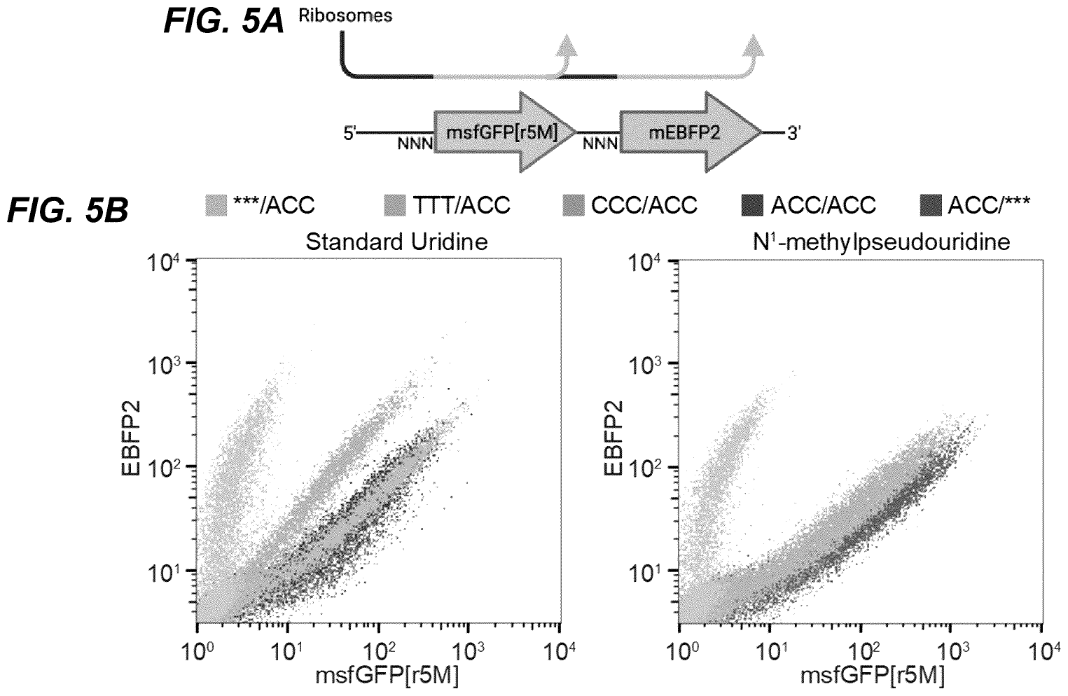

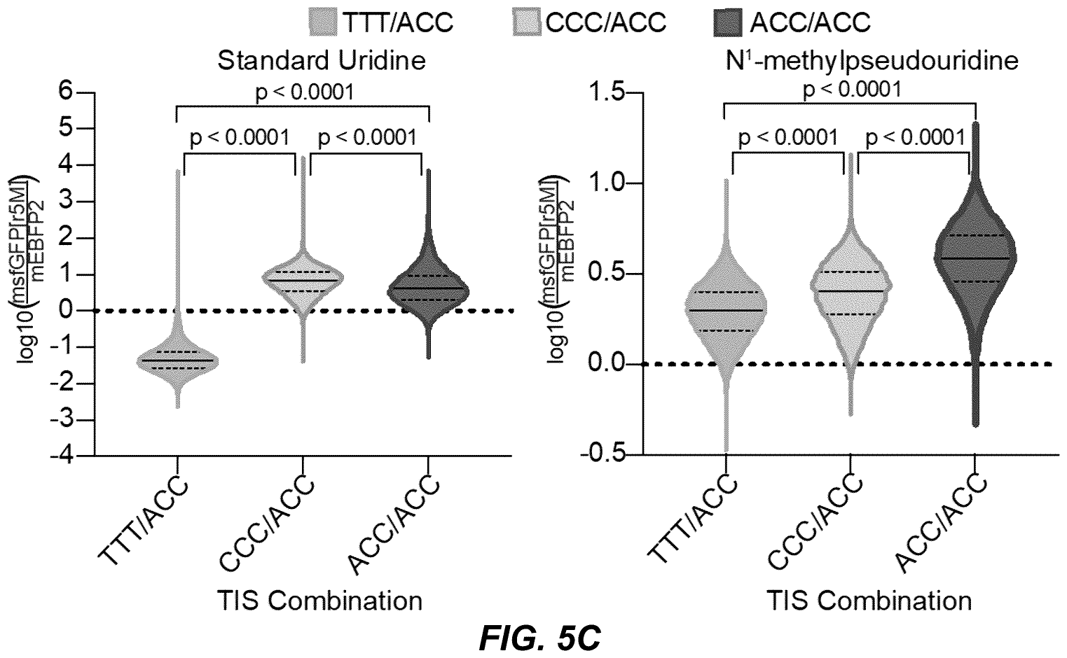

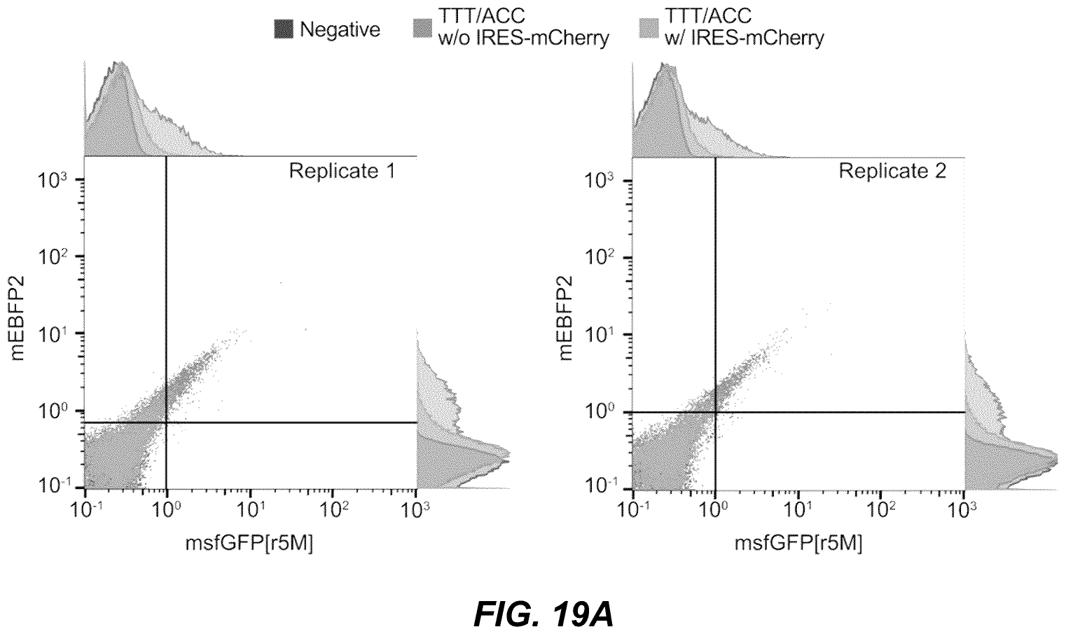

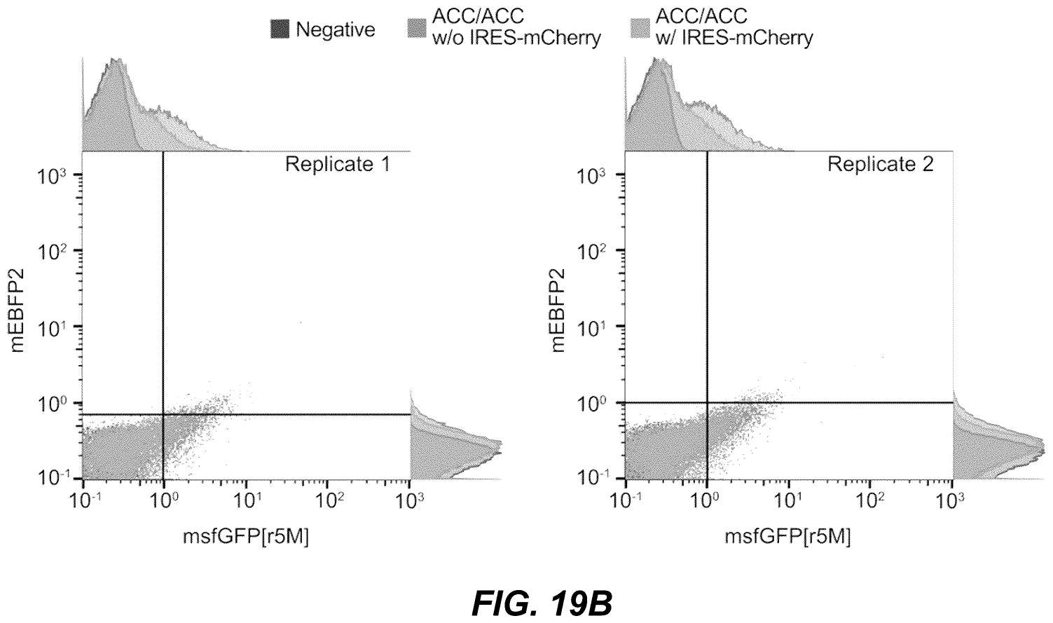

With accelerating advancements in mRNA vaccine technology and growing development efforts in mRNA-based therapeutics, next it was sought to demonstrate the efficacy of the SEMPER 2-ORF system to tune relative protein expression stoichiometries using mRNA-based expression systems (FIG. 5A). The in vitro transcribed (IVT) mRNA constructs employed do not include IRES-mCherry, as reduced expression of disclosed GOIs in IVT mRNA constructs containing this element was observed (FIG. 19).

Through IVT and subsequent purification steps, concentrated mRNA samples with the same TIS combinations and ORFs as tested for the plasmid-based 2-ORF SEMPER constructs were produced. As nucleotide chemistry has been shown to impact translation efficiency, it was hypothesized that changing the nucleotide chemistry of the IVT mRNA may lead to changes in expression of the two ORFs. Therefore, a set of mRNA samples was created using standard uridine triphosphate (UTP) nucleotides in the IVT reaction, while another set was made using N1-methylpseudouridine-5′-triphosphate (m1Ψ), commonly used to manufacture mRNA for clinical use. These mRNA samples were then transiently transfected into HEK293T cells and screened them using flow cytometry. Additionally, transfection and collection of flow cytometry data for two plasmid-based single-color controls for compensation was performed.

Flow cytometry showed that some TIS combinations yielded unique relationships between msfGFP [r5M] and mEBFP2 fluorescence (FIGS. 5B-5C). When using standard UTPs, the relationship for TTT/ACC was significantly different than those of CCC/ACC and ACC/ACC, demonstrating that multiple product stoichiometries are accessible from IVT mRNA SEMPER constructs. However, the specific expression ratios for CCC/ACC and ACC/ACC were not as distinct as those achieved using plasmid constructs, suggesting that further IVT-specific tuning is required, potentially in combination with additional mRNA engineering to improve expression efficiency (FIG. 19). In mRNA constructs produced with m1Ψ, the mEBFP2 vs msfGFP [r5M] relationships for TTT/ACC, CCC/ACC, and ACC/ACC were less distinct, confirming that nucleotide chemistry impacts translation initiation. The differences observed in TTT/ACC samples between the standard UTP and m1Ψ conditions directly suggest that the incorporation of m1Ψ over standard uridine increases the translation initiation strength of the TTT TIS, which is encoded by UUU at the mRNA-level.

### Basic Performance of the SEMPER System is Captured by a Simple Monte Carlo Model

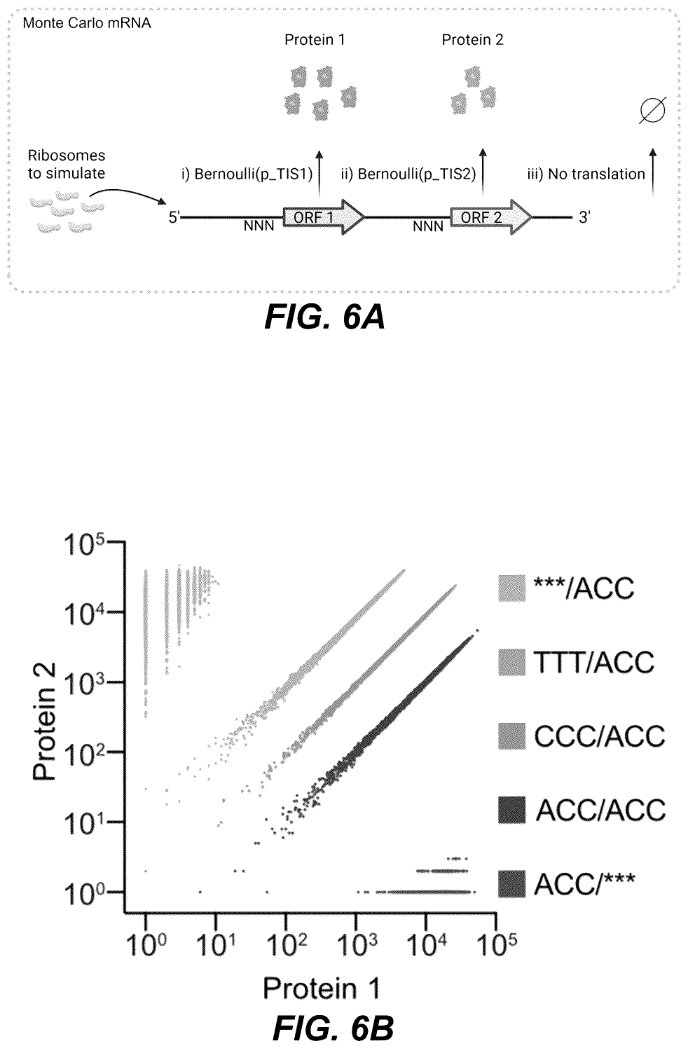

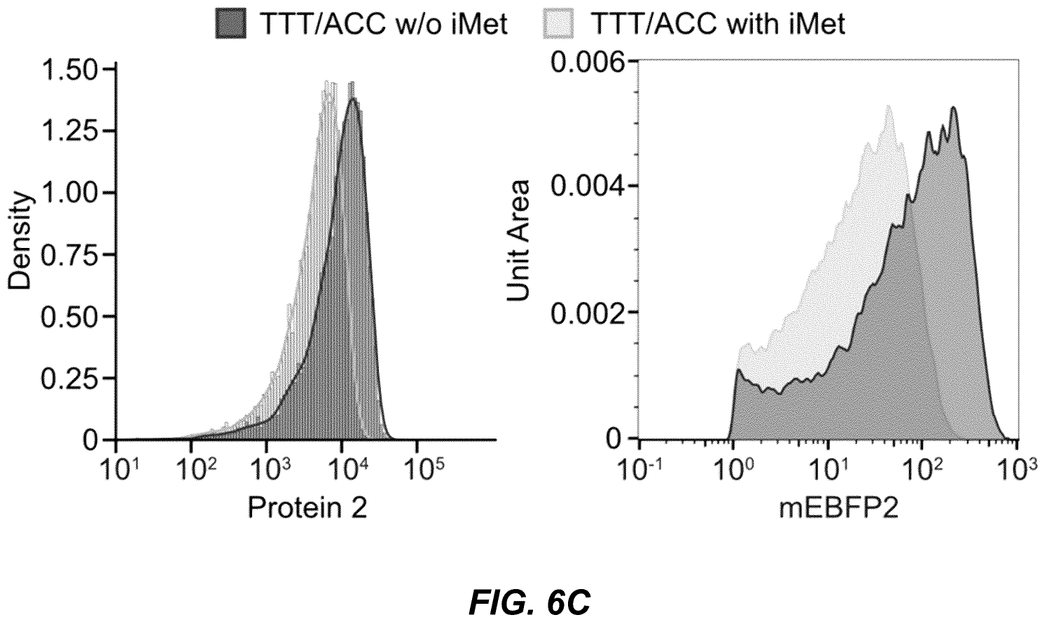

To provide insights on the essential SEMPER mechanism and facilitate future construct design, a simple Monte Carlo simulation was developed that captures the probabilistic nature of tunable, multi-ORF expression based on the LRS model. Briefly, for each Monte Carlo mRNA simulation, the k^{th} scanning ribosome can be consumed by one of three cases (i, ii, iii) as it traverses from the 5′ end to the 3′ end of the simulated transcript (FIG. 6A). Implemented using sequential Boolean statements, the k^{th} scanning ribosome will first encounter i) a TIS in front of the first ORF. A Bernoulli random variable is then sampled with success probability p_TIS1 dictated by the strength of the TIS. This is equivalent to flipping a biased coin. If the instance of the random variable denotes successful initiation, the ribosome translates the first ORF, producing a single unit of Protein 1 and the simulation moves on to load the next k+1 scanning ribosome. However, if the instance of the random variable denotes non-initiation, the k^{th} scanning ribosome moves on to ii) the TIS sequence in front of the second ORF. Similarly, by sampling a Bernoulli random variable with success probability p_TIS2, the scanning ribosome will either translate a single unit of Protein 2, advancing the simulation to the k+1 iteration, or it will fail to initiate translation. In the absence of initiation, the k^{th} scanning ribosome is considered to iii) produce no translation product and the simulation moves on to load the next k+1 scanning ribosome. The aggregate totals of Protein 1 and Protein 2 from each simulated mRNA were then stored. The model does not implement IRES-mCherry expression as this mCherry was only used for experimental normalization.

p_TIS success probabilities for ***, TTT, CCC, and ACC were chosen as 0.0001, 0.1, 0.5, and 0.9 respectively (See Model Note below). p_TIS for *** was chosen to be nonzero to represent minute translation of functional protein initiated at in-frame, non-canonical start sites. For each TIS combination depicted in FIG. 1, nearly 500,000 Monte Carlo mRNA simulations were conducted (See Model Note below). An average of 100 scanning ribosomes were simulated for each Monte Carlo mRNA simulation. Using standard resampling techniques, the simulated expression results of 100 mRNAs, on average, were then aggregated together into approximately 10,000 “single cells” to model transient transfection and then transcription of plasmid DNA. The results of the Monte Carlo simulations depicted in FIG. 6B capture the major observed expression differences seen in FIG. 1C caused by changing the TIS in front of the first ORF.

To extend the model's ability to capture practical design rules, results were simulated for a TTT/ACC construct containing one internal methionine (iMet) within ORF 1. As seen in FIG. 1B, internal methionines within ORF 1 reduced the flux of scanning ribosomes to ORF 2, thus shifting the expression of Protein 2 (mEBFP2) to lower values. To implement this in the simulation, an additional TIS (with p_TIS=0.5) was added between ORF 1 and ORF 2 in the model to represent an iMet within ORF 1. As expected, the presence of an iMet shifted the expression distribution of Protein 2 to lower values (FIG. 6C, Left). For experimental validation, one canonical methionine was added back into the msfGFP [r5M] CDS, producing msfGFP [r4M]. The TIS created by the inclusion of this iMet was predicted to have a TIS strength similar to that of CCC based on quantitative results and modeling conducted by Noderer et al. In HEK293T cells, two TTT/ACC plasmids were then compared, both of which contained mEBFP2 in ORF 2, while ORF 1 was varied between msfGFP [r5M] and msfGFP [r4M]. Consistent with modeling, the construct encoding msfGFP [r4M] yielded less mEBFP2 expression compared to the one without iMet (FIG. 6C, Right), although differences in the magnitude of this effect suggest that future optimization of p_TIS values and other parameters in the model are needed to improve its quantitative prediction accuracy. For more detailed descriptions of model architecture, design choices, parameters, and assumptions, see Model Note below.

### DISCUSSION

These results demonstrate that SEMPER enables tunable expression of multiple recombinant ORFs from single transcripts in mammalian cells by using leaky ribosomal scanning and TISs of varying strength. The SEMPER paradigm is applicable across cells from multiple species and can yield a range of user-tunable expression stoichiometries for at least three proteins on a single compact mRNA. These results with SEMPER mARGs in GV production exemplify its application in encoding complex multi-gene constructs, allowing for precise modulation of expression ratios to optimize output while reducing cellular toxicity. Such tunability can have similar value in other scenarios demanding specific subunit/chaperone or toxin/antitoxin ratios, where co-transfection would result in a wider distribution of ratios. Additionally, the application of SEMPER to secreted mAb expression highlights the method's versatility and utility in therapeutic production. These results also show that the SEMPER framework can be applied directly to mRNA-based genetic circuits by demonstrating polycistronic expression with 2-ORF SEMPER mRNA constructs using IVT. Furthermore, the results show that nucleotide chemistry can alter the translation levels of encoded ORFs in IVT mRNA, opening new avenues to further tune protein stoichiometries with synthetic mRNA. The basic performance of SEMPER is captured by a relatively simple Monte Carlo model, which will help elucidate core design rules for future users.

While SEMPER has demonstrated tunability and rank-order relationships between TIS sets across multiple cell types, gene delivery methods, and protein outputs, the precise expression levels are context-dependent. Differences in these levels may arise from complex gene expression dynamics influenced by factors such as ORF length, RNA secondary structure and RNA processing. Nevertheless, the consistent rank order of expression ratio maintained across the set of TIS combinations tested in this work provides a convenient starting point for construct design, necessitating only limited empirical screening (which is required in most other synthetic biology approaches) in some embodiments and is described herein. The ability of SEMPER to provide tunability of polycistronic expression within a defined range is supported by the disclosed experiments with diverse ORFs, including fluorescent proteins, prokaryotic GVs, and the heavy and light chains of recombinant IgG.

The translation-based stoichiometry control provided by SEMPER complements transcription-based approaches that control relative expression using synthetic promoters and programmable transcriptional systems. SEMPER's translation-focused approach allows tuning to be more compact and encodable at both DNA and mRNA levels. Combining the two classes of methods would enable the construction of gene circuits with greater complexity if needed for synthetic biology applications. SEMPER can also be combined with advanced circuits designed to overcome resource limitation and DNA copy number variability. The genetic constructs described in this work should be immediately useful in a variety of contexts.

SEMPER represents a significant advancement in the field of recombinant multi-protein expression and synthetic biology, enabling compact, user-friendly tuning of multiple proteins within mammalian systems. Researchers can readily adopt this framework to rapidly screen libraries relevant to expression and tuning of enzymatic pathways and circuits, assembly of multimeric protein structures, and other endeavors. As demonstrated herein, this technology can enable simultaneous expression of a protein of interest and its folding chaperones from a single transcriptional unit at an optimal ratio, offering a novel strategy to tackle protein misfolding precisely. Moreover, as shown in some of the constructs used in this study, SEMPER can be used in combination with IRES and 2A elements for versatile encoding of more complex genetic constructs.

SEMPER also holds potential in the field of RNA vaccines and therapeutics. It can enhance the development of polyvalent RNA vaccines as well as the expression of mosaic virus-like particles from delivered mRNA, thereby broadening the immune response against highly variable viruses. Further, it can improve RNA-delivered monoclonal antibody therapeutics by optimizing the ratio of heavy and light chain production in each cell, and by allowing for simultaneous production of multiple or bi-specific antibodies from a single mRNA. This technology also enables production of cytokine cocktails through the co-expression of multiple cytokines from a single mRNA, which can offer synergistic effects to modulate immune responses. Taken together, SEMPER provides an option that can always be considered in developing polycistronic constructs for synthetic biology and medicine.

TABLE 1A
Post hoc Games-Howell's multiple comparisons test for 2-ORF
HEK293T mCherry distributions (Referenced in FIG. 7A)

| Games-Howell's multiple | | | |
|---|---|---|---|
| comparisons test | Adjusted P Value | n1 | n2 |
| ***/ACC vs. ACC/*** | <0.0001 | 64886 | 23934 |
| ***/ACC vs. ACC/ACC | <0.0001 | 64886 | 72256 |
| ***/ACC vs. CCC/ACC | 0.0044 | 64886 | 74879 |
| ***/ACC vs. TTT/ACC | <0.0001 | 64886 | 81383 |
| ACC/*** vs. ACC/ACC | <0.0001 | 23934 | 72256 |
| ACC/*** vs. CCC/ACC | <0.0001 | 23934 | 74879 |
| ACC/*** vs. TTT/ACC | <0.0001 | 23934 | 81383 |
| ACC/ACC vs. CCC/ACC | <0.0001 | 72256 | 74879 |
| ACC/ACC vs. TTT/ACC | <0.0001 | 72256 | 81383 |
| CCC/ACC vs. TTT/ACC | <0.0001 | 74879 | 81383 |

TABLE 1B
Post hoc Games-Howell's multiple comparisons test for
2-ORF CHO-K1 mCherry distributions (Referenced in FIG. 7B)

| Games-Howell's multiple comparisons | | | |
|---|---|---|---|
| test | Adjusted P Value | n1 | n2 |
| **/ACC vs. ACC/*** | <0.0001 | 32253 | 12605 |
| ***/ACC vs. ACC/ACC | <0.0001 | 32253 | 15042 |
| ***/ACC vs. CCC/ACC | 0.0294 | 32253 | 32967 |
| ***/ACC vs. TTT/ACC | 0.7558 | 32253 | 35457 |
| ACC/*** vs. ACC/ACC | <0.0001 | 12605 | 15042 |
| ACC/*** vs. CCC/ACC | <0.0001 | 12605 | 32967 |
| ACC/*** vs. TTT/ACC | <0.0001 | 12605 | 35457 |
| ACC/ACC vs. CCC/ACC | <0.0001 | 15042 | 32967 |
| ACC/ACC vs. TTT/ACC | <0.0001 | 15042 | 35457 |
| CCC/ACC vs. TTT/ACC | 0.0001 | 32967 | 35457 |

TABLE 1C
Post-hoc Games-Howell's multiple comparisons test for
3-ORF HEK293T mCherry distributions (Referenced in FIG. 7C)

| Games-Howell's multiple comparisons | | | |
|---|---|---|---|
| test | Adjusted P Value | n1 | n2 |
| ***/***/ACC vs. ***/ACC/*** | <0.0001 | 91946 | 79348 |
| ***/***/ACC vs. ACC/***/*** | <0.0001 | 91946 | 67093 |
| ***/***/ACC vs. ACC/ACC/ACC | <0.0001 | 91946 | 73751 |
| ***/***/ACC vs. ACC/TTT/ACC | <0.0001 | 91946 | 72039 |
| ***/***/ACC vs. CCC/TTT/ACC | <0.0001 | 91946 | 71607 |
| ***/***/ACC vs. TTT/ACC/ACC | <0.0001 | 91946 | 81700 |
| ***/***/ACC vs. TTT/CCC/ACC | <0.0001 | 91946 | 86621 |
| ***/***/ACC vs. TTT/TTT/ACC | <0.0001 | 91946 | 59893 |
| ***/ACC/*** vs. ACC/***/*** | <0.0001 | 79348 | 67093 |
| ***/ACC/*** vs. ACC/ACC/ACC | <0.0001 | 79348 | 73751 |
| ***/ACC/*** vs. ACC/TTT/ACC | <0.0001 | 79348 | 72039 |
| ***/ACC/*** vs. CCC/TTT/ACC | <0.0001 | 79348 | 71607 |
| ***/ACC/*** vs. TTT/ACC/ACC | <0.0001 | 79348 | 81700 |
| **/ACC/*** vs. TTT/CCC/ACC | <0.0001 | 79348 | 86621 |
| ***/ACC/*** vs. TTT/TTT/ACC | <0.0001 | 79348 | 59893 |
| ACC/***/*** vs. ACC/ACC/ACC | 0.0016 | 67093 | 73751 |
| ACC/***/*** vs. ACC/TTT/ACC | <0.0001 | 67093 | 72039 |
| ACC/***/*** vs. CCC/TTT/ACC | <0.0001 | 67093 | 71607 |
| ACC/***/*** vs. TTT/ACC/ACC | <0.0001 | 67093 | 81700 |
| ACC/***/*** vs. TTT/CCC/ACC | <0.0001 | 67093 | 86621 |
| ACC/***/*** vs. TTT/TTT/ACC | <0.0001 | 67093 | 59893 |
| ACC/ACC/ACC vs. ACC/TTT/ACC | 0.2084 | 73751 | 72039 |
| ACC/ACC/ACC vs. CCC/TTT/ACC | <0.0001 | 73751 | 71607 |
| ACC/ACC/ACC vs. TTT/ACC/ACC | <0.0001 | 73751 | 81700 |
| ACC/ACC/ACC vs. TTT/CCC/ACC | <0.0001 | 73751 | 86621 |
| ACC/ACC/ACC vs. TTT/TTT/ACC | <0.0001 | 73751 | 59893 |
| ACC/TTT/ACC vs. CCC/TTT/ACC | <0.0001 | 72039 | 71607 |
| ACC/TTT/ACC vs. TTT/ACC/ACC | <0.0001 | 72039 | 81700 |
| ACC/TTT/ACC vs. TTT/CCC/ACC | <0.0001 | 72039 | 86621 |
| ACC/TTT/ACC vs. TTT/TTT/ACC | <0.0001 | 72039 | 59893 |
| CCC/TTT/ACC vs. TTT/ACC/ACC | <0.0001 | 71607 | 81700 |
| CCC/TTT/ACC vs. TTT/CCC/ACC | 0.0006 | 71607 | 86621 |
| CCC/TTT/ACC vs. TTT/TTT/ACC | <0.0001 | 71607 | 59893 |
| TTT/ACC/ACC vs. TTT/CCC/ACC | <0.0001 | 81700 | 86621 |
| TTT/ACC/ACC vs. TTT/TTT/ACC | <0.0001 | 81700 | 59893 |
| TTT/CCC/ACC vs. TTT/TTT/ACC | <0.0001 | 86621 | 59893 |

TABLE 1D
Post-hoc Games-Howell's multiple comparisons test for 3-ORF
CHO-K1 mCherry distributions (Referenced in FIG. 7D)

| Games-Howell's multiple | | | |
|---|---|---|---|
| comparisons test | Adjusted P Value | n1 | n2 |
| ***/***/ACC vs. ***/ACC/*** | 0.0017 | 47094 | 22572 |
| ***/***/ACC vs. ACC/***/*** | <0.0001 | 47094 | 5153 |
| ***/***/ACC vs. ACC/ACC/ACC | 0.0058 | 47094 | 17132 |
| ***/***/ACC vs. ACC/TTT/ACC | 0.8506 | 47094 | 14357 |
| ***/***/ACC vs. CCC/TTT/ACC | <0.0001 | 47094 | 26154 |
| ***/***/ACC vs. TTT/ACC/ACC | <0.0001 | 47094 | 23233 |
| ***/***/ACC vs. TTT/CCC/ACC | <0.0001 | 47094 | 29944 |
| ***/***/ACC vs. TTT/TTT/ACC | <0.0001 | 47094 | 36965 |
| ***/ACC/*** vs. ACC/***/*** | <0.0001 | 22572 | 5153 |
| ***/ACC/*** vs. ACC/ACC/ACC | >0.9999 | 22572 | 17132 |
| ***/ACC/*** vs. ACC/TTT/ACC | 0.0001 | 22572 | 14357 |
| ***/ACC/*** vs. CCC/TTT/ACC | 0.9914 | 22572 | 26154 |
| ***/ACC/*** vs. TTT/ACC/ACC | <0.0001 | 22572 | 23233 |
| ***/ACC/*** vs. TTT/CCC/ACC | <0.0001 | 22572 | 29944 |
| ***/ACC/*** vs. TTT/TTT/ACC | 0.0063 | 22572 | 36965 |
| ACC/***/*** vs. ACC/ACC/ACC | <0.0001 | 5153 | 17132 |
| ACC/***/*** vs. ACC/TTT/ACC | <0.0001 | 5153 | 14357 |
| ACC/***/*** vs. CCC/TTT/ACC | <0.0001 | 5153 | 26154 |
| ACC/***/*** vs. TTT/ACC/ACC | <0.0001 | 5153 | 23233 |
| ACC/***/*** vs. TTT/CCC/ACC | <0.0001 | 5153 | 29944 |
| ACC/***/*** vs. TTT/TTT/ACC | <0.0001 | 5153 | 36965 |
| ACC/ACC/ACC vs. ACC/TTT/ACC | 0.0005 | 17132 | 14357 |
| ACC/ACC/ACC vs. CCC/TTT/ACC | 0.9649 | 17132 | 26154 |
| ACC/ACC/ACC vs. TTT/ACC/ACC | <0.0001 | 17132 | 23233 |
| ACC/ACC/ACC vs. TTT/CCC/ACC | <0.0001 | 17132 | 29944 |
| ACC/ACC/ACC vs. TTT/TTT/ACC | 0.0027 | 17132 | 36965 |
| ACC/TTT/ACC vs. CCC/TTT/ACC | <0.0001 | 14357 | 26154 |
| ACC/TTT/ACC vs. TTT/ACC/ACC | <0.0001 | 14357 | 23233 |
| ACC/TTT/ACC vs. TTT/CCC/ACC | <0.0001 | 14357 | 29944 |
| ACC/TTT/ACC vs. TTT/TTT/ACC | <0.0001 | 14357 | 36965 |
| CCC/TTT/ACC vs. TTT/ACC/ACC | <0.0001 | 26154 | 23233 |
| CCC/TTT/ACC vs. TTT/CCC/ACC | <0.0001 | 26154 | 29944 |
| CCC/TTT/ACC vs. TTT/TTT/ACC | 0.1211 | 26154 | 36965 |
| TTT/ACC/ACC vs. TTT/CCC/ACC | 0.2202 | 23233 | 29944 |
| TTT/ACC/ACC vs. TTT/TTT/ACC | <0.0001 | 23233 | 36965 |
| TTT/CCC/ACC vs. TTT/TTT/ACC | <0.0001 | 29944 | 36965 |

TABLE 2
Post-hoc Dunnett's T3 multiple comparisons tests for 2-ORF HEK293T
constructs depicted in FIG. 1D (N = 4 for all conditions)

| | Low Bin | | Medium Bin | | High Bin | |
|---|---|---|---|---|---|---|
| | mEBFP2/ | msfGFP[r5M]/ | mEBFP2/ | msfGFP[r5M]/ | mEBFP2/ | msfGFP[r5M]/ |
| | mCherry | mCherry | mCherry | mCherry | mCherry | mCherry |
| One-way Anova P-value | <0.0001 | <0.0001 | <0.0001 | <0.0001 | <0.0001 | <0.0001 |
| Dunnett's T3 multiple | Adjusted | Adjusted | Adjusted | Adjusted | Adjusted | Adjusted |
| comparisons test | P Value | P Value | P Value | P Value | P Value | P Value |
| ***/ACC vs. TTT/ACC | 0.0047 | 0.0022 | 0.248 | 0.0034 | 0.0253 | 0.0022 |
| ***/ACC vs. CCC/ACC | <0.0001 | 0.01 | 0.0006 | 0.0065 | 0.0004 | 0.0019 |
| ***/ACC vs. ACC/ACC | <0.0001 | 0.0082 | 0.0017 | 0.0026 | 0.0008 | 0.0004 |
| ***/ACC vs. ACC/*** | 0.0005 | 0.0021 | 0.0014 | 0.0029 | 0.0008 | 0.0004 |
| TTT/ACC vs. CCC/ACC | 0.0001 | 0.0373 | 0.0019 | 0.0244 | <0.0001 | 0.002 |
| TTT/ACC vs. ACC/ACC | 0.0001 | 0.016 | 0.0026 | 0.0054 | 0.0005 | 0.0001 |
| TTT/ACC vs. ACC/*** | 0.0005 | 0.0037 | 0.0019 | 0.0064 | 0.0004 | 0.0001 |
| CCC/ACC vs. ACC/ACC | 0.014 | 0.0594 | 0.0213 | 0.0128 | 0.0024 | 0.0006 |
| CCC/ACC vs. ACC/*** | 0.0111 | 0.0025 | 0.0099 | 0.0304 | 0.0017 | 0.0015 |
| ACC/ACC vs. ACC/*** | 0.0369 | 0.5537 | 0.0061 | 0.9652 | 0.0112 | 0.6513 |

TABLE 3
Dunnett's T3 multiple comparisons tests for 2-ORF CHO-K1 constructs
depicted in FIG. 11 (N = 4 for all conditions)

| | Low Bin | | Medium Bin | | High Bin | |
|---|---|---|---|---|---|---|
| | mEBFP2/ | msfGFP[r5M]/ | mEBFP2/ | msfGFP[r5M]/ | mEBFP2/ | msfGFP[r5M]/ |
| | mCherry | mCherry | mCherry | mCherry | mCherry | mCherry |
| One-way ANOVA P-value | <0.0001 | <0.0001 | <0.0001 | <0.0001 | <0.0001 | <0.0001 |
| Dunnett's T3 multiple | Adjusted | Adjusted | Adjusted | Adjusted | Adjusted | Adjusted |
| comparisons test | P Value | P Value | P Value | P Value | P Value | P Value |
| ***/ACC vs. TTT/ACC | <0.0001 | 0.0001 | 0.0022 | 0.0001 | 0.0011 | <0.0001 |
| ***/ACC vs. CCC/ACC | <0.0001 | 0.0002 | 0.0004 | <0.0001 | <0.0001 | <0.0001 |
| ***/ACC vs. ACC/ACC | <0.0001 | 0.0002 | 0.0003 | <0.0001 | <0.0001 | <0.0001 |
| ***/ACC vs. ACC/*** | <0.0001 | 0.0004 | 0.0001 | 0.0004 | <0.0001 | <0.0001 |
| TTT/ACC vs. CCC/ACC | <0.0001 | 0.0001 | <0.0001 | <0.0001 | <0.0001 | <0.0001 |
| TTT/ACC vs. ACC/ACC | <0.0001 | 0.0005 | <0.0001 | <0.0001 | <0.0001 | <0.0001 |
| TTT/ACC vs. ACC/*** | <0.0001 | 0.0017 | <0.0001 | 0.0014 | <0.0001 | <0.0001 |
| CCC/ACC vs. ACC/ACC | 0.0006 | 0.0008 | 0.0002 | <0.0001 | <0.0001 | <0.0001 |
| CCC/ACC vs. ACC/*** | <0.0001 | 0.2331 | <0.0001 | 0.0093 | <0.0001 | 0.0002 |
| ACC/ACC vs. ACC/*** | <0.0001 | 0.0033 | <0.0001 | 0.8013 | <0.0001 | 0.0148 |

TABLE 4
Post-hoc Dunnett's T3 multiple comparisons tests for 3-ORF HEK293T
constructs depicted in FIG. 2 (N = 4 for all conditions)

| | Low Bin | | | Medium Bin | |
|---|---|---|---|---|---|
| | msfBFP[r5M]/ | msfGFP[r5M]/ | emiRFP670/ | msfBFP[r5M]/ | msfGFP[r5M]/ |
| | mCherry | mCherry | mCherry | mCherry | mCherry |
| One-way ANOVA P-value | <0.0001 | <0.0001 | <0.0001 | <0.0001 | <0.0001 |
| Dunnett's T3 multiple | Adjusted | Adjusted | Adjusted | Adjusted | Adjusted |
| comparisons test | P Value | P Value | P Value | P Value | P Value |
| ACC/ACC/ACC vs. | 0.5759 | 0.0027 | 0.0008 | 0.3098 | 0.0036 |
| ACC/TTT/ACC | | | | | |
| ACC/ACC/ACC vs. | <0.0001 | 0.0075 | 0.0038 | <0.0001 | 0.008 |
| CCC/TTT/ACC | | | | | |
| ACC/ACC/ACC vs. | <0.0001 | 0.0002 | 0.122 | 0.0001 | <0.0001 |
| TTT/ACC/ACC | | | | | |
| ACC/ACC/ACC vs. | 0.0001 | 0.0013 | 0.0018 | 0.0001 | 0.0039 |
| TTT/CCC/ACC | | | | | |
| ACC/ACC/ACC vs. | 0.0001 | 0.0124 | 0.0014 | 0.0001 | 0.0117 |
| TTT/TTT/ACC | | | | | |
| ACC/ACC/ACC vs. | <0.0001 | 0.0017 | 0.0108 | <0.0001 | 0.0023 |
| ***/***/ACC | | | | | |
| ACC/ACC/ACC vs. | <0.0001 | 0.0002 | 0.0125 | <0.0001 | 0.0002 |
| ***/ACC/*** | | | | | |
| ACC/ACC/ACC vs. | 0.0063 | 0.0017 | 0.0236 | 0.0062 | 0.0023 |
| ACC/***/*** | | | | | |
| ACC/TTT/ACC vs. | 0.0001 | 0.0006 | 0.0161 | <0.0001 | 0.0005 |
| CCC/TTT/ACC | | | | | |
| ACC/TTT/ACC vs. | 0.0001 | 0.0004 | 0.0021 | <0.0001 | 0.0003 |
| TTT/ACC/ACC | | | | | |
| ACC/TTT/ACC vs. | 0.0002 | 0.0015 | >0.9999 | <0.0001 | 0.0014 |
| TTT/CCC/ACC | | | | | |
| ACC/TTT/ACC vs. | 0.0001 | 0.0002 | 0.0012 | <0.0001 | 0.0012 |
| TTT/TTT/ACC | | | | | |
| ACC/TTT/ACC vs. | <0.0001 | <0.0001 | 0.03 | <0.0001 | 0.0002 |
| ***/***/ACC | | | | | |
| ACC/TTT/ACC vs. | <0.0001 | 0.0007 | 0.0014 | <0.0001 | 0.0005 |
| ***/ACC/*** | | | | | |
| ACC/TTT/ACC vs. | 0.0366 | <0.0001 | 0.0748 | 0.0477 | <0.0001 |
| ACC/***/*** | | | | | |
| CCC/TTT/ACC vs. | 0.0012 | 0.0005 | 0.0029 | 0.0008 | 0.0004 |
| TTT/ACC/ACC | | | | | |
| CCC/TTT/ACC vs. | 0.001 | 0.0022 | 0.0161 | 0.0008 | 0.0021 |
| TTT/CCC/ACC | | | | | |
| CCC/TTT/ACC vs. | 0.0016 | 0.0216 | 0.2065 | 0.001 | 0.0242 |
| TTT/TTT/ACC | | | | | |
| CCC/TTT/ACC vs. | 0.0014 | 0.0006 | 0.0552 | 0.0043 | 0.0005 |
| ***/***/ACC | | | | | |
| CCC/TTT/ACC vs. | 0.0006 | 0.0008 | 0.0023 | 0.0006 | 0.0006 |
| ***/ACC/*** | | | | | |
| CCC/TTT/ACC vs. | 0.0014 | 0.0006 | 0.0014 | 0.0021 | 0.0006 |
| ACC/***/*** | | | | | |
| TTT/ACC/ACC vs. | >0.9999 | 0.0003 | 0.0044 | >0.9999 | 0.0002 |
| TTT/CCC/ACC | | | | | |
| TTT/ACC/ACC vs. | >0.9999 | 0.0005 | 0.0011 | >0.9999 | 0.0004 |
| TTT/TTT/ACC | | | | | |
| TTT/ACC/ACC vs. | 0.9215 | 0.0003 | 0.0098 | 0.9227 | 0.0003 |
| ***/***/ACC | | | | | |
| TTT/ACC/ACC vs. | 0.5496 | 0.0242 | <0.0001 | 0.5573 | 0.0168 |
| ***/ACC/*** | | | | | |
| TTT/ACC/ACC vs. | 0.0005 | 0.0004 | 0.0266 | 0.0007 | 0.0003 |
| ACC/***/*** | | | | | |
| TTT/CCC/ACC vs. | >0.9999 | 0.0027 | 0.0005 | >0.9999 | 0.0025 |
| TTT/TTT/ACC | | | | | |
| TTT/CCC/ACC vs. | 0.8993 | 0.0012 | 0.0294 | 0.9473 | 0.0011 |
| ***/***/ACC | | | | | |
| TTT/CCC/ACC vs. | 0.5381 | 0.0003 | 0.0028 | 0.6143 | 0.0002 |
| ***/ACC/*** | | | | | |
| TTT/CCC/ACC vs. | 0.0007 | 0.0012 | 0.1181 | 0.0007 | 0.0011 |
| ACC/***/*** | | | | | |
| TTT/TTT/ACC vs. | 0.8786 | 0.0004 | 0.1422 | 0.9228 | 0.0003 |
| ***/***/ACC | | | | | |
| TTT/TTT/ACC vs. | 0.4981 | 0.0009 | 0.0009 | 0.5627 | 0.0007 |
| ***/ACC/*** | | | | | |
| TTT/TTT/ACC vs. | 0.0006 | 0.0004 | 0.0002 | 0.0007 | 0.0004 |
| ACC/***/*** | | | | | |
| ***/***/ACC vs. | >0.9999 | 0.0006 | 0.0085 | >0.9999 | 0.0005 |
| ***/ACC/*** | | | | | |
| ***/***/ACC vs. | 0.0002 | 0.4418 | 0.0199 | <0.0001 | 0.1868 |
| ACC/***/*** | | | | | |
| ***/ACC/*** vs. | 0.0001 | 0.0006 | 0.0141 | 0.0001 | 0.0005 |
| ACC/***/*** | | | | | |

| | Medium Bin | High Bin | | |
|---|---|---|---|---|
| | emiRFP670/ | msfBFP[r5M]/ | msfGFP[r5M]/ | emiRFP670/ |
| | mCherry | mCherry | mCherry | mCherry |
| One-way ANOVA P-value | <0.0001 | <0.0001 | <0.0001 | <0.0001 |
| Dunnett's T3 multiple | Adjusted | Adjusted | Adjusted | Adjusted |
| comparisons test | P Value | P Value | P Value | P Value |
| ACC/ACC/ACC vs. | <0.0001 | 0.0022 | 0.004 | <0.0001 |
| ACC/TTT/ACC | | | | |
| ACC/ACC/ACC vs. | 0.0004 | <0.0001 | 0.0059 | 0.0055 |
| CCC/TTT/ACC | | | | |
| ACC/ACC/ACC vs. | 0.4009 | <0.0001 | 0.0001 | 0.0953 |
| TTT/ACC/ACC | | | | |
| ACC/ACC/ACC vs. | 0.0018 | <0.0001 | 0.5885 | 0.0029 |
| TTT/CCC/ACC | | | | |
| ACC/ACC/ACC vs. | 0.0001 | <0.0001 | 0.0072 | 0.0013 |
| TTT/TTT/ACC | | | | |
| ACC/ACC/ACC vs. | 0.0069 | <0.0001 | 0.0027 | 0.0054 |
| ***/***/ACC | | | | |
| ACC/ACC/ACC vs. | 0.0258 | <0.0001 | <0.0001 | 0.0033 |
| ***/ACC/*** | | | | |
| ACC/ACC/ACC vs. | 0.0131 | <0.0001 | 0.0029 | 0.0028 |
| ACC/***/*** | | | | |
| ACC/TTT/ACC vs. | 0.0109 | 0.0003 | 0.0035 | 0.257 |
| CCC/TTT/ACC | | | | |
| ACC/TTT/ACC vs. | 0.0002 | <0.0001 | 0.0003 | 0.0005 |
| TTT/ACC/ACC | | | | |
| ACC/TTT/ACC vs. | 0.8692 | <0.0001 | 0.0014 | 0.0002 |
| TTT/CCC/ACC | | | | |
| ACC/TTT/ACC vs. | 0.0007 | <0.0001 | 0.0007 | >0.9999 |
| TTT/TTT/ACC | | | | |
| ACC/TTT/ACC vs. | 0.0214 | 0.0004 | 0.0005 | 0.0882 |
| ***/***/ACC | | | | |
| ACC/TTT/ACC vs. | 0.0008 | <0.0001 | 0.0004 | 0.0004 |
| ***/ACC/*** | | | | |
| ACC/TTT/ACC vs. | >0.9999 | <0.0001 | 0.0001 | 0.1417 |
| ACC/***/*** | | | | |
| CCC/TTT/ACC vs. | 0.0018 | 0.0007 | 0.0003 | 0.0045 |
| TTT/ACC/ACC | | | | |
| CCC/TTT/ACC vs. | 0.0038 | 0.0007 | 0.002 | 0.0171 |
| TTT/CCC/ACC | | | | |
| CCC/TTT/ACC vs. | 0.1509 | 0.0008 | 0.0489 | 0.4507 |
| TTT/TTT/ACC | | | | |
| CCC/TTT/ACC vs. | 0.0341 | 0.0044 | 0.0008 | 0.0216 |
| ***/***/ACC | | | | |
| CCC/TTT/ACC vs. | 0.0014 | 0.0004 | 0.0005 | 0.0036 |
| ***/ACC/*** | | | | |
| CCC/TTT/ACC vs. | 0.0345 | 0.9979 | 0.001 | 0.0173 |
| ACC/***/*** | | | | |
| TTT/ACC/ACC vs. | 0.0046 | >0.9999 | 0.0002 | 0.0065 |
| TTT/CCC/ACC | | | | |
| TTT/ACC/ACC vs. | 0.0006 | >0.9999 | 0.0003 | 0.001 |
| TTT/TTT/ACC | | | | |
| TTT/ACC/ACC vs. | 0.0063 | 0.9393 | 0.0002 | 0.0049 |
| ***/***/ACC | | | | |
| TTT/ACC/ACC vs. | 0.0059 | 0.4785 | 0.0097 | 0.0002 |
| ***/ACC/*** | | | | |
| TTT/ACC/ACC vs. | 0.01 | 0.0018 | 0.0002 | 0.0025 |
| ACC/***/*** | | | | |
| TTT/CCC/ACC vs. | 0.0002 | >0.9999 | 0.0024 | 0.0005 |
| TTT/TTT/ACC | | | | |
| TTT/CCC/ACC vs. | 0.0192 | 0.951 | 0.001 | 0.0052 |
| ***/***/ACC | | | | |
| TTT/CCC/ACC vs. | 0.0028 | 0.5133 | 0.0002 | 0.0043 |
| ***/ACC/*** | | | | |
| TTT/CCC/ACC vs. | 0.9328 | 0.0019 | 0.0011 | 0.0032 |
| ACC/***/*** | | | | |
| TTT/TTT/ACC vs. | 0.1258 | 0.9261 | 0.0004 | 0.0809 |
| ***/***/ACC | | | | |
| TTT/TTT/ACC vs. | 0.0005 | 0.4582 | 0.0005 | 0.0009 |
| ***/ACC/*** | | | | |
| TTT/TTT/ACC vs. | 0.0047 | 0.002 | 0.0005 | 0.0956 |
| ACC/***/*** | | | | |
| ***/***/ACC vs. | 0.0055 | >0.9999 | 0.0004 | 0.0043 |
| ***/ACC/*** | | | | |
| ***/***/ACC vs. | 0.0107 | 0.0205 | 0.0122 | 0.9144 |
| ACC/***/*** | | | | |
| ***/ACC/*** vs. | 0.0068 | 0.0017 | 0.0004 | 0.0022 |
| ACC/***/*** | | | | |

TABLE 5
Post-hoc Dunnett's T3 multiple comparisons tests for 3-ORF CHO-K1
constructs depicted in FIG. 15 (N = 4 for all conditions)

| | Low Bin | | | Medium Bin | |
|---|---|---|---|---|---|
| | msfBFP[r5M]/ | msfGFP[r5M]/ | emiRFP670/ | msfBFP[r5M]/ | msfGFP[r5M]/ |
| | mCherry | mCherry | mCherry | mCherry | mCherry |
| One-way ANOVA P-value | <0.0001 | <0.0001 | <0.0001 | <0.0001 | <0.0001 |
| Dunnett's T3 multiple | Adjusted | Adjusted | Adjusted | Adjusted | Adjusted |
| comparisons test | P Value | P Value | P Value | P Value | P Value |
| ACC/ACC/ACC vs. | 0.023 | 0.0001 | 0.022 | 0.0053 | <0.0001 |
| ACC/TTT/ACC | | | | | |
| ACC/ACC/ACC vs. | 0.0159 | 0.0001 | 0.0172 | 0.0557 | <0.0001 |
| CCC/TTT/ACC | | | | | |
| ACC/ACC/ACC vs. | 0.0191 | 0.0004 | 0.4576 | 0.0036 | 0.0048 |
| TTT/ACC/ACC | | | | | |
| ACC/ACC/ACC vs. | <0.0001 | 0.0001 | 0.6562 | 0.0011 | <0.0001 |
| TTT/CCC/ACC | | | | | |
| ACC/ACC/ACC vs. | <0.0001 | 0.0002 | 0.011 | 0.0031 | <0.0001 |
| TTT/TTT/ACC | | | | | |
| ACC/ACC/ACC vs. | 0.0002 | <0.0001 | 0.0738 | 0.0004 | <0.0001 |
| ***/***/ACC | | | | | |
| ACC/ACC/ACC vs. | 0.003 | <0.0001 | 0.0518 | <0.0001 | <0.0001 |
| ***/ACC/*** | | | | | |
| ACC/ACC/ACC vs. | 0.0657 | <0.0001 | 0.0637 | 0.0004 | <0.0001 |
| ACC/***/*** | | | | | |
| ACC/TTT/ACC vs. | 0.3905 | 0.0507 | >0.9999 | 0.3854 | 0.0026 |
| CCC/TTT/ACC | | | | | |
| ACC/TTT/ACC vs. | 0.0838 | <0.0001 | 0.0115 | 0.0148 | 0.0002 |
| TTT/ACC/ACC | | | | | |
| ACC/TTT/ACC vs. | 0.0002 | 0.0002 | 0.1026 | 0.0033 | <0.0001 |
| TTT/CCC/ACC | | | | | |
| ACC/TTT/ACC vs. | 0.0002 | 0.0001 | >0.9999 | 0.0032 | <0.0001 |
| TTT/TTT/ACC | | | | | |
| ACC/TTT/ACC vs. | 0.0009 | <0.0001 | 0.6133 | 0.0003 | <0.0001 |
| ***/***/ACC | | | | | |
| ACC/TTT/ACC vs. | 0.0095 | <0.0001 | 0.0094 | <0.0001 | <0.0001 |
| ***/ACC/*** | | | | | |
| ACC/TTT/ACC vs. | 0.5387 | 0.0018 | 0.6389 | 0.0041 | 0.0003 |
| ACC/***/*** | | | | | |
| CCC/TTT/ACC vs. | 0.379 | <0.0001 | <0.0001 | <0.0001 | 0.0003 |
| TTT/ACC/ACC | | | | | |
| CCC/TTT/ACC vs. | 0.036 | 0.0002 | 0.0025 | 0.0001 | <0.0001 |
| TTT/CCC/ACC | | | | | |
| CCC/TTT/ACC vs. | 0.0207 | <0.0001 | 0.9791 | <0.0001 | 0.0028 |
| TTT/TTT/ACC | | | | | |
| CCC/TTT/ACC vs. | 0.004 | <0.0001 | 0.0844 | <0.0001 | 0.0002 |
| ***/***/ACC | | | | | |
| CCC/TTT/ACC vs. | 0.0164 | <0.0001 | 0.0005 | 0.0001 | <0.0001 |
| ***/ACC/*** | | | | | |
| CCC/TTT/ACC vs. | >0.9999 | <0.0001 | 0.2869 | 0.0013 | <0.0001 |
| ACC/***/*** | | | | | |
| TTT/ACC/ACC vs. | 0.9956 | <0.0001 | 0.0126 | >0.9999 | 0.0004 |
| TTT/CCC/ACC | | | | | |
| TTT/ACC/ACC vs. | 0.9911 | <0.0001 | 0.0002 | 0.0861 | 0.0003 |
| TTT/TTT/ACC | | | | | |
| TTT/ACC/ACC vs. | 0.3421 | <0.0001 | 0.0056 | 0.0037 | 0.0002 |
| ***/***/ACC | | | | | |
| TTT/ACC/ACC vs. | 0.6652 | 0.0293 | 0.0591 | 0.0121 | 0.0441 |
| ***/ACC/*** | | | | | |
| TTT/ACC/ACC vs. | 0.6272 | <0.0001 | 0.0012 | 0.9005 | 0.0002 |
| ACC/***/*** | | | | | |
| TTT/CCC/ACC vs. | >0.9999 | 0.0003 | 0.0001 | 0.8554 | <0.0001 |
| TTT/TTT/ACC | | | | | |
| TTT/CCC/ACC vs. | 0.2133 | <0.0001 | <0.0001 | 0.0041 | <0.0001 |
| ***/***/ACC | | | | | |
| TTT/CCC/ACC vs. | 0.7103 | <0.0001 | <0.0001 | 0.0026 | <0.0001 |
| ***/ACC/*** | | | | | |
| TTT/CCC/ACC vs. | 0.1384 | <0.0001 | 0.1201 | 0.9148 | <0.0001 |
| ACC/***/*** | | | | | |
| TTT/TTT/ACC vs. | 0.2524 | <0.0001 | 0.0141 | 0.0122 | <0.0001 |
| ***/***/ACC | | | | | |
| TTT/TTT/ACC vs. | 0.8134 | <0.0001 | <0.0001 | 0.0 | <0.0001 |
| ***/ACC/*** | | | | | |
| TTT/TTT/ACC vs. | 0.1304 | <0.0001 | 0.1349 | 0.2487 | <0.0001 |
| ACC/***/*** | | | | | |
| ***/***/ACC vs. | >0.9999 | <0.0001 | <0.0001 | 0.9397 | <0.0001 |
| ***/ACC/*** | | | | | |
| ***/***/ACC vs. | 0.0271 | 0.0157 | >0.9999 | 0.002 | 0.0309 |
| ACC/***/*** | | | | | |
| ***/ACC/*** vs. | 0.0498 | <0.0001 | 0.0034 | 0.0014 | <0.0001 |
| ACC/***/*** | | | | | |

| | Medium Bin | High Bin | | |
|---|---|---|---|---|
| | emiRFP670/ | msfBFP[r5M]/ | msfGFP[r5M]/ | emiRFP670/ |
| | mCherry | mCherry | mCherry | mCherry |
| One-way ANOVA P-value | <0.0001 | <0.0001 | <0.0001 | <0.0001 |
| Dunnett's T3 multiple | Adjusted | Adjusted | Adjusted | Adjusted |
| comparisons test | P Value | P Value | P Value | P Value |
| ACC/ACC/ACC vs. | <0.0001 | <0.0001 | <0.0001 | <0.0001 |
| ACC/TTT/ACC | | | | |
| ACC/ACC/ACC vs. | <0.0001 | <0.0001 | <0.0001 | <0.0001 |
| CCC/TTT/ACC | | | | |
| ACC/ACC/ACC vs. | 0.0011 | <0.0001 | 0.0002 | 0.0129 |
| TTT/ACC/ACC | | | | |
| ACC/ACC/ACC vs. | 0.0107 | <0.0001 | 0.001 | 0.1315 |
| TTT/CCC/ACC | | | | |
| ACC/ACC/ACC vs. | <0.0001 | <0.0001 | <0.0001 | 0.0004 |
| TTT/TTT/ACC | | | | |
| ACC/ACC/ACC vs. | <0.0001 | <0.0001 | <0.0001 | <0.0001 |
| ***/***/ACC | | | | |
| ACC/ACC/ACC vs. | 0.0011 | <0.0001 | <0.0001 | <0.0001 |
| ***/ACC/*** | | | | |
| ACC/ACC/ACC vs. | 0.0118 | <0.0001 | <0.0001 | 0.7929 |
| ACC/***/*** | | | | |
| ACC/TTT/ACC vs. | 0.0328 | 0.0063 | 0.0001 | 0.0012 |
| CCC/TTT/ACC | | | | |
| ACC/TTT/ACC vs. | <0.0001 | 0.9562 | <0.0001 | <0.0001 |
| TTT/ACC/ACC | | | | |
| ACC/TTT/ACC vs. | <0.0001 | >0.9999 | <0.0001 | <0.0001 |
| TTT/CCC/ACC | | | | |
| ACC/TTT/ACC vs. | 0.0599 | 0.9999 | <0.0001 | 0.0046 |
| TTT/TTT/ACC | | | | |
| ACC/TTT/ACC vs. | 0.0131 | 0.0062 | <0.0001 | 0.0031 |
| ***/***/ACC | | | | |
| ACC/TTT/ACC vs. | 0.0001 | 0.0048 | <0.0001 | <0.0001 |
| ***/ACC/*** | | | | |
| ACC/TTT/ACC vs. | 0.253 | 0.3322 | 0.0023 | 0.0643 |
| ACC/***/*** | | | | |
| CCC/TTT/ACC vs. | <0.0001 | 0.0091 | <0.0001 | 0.0006 |
| TTT/ACC/ACC | | | | |
| CCC/TTT/ACC vs. | 0.0006 | 0.0004 | <0.0001 | 0.0002 |
| TTT/CCC/ACC | | | | |
| CCC/TTT/ACC vs. | 0.9216 | 0.0002 | <0.0001 | 0.1538 |
| TTT/TTT/ACC | | | | |
| CCC/TTT/ACC vs. | 0.9509 | <0.0001 | <0.0001 | 0.9939 |
| ***/***/ACC | | | | |
| CCC/TTT/ACC vs. | 0.0002 | <0.0001 | <0.0001 | <0.0001 |
| ***/ACC/*** | | | | |
| CCC/TTT/ACC vs. | 0.1044 | 0.0003 | 0.0002 | 0.3346 |
| ACC/***/*** | | | | |
| TTT/ACC/ACC vs. | <0.0001 | 0.7414 | 0.0001 | 0.0524 |
| TTT/CCC/ACC | | | | |
| TTT/ACC/ACC vs. | <0.0001 | 0.3656 | <0.0001 | 0.0031 |
| TTT/TTT/ACC | | | | |
| TTT/ACC/ACC vs. | <0.0001 | 0.0019 | <0.0001 | 0.0005 |
| ***/***/ACC | | | | |
| TTT/ACC/ACC vs. | 0.0033 | 0.0014 | 0.1314 | 0.0084 |
| ***/ACC/*** | | | | |
| TTT/ACC/ACC vs. | 0.0069 | 0.0752 | <0.0001 | 0.0617 |
| ACC/***/*** | | | | |
| TTT/CCC/ACC vs. | <0.0001 | 0.7539 | <0.0001 | <0.0001 |
| TTT/TTT/ACC | | | | |
| TTT/CCC/ACC vs. | <0.0001 | <0.0001 | <0.0001 | <0.0001 |
| ***/***/ACC | | | | |
| TTT/CCC/ACC vs. | 0.0002 | <0.0001 | <0.0001 | <0.0001 |
| ***/ACC/*** | | | | |
| TTT/CCC/ACC vs. | 0.0172 | 0.0039 | <0.0001 | 0.4548 |
| ACC/***/*** | | | | |
| TTT/TTT/ACC vs. | 0.0636 | <0.0001 | <0.0001 | 0.0302 |
| ***/***/ACC | | | | |
| TTT/TTT/ACC vs. | <0.0001 | <0.0001 | <0.0001 | <0.0001 |
| ***/ACC/*** | | | | |
| TTT/TTT/ACC vs. | 0.1214 | 0.0182 | <0.0001 | 0.5772 |
| ACC/***/*** | | | | |
| ***/***/ACC vs. | <0.0001 | 0.9997 | <0.0001 | <0.0001 |
| ***/ACC/*** | | | | |
| ***/***/ACC vs. | 0.084 | 0.001 | 0.001 | 0.271 |
| ACC/***/*** | | | | |
| ***/ACC/*** vs. | 0.0046 | <0.0001 | <0.0001 | 0.0113 |
| ACC/***/*** | | | | |

TABLE 6
Dunn's multiple comparisons test results of Emerald/mCherry values
of SEMPER-mARG expressing HEK293T cells (Referenced in FIG. 3B)

| Dunn's multiple comparisons | Adjusted P | | |
|---|---|---|---|
| test | Value | n1 | n2 |
| Two Vector vs. ACC/ACC | <0.0001 | 9793 | 9793 |
| Two Vector vs. CCC/ACC | <0.0001 | 9793 | 9793 |
| Two Vector vs. GGG/ACC | <0.0001 | 9793 | 9793 |
| Two Vector vs. TTT/ACC | <0.0001 | 9793 | 9793 |
| Two Vector vs. ***/ACC | <0.0001 | 9793 | 9793 |
| Two Vector vs. IRES-mCherry | <0.0001 | 9793 | 5278 |
| ACC/ACC vs. CCC/ACC | <0.0001 | 9793 | 9793 |
| ACC/ACC vs. GGG/ACC | <0.0001 | 9793 | 9793 |
| ACC/ACC vs. TTT/ACC | <0.0001 | 9793 | 9793 |
| ACC/ACC vs. ***/ACC | <0.0001 | 9793 | 9793 |
| ACC/ACC vs. IRES-mCherry | <0.0001 | 9793 | 5278 |
| CCC/ACC vs. GGG/ACC | <0.0001 | 9793 | 9793 |
| CCC/ACC vs. TTT/ACC | <0.0001 | 9793 | 9793 |
| CCC/ACC vs. ***/ACC | <0.0001 | 9793 | 9793 |
| CCC/ACC vs. IRES-mCherry | <0.0001 | 9793 | 5278 |
| GGG/ACC vs. TTT/ACC | <0.0001 | 9793 | 9793 |
| GGG/ACC vs. ***/ACC | <0.0001 | 9793 | 9793 |
| GGG/ACC vs. IRES-mCherry | <0.0001 | 9793 | 5278 |
| TTT/ACC vs. ***/ACC | <0.0001 | 9793 | 9793 |
| TTT/ACC vs. IRES-mCherry | <0.0001 | 9793 | 5278 |
| **/ACC vs. IRES-mCherry | <0.0001 | 9793 | 5278 |

TABLE 7
Multiple comparisons test results of BURST ultrasound signal
from HEK293T cells expressing SEMPER-mARG gas vesicles
(Referenced in FIG. 3D)

| | Individual P | | |
|---|---|---|---|
| Uncorrected Fisher's LSD | Value | n1 | n2 |
| Two-vector vs. ACC/ACC | <0.0001 | 4 | 4 |
| Two-vector vs. CCC/ACC | <0.0001 | 4 | 4 |
| Two-vector vs. TTT/ACC | <0.0001 | 4 | 4 |
| Two-vector vs. ***/ACC | <0.0001 | 4 | 4 |
| ACC/ACC vs. CCC/ACC | 0.001 | 4 | 4 |
| ACC/ACC vs. TTT/ACC | <0.0001 | 4 | 4 |
| ACC/ACC vs. ***/ACC | <0.0001 | 4 | 4 |
| CCC/ACC vs. TTT/ACC | <0.0001 | 4 | 4 |
| CCC/ACC vs. ***/ACC | <0.0001 | 4 | 4 |
| TTT/ACC vs. ***/ACC | <0.0001 | 4 | 4 |

TABLE 8
Multiple comparisons test results of secreted human IgG ELISA
of SEMPER-b12 mAb expressing HEK293T cells (Referenced in FIG. 4B)

| | Individual P | | |
|---|---|---|---|
| Uncorrected Fisher's LSD | Value | n1 | n2 |
| ACC/*** vs. ACC/ACC | <0.0001 | 6 | 6 |
| ACC/*** vs. GGG/ACC | <0.0001 | 6 | 6 |
| ACC/*** vs. CCC/ACC | <0.0001 | 6 | 6 |
| ACC/*** vs. TTC/ACC | <0.0001 | 6 | 6 |
| ACC/*** vs. TTT/ACC | 0.0016 | 6 | 6 |
| ACC/*** vs. ***/ACC | 0.5476 | 6 | 6 |
| ACC/*** vs. no plasmid | 0.6651 | 6 | 6 |
| ACC/*** vs. ACC/*** + ***/ACC | <0.0001 | 6 | 6 |
| ACC/ACC vs. GGG/ACC | <0.0001 | 6 | 6 |
| ACC/ACC vs. CCC/ACC | <0.0001 | 6 | 6 |
| ACC/ACC vs. TTC/ACC | 0.0002 | 6 | 6 |
| ACC/ACC vs. TTT/ACC | 0.0001 | 6 | 6 |
| ACC/ACC vs. ***/ACC | <0.0001 | 6 | 6 |
| ACC/ACC vs. no plasmid | <0.0001 | 6 | 6 |
| ACC/ACC vs. ACC/*** + ***/ACC | <0.0001 | 6 | 6 |
| GGG/ACC vs. CCC/ACC | 0.0012 | 6 | 6 |
| GGG/ACC vs. TTC/ACC | <0.0001 | 6 | 6 |
| GGG/ACC vs. TTT/ACC | <0.0001 | 6 | 6 |
| GGG/ACC vs. ***/ACC | <0.0001 | 6 | 6 |
| GGG/ACC vs. no plasmid | <0.0001 | 6 | 6 |
| GGG/ACC vs. ACC/*** + ***/ACC | <0.0001 | 6 | 6 |
| CCC/ACC vs. TTC/ACC | <0.0001 | 6 | 6 |
| CCC/ACC vs. TTT/ACC | <0.0001 | 6 | 6 |
| CCC/ACC vs. ***/ACC | <0.0001 | 6 | 6 |
| CCC/ACC vs. no plasmid | <0.0001 | 6 | 6 |
| CCC/ACC vs. ACC/*** + ***/ACC | <0.0001 | 6 | 6 |
| TTC/ACC vs. TTT/ACC | <0.0001 | 6 | 6 |
| TTC/ACC vs. ***/ACC | <0.0001 | 6 | 6 |
| TTC/ACC vs. no plasmid | <0.0001 | 6 | 6 |
| TTC/ACC vs. ACC/*** + ***/ACC | <0.0001 | 6 | 6 |
| TTT/ACC vs. ***/ACC | 0.0003 | 6 | 6 |
| TTT/ACC vs. no plasmid | 0.0004 | 6 | 6 |
| TTT/ACC vs. ACC/*** + ***/ACC | <0.0001 | 6 | 6 |
| ***/ACC vs. no plasmid | 0.8656 | 6 | 6 |
| ***/ACC vs. ACC/*** + ***/ACC | <0.0001 | 6 | 6 |
| no plasmid vs. ACC/*** + ***/ACC | <0.0001 | 6 | 6 |

TABLE 9
Genetic parts, primers, and plasmid sequences used to construct plasmids and IVT
mRNA

| Sequence | Sequence | | |
|---|---|---|---|
| Type | Identifier | Description | Sequence (5′ → 3′) |
| Primer | p101 | Forward primer to generate linear IVT templates. | SEQ ID NO: 1 |
| | | Additionally, this primer ensures there is an AG | |
| | | dinucleotide at the beginning of the transcript. | |
| Ultramer | p54 | Reverse primer to generate linear IVT templates. This | SEQ ID NO: 2 |
| | | primer ensures the linear template contains a 120 bp | |
| | | poly A tail. | |
| 5′ | 5′UTR (for 2-ORF | The 5′UTR used for 2-ORF and 3-ORF plasmids and | SEQ ID NO: 3 |
| Untranslated | and 3-ORF | mRNA SEMPER constructs employing only | |
| Region | plasmids for FP | fluorescent proteins. The trinucleotide NNN that | |
| | expression) | changes the TIS of the first ORF is not included in this | |
| | | 5′UTR | |
| 5′ | 5′UTR for | The following 5′UTR was used for SEMPER mARG | SEQ ID NO: 4 |
| Untranslated | SEMPER mARG | plasmids | |
| Region | plasmids | | |
| 3′ | 3′UTR (plasmid) | The 3′UTR for plasmid-based expression notably | SEQ ID NO: 5 |
| Untranslated | | includes a Woodchuck Hepatitis Virus Post- | |
| Region | | transcriptional Regulatory Element to extend transcript | |
| | | half-life, and an hGH polyA signal. | |
| 3′ | 3′ UTR (mRNA) | The 3′UTR sequence used in IVT mRNA transcripts | SEQ ID NO: 6 |
| Untranslated | | (not including poly A tail). | |
| Region | | | |
| ORF | msfGFP[r5M] | Monomeric Superfolder GFP with methionines | SEQ ID NO: 7 |
| | | mutated to other amino acids. Sequence is from start to | |
| | | stop codon of ORF. | |
| ORF | msfBFP[r5M] | Monomeric Superfolder BFP with methionines | SEQ ID NO: 8 |
| | | mutated to other amino acids. Sequence is from start to | |
| | | stop codon of ORF. | |
| ORF | mEBFP2 | Monomeric enhanced BFP. Sequence is from start to | SEQ ID NO: 9 |
| | | stop codon of ORF. | |
| ORF | emiRFP670 | Far red fluorescent protein with additional +1 Valine | SEQ ID NO: 10 |
| | | mutation to make +1 nucleotide after start codon a | |
| | | guanine nucleotide. Sequence is from start to stop | |
| | | codon of ORF. | |
| Internal | IRES | This IRES sequence was used in all constructs that | SEQ ID NO: 11 |
| Ribosome | | contained mCherry. | |
| Entry Site | | | |
| Enhancer | CMV Enhancer | Enhancer region to drive expression from CMV | SEQ ID NO: 12 |
| | | promoter. | |
| Promoter | CMV Promoter | CMV Promoter to drive transcription of plasmid-based | SEQ ID NO: 13 |
| | | systems. | |
| Promoter | T7 Promoter | T7 Promoter encoded in IVT plasmid backbones. An | SEQ ID NO: 14 |
| | | additional “A” nucleotide is added when PCR | |
| | | amplifying with p101 for capping purposes. | |
| Full | 2-ORF SEMPER | SEMPER plasmid encoding msfGFP[r5M] and | SEQ ID NO: 15 |
| Plasmid | TTT/ACC | mEBFP2 in TTT/ACC ratio used in transfection. | |
| Full | 3-ORF SEMPER | 3-ORF SEMPER plasmid used in transient transfection | SEQ ID NO: 16 |
| Plasmid | Plasmid | experiments. | |
| | ACC/***/*** | | |
| Full | SEMPER mARG | Full sequence for SEMPER mARG plasmid for the | SEQ ID NO: 17 |
| Plasmid | | ACC/ACC TIS combination. | |
| Full | IVT backbone | Plasmids with this architecture were used to amplify | SEQ ID NO: 18 |
| Plasmid | plasmid | linear DNA templates for IVT reactions. This plasmid | |
| | | was used for creating linear templates for ACC/ACC 2- | |
| | | ORF. | |

### MATERIALS AND METHODS

### Vector Construction

Plasmids were constructed using standard cloning techniques, including Gibson assembly and conventional restriction and ligation. All final sequences were verified using whole-plasmid sequencing through Primordium Labs. Plasmids containing two or three fluorescent protein ORFs were constructed in the following way: Individual FP sequences were ordered from IDT or TWIST Biosciences as synthetic gBlocks. Modified TIS sequences and the *** sequence were introduced using overhang PCR primers. Finally, all components were subcloned into pCMV-Sport-gvpA-IRES-mCherry using NEB HiFi DNA Assembly replacing the gvpA ORF. Some modifications were made to the 5′ UTR sequences. Plasmid sequences for the 2-ORF TTT/ACC construct and the 3-ORF ACC/***/*** construct as well as sequences for individual genetic parts in these plasmids are provided in Table 9 to aid in reproduction of this work.

SEMPER mARG plasmids containing gvpA and gvpNJKFGW-EmGFP were constructed as follows: The ORF containing gvpNJKFGW-EmGFP was PCR amplified and subcloned into pCMV-Sport-gvpA-IRES-mCherry using NEB HiFi DNA Assembly between gvpA and IRES. Different gvpA TIS sequences were introduced using single-stranded oligo bridge primers with NEB HiFi Assembly.

To produce plasmids amenable for in vitro transcription of mRNA, sequences containing the 5′UTR through the stop codon of the mEBFP2 ORF from the 2-ORF SEMPER plasmids were cloned into an IVT plasmid backbone containing a T7 promoter, a synthetic 3′UTR sequence, and a 100 bp polyA track (Table 9) using PCR amplification and Gibson assembly methods.

Production of mRNA by In Vitro Transcription

Linear templates were PCR amplified from the IVT plasmids (described in Vector construction) using primer p101 and “Ultramer” p54 synthesized by Integrated DNA Technologies. Primer p101 introduced an AG dinucleotide following the T7 promoter sequence on linear templates to ensure efficient 5′ capping of IVT mRNA. Linear templates were purified using standard gel electrophoresis and DNA cleanup methods. IVT mRNA was produced using the HiScribe T7 High Yield RNA Synthesis Kit (NEB #E2040) along with 500 ng of linear template and 4 mM CleanCap AG Reagent (Trilink, N-7113). 5 mM N1-Methylpseudouridine-5′-triphosphate (Trilink, N-1081) was substituted for the standard uridine triphosphate included in the HiScribe Kit for certain reactions. Following the IVT incubation, reactions were additionally incubated with 2 units of DNAse I to remove remaining DNA template. Purification of IVT mRNA was conducted using the Monarch RNA Cleanup Kit (NEB #T2040L). To confirm IVT mRNA were of the correct lengths, aliquots of purified IVT mRNA were denatured at 70° C. for 15 minutes and then subjected to gel electrophoresis on a 1% agarose gel in Tris acetate EDTA (TAE) stained with SYBR Safe. ssRNA ladder (NEB #N0362S) was used to confirm band sizes. mRNA concentrations for each IVT mRNA were quantified using a Qubit Fluorometer (Invitrogen). Completed IVT mRNA samples were stored at −80° C.

### Cell Culture and Transfection

HEK293T cells (American Type Culture Collection (ATCC), CLR-3216) and CHO-K1 cells (American Type Culture Collection (ATCC), CCL-61) were cultured in 24-well plates at 37° C. and 5% CO₂ in a humidified incubator in 0.5 mL of DMEM (Corning, 10-013-CV) with 10% FBS (Gibco) and 1× penicillin-streptomycin until about 80% confluency before transfection with plasmid DNA or IVT mRNA.

For plasmid DNA transfection, transient transfection mixtures were created by mixing 500 ng of plasmid with polyethyleneimine (PEI-MAX, linear 40 kD #24765-2, Polysciences) at 4.12 μg of polyethyleneimine per microgram of DNA in 150 mM NaCl. The mixture was incubated for 12 minutes at room temperature and added dropwise to HEK293T or CHO-K1 adherent cells. Media was changed after 12-16 hours and daily thereafter. Cells were analyzed with flow-cytometry 48 hours post-transfection. Deviations from this protocol are described in the supplementary information where applicable.

For IVT mRNA transfection, transient transfection mixtures were created by mixing 500 ng of mRNA, 1.5 μL of Lipofectamine MessengerMAX Reagent (Invitrogen #LMRNA008), and 50 μL of Opti-Mem media (Gibco #31985070) according to the Lipofectamine MessengerMAX Reagent standard operating procedure. The mixture was incubated for 5 minutes at room temperature and added dropwise to HEK293T cells. Media was changed after 12-16 hours and daily thereafter. Cells were analyzed with flow-cytometry 48 hours post-transfection.

For transfection of plasmids encoding mARGs, transient transfection mixtures were created as above except that 600 ng of total DNA was mixed together as follows: 56 fmol of gvpA-IRES-mCherry plasmid with or without 14 fmol gvpNJKFGW-EmGFP plasmid or 56 fmol of a 2-ORF SEMPER mARG plasmid. Plasmid mixtures were normalized with addition of pUC19 up to 600 ng before complexing with PEI-MAX. The mixture was incubated for 12 minutes at room temperature and added dropwise to HEK293T adherent cells. Media was changed after 12-16 hours and daily thereafter. Cells were harvested 3-days post-transfection.

### Recombinant b12 IgG Expression in HEK293T Cells

HEK293T cells were plated at a density of 200,000 cells per well in 48-well plates using 250 μL of Dulbecco's Modified Eagle Medium (DMEM) supplemented with 10% fetal bovine serum (FBS), 1× penicillin/streptomycin, 20 mM HEPES (pH 7.0), 1×MEM Non-Essential Amino Acids (NEAA), and 1× GlutaMAX™. The cells were cultured at 37° C. in a humidified atmosphere containing 5% CO₂ for 24 hours prior to transfection.

A DNA-polyethylenimine (PEI) Max transfection mixture was prepared by dissolving 206 mg of PEI-MAX (40 kDa, Polysciences, #24765-2) in 500 mL of tissue culture (TC)-grade phosphate-buffered saline (PBS) to achieve a final concentration of 0.412 mg/mL (10.3 μM). The solution was adjusted to pH 7.0 with NaOH and filter-sterilized. Aliquots were stored at −80° C. until needed. For each transfection, 350 ng of the SEMPER-b12 plasmid (7485 bp) was combined with 17.5 μL of a sterile 150 mM NaCl solution. Separately, the PEI-Max solution was diluted with 150 mM NaCl in a 1:4 ratio, and 17.5 μL of this dilution was added to the plasmid-NaCl mixture. The combined mixture was gently mixed and incubated at room temperature for 12 minutes to allow complex formation, after which it was added dropwise to each well. For the two-plasmid control, 175 ng of heavy chain (HC) and light chain (LC) plasmids were combined to total 350 ng and then complexed with the PEI-Max solution as described above.

The media in the wells were replaced after 16 hours with fresh DMEM. An additional 250 μL of the same medium was supplemented after another 24 hours, bringing the total volume in each well to 500 μL. Three days post-transfection, 5 μL of the cell culture supernatant was transferred to 95 μL of assay buffer.

IgG yields were quantified using the Human Total IgG Coated ELISA Kit (Invitrogen, #BMS2091), adhering to the manufacturer's protocol. Absorbance was measured at 450 nm with a reference wavelength of 620 nm using the Tecan SPARK plate reader.

### Flow Cytometry and Apoptosis Assays

To harvest the cells, cells were dissociated using trypsin/EDTA and centrifuged at 300 g for 6 minutes at room temperature. For experiments involving mARGs, 2-ORF SEMPER plasmid transfections, and 2-ORF SEMPER mRNA transfections, cells were analyzed with MACSQuant VYB (Miltenyi Biotec) except for data depicted in FIG. 6C (Right) which was gathered using a MACSQuant10 (Miltenyi Biotec). For experiments involving 3-ORF SEMPER, cells were analyzed using Cytoflex S (Beckman Coulter). Single-color controls were used to allow for compensation. In plasmid transfection experiments, cells were gated for size and doublet-discriminated before being gated and further binned by mCherry fluorescence. For IVT mRNA transfection experiments cells were gated as above except they were gated for msfGFP [r5M] and mEBFP2 double-positivity instead of by mCherry fluorescence.

For the Annexin V apoptosis assay, cells were transfected as described above but did not perform media changes. For the positive control, cells transfected with pgvpA-IRES-mCherry (w/start codon removed in front of gvpA) were treated with 10 μM Raptinal 18 hours before harvest. Cells were harvested two days after transfection. To do this, supernatant was collected from cells to recover the dead cells. The adhered cells were then trypsinized and added this trypsinized cell fraction to the original supernatant for maximum cell recovery. Then, the cells were spun down at 300 g for 6 minutes, carefully removed the supernatant, and resuspended the cells in 90 μL of cold annexin binding buffer with no EDTA and supplemented Ca²⁺, followed by 10 μL of Annexin V Pacific Blue stain (A35122). Following a 15-minute incubation, 150 μL of the same annexin binding buffer was added and then performed flow cytometry to obtain fluorescence values for the pacific blue stain. Gating in FlowJo was performed by selecting the largest population in the FCS/SSC plot, keeping the lower left corner in the cell gate to include smaller apoptotic cells. A 10⁴ threshold was selected for the Pacific Blue Annexin V stain, corresponding to the right tail of the unstained control so that the Annexin V+ population of the unstained control was around 0%. Values were normalized by setting the mean value of Raptinal-treated samples to 100%.

### In Vitro Ultrasound Imaging of Transient Expression of GV's in HEK293T Cells Suspended in Agarose Phantoms

Cells were harvested as described above. Cells were resuspended with 1% low-melt agarose (GoldBio) in PBS at 40° C. at concentrations of ˜15 million cells per milliliter and then loaded into wells of pre-formed phantoms consisting of 1% molecular biology-grade agarose (Bio-Rad) in PBS. Phantoms were imaged using L22-14vX transducer (Verasonics) at 15.625 MHz while submerged in PBS on top of an acoustic absorber pad. For BURST imaging, wells were centered around the 8 mm natural focus of the transducer and a BURST pulse sequence was applied in pAM acquisition mode with the focus set to 8 mm, and the voltage was set to 2V for the first 10 frames and 15V for the remaining frames.

BURST images were produced by pixel-wise subtraction of the 11th (collapse) frame from the 54th (post-collapse) frame. Resulting differences were divided by 100. Images were quantified as follows: The sample ROIs were drawn inside the well of the agarose phantom. Average pixel value inside the ROI was calculated for each replicate.

### Simulations and Code Availability

Simulations were conducted using standard python libraries. The RAM and CPU power of a standard laptop computer were sufficient to run the simulations depicted in FIG. 6. Example code and a list of dependencies are accessible through GitHub.

### Model Note: Simulations of Semper Circuits.

### Assumptions of the Model

The model makes certain simplifications and biological assumptions including:
(1) Scanning ribosomes (also referred to below as just “ribosomes”) load at the 5′end of an mRNA transcript and will only move across the mRNA from 5′ to 3′. This is in direct agreement with the canonical 5′ cap-dependent eukaryotic scanning mechanism for translation, which has been heavily researched. The loading of ribosomes at the 5′cap is considered as a fast step and do not directly consider the kinetics of this step in the model.
(2) The scanning ribosome (a.k.a. 43S PIC) is a subunit of the full translating ribosome. Upon recognizing a TIS, the 43S PIC interacts with the 60S subunit to form the 80S ribosome which conducts translation. The formation of the 80S ribosome is assumed is a kinetically fast step and the rate-limiting step is the interaction of the 43S PIC with the TIS sequence. Therefore, it does not consider a “pooled” model where there may become resource limitations and competition for available 60S subunits. Resource abundances and concentrations in the local environment of the mRNA may also explain boosted first ORF expression observations in FIG. 2. However, these effects are also not considered in the model.
(3) The rate-limiting step in the model is the interaction of the 43S PIC with the TIS sequence and its ability to recognize this TIS as a start site for recruitment of the 60s subunit and subsequent translation. The relative frequency with which a 43S PIC initiates translation at a given TIS are parameters in the model is called p_TIS values. p_TIS takes on a value between 0 and 1. The p_TIS values for ***, TTT, CCC, and ACC are described herein and were estimated based on observed results herein and the results of Ferreira et al. and Noderer et al. As the model is probabilistic in nature, it does not consider any other rate or kinetic parameters. Estimates of these relative p_TIS values can be made for other TIS sequences through analysis of the predicted and measured strengths of TISs made by Noderer et al.
(4) The model assumes there is inherent stochasticity with the number of scanning ribosomes that will traverse an mRNA and the number of mRNA that may exist in a cell. It captures this stochasticity by sampling these values from probabilistic distributions. Specifically, normality was used to describe these processes. However, other distributions such as the Poisson distribution, negative binomial distribution, and gamma distribution could also be considered. The code as written can easily be implemented with these different distributions if a user so desires.
(5) As mentioned, the model does not consider “non-linear” effects, such as the increased translation of the first ORF as observed in the disclosed screen of the 3-ORF library.
(6) The model does not consider the sequence identity of the ORFs other than the TIS sequence in front of it. The model does not account for ribosomes falling off the transcript during scanning, the length of the transcript, the length of the 5′UTR, local structure of the mRNA, or global structure of the mRNA.

### Model Architecture

The pipeline for producing the results in FIGS. 6B-6C are as follows:
(1) Choosing the number of mRNA to model. The number of unique mRNA simulation results needed to fill the desired number of cells to be modeled is first estimated with some excess. This is an upper bound estimate to allow the simulation to proceed.
(2) Estimating the number of ribosomes that will traverse each mRNA. Once the number of mRNA are determined, the number of ribosomes that will traverse each mRNA are sampled from a normal distribution. A normal distribution was chosen as it is a commonly used distribution in modeling biological systems. Here, it samples from a normal distribution with mean and standard deviation both equal to 100. The large standard deviation was chosen to introduce greater variation and randomness into the model. Future users may use the code to easily implement sampling from other distributions if desired. The mean value of 100 was motivated by estimates of the number of observed translation events from single CD147 mRNAs in mammalian cells. The mean value of 100 is likely an underestimate, so further parameter optimization may be considered in future.
(3) Simulating ribosomes traversing an mRNA. Each mRNA then undergoes a Monte Carlo simulation where R ribosomes will traverse the mRNA. To do this it iterates through a set of Boolean conditions k=1: R times. It initializes variables protein1 and protein2 and set both equal to 0 before this loop begins. It says the k^{th} ribosome has been loaded onto the 5′end of the mRNA, when the k^{th} iteration of this loop begins. On the k^{th} iteration of the loop, the following steps/cases are considered:
(i) The model samples a value for a Bernoulli random variable with success probability p_TIS1. If Bernoulli (p_TIS1) equals 1, then the k^{th} ribosome is considered to have succeeded in translating ORF 1, producing one unit of Protein 1. The variable protein1 is incremented up by 1 and the simulation moves to the k+1 iteration. However, if the sampled value from Bernoulli (p_TIS1) equals 0, then the k^{th} ribosome will encounter the next Boolean condition (ii).
(ii) The model samples a value for a Bernoulli random variable with success probability p_TIS2. If Bernoulli (p_TIS2) equals 1, then the k^{th} ribosome is considered to have succeeded in translating ORF 2, producing one unit of Protein 2. The variable protein2 is incremented up by 1 and the simulation moves to the k+1 iteration. However, if the sampled value from Bernoulli (p_TIS2) equals 0, then the k^{th} ribosome will encounter the next Boolean condition (iii).
(iii) The final condition is implicit. If the k^{th} ribosome does not initiate translation at either TIS, the ribosome will not create any protein product. The simulation moves onto the k+1 iteration, where the k+1 ribosome is loaded and will encounter these cases (i, ii, iii).
(4) Loading aggregating mRNA simulation results into cells. Once all mRNAs have been simulated, results of individual mRNA simulations were aggregated together as a proxy for transient transfection and implicit transcription of plasmid-DNA. Here, the number of individual mRNA simulation results that are aggregated together is sampled from a normal distribution with mean 100 and standard deviation of 100 to capture the stochasticity of transient transfection. This mean value is motivated by observed CD147 mRNA values in single cells. It should be noted the per cell number of CD147 mRNAs measured by Albayrak et al. are not from transient transfection but endogenous expression. Resampling of the mRNA simulations was used in this step as mRNA simulations were computationally expensive to calculate. Specifically, from ˜500,000 individual mRNA simulations, 2500 cells were “loaded” with aggregated results. The simulations were then randomized, and another 2500 cells were loaded. This process was conducted four times to yield 10,000 simulated cells. The mean, variance, and choice of distribution to model transient transfection are additional parameters that future researchers can further optimize. The code that was written can easily accommodate the use of different distributions. The quantities of Protein 1 and Protein 2 across all mRNA simulations within a particular group or “cell” are aggregated and then plotted in FIG. 6B. This provides a basis for comparison with other flow cytometry results gathered in this study.

### Scaling the Model to Consider More OREs or to Consider Internal Methionines

The model can readily be used to consider systems with more than two ORFs by adding additional p_TIS values in the input parameters. The addition of a p_TIS can be considered as the addition of a desired ORF or an internal methionine within an ORF. The model does not differentiate between a TIS that may exist at the canonical 5′ end of an ORF or a TIS that may be encoded in the middle of an ORF.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
a polynucleotide comprising a first nucleic acid unit and a second nucleic acid unit,
wherein the first nucleic acid unit encodes one or more first unit payload protein(s),
wherein the second nucleic acid unit encodes one or more second unit payload protein(s),
wherein the first nucleic acid unit and the second nucleic acid unit each comprise an engineered translation initiation site (eTIS) comprising a three-nucleotide tunable element immediately upstream of a start codon,
wherein the polynucleotide is capable of being translated to generate the one or more first unit payload protein(s) and the one or more second unit payload protein(s), and wherein the polynucleotide is a polycistronic transcript,
wherein the eTIS of each of the first nucleic acid unit and the second nucleic acid unit is configured to achieve a predetermined stoichiometry of the one or more first unit payload protein(s) and one or more second unit payload protein(s) in a cell or cell-like environment, and
wherein:
(i) the nucleic acid composition comprises at least one modified nucleotide and/or at least one nucleotide analogue or nucleotide derivative; and/or
(ii) the polynucleotide comprises an eTIS combination of a first eTIS of the first nucleic acid unit and a second eTIS of the second nucleic acid unit, and wherein the tunable element of the first eTIS and the second eTIS are independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

2. A nucleic acid composition, comprising:
a promoter operably linked to a polynucleotide comprising a first nucleic acid unit and a second nucleic acid unit,
wherein the first nucleic acid unit encodes one or more first unit payload protein(s),
wherein the second nucleic acid unit encodes one or more second unit payload protein(s),
wherein the first nucleic acid unit and the second nucleic acid unit each comprise an engineered translation initiation site (eTIS) comprising a three-nucleotide tunable element immediately upstream of a start codon,
wherein the promoter is capable of inducing transcription of the first nucleic acid unit and the second nucleic acid unit to generate a polycistronic transcript,
wherein the polycistronic transcript is capable of being translated to generate the one or more first unit payload protein(s) and the one or more second unit payload protein(s),
wherein the eTIS of each of the first nucleic acid unit and the second nucleic acid unit is configured to achieve a predetermined stoichiometry of the one or more first unit payload protein(s) and one or more second unit payload protein(s) in a cell or cell-like environment, and
wherein:
(i) the nucleic acid composition comprises at least one modified nucleotide and/or at least one nucleotide analogue or nucleotide derivative; and/or
(ii) the polynucleotide comprises an eTIS combination of a first eTIS of the first nucleic acid unit and a second eTIS of the second nucleic acid unit, and wherein the tunable element of the first eTIS and the second eTIS are independently selected from the group consisting of AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TTG, TCA, TCT, TCC, TCG, TGA, TGT, TGC, TGG, CAA, CAT, CAC, CAG, CTA, CTT, CTC, CTG, CCA, CCT, CCC, CCG, CGA, CGT, CGC, CGG, GAA, GAT, GAC, GAG, GTA, GTT, GTC, GTG, GCA, GCT, GCC, GCG, GGA, GGT, GGC, GGG, AAU, AUA, AUU, AUC, AUG, ACU, AGU, UAA, UAU, UAC, UAG, UUA, UUU, UUC, UUG, UCA, UCU, UCC, UCG, UGA, UGU, UGC, UGG, CAU, CUA, CUU, CUC, CUG, CCU, CGU, GAU, GUA, GUU, GUC, GUG, GCU, GGU, or any combination thereof.

3. The nucleic acid composition of claim 1, wherein:
the eTIS comprises at least one modified nucleotide and/or at least one nucleotide analogue or nucleotide derivative;
the at least one modified nucleotide and/or at least one nucleotide analogues is selected from a backbone modified nucleotide, a sugar modified nucleotide and/or a base modified nucleotide, or any combination thereof; and/or
the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, N-1-methylpseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl) uridine, 5-(isopentenylaminomethyl)-2-thiouridine, 2-aminoadenosine or 5-(isopentenylaminomethyl)-2′-O-methyluridine or 2-thiothymidine, pyrrolo-pyrimidine, 3-methyl adenosine, C5 propynyl-cytidine, C5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine or 0(6)-methylguanine.

4. The nucleic acid composition of claim 1, wherein the eTIS combination comprises:
(a) a first eTIS comprising a tunable element consisting of TTT or UUU, and a second eTIS comprising a tunable element consisting of ACC;
(b) a first eTIS comprising a tunable element consisting of CCC, and a second eTIS comprising a tunable element consisting of ACC;
(c) a first eTIS comprising a tunable element consisting of ACC, and a second eTIS comprising a tunable element consisting of ACC;
(d) a first eTIS comprising a tunable element consisting of TTC or UUC, and a second eTIS comprising a tunable element consisting of ACC; or
(e) a first eTIS comprising a tunable element consisting of GGG, and a second eTIS comprising a tunable element consisting of ACC.

5. The nucleic acid composition of claim 1,
wherein the polynucleotide further comprises n supplemental nucleic acid unit(s), wherein n is an integer greater than zero,
wherein each supplemental nucleic acid unit encodes one or more supplemental unit payload protein(s),
wherein each supplemental nucleic acid unit comprises an engineered translation initiation site (eTIS) comprising a three-nucleotide tunable element immediately upstream of a start codon,
wherein the polycistronic transcript is capable of being translated to generate
the one or more first unit payload protein(s),
the one or more second unit payload protein(s), and
the one or more supplemental unit payload protein(s) encoded by each of the n supplemental nucleic acid unit(s), and
wherein the eTIS of each of the first nucleic acid unit, the second nucleic acid unit, and the n supplemental nucleic acid unit(s) is configured to achieve a predetermined stoichiometry of
the one or more first unit payload protein(s),
the one or more second unit payload protein(s), and
the one or more supplemental unit payload protein(s) encoded by each of the n supplemental nucleic acid unit(s)
in a cell or cell-like environment.

6. The nucleic acid composition of claim 5, wherein the first nucleic acid unit, the second nucleic acid unit, and the n supplemental nucleic acid unit(s) each comprise an open reading frame (ORF).

7. The nucleic acid composition of claim 1,
wherein the tunable element modulates the strength of an eTIS of a nucleic acid unit, and wherein the strength of an eTIS of a nucleic acid unit is related to the fraction of the 43S preinitiation complex (PIC) scanning the polycistronic transcript that initiate and translate the open reading frame of said nucleic acid unit by engaging with the 60S ribosomal subunit upon reaching said eTIS;
wherein the expression level of a unit payload protein is inversely related to the number and strength of eTIS situated upstream of the nucleic acid unit from which it derives on the polycistronic transcript;
wherein the strength of the eTIS of the first nucleic acid unit is inversely proportional to the expression level of the second unit payload protein(s);
wherein the expression level of the second unit payload protein(s) is inversely related to the fraction of the ribosomes initiating and translating the open reading frame of the first nucleic acid unit; and/or
wherein the strength of the eTIS of the second nucleic acid unit is greater than the strength of the eTIS of the first nucleic acid unit, and thereby the eTIS of the second nucleic acid unit efficiently captures the ribosomal translational activity that fails to initiate at the eTIS of the first nucleic acid unit.

8. The nucleic acid composition of claim 5, wherein the n supplemental nucleic acid unit(s) comprise one or more of a third nucleic acid unit, a fourth nucleic acid unit, a fifth nucleic acid unit, a sixth nucleic acid unit, a seventh nucleic acid unit, an eight nucleic acid unit, and a ninth nucleic acid unit.

9. The nucleic acid composition of claim 8, wherein the eTIS combination further comprises a third eTIS of the third nucleic acid unit, and wherein the eTIS combination comprises:
(a) a first eTIS comprising a tunable element consisting of ACC, a second eTIS comprising a tunable element consisting of ACC, and a third eTIS comprising a tunable element consisting of ACC;
(b) a first eTIS comprising a tunable element consisting of ACC, a second eTIS comprising a tunable element consisting of TTT or UUU, and a third eTIS comprising a tunable element consisting of ACC;
(c) a first eTIS comprising a tunable element consisting of CCC, a second eTIS comprising a tunable element consisting of TTT or UUU, and a third eTIS comprising a tunable element consisting of ACC;
(d) a first eTIS comprising a tunable element consisting of TTT or UUU, a second eTIS comprising a tunable element consisting of ACC, and a third eTIS comprising a tunable element consisting of ACC;
(e) a first eTIS comprising a tunable element consisting of TTT or UUU, a second eTIS comprising a tunable element consisting of CCC, and a third eTIS comprising a tunable element consisting of ACC; or
(f) a first eTIS comprising a tunable element consisting of TTT or UUU, a second eTIS comprising a tunable element consisting of TTT or UUU, and a third eTIS comprising a tunable element consisting of ACC.

10. The nucleic acid composition of claim 1, wherein the first eTIS and/or the second eTIS comprise a start codon and about 3-10 neighboring nucleotides.

11. The nucleic acid composition of claim 1, wherein one or more of the first unit payload protein(s) and the second unit payload protein(s) is a payload, is a component of a payload, or chaperones the assembly of a payload.

12. The nucleic acid composition of claim 1, wherein two or more of the first unit payload protein(s) and the second unit payload protein(s) are components of a multimeric protein complex.

13. The nucleic acid composition of claim 1,
wherein one or more of the first unit payload protein(s) and the second unit payload protein(s) is a structural protein, and wherein one or more of the first unit payload protein(s) and the second unit payload protein(s) is a chaperone;
wherein two or more of the first unit payload protein(s) and the second unit payload protein(s) are capable of forming an antibody or antigen-binding fragment thereof;
wherein two or more of the first unit payload protein(s) and the second unit payload protein(s) are configured for secretion; and/or
wherein one or more of the first unit payload protein(s) and the second unit payload protein(s) is a toxin, and wherein one or more of the first unit payload protein(s) and the second unit payload protein(s) is an antitoxin.

14. The nucleic acid composition of claim 1, wherein two or more of the first unit payload protein(s) and the second unit payload protein(s) are capable of one or more of: (i) forming mosaic virus-like particles; (ii) forming all or a portion of a metabolic pathway; (iii) complex and/or polyclonal antigen(s); (iv) a cytokine cocktail; (v) an immunomodulatory cocktail; (vi) all or a portion of an enzymatic pathway; and (vii) ex vivo and/or in vivo cell fate determination and/or reprogramming.

15. The nucleic acid composition of claim 1, wherein the nucleic acid composition is capable of causing less cellular burden, toxicity, and/or apoptosis as compared to a nucleic acid composition wherein the expression of the first unit payload protein(s) and the second unit payload protein(s) is achieved via two or more separate polynucleotides.

16. The nucleic acid composition of claim 1, wherein difference between the steady-state levels of one or more of the first unit payload protein(s) and the second unit payload protein(s) is greater than about one order of magnitude.

17. The nucleic acid composition of claim 1,
wherein the eTIS comprises a G nucleotide immediately downstream of the start codon;
wherein one or both of the first nucleic acid unit and the second nucleic acid unit do not comprise an internal start codon;
wherein one or both of the first nucleic acid unit and the second nucleic acid unit do not comprise an out-of-frame AUG;
wherein the polycistronic transcript does not comprise an upstream ORF (uORF);
wherein the first unit payload protein(s) is not less than about 30, about 25, about 20, about 15, about 10, or about 5, amino acids in length;
wherein one or more of the first unit payload protein(s) and the second unit payload protein(s) do not comprise an internal methionine residue; and/or
wherein one or more of the first unit payload protein(s) and the second unit payload protein(s) do not comprise non-native amino acid residues.

18. The nucleic acid composition of claim 1, wherein one or both of the first nucleic acid unit and the second nucleic acid unit do not comprise a tandem gene expression element.

19. Engineered cells comprising the nucleic acid composition of claim 1.

20. A method of treating or preventing a disease or disorder in a subject in need thereof, comprising:
administering to the subject an effective amount of the nucleic acid composition of claim 1, thereby treating or preventing the disease or disorder in the subject.
